(19) United States
(12) Patent Application Publication
(10) Pub. No.: US 2025/0270579 A1
(43) Pub. Date: Aug. 28, 2025

(54) PLANTS WITH ALTERED ROOT AND SHOOT ARCHITECTURE AND METHODS FOR OBTAINING AND USING SAME

(71) Applicant: IOWA STATE UNIVERSITY RESEARCH FOUNDATION, INC., Ames, IA (US)

(72) Inventors: Dior Rose KELLEY, Ames, IA (US); Craig L. COWLING, Ames, IA (US)

(21) Appl. No.: 19/067,479

(22) Filed: Feb. 28, 2025

Related U.S. Application Data
(60) Provisional application No. 63/559,014, filed on Feb. 28, 2024.

(51) Int. Cl. C12N 15/82; C12N 9/22; C12N 15/11; C12Q 1/6895

(52) U.S. Cl. CPC C12N 15/8262; C12N 9/22; C12N 15/11; C12Q 1/6895; C12N 2310/20; C12Q 2600/13; C12Q 2600/156

(57) ABSTRACT

This disclosure relates to methods of altering crown root architecture, lateral root formation, and/or stalk height in plants by reducing or eliminating expression of a PIN-likes 6 (PILS6) gene in a plant or a plant cell. Also disclosed are plant cells, plants comprising the plant cells, nucleic acid constructs, expression vectors, and breeding methods.

This application claims the priority benefit of U.S. Provisional Patent Application Ser. No. 63/559,014, filed Feb. 28, 2024, which is hereby incorporated by reference in its entirety.

This invention was made with government support under 2020-67013-30914 awarded by the USDA/NIFA. The government has certain rights in the invention.

### FIELD

This disclosure relates to plant root and shoot architecture modulation using genes involved in auxin transport.

### SEQUENCE LISTING STATEMENT

This application contains a computer readable Sequence Listing which has been submitted electronically in XML format and is hereby incorporated by reference in its entirety. The XML file was created on Feb. 28, 2025, is named 147426001551.xml, and is 165,385 bytes in size.

### BACKGROUND

Crop architecture traits affecting plant shoot and root systems play a pivotal role in plant physiology and exhibit diverse phenotypic traits. Understanding the genetic mechanisms governing shoot and root growth and development in model plants like maize is crucial for enhancing crop resilience to drought, environmental, and nutrient limitations.

Crop architecture traits can be modulated via hormone pathways and can impact yield. For example, manipulation of gibberellic acid levels in semi-dwarf wheat, rice and sorghum plant varieties led to increased yield and reduced lodging in these cereal crops during the 20th century, which was largely responsible for the Green Revolution. Another example of potentially valuable crop architecture traits involves alteration of roots to have reduced network area and suppressed lateral root formation. This phenotype is desirable for the “steep, cheap, and deep” ideotype. The concept of the “steep, cheap, and deep” ideotype is described in Lynch, “Steep, Cheap and Deep: An Ideotype to Optimize Water and N Acquisition by Maize Root Systems Outlines the Ideal Characteristics of a Maize Plant for Maximizing Yield Potential,” Ann. Bot. 112 (2): 347-357 (2013) and is based on the typical infiltration of water and nitrate into deeper soil strata over time along with their initial depletion in surface soil strata. Root systems that rapidly exploit deep soil would optimize water and N capture in most maize production environments.

Lynch suggests specific phenotypes for this ideotype contributing to rooting depth in maize, such as a large diameter primary root with few but long laterals, many seminal roots with shallow growth angles, and an intermediate number of crown roots with steep growth angles. This ideotype is also relevant to other cereal root systems as well. An outstanding question is how the transport of auxin, a key phytohormone, can influence maize root architecture. Discovery of genes that confer beneficial crop architecture alterations are needed for improving the resilience and yield potential of maize crops, thereby offering substantial benefits for both farmers and the broader agricultural industry.

This disclosure is directed to overcoming these and other deficiencies in the art.

### SUMMARY

One aspect of the present disclosure relates to a method of altering crown root architecture, lateral root formation, and/or stalk height in a Poaceae plant. This method involves reducing or eliminating expression of a PIN-likes 6 (PILS6) gene in a plant or a plant cell of the Poaceae family, where said reducing or eliminating is effective to reduce crown root architecture, lateral root formation, and/or stalk height in the plant or a plant produced from the plant cell, as compared to a wild type plant.

Another aspect of the present disclosure relates to a method of increasing salt tolerance in a Poaceae plant. This method involves reducing or eliminating expression of a PIN-likes 6 (PILS6) gene in a plant or a plant cell of the Poaceae family, where said reducing or eliminating is effective to increase salt tolerance in the plant or a plant produced from the plant cell, as compared to a wild type plant.

Yet another aspect of the present disclosure relates to a Poaceae plant cell comprising a non-naturally occurring loss of function mutation in a PIN-likes 6 (PILS6) gene encoding a PILS6 protein or fragment thereof. The expression and/or activity of the PILS6 gene or protein is reduced or eliminated in the plant cell as compared to plant cell without the mutation.

A further aspect of the present disclosure relates to a Poaceae plant cell comprising an inhibitory polynucleotide targeting a PIN-likes 6 (PILS6) gene encoding a PILS6 protein, where expression and/or activity of the PILS6 gene is reduced or eliminated in the plant cell compared to a plant cell without the inhibitory polynucleotide.

Another aspect of the present disclosure relates to a nucleic acid construct comprising a nucleotide sequence targeting a PILS6 gene comprising (i) a guide RNA or (ii) an inhibitory polynucleotide; a 5′ heterologous DNA promoter sequence; and a 3′ terminator sequence.

A further aspect of the present disclosure relates to a method of breeding for reduced crown root architecture, reduced lateral root formation, or reduced stalk height in a Poaceae plant. This method involves providing a candidate plant or plant part having a non-naturally occurring loss of function mutation in a PIN-likes 6 (PILS6) gene, or an inhibitory polynucleotide reducing or eliminating expression of a PIN-likes 6 (PILS6) gene; analyzing the candidate plant or plant part for the presence, in its genome, of the mutation in the PILS6 gene or the inhibitory polynucleotide; identifying, based on said analyzing, a candidate plant suitable for breeding, where the candidate plant comprises the mutation in the PILS6 gene or the inhibitory polynucleotide; and breeding the candidate plant with at least one other plant.

Among other things, the present disclosure describes a genetic determinant of maize height and root system architecture, referred to as ZmPILS6 (Zm00001eb149720). Decreases in ZmPILS6 expression is shown to lead to reduced plant height and crown root traits, which can be leveraged by plant breeders for, e.g., improved harvest, increased planting density, and crop resilience.

In some embodiments, ZmPILS6 is shown to be a key regulator of various crown root traits in maize. ZmPILS6 mutant roots are shown to display reduced network area and suppressed lateral root formation, which are desirable traits for the “steep, cheap, and deep” ideotype. As shown herein, ZmPILS6 localizes to the endoplasmic reticulum and plays an important role in controlling the spatial distribution of indole-3-acetic acid (IAA or “auxin”) in primary roots. Also shown herein is that ZmPILS6 can actively efflux IAA when expressed in yeast. Furthermore, the loss of ZmPILS6 resulted in significant proteome remodeling in maize roots, particularly affecting hormone signaling pathways. To identify potential interacting partners of ZmPILS6, a weighted gene co-expression analysis (WGCNA) was performed. Altogether, the present disclosure contributes to the growing knowledge of essential genetic determinants governing maize root morphogenesis, which can be used to guide agricultural improvement strategies.

Reverse genetic screens are an effective approach for linking gene to function in eukaryotic systems (Page and Grossniklaus, “The Art and Design of Genetic Screens: Arabidopsis thaliana,” Nat. Rev. Genet. 3:124-136 (2002), which is hereby incorporated by reference in its entirety). Here, an integrated gene expression atlas was leveraged (Walley et al., “Integration of Omic Networks in a Developmental Atlas of Maize,” Science 353:814-818 (2016), which is hereby incorporated by reference in its entirety) to identify putative auxin transporters that are required for maize root formation. By focusing on candidate annotated auxin efflux carriers with enriched protein abundance in maize primary roots and utilizing publicly available transposon insertion lines (Williams-Carrier et al., “Use of Illumina Sequencing to Identify Transposon Insertions Underlying Mutant Phenotypes in High-Copy Mutator Lines of Maize,” Plant J. 63:167-177 (2010); Settles et al., “Sequence-Indexed Mutations in Maize Using the UniformMu Transposon-Tagging Population,” BMC Genomics 8:116 (2007); McCarty et al., “Mu-seq: Sequence-Based Mapping and Identification of Transposon Induced Mutations,” PLOS ONE 8: e77172 (2013), each of which is hereby incorporated by reference in its entirety) this screen identified ZmPILS6 (Zm00001cb149720, also previously called ZmPILS4) as an influencer of root morphogenesis.

Based on phylogenetic analysis of Arabidopsis and maize PILS proteins, Zm00001cb149720 is most closely related to AtPILS6 (At5g01990) and is thus designated ZmPILS6. Loss of ZmPILS6 leads to reduced crown root architecture and diminished lateral root formation. In addition, ZmPILS6 is localized to the endoplasmic reticulum and is required for proper transport of IAA. A proteomic analysis revealed that several decreased proteins in mutant zmpils6 relative to wild-type W22 are involved in hormone pathways, including auxin. Using these data, a weighted gene co-expression network analysis (WGCNA) was performed to identify candidate proteins that may act with ZmPILS6 to influence maize root development. Altogether this novel work establishes roles for auxin transport in maize root formation and suggests PILS proteins may have opposing functions in monocots and eudicots.

A reverse genetic screen in maize identified the evolutionarily conserved auxin transporter, ZmPILS6, as a positive regulator of root and shoot traits that underpin plant physiology. Maize plants with reduced ZmPILS6 expression displayed short stature, reduced crown root architecture, increased salt tolerance, and impaired auxin sensitivity. It is surprisingly shown herein that maize PILS6 has a contrasting role in driving root and shoot morphogenesis compared to the Arabidopsis ortholog, emphasizing the need for functional characterization of candidate genes directly within crops of interest.

### BRIEF DESCRIPTION OF THE DRAWINGS

FIGS. 1A-J show the characterization of ZmPILS6 loss-of-function alleles. FIG. 1A is a schematic illustration of the ZmPILS6 gene (Zm00001eb149720). White boxes indicate untranslated regions (UTR), exons are indicated in black boxes, and introns are shown as lines. The location of ZmPILS6 transposon alleles is shown with white inverted triangles. The alleles characterized in this disclosure are designated as pils6-1 (mu1047700), pils6-2 (mu1090629), and pils6-3 (mu-ill 221882.6). Scale bar=500 bp. FIG. 1B is a graph of ZmPILS6 expression measured by RT-qPCR in W22, pils6-1, pils6-2, B73, and pils6-3 roots. FIG. 1C is a graph of box and whisker plots indicating primary root length of W22, B73, pils6-1, and pils6-3 7-day-old seedlings grown without and with 10 μM indole-3-acetic acid (IAA). P values from a one-way ANOVA, N=8-19. FIG. 1D is a graph showing the percent inhibition by IAA on primary root length. Percent inhibition calculated as (length of mock-length of treated)/length of mock*100. FIGS. 1E-H show representative photographic images of crown root systems of V7 greenhouse grown (FIG. 1E) W22, (FIG. 1F) pils6-1, (FIG. 1G) B73, and (FIG. 1H) pils6-3. Scale bars=5 cm. FIG. 1I is a graph of box and whisker plots indicating total root length of V7 plants. P values from a t test, N=4-7. FIG. 1J is a graph of root network area of V7 plants. P values from a t test, N=4-7.

FIGS. 2A-F show that lateral root formation is reduced in pils6 primary roots. FIGS. 2A-D are a series of photographs showing Feulgen stained six-day-old primary roots. Scale bars=2 mm. FIGS. 2E-F and graphs of histograms of lateral root primordia density (calculated as the number of lateral roots per total length of primary root) in pils6 alleles compared to their respective inbred controls. Scale bars=2 mm.

FIGS. 3A-D show that ZmPILS6 is an endoplasmic reticulum (“ER”) localized auxin efflux protein. FIG. 3A is a graph showing indole-3-acetic acid (IAA) levels in five-day-old W22 primary roots dissected into the meristematic zone (MZ), elongation zone (EZ), cortex, and stele. N=6. FIG. 3B is a graph showing levels of 3H-IAA in dissected regions of primary W22 and pils6-1 roots. P values shown from ANOVA; ns=not significant. FIG. 3C is a series of photographs showing transient expression in Nicotiana benthamiana epidermal cells of pUBII: PILS6-GSyellow and p35S: CFP-HDEL, a marker for the endoplasmic reticulum (ER). Overlay of both fluorescence channels is shown as “merged”. Scale bars=50 μm. FIG. 3D is a graph showing levels of 3H-IAA in yeast expressing an empty vector or ZmPILS6. P values calculated from t test, N=12.

FIGS. 4A-D show proteome changes in the absence of ZmPILS6. FIG. 4A is an illustration showing hierarchical clustering of differentially expressed (DE) proteins in W22 and pils6-1, following a 1-hour treatment with control (“mock”) or 10 μM indole-3-acetic acid (IAA). FIG. 4B is a graph of an upset plot of DE proteins indicates extent of overlap among DE proteins across genotypes and treatments. FIG. 4C is a graph showing Enriched Gene Ontology (GO) biological process terms among DE proteins in W22 and pils6-1 in the presence or absence of auxin treatment (IAA). FIG. 4D is a graph of a ZmPILS6 co-expression protein network reconstructed from weighted gene co-expression network analysis (WGCNA) consisting of 415 nodes and 414 edges. Nodes are colored according to PANTHER protein class. ZmPILS6 is the central green node, and predicted interacting proteins include vesicle coat proteins (reddish purple nodes), ABC transporters (orange nodes), SNARE proteins (vermillion nodes), kinases (yellow nodes), membrane traffic proteins (sky blue nodes), and phosphatases (blue nodes).

FIG. 5 is a graph showing ZmPILS6 protein abundance data. Distributed normalized spectral abundance factor (dNSAF) values for maize PILS6 protein across different tissues labeled on the x-axis. dNSAF data was obtained from qTeller at MaizeGDB.

FIG. 6 is a phylogenetic tree of PILS proteins from maize and Arabidopsis. Tree constructed using the PLAZA 5.0 phylogenetic tool. Bootstrap values are indicated at each node as a percentage. Protein sequences obtained from PLAZA.

FIG. 7 shows a sequence alignment of AtPILS6 (SEQ ID NO:6) and ZmPILS6 (SEQ ID NO:3) primary amino acid sequences. Primary amino acid sequences of AtPILS6 (At5G01990) and ZmPILS6 (Zm00001eb149720) obtained from TAIR and MaizeGDB, respectively, each of which is hereby incorporated by reference in its entirety. Conserved transmembrane domains (abbreviated TMD) are numbered sequentially 1-10 from the N-terminus to C-terminus.

FIGS. 8A-B show crown root traits calculated with Rhizo Vision. FIG. 8A is a graph showing the number of root branch points crown root systems of V7 greenhouse grown plants. P values calculated from ANOVA. N=4-7 biological replicates. FIG. 8B is a graph of the number of root tips in crown root systems of V7 greenhouse grown plants. P values calculated from ANOVA. N=4-7 biological replicates.

FIGS. 9A-E show shoot phenotypes of pils6 alleles. FIGS. 9A-D are representative photographic images of greenhouse grown W22 (FIG. 9A), pils6-1 (FIG. 9B), B73 (FIG. 9C), and pils6-3 plants (FIG. 9D) at stage V7. Scale bar=6 cm. FIG. 9E is a graph of plant height of stage V7 plants. P value determined by t test, N=5.

FIG. 10 shows a ZmPILS6 WGCNA cluster dendogram. The height relates to the distance metric used for clustering. Dynamic Tree Cut and Merged Dynamic outputs for detecting clusters in the ZmPILS6 co-expression network.

FIG. 11 is a photograph of a field grown pils6-1 mutant in comparison to a wild type control (W22). pils6 mutants have reduced crown root architecture compared to their respective controls.

FIGS. 12A-B show that mature pils6-3 plants are shorter than their respective control. FIG. 12A is a schematic diagram of mature maize plant and measurements that were taken. FIG. 12B is a graph showing height measurements of mature plants. Statistical analysis was done using a one-way ANOVA followed by Tukey's multiple comparisons test.

FIG. 13 is a graph showing that mature pils6-1 plants have higher ear placement than their respective control. Ear height measurements of mature plants are shown. Statistical analysis was done using a one-way ANOVA followed by Tukey's multiple comparisons test. Hm—homozygous for the mutation; Het—heterozygous for the mutation.

FIG. 14 is a graph showing that mature pils6 plants have higher ear placement ratios than their respective controls. The ear placement ratio was determined as (ear height/plant height)×100%. Statistical analysis was done using a one-way ANOVA followed by Tukey's multiple comparisons test. Hm—homozygous for the mutation; Het—heterozygous for the mutation.

FIG. 15 is a graph showing that mature pils6 stalks are not significantly smaller than their respective controls. The graph shows the measured stalk base circumference of mature plants. The circumference of the lowest internode is shown. Statistical analysis was done using a one-way ANOVA followed by Tukey's multiple comparisons test. Hm—homozygous for the mutation; Het—heterozygous for the mutation.

FIG. 16 is a series of photographs showing that pils6 mutants have reduced crown root architecture compared to their respective control. Plants were grown to V7 stage (˜37 days) under natural conditions at the Iowa State University Ag Engineering and Agronomy Research Farm in Boone, Iowa. Scale bar=5 cm.

FIG. 17 is a heatmap diagram containing a subset of differentially expressed (DE) proteins in W22 and pils6-1, following a 1-hour treatment with control (“Mock”) or 10 μM indole-3-acetic acid (IAA). DE proteins were chosen for a functional annotation related to phytohormones and cell proliferation. Abbreviations used: Abscisic acid (ABA), Gibberellic acid (GA), and Jasmonic acid (JA). Proteomics data from Cowling et al., “ZmPILS6 is an Auxin Efflux Carrier Required for Maize Root Morphogenesis,” PNAS 121 (22) e2313216121 (2024), which is hereby incorporated by reference in its entirety. Examples of DE ABA genes include AASR and RAB, examples of DE Auxin genes include PIN/PILS and AAAP, examples of DE cell wall genes include Expansin and Transferases, examples of Gibberellic acid genes include GRAS and GAR transcription factors, and examples of cell proliferation genes include cyclins and CDK genes.

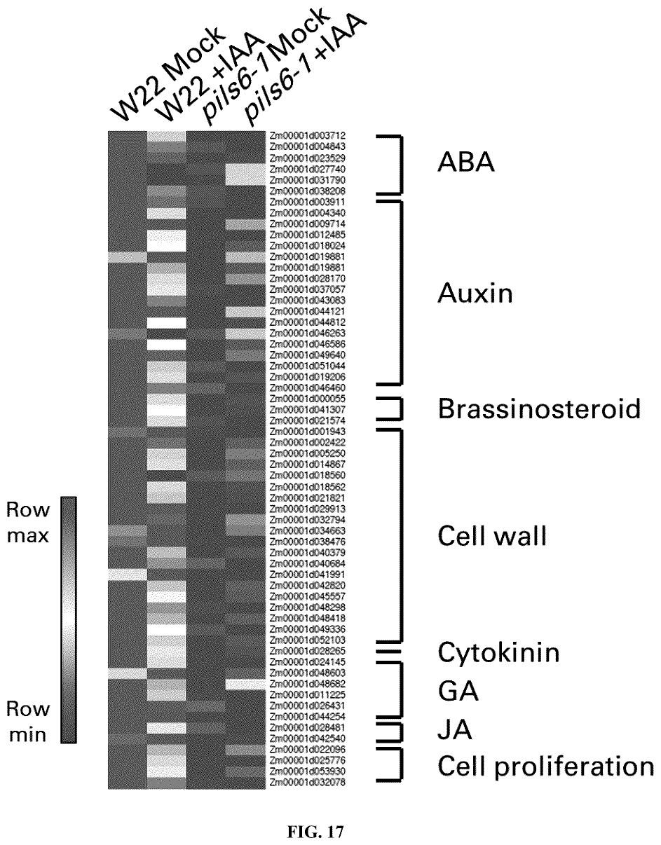

FIG. 18 is a graph showing that pils6-1 plants are less sensitive to salt stress. Seedlings were grown under control (0.5×LS media, no extra NaCl) conditions for 5 days, then grown with control or salt-stressed (0.5×LS media, plus 200 mM NaCl) conditions for an additional 5 days of growth. Statistical analysis was done using a two-way ANOVA.

FIGS. 19A-B are graphs showing that pils6-1 plants are less sensitive to salt stress. FIG. 19A shows the primary root lengths of seedlings grown under mock (0.5×LS media, no extra NaCl) conditions for 5 days, then grown with control or salt (0.5×LS media, plus 200 mM NaCl) conditions for an additional 5 days of growth. Statistical analysis was done using a one-way ANOVA followed by Tukey's multiple comparisons test. FIG. 19B shows the relative primary root growth of salt treated seedlings compared to mock. Relative growth determined as (salt/mock)×100%.

FIG. 20 is a graph showing that pils6 seedlings are less sensitive to salt stress based on primary root length (cm) of salt treated pils6-1 and pils6-3 seedlings compared to their wild type controls (W22 and B73, respectively). Primary root lengths of seedlings grown under mock (0.5×LS media, no extra NaCl) conditions for 5 days, then grown with control or salt (0.5×LS media, plus 200 mM NaCl) conditions for an additional 5 days of growth. Statistical analysis was done using a one-way ANOVA followed by Tukey's multiple comparisons test.

FIG. 21 is a graph showing maize genotypes that are insensitive to salt stress (PHN34, ICI740 and PHW17) and maize genotypes sensitive to salt stress (DKPB80, W1533R, B73, NC310, LP215D). Primary root lengths of seedlings grown under mock (0.5×LS media, no extra NaCl) conditions for 5 days, then grown with control or salt (0.5×LS media, plus 200 mM NaCl) conditions for an additional 5 days of growth. Statistical analysis was done using a one-way ANOVA followed by Tukey's multiple comparisons test.

FIGS. 22A-B illustrate that the root meristem size is reduced in size in pils6 compared to their wild type controls. FIG. 22A is a series of photographs of longitudinal sections of primary root tips taken from 5-day-old seedlings and stained with 0.025% Toluidine blue. Individual images were photo merged using Adobe Photoshop. Orange triangles indicate the beginning and end of the meristematic zone. The scale bar is equal to 1 mm. FIG. 22B is a graph of the length of the meristematic zone in the primary root. P values from unpaired t test.

FIG. 23 is a phylogenetic tree showing PILS6 orthologs in core eudicots including versus PILS2, PILS1/PILS3/PILS4 and PILS5/PILS7 clades.

### DETAILED DESCRIPTION

The present disclosure relates to methods of altering crown root architecture, altering lateral root architecture (e.g., lateral root formation), reducing stalk height, and increasing salt tolerance in plants, including plants of the Poaceae family, and compositions useful in imparting altered crown root architecture, altered lateral root architecture, reduced stalk height and increased salt tolerance in plants.

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” includes being within a statistically meaningful range of a value. Such a range can be within an order of magnitude, such as within 10% or within 5% of a given value or range.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps.

A “gene” refers to an assembly of nucleotides that encode a functional RNA or a polypeptide and includes cDNA and genomic DNA nucleic acids. “Gene” also refers to a nucleic acid fragment that expresses a specific protein or polypeptide, optionally including regulatory sequences preceding (5′ noncoding sequences) and following (3′ noncoding sequences) the coding sequence. “Endogenous gene” refers to a native gene in its natural location in the genome of an organism. A “foreign” gene, “heterologous” gene, or “exogenous” gene refers to a gene not normally found in the host organism, but that is introduced into the host organism by gene transfer. Heterologous genes can comprise native genes inserted into a non-native organism, or chimeric genes, such as a native gene under control of a different promoter than its endogenous promoter. A “transgene” is a gene that has been introduced into the genome by a transformation or transfection procedure.

A “coding region” is a portion of a nucleic acid, which is transcribed and translated into a polypeptide or protein.

A “polypeptide” is a naturally occurring or synthetic peptide, oligopeptide, polypeptide, gene product, expression product, or protein comprising an amino acid sequence, where the amino acids are joined to each other by peptide bonds or modified peptide bonds.

The term “fragment” when referring to a polynucleotide or polypeptide will be understood to mean a nucleotide or polypeptide sequence of reduced length relative to the reference nucleic acid or polypeptide sequence (e.g., a wild type) and comprising, over the common portion, a nucleotide or polypeptide sequence identical to the reference sequence. Such a fragment according to the present disclosure may be, where appropriate, included in a larger polynucleotide or polypeptide of which it is a constituent. Such fragments comprise, or alternatively consist of, oligonucleotides or polypeptides ranging in length from at least 8, 10, 12, 15, 18, 20 to 25, 30, 40, 50, 70, 80, 100, 200, 500, 1000, 1500, or any number or range therein, consecutive nucleotides of a nucleic acid or amino acids of a polypeptide described herein.

A “reference sequence” means a nucleic acid or amino acid sequence used as a comparator for another nucleic acid or amino acid sequence, respectively, when determining sequence identity. A reference sequence can be a wild type sequence or a sequence that has not been modified as described herein.

“Sequence identity,” “percent identity,” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis). Sequences may also be aligned using algorithms known in the art including, but not limited to, CLUSTAL V algorithm or the BLASTN or BLAST 2 sequence programs.

As used herein, the term “plant cell” includes cells, protoplasts, cell tissue cultures from which plants can be regenerated, calli, clumps, and cells that are intact in plants or parts of plants (plant parts) including, but not limited to seeds, leaves, stems, roots, vegetative buds, floral buds, meristems, embryos, hypocotyls, cotyledons, endosperm, sepals, petals, pistils, carpels, stamens, anthers, microspores, pollen, pollen tubes, ovules, nucellar tissue, ovaries, and other plant tissue or cells. In some embodiments, the plant cell is a protoplast.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### PIN-Likes 6

Plant roots are an important organ for anchorage and uptake of water and nutrients. Root development is influenced by plant hormone transport dynamics to influence growth and differentiation (Tanaka et al., “Spatiotemporal Asymmetric Auxin Distribution: A Means to Coordinate Plant Development,” Cell. Mol. Life Sci. 63:2738-2754 (2006); Carrillo-Carrasco et al., “The Birth of a Giant: Evolutionary Insights into the Origin of Auxin Responses in Plants,” The EMBO Journal 42: e113018 (2023); Friml, “Fourteen Stations of Auxin,” Cold Spring Harb. Perspect. Biol. 14: a039859 (2022); Roychoudhry and Kepinski, “Auxin in Root Development,” Cold Spring Harb. Perspect. Biol. 14: a039933 (2022), each of which is hereby incorporated by reference in its entirety).

In Arabidopsis, an asymmetric distribution of indole-3-acetic acid (IAA, or “auxin”) across the primary root is required for proper cell division and patterning (Torres-Martínez et al., “From One Cell to Many: Morphogenetic Field of Lateral Root Founder Cells in Arabidopsis thaliana is Built by Gradual Recruitment,” Proc. Natl. Acad. Sci. USA 117:20943-20949 (2020); Fandino and Hardtke, “Auxin Transport in Developing Protophloem: A Case Study in Canalization,” J. Plant Physiol. 269:153594 (2022); Arieti and Staiger, “Auxin-Induced Actin Cytoskeleton Rearrangements Require AUX1,” New Phytol. 226:441-459 (2020); Moore et al., “Spatiotemporal Modelling of Hormonal Crosstalk Explains the Level and Patterning of Hormones and Gene Expression in Arabidopsis thaliana Wild-Type and Mutant Roots,” New Phytol. 207:1110-1122 (2015); Löfke et al., “Tricho- and Atrichoblast cell Files Show Distinct PIN2 Auxin Efflux Carrier Exploitations and are Jointly Required for Defined Auxin-Dependent Root Organ Growth,” J. Exp. Bot. 66:5103-5112 (2015); Band et al., “Systems Analysis of Auxin Transport in the Arabidopsis Root Apex,” Plant Cell 26:862-875 (2014); Cazzonelli et al., “Role of the Arabidopsis PIN6 Auxin Transporter in Auxin Homeostasis and Auxin-Mediated Development,” PLOS One 8: e70069 (2013); Band and King, “Multiscale Modelling of Auxin Transport in the Plant-Root Elongation Zone,” J. Math. Biol. 65:743-785 (2012); Péret et al., “AUX/LAX Genes Encode a Family of Auxin Influx Transporters that Perform Distinct Functions During Arabidopsis Development,” Plant Cell 24:2874-2885 (2012); Ganguly et al., “Differential Auxin-Transporting Activities of Pin-Formed Proteins in Arabidopsis Root Hair Cells,” Plant Physiol. 153:1046-1061 (2010); Grieneisen et al., “Auxin Transport is Sufficient to Generate a Maximum and Gradient Guiding Root Growth,” Nature 449:1008-1013 (2007), each of which is hereby incorporated by reference in its entirety).

Auxin transport is carried out by several evolutionarily conserved proteins, including the plant-specific PIN-FORMED (PIN) family, PIN-likes (PILS), ATP-binding cassette (ABC) B-type (ABCB) family, and AUX1/LAX family (Sauer and Kleine-Vehn, “PIN-FORMED and PIN-LIKES Auxin Transport Facilitators,” Development 146: dev168088 (2019); Zažímalová et al., “Auxin Transporters—Why So Many?,” Cold Spring Harb. Perspect. Biol. 2: a001552 (2010), each of which is hereby incorporated by reference in its entirety). In maize, the roles of auxin transport during embryogenesis, leaf development, and inflorescence architecture have been described (Knöller et al., “Brachytic2/ZmABCB1 Functions in IAA Export from Intercalary Meristems,” J Exp Bot 61:3689-3696 (2010); Li et al., “Enhancing Auxin Accumulation in Maize Root Tips Improves Root Growth and Dwarfs Plant Height,” Plant Biotechnology Journal 16:86-99 (2018); Carraro et al., “ZmPINla and ZmPIN1b Encode Two Novel Putative Candidates for Polar Auxin Transport and Plant Architecture Determination of Maize,” Plant Physiology 142:254-264 (2006); Gallavotti et al., “The Relationship Between Auxin Transport and Maize Branching,” Plant Physiology 147:1913-1923 (2008); Robil and McSteen, “Hormonal Control of Medial-Lateral Growth and Vein Formation in the Maize Leaf,” New Phytologist 238:125-141 (2023), each of which is hereby incorporated by reference in its entirety).

While auxin signaling plays significant roles in maize root biology (Yu et al., “Type-Specific Gene Expression Analyses by RNA Sequencing Reveal Local High Nitrate-Triggered Lateral Root Initiation in Shoot-Borne Roots of Maize by Modulating Auxin-Related Cell Cycle Regulation 1,” Plant Physiol. 169:690-704 (2015); Zhang et al., “LATERAL ROOT PRIMORDIA 1 of Maize Acts as a Transcriptional Activator in Auxin Signaling Downstream of the Aux/IAA Gene Rootless with Undetectable Meristem 1,” J. Exp. Bot. 66:3855-3863 (2015); Majer et al., “Molecular Interactions of ROOTLESS CONCERNING CROWN AND SEMINAL ROOTS, a LOB Domain Protein Regulating Shoot-Borne Root Initiation in Maize (Zea mays L.),” Philos. Trans. R Soc. Lond. B. Biol. Sci. 367:1542-1551 (2012); Hochholdinger et al., “Proteomics of Maize Root Development,” Front. Plant Sci. 9:143 (2018); McReynolds, et al., “Temporal and Spatial Auxin Responsive Networks in Maize Primary Root,” Quantitative Plant Biology 3: e21 (2022); Saleem, et al., “Specification of Cortical Parenchyma and Stele of Maize Primary Roots by Asymmetric Levels of Auxin, Cytokinin, and Cytokinin-Regulated Proteins,” Plant Physiol. 152:4-18 (2010), each of which is hereby incorporated by reference in its entirety) the roles of auxin transport in maize root morphogenesis are not well understood.

Orthologs of Arabidopsis PIN1, ZmPIN1 genes, are polarly localized and required for organ formation (Carraro et al., “ZmPIN1a and ZmPIN1b Encode Two Novel Putative Candidates for Polar Auxin Transport and Plant Architecture Determination of Maize,” Plant Physiology 142:254-264 (2006); Carraro and Peer, “Immunolocalization of PIN and ABCB Transporters in Plants,” Methods Mol. Biol. 1398:55-67 (2016), each of which is hereby incorporated by reference in its entirety). The four maize ZmPIN1 genes exhibit tissue-specific expression patterns, suggesting sub-functionalization among the family members (Li et al., “Expression Analysis of PIN-Formed Auxin Efflux Transporter Genes in Maize,” Plant Signaling and Behavior 14 (2019); O'Connor et al., “A Division in PIN-Mediated Auxin Patterning during Organ Initiation in Grasses,” PLOS Comput. Biol. 10: e1003447 (2014), each of which is hereby incorporated by reference in its entirety).

In Arabidopsis, PIN proteins are typically associated with the plasma membrane while PILS and non-canonical PINs are localized to the endoplasmic reticulum (Sauer and Kleine-Vehn, “PIN-FORMED and PIN-LIKES Auxin Transport Facilitators,” Development 146: dev168088 (2019), which is hereby incorporated by reference in its entirety). These proteins control intra- and intercellular IAA transport and are required for proper root growth and development (Barbez et al., “A novel Putative Auxin Carrier Family Regulates Intracellular Auxin Homeostasis in Plants,” Nature 485:119-122 (2012); Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019), each of which is hereby incorporated by reference in its entirety). Loss of PILS6 in Arabidopsis leads to increased primary root length and elevated expression of CYCB1; 1: GUS expression, indicating a negative role for PILS in root morphogenesis (Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019), which is hereby incorporated by reference in its entirety). Arabidopis PILS proteins have also been linked to ER stress responses (Waidmann et al., “Endoplasmic Reticulum Stress Controls PIN-LIKES Abundance and Thereby Growth Adaptation,” Proc. Natl. Acad. Sci. U.S.A. 120: e2218865120 (2023), which is hereby incorporated by reference in its entirety).

Although PILS proteins are evolutionarily conserved among land plants, their roles outside of eudicots are yet to be determined (Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019); Balzan et al., “The Role of Auxin Transporters in Monocots Development,” Front. Plant Sci. 5:393 (2014); Matthes et al., “Auxin EvoDevo: Conservation and Diversification of Genes Regulating Auxin Biosynthesis, Transport, and Signaling,” Mol. Plant 12:298-320 (2019), each of which is hereby incorporated by reference in its entirety). Several of the nine annotated ZmPILS genes are induced in maize roots in response to abiotic stress (Yue et al., “Genome-Wide Identification and Expression Profiling Analysis of ZmPIN, ZmPILS, ZmLAX and ZmABCB Auxin Transporter Gene Families in Maize (Zea mays L.) under Various Abiotic Stresses,” PLOS ONE 10: e0118751 (2015), which is hereby incorporated by reference in its entirety) and many of the family members exhibit tissue specific expression patterns (Walley et al., “Integration of Omic Networks in a Developmental Atlas of Maize,” Science 353:814-818 (2016), which is hereby incorporated by reference in its entirety). ZmPILS4 was identified as a major QTL of xylem traits in maize (Chen et al., “Plasticity of Root Anatomy During Domestication of a Maize-Teosinte Derived Population,” Journal of Experimental Botany 73:139-153 (2022), which is hereby incorporated by reference in its entirety).

In some embodiments, methods of the present disclosure comprise reducing or eliminating the expression and/or activity of an PIN-likes 6 (“PILS6”) protein. PILS6 is a member of the PIN-likes transporter family involved in phytohormone auxin signaling. Accordingly, some embodiments of the present disclosure involve modifying a plant or a plant cell to reduce or eliminate the expression and/or activity of PILS6 gene or protein as described infra relative to an unmodified, or wild-type PILS6 gene or protein.

Endogenous PILS6 from Zea mays genes, coding sequences, and amino acid sequences are disclosed herein as SEQ ID NOs: 1-3. These endogenous (which may also be referred to as unmodified, wild type, target, or reference) sequences are used in various embodiments of the disclosure to generate modified PILS6 sequences that reduce or eliminate the expression and/or activity of PILS6.

In some embodiments, the PILS6 gene is a ZmPILS6 from Zea mays. The gene sequence for ZmPILS6 is provided in the Maize Genome Database Browser (maizegdb.org/gene_center/gene/Zm00001eb149720) as Zm-B73-REFERENCE-NAM-5.0: Zm00001eb149720_T003 and MaizeGDB: Zm-B73-REFERENCE-GRAMENE-4.0: Zm00001d043083_T003, each of which is hereby incorporated by reference in its entirety. The mRNA sequence for ZmPILS6 is LOC103651075, GenBank Accession No. NM_001326545, which is hereby incorporated by reference in its entirety. The genomic sequence (SEQ ID NO:1), coding sequence (SEQ ID NO:2), and amino acid sequence (SEQ ID NO:3) of Zea mays ZmPILS6 are provided below.

The genomic sequence for ZmPILS6 is SEQ ID NO:1. Noncoding regions are depicted in uppercase italicized text, introns are depicted in lowercase text, exons are depicted in uppercase bold text and the position and identity of Mu transposon insertions are indicated in brackets within the sequence.

ZmPILS6 genomic sequence (SEQ ID NO: 1):
GGATATAAAATGCGCGTGTATTATCTAGTCCAGTCCAACCGACCCGGCATCGTCTCGCG
AGCCGCGACTCGTCTCGTCATTTCCATCGAACGCCCAGATCCCACATTCCCACCTAGCT
CTGCGCGATCCATCCCGGCCGCCACGCCAAGGAGGCCCTCGCCTGACCTCGGATCAAAG
CCTCCTCCATCCGCCTTCATCCCGGCCGGTGGTTCGGGCCGTCACGGGAGGGGTCCATC
ACTGGTTACTCGGTGGAGAGCTCGGGGCTCGGCTCCCATCTCACGATTCTCACCCCACA
CGCGCACCGGGTCACCTCGATTCGGCAGCCTCGCGGTGAGTTGAGTTCTTCGCTTGTCG
GGCCAGTCCGCCTCTCGCTTGGCGCGGTTATGGATTCGCGGGTGGCGTGGCGTCGTCCG
CGATCGCTCTAGCTCGCATTGAGCGATTCGCGAGTTGCGACTCCTTGTTTTGTTTGCTC
TCTGTTTTTTTTTATGTCGATGGTGATATGTGGCGGCGGGATTCTGAGCTCGGTTGCTT
CTAACCTTCCGCGGAGTGGTTTAGCTAATCGGTTTTGAAGCTGTTGATATGGATGTGCC
GTGTGCGTGACGAATCGAATGGGCCGCAGCAGGATTCCTACGCAGCTCGGCAGCAGGGG
GAATTGGATCCTGCGACCATGCGAATTTGGAACCCTGGAGCAGTGGAGTGCTAGGAGGT
CTCGGTGTCGTCATTTTTTTCTAGGGGCGTAGGAGTCGACTTGGTTGGAATCTAGTTAG
TCATAATTTC[mu1047700]TCTTTCTGATTTCATTGGTCTGTCCATGCGTCTGTTTT
CGTCAAACAAGAAGTTTGTTATTGCCAAGCATCCCGCTATGGAGGAAGCTGCCTGCCGT
ATTGTGTTACGCGAATCCGGTGAGCTGGGCTCTGCGCGTTCCTCTTGACGCCCTTTGCG
CGAACGACGAGCTTTCTTCCAGCTCGGTTGCCCCTGAACAACCAACGCCAAAAGCAGCT
CCAGAATTTTCCTGATGTCGTCTCTATATTTTTGCCTTCTAAGTCATGCTTAATGGCAG
AGTCCTTGTTGCGTGGGTCACTGCCTCACTGGGTTCTGAGGCAAGGAAAAG[mu1090629]
AAGGCTTCAGTAGTCAGGTCTGGGTCAAGCCATGATGGAGAGATCGCTGCTGGAGG
TGCTGGCCACGGCGGCGCAAGGAGGGACCGAGGGGACGTCAGTGCTGAGCATGCTCAAG
TACGCCGTGC[mu_ill_221882.6]TGCCCATCGCCAAGGTGTTCACTGTCTGCTTC
ATGGGGTTCCTTATGGCCTCCAAGTACGTCAACATTCTCCAGCCCAACGGCCGCAAGCT
TCTCAATGGGgtgagctttgtctcttgtaataactaataagtgctagctaatggaaggt
tgtgtaatggacaattgtttttctattgaggaattattttattaagtcaagatgattac
ccatcttggattttgtttaaagtaggaatatgtgaagcagtagaggagttgattgtctc
atttgttttctcgctgtatcaaattagatataacattttcactgtttgttgcagCTTGT
GTTTTCCCTTCTGCTTCCATGCCTTATATTTTCCCAATTGGGTAGAGCAATCACTATCG
AGAAGATGATACAATGgtaaagataattttgattgttatcgtacttcagttttgttatt
ataaacctagtgttatgcttcatccaagcgtgctatatttgctcttgattttttgatgt
gtgagttattgaccgagatgttgttttcagctaatcatctatttgtatatgctgctgac
cctctataagtaatacatcctacaaatattatattactgaccacacttgattggtgtgc
tattgtttatctttctatttggatcaatctttgagtaattaggttccatatggaacaaa
ttagttttagaagctctagctttctacaaaaggacaaagaaaagactacctgaaggtga
agaaaatcagcgctcaagcattaatttggttccagttgaatttatggatcaatatggga
ttgacttcagattccaggattaatttagtagcactactttatgtttaactttttgaata
ctgcagtaaagattggagacactattccaagtagtttgtttccatatgatatagtaacc
tatatttttttcttactggcaatttatatcttgttgaaaatgatagaaatttatgtaat
ggactattcactattctgtcctatattttgacatacccctggattctttcccccttctg
gtgtgtatatgtataacaagtgtccacttttgtgcataagcttctttaggagtgtgtgt
cctggaaattttcctttgtcaaataactgttctgcacttctgttctgcttttacttttt
tatatcatctatacttgcatgtttgcagGTGGTATATTCCAGTAAATATTGTTGTTGGC
GCAGTATCGGGCTCTTTGATTGGATTTGTTGTGGCATCTATCATCAGACCTCCATATCC
ATACTTCAAGTTCACTGTTATCCATATAGGAATAGgtaccacttttctgttttgaaaat
aagttttatttttatctatcatttgtttatccaataccaatccaggttctttttttgca
gGAAATATTGGAAATATACCTCTGGTCCTCATTGCAGCGTTATGTCGTGATCCTTCTAA
CCCATTTGGCGACTCTGATAAATGCAATCAAGATGGGAATGCATATATCTCATTTGGCC
AATGGgtacgccatacttttctcttttctcggcttgtacatgctcatttttactcatgt
cagcatagtgaccttgttgtttaaagattgccacttatcacctagactatgcatacaat
tagctgttgaggttattcactcggtgagtcctctatatgttgcagGTTGGTGCAATTAT
TGTTTACACATATGTATTCAAAATGCTTGCTCCACCACCAGGACAGACCTTTGATGGTT
CTGAAGAGGATGGAATCCCAATCAAGGCATCTGGAGAGAATACAGTGCCCCAAGTAGGA
AAATATCCTATGAACACTAACAGTAGTACTGTACCAGAGAATGAACCTTTGTTATCTGC
TGGGGAAGTTCAAAAGGAGCGTGCCACTTCTGTAGGAACAAAGgtaagaaattctaata
gtggagctgatttcgaatatccatttggtgcgtttacttcccactgcaaaactaaatga
ttttgttgttttatgcagATAATGGGCTATGTTAAATGTGTGGTTAAGTTTCTGAAAGA
CAAGCAGCTTCTCCAGCCACCAATTATTGCATCTgtatgtattgctgctgtagtgttct
actagcattttttaaaccaattttttgagtaattatttatatactaactctacgtctt
tttttcttttggccattgtttaacacagGCTTTTGCAATTGCAATCGGTGTTATCCCAT
TCTTGAAGAATTTTGTACTTACGGATGATGCTCCTCTGTTCTTTTTCACAGACAGCTGC
CTCATTCTTGGgtatctacatgtttcatttctattttgtttttgcgaaaacatctctct
tgccaataaagttatcaaactgagatgaatgtatctatcacctaattctgtggcatact
tttcagAGAAGCTATGATCCCATGCATTTTACTTGCTGTTGGGGGCAATCTTGTCGATG
gtaagtagctattttagttaaaactttggagaattgcatggggtgcgataatccttaga
atgaccatctttattggtttagagatatttctaatgtttttgcctttacccatgttttt
ttatggaatcagGCCCTGGTGAAGGAAGTAAGAGGCTTGGCGTGCGTACCACTGTTGCT
ATAATTTTTGCACGGTTGGTCTTGGTCCCTCTTGCTGGGGTTGGCATTACCATGTTAGT
TGATAAACTTGGTTTCATTCCCGAAGGTGATAGAATGTTCAAGTTTGTCCTGCTACTGC
AGCATTCTATGCCCACATCAGTGTTGTCAGgtatgtgaagccaacaagggatgtatott
atcagctatcacttgtcttcatgtgctcatgcgtacagattcttgttaacaattttgtg
tgttgagatctgtaaggaaaccacattatggactctgacgataggattgtttgttgcag
GTGCTGTTGCAAATCTGAGAGGTTGTGGAAAAGAATCAGCTGCAATTTTGTTCTGGGTT
CACATTTTTGCTGTGTTCTCCATGGCGGGATGGATTATATTCTATCTGAGTTTGCTCTT
CTAAGTATGTCACGCCCTGCTACCACAACAATCTGCAATTTGTGCCCGCATTCCAGCCT
GTCAGATGTAACTGCATTTTCTTGCTAGATAATGTTACTTGATCTGATTTCAGTCCGCT
GGTTCAGATGCCTTACTGCGCAGGAATATCTTTGAGATCAAATATGGTCCAAAATGTTG
AGGAAGAAACCAGTATCATGAACTGTACAGATATATCGTTGCAGAGGTACAAGTGTATC
TGTACAGATATATGGATACGGTCCCTCCTATAATCCCATATTACCGTGGTCTCCTGCTG
CTACAAGCCGTGAGCGATGGGAACACTTTCCCTTTCCCTCCACGTATCAACAGGTTCAC
ACCCAACCCTAGATTGGGATGCTGGCCTTCTTAGTTCTCCATAATATTATTTCATTTGT
ACTTCCTGTATATTGTAAACCTTGAGATGGCTGTGTTCAAATAGTTTCTTATTGGGCGA
GGGCTGAAGTAGTAGTATAGATGTATTATACTACCTATTTGAGTGTTCGGTTATGCCGC
AAATGATGCTTTATTATGCAGAACTTGTGCCCGCTGCTGGCCCGTCGGTCGTACTAACT
AAAATTGCCAATCGCGGAGACTTGCATGTTGCAAGCTCAGGCCGGCGCCCAGCGGTTTG
CAGCAAATTTTTTTTTTTGACTCGGCTATCTCCAATAGAATACTCAACTCAATATTTAT
ATTTAAACTCTACTCTATC

In some embodiments, the PILS6 gene is ZmPILS6 and comprises a nucleotide sequence of SEQ ID NO:1 or a nucleotide sequence having at least 95% sequence identity to the nucleotide sequence of SEQ ID NO:1. In some embodiments, the PILS6 gene comprises a nucleotide sequence having at least 70%, 75%, 80%, 85%, 90%, 95%, 96%, 97%, 98%, or 99% (or any number or range therein) sequence identity to the nucleotide sequence of SEQ ID NO:1.

The coding sequence of ZmPILS6 (SEQ ID NO:2) is as follows:

ATGATGGAGAGATCGCTGCTGGAGGTGCTGGCCACGGCGGCGCAAGGAGGGACCGAGGG
GACGTCAGTGCTGAGCATGCTCAAGTACGCCGTGCTGCCCATCGCCAAGGTGTTCACTG
TCTGCTTCATGGGGTTCCTTATGGCCTCCAAGTACGTCAACATTCTCCAGCCCAACGGC
CGCAAGCTTCTCAATGGGCTTGTGTTTTCCCTTCTGCTTCCATGCCTTATATTTTCCCA
ATTGGGTAGAGCAATCACTATCGAGAAGATGATACAATGGTGGTATATTCCAGTAAATA
TTGTTGTTGGCGCAGTATCGGGCTCTTTGATTGGATTTGTTGTGGCATCTATCATCAGA
CCTCCATATCCATACTTCAAGTTCACTGTTATCCATATAGGAATAGGAAATATTGGAAA
TATACCTCTGGTCCTCATTGCAGCGTTATGTCGTGATCCTTCTAACCCATTTGGCGACT
CTGATAAATGCAATCAAGATGGGAATGCATATATCTCATTTGGCCAATGGGTTGGTGCA
ATTATTGTTTACACATATGTATTCAAAATGCTTGCTCCACCACCAGGACAGACCTTTGA
TGGTTCTGAAGAGGATGGAATCCCAATCAAGGCATCTGGAGAGAATACAGTGCCCCAAG
TAGGAAAATATCCTATGAACACTAACAGTAGTACTGTACCAGAGAATGAACCTTTGTTA
TCTGCTGGGGAAGTTCAAAAGGAGCGTGCCACTTCTGTAGGAACAAAGATAATGGGCTA
TGTTAAATGTGTGGTTAAGTTTCTGAAAGACAAGCAGCTTCTCCAGCCACCAATTATTG
CATCTGCTTTTGCAATTGCAATCGGTGTTATCCCATTCTTGAAGAATTTTGTACTTACG
GATGATGCTCCTCTGTTCTTTTTCACAGACAGCTGCCTCATTCTTGGAGAAGCTATGAT
CCCATGCATTTTACTTGCTGTTGGGGGCAATCTTGTCGATGGCCCTGGTGAAGGAAGTA
AGAGGCTTGGCGTGCGTACCACTGTTGCTATAATTTTTGCACGGTTGGTCTTGGTCCCT
CTTGCTGGGGTTGGCATTACCATGTTAGTTGATAAACTTGGTTTCATTCCCGAAGGTGA
TAGAATGTTCAAGTTTGTCCTGCTACTGCAGCATTCTATGCCCACATCAGTGTTGTCAG
GTGCTGTTGCAAATCTGAGAGGTTGTGGAAAAGAATCAGCTGCAATTTTGTTCTGGGTT
CACATTTTTGCTGTGTTCTCCATGGCGGGATGGATTATATTCTATCTGAGTTTGCTCTT
CTAA

In some embodiments, the PILS6 coding sequence is ZmPILS6 and comprises a nucleotide sequence of SEQ ID NO:2 or a coding sequence having at least 95% sequence identity to the coding sequence of SEQ ID NO:2. In some embodiments, the PILS6 coding sequence has at least 70%, 75%, 80%, 85%, 90%, 95%, 96%, 97%, 98%, or 99% (or any number or range therein) sequence identity to the coding sequence of SEQ ID NO:2.

The amino acid sequence of ZmPILS6 (SEQ ID NO:3) is as follows:

MMERSLLEVLATAAQGGTEGTSVLSMLKYAVLPIAKVFTVCFMGFLMASKYVNILQPNG
RKLLNGLVFSLLLPCLIFSQLGRAITIEKMIQWWYIPVNIVVGAVSGSLIGFVVASIIR
PPYPYFKFTVIHIGIGNIGNIPLVLIAALCRDPSNPFGDSDKCNQDGNAYISFGQWVGA
IIVYTYVFKMLAPPPGQTFDGSEEDGIPIKASGENTVPQVGKYPMNTNSSTVPENEPLL
SAGEVQKERATSVGTKIMGYVKCVVKFLKDKQLLQPPIIASAFAIAIGVIPFLKNFVLT
DDAPLFFFTDSCLILGEAMIPCILLAVGGNLVDGPGEGSKRLGVRTTVAIIFARLVLVP
LAGVGITMLVDKLGFIPEGDRMFKFVLLLQHSMPTSVLSGAVANLRGCGKESAAILFWV
HIFAVFSMAGWIIFYLSLLF

In some embodiments, the PILS6 gene is ZmPILS6 and comprises an amino acid sequence of SEQ ID NO:3 or an amino acid sequence having at least 95% sequence identity to the amino acid sequence of SEQ ID NO:3. In some embodiments, the PILS6 gene encodes an amino acid sequence having at least 70%, 75%, 80%, 85%, 90%, 95%, 96%, 97%, 98%, or 99% (or any number or range therein) sequence identity to the amino acid sequence of SEQ ID NO:3.

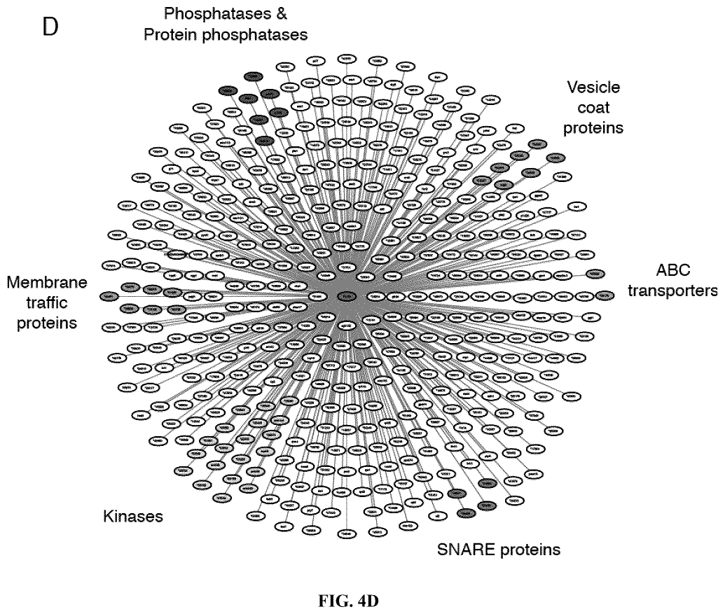

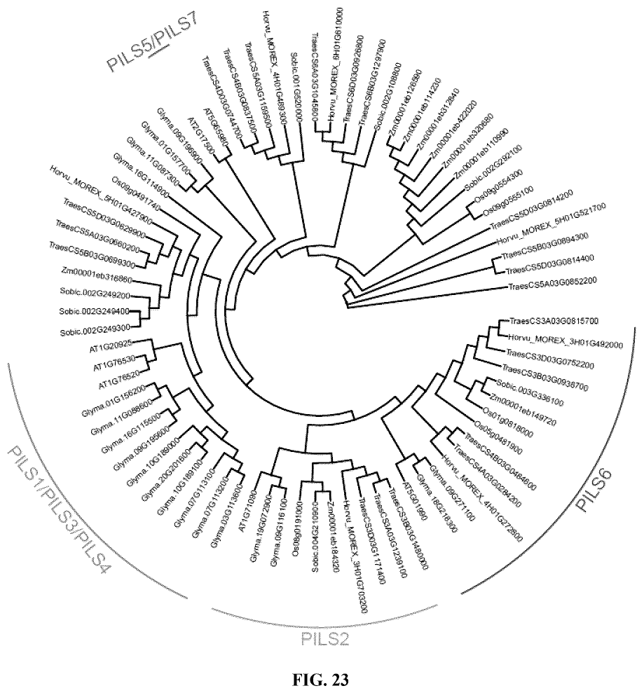

Additional exemplary PILS6 sequences useful in embodiments of the present disclosure include, without limitation, PILS6 sequences from maize, wheat, oats, barley, maize, rice, wheat, barley, sugarcane, sorghum, millet, switchgrass, rye, oats, bamboo, Bermuda grass, fescue, elephant grass, ryegrass, and Kentucky bluegrass. In some embodiments, identifying a PILS6 polynucleotide or amino acid sequence in a member of a Poaceae family is achieved by identifying a sequence based on sequence identity to the sequences set forth herein, as a non-limiting example. Sequence identity can be determined by any method known in the art, such as BLAST sequence alignments to the nucleotide or amino acid sequences described herein. Phylogenetic analysis such as that described in FIGS. 6 and 23 can also be used to identify PILS6 for use in the methods and compositions described herein.

For example, Sorghum bicolor nucleotide sequences for PILS6 include Gene model SORBI_3003G336100; GenBank Locus No. LOC8055297 (GenBank Accession No. NC_012872.2:65937697-65942524), which is hereby incorporated by reference in its entirety, which is set forth in Table 5 infra as SEQ ID NO:7. In some embodiments, the PILS6 gene is SbPILS6 and comprises a nucleotide sequence of SEQ ID NO: 7 or a nucleotide sequence having at least 95% sequence identity to the nucleotide sequence of SEQ ID NO:7. In some embodiments, the PILS6 gene comprises a nucleotide sequence having at least 70%, 75%, 80%, 85%, 90%, 95%, 96%, 97%, 98%, or 99% (or any number or range therein) sequence identity to the nucleotide sequence of SEQ ID NO:7.

Sorghum bicolor PILS6 mRNA from GenBank Accession Nos. XM_021457128 (SEQ ID NO:8), XM_021457127 (SEQ ID NO:9), and XM_002456491 (SEQ ID NO:10) (each of which is hereby incorporated by reference in its entirety) and SbPILS6 coding region (SEQ ID NO:11) are set forth in Table 5 infra. In some embodiments, the PILS6 coding sequence is SbPILS6 and comprises a nucleotide sequence of SEQ ID NO: 11 or a coding sequence having at least 95% sequence identity to the coding sequence of SEQ ID NO: 11. In some embodiments, the PILS6 coding sequence has at least 70%, 75%, 80%, 85%, 90%, 95%, 96%, 97%, 98%, or 99% (or any number or range therein) sequence identity to the coding sequence of SEQ ID NO:11.

The amino acid sequence for SbPILS6 (SEQ ID NO:12) is set forth in Table 5 infra. In some embodiments, the PILS6 gene is SbPILS6 and comprises an amino acid sequence of SEQ ID NO:12 or an amino acid sequence having at least 95% sequence identity to the amino acid sequence of SEQ ID NO: 12. In some embodiments, the PILS6 gene encodes an amino acid sequence having at least 70%, 75%, 80%, 85%, 90%, 95%, 96%, 97%, 98%, or 99% (or any number or range therein) sequence identity to the amino acid sequence of SEQ ID NO: 12.

Oryza sativa (japonica) nucleotide sequences for PILS6 include Oryza sativa PILS6-1 corresponding to gene modal BGIOSGA004692, GenBank Locus No. LOC4327495 (Accession No. NC_089035 REGION: 35441596 . . . 35446895), which is hereby incorporated by reference in its entirety and is set forth as SEQ ID NO:13 in Table 5 infra. In some embodiments, the PILS6 gene is OsPILS6-1 and comprises a nucleotide sequence of SEQ ID NO: 13 or a nucleotide sequence having at least 95% sequence identity to the nucleotide sequence of SEQ ID NO: 13. In some embodiments, the PILS6 gene comprises a nucleotide sequence having at least 70%, 75%, 80%, 85%, 90%, 95%, 96%, 97%, 98%, or 99% (or any number or range therein) sequence identity to the nucleotide sequence of SEQ ID NO: 13.

Oryza sativa (japonica) nucleotide sequences for PILS6 also include Oryza sativa PILS6-2 corresponding to GenBank Locus No. LOC4339130, (Accession No. NC_089039 REGION: complement (23811988 . . . 23815523)), which is hereby incorporated by reference in its entirety and is set forth as SEQ ID NO:18 in Table 5 infra. In some embodiments, the PILS6 gene is OsPILS6-2 and comprises a nucleotide sequence of SEQ ID NO: 18 or a nucleotide sequence having at least 95% sequence identity to the nucleotide sequence of SEQ ID NO: 18. In some embodiments, the PILS6 gene comprises a nucleotide sequence having at least 70%, 75%, 80%, 85%, 90%, 95%, 96%, 97%, 98%, or 99% (or any number or range therein) sequence identity to the nucleotide sequence of SEQ ID NO: 18.

Oryza sativa PILS6-1 mRNA sequences from GenBank Accession Nos. XM_015765602 and XR_003239989, each of which is hereby incorporated by reference in its entirety, are set forth as SEQ ID NOs: 14-15 in Table 5 infra. The coding sequence for OsPILS6-1 is set forth as SEQ ID NO: 16 in Table 5 infra. In some embodiments, the PILS6 coding sequence is OsPILS6-1 and comprises a nucleotide sequence of SEQ ID NO: 16 or a coding sequence having at least 95% sequence identity to the coding sequence of SEQ ID NO: 16. In some embodiments, the PILS6 coding sequence has at least 70%, 75%, 80%, 85%, 90%, 95%, 96%, 97%, 98%, or 99% (or any number or range therein) sequence identity to the coding sequence of SEQ ID NO:16.

Oryza sativa PILS6-2 mRNA sequence from GenBank Accession No. XM_015782757.3, which is hereby incorporated by reference in its entirety, is set forth as SEQ ID NO:19 in Table 5 infra. The OsPILS6-2 coding sequence is set forth as SEQ ID NO: 20 in Table 5 infra. In some embodiments, the PILS6 coding sequence is OsPILS6-2 and comprises a nucleotide sequence of SEQ ID NO:20 or a coding sequence having at least 95% sequence identity to the coding sequence of SEQ ID NO:20. In some embodiments, the PILS6 coding sequence has at least 70%, 75%, 80%, 85%, 90%, 95%, 96%, 97%, 98%, or 99% (or any number or range therein) sequence identity to the coding sequence of SEQ ID NO:20.

The amino acid sequences for OsPILS6-1 (SEQ ID NO:17) and OsPILS6-2 (SEQ ID NO:21) are set forth in Table 5 infra. In some embodiments, the PILS6 gene is OsPILS6-1 and comprises an amino acid sequence of SEQ ID NO:17 or an amino acid sequence having at least 95% sequence identity to the amino acid sequence of SEQ ID NO: 17. In some embodiments, the PILS6 gene encodes an amino acid sequence having at least 70%, 75%, 80%, 85%, 90%, 95%, 96%, 97%, 98%, or 99% (or any number or range therein) sequence identity to the amino acid sequence of SEQ ID NO: 17. In some embodiments, the PILS6 gene is OsPILS6-2 and comprises an amino acid sequence of SEQ ID NO:21 or an amino acid sequence having at least 95% sequence identity to the amino acid sequence of SEQ ID NO:21. In some embodiments, the PILS6 gene encodes an amino acid sequence having at least 70%, 75%, 80%, 85%, 90%, 95%, 96%, 97%, 98%, or 99% (or any number or range therein) sequence identity to the amino acid sequence of SEQ ID NO:21.

Setaria italica (Foxtail millet) nucleotide sequences for PILS6 include Gene model SETIT_001507 mg (GenBank Accession No. NC_028454 REGION: 39919686 . . . 39924250), which is hereby incorporated by reference in its entirety, and is set forth as SEQ ID NO:22 in Table 5 infra. In some embodiments, the PILS6 gene is SiPILS6 and comprises a nucleotide sequence of SEQ ID NO:22 or a nucleotide sequence having at least 95% sequence identity to the nucleotide sequence of SEQ ID NO:22. In some embodiments, the PILS6 gene comprises a nucleotide sequence having at least 70%, 75%, 80%, 85%, 90%, 95%, 96%, 97%, 98%, or 99% (or any number or range therein) sequence identity to the nucleotide sequence of SEQ ID NO:22.

Setaria italica PILS6 mRNA sequence from GenBank Accession No. XM_004970317.4, which is hereby incorporated by reference in its entirety, is set forth as SEQ ID NO:23 in Table 5 infra. The coding sequence of SiPILS6 is set forth as SEQ ID NO: 24 in Table 5 infra. In some embodiments, the PILS6 coding sequence is SiPILS6 and comprises a nucleotide sequence of SEQ ID NO:24 or a coding sequence having at least 95% sequence identity to the coding sequence of SEQ ID NO:24. In some embodiments, the PILS6 coding sequence has at least 70%, 75%, 80%, 85%, 90%, 95%, 96%, 97%, 98%, or 99% (or any number or range therein) sequence identity to the coding sequence of SEQ ID NO:24.

The amino acid sequence for SiPILS6 (SEQ ID NO:25) is set forth in Table 5 infra. In some embodiments, the PILS6 gene is SiPILS6 and comprises an amino acid sequence of SEQ ID NO:25 or an amino acid sequence having at least 95% sequence identity to the amino acid sequence of SEQ ID NO:25. In some embodiments, the PILS6 gene encodes an amino acid sequence having at least 70%, 75%, 80%, 85%, 90%, 95%, 96%, 97%, 98%, or 99% (or any number or range therein) sequence identity to the amino acid sequence of SEQ ID NO:25.

Brachypodium distachyon nucleotide sequences for PILS6 include gene model BRADI_2g53360v3 (GenBank Accession No. NC_016132 REGION: 52289841 . . . 52295181), which is hereby incorporated by reference in its entirety, and is set forth as SEQ ID NO:26 in Table 5 infra. In some embodiments, the PILS6 gene is BdPILS6 and comprises a nucleotide sequence of SEQ ID NO:26 or a nucleotide sequence having at least 95% sequence identity to the nucleotide sequence of SEQ ID NO: 26. In some embodiments, the PILS6 gene comprises a nucleotide sequence having at least 70%, 75%, 80%, 85%, 90%, 95%, 96%, 97%, 98%, or 99% (or any number or range therein) sequence identity to the nucleotide sequence of SEQ ID NO:26.

The Brachypodium distachyon PILS6 coding sequence is set forth as SEQ ID NO: 27 in Table 5 infra. In some embodiments, the PILS6 coding sequence is BdPILS6 and comprises a nucleotide sequence of SEQ ID NO:27 or a coding sequence having at least 95% sequence identity to the coding sequence of SEQ ID NO:27. In some embodiments, the PILS6 coding sequence has at least 70%, 75%, 80%, 85%, 90%, 95%, 96%, 97%, 98%, or 99% (or any number or range therein) sequence identity to the coding sequence of SEQ ID NO:27.

The amino acid sequence for BdPILS6 (SEQ ID NO:28) is set forth in Table 5 infra. In some embodiments, the PILS6 gene is BdPILS6 and comprises an amino acid sequence of SEQ ID NO:28 or an amino acid sequence having at least 95% sequence identity to the amino acid sequence of SEQ ID NO:28. In some embodiments, the PILS6 gene encodes an amino acid sequence having at least 70%, 75%, 80%, 85%, 90%, 95%, 96%, 97%, 98%, or 99% (or any number or range therein) sequence identity to the amino acid sequence of SEQ ID NO:28.

### Modifications of PILS6: Mutagenesis & Genome Editing

One aspect of the present disclosure relates to a Poaceae plant cell comprising a non-naturally occurring loss of function mutation in a PIN-likes 6 (PILS6) gene encoding a PILS6 protein or fragment thereof. The expression and/or activity of the PILS6 gene or protein is reduced or eliminated in the plant cell as compared to a wild type plant cell.

In some embodiments, reducing or eliminating the expression and/or activity of PILS6 gene comprises: (i) introducing a loss-of-function mutation into a coding sequence and/or a regulatory sequence of an PILS6 gene; and/or (ii) introducing into the plant or plant cell an inhibitory polynucleotide targeting the PILS6 gene, thereby decreasing expression of the PILS6 gene. As used herein, a “regulatory sequence” is a sequence that regulates the expression and/or activity of a gene or protein.

In some embodiments, the expression and/or activity of PILS6 can be reduced or eliminated by mutating a PILS6 gene. In some embodiments, the PILS6 gene in a plant or plant cell has a mutation that inhibits normal expression and/or activity of the gene or the protein encoded by the gene.

The terms “mutation” or “genome edit” mean a human-induced change in the genetic sequence compared to a wild type or reference sequence (i.e., a sequence that exists in nature). In some embodiments, mutations are those that cause the gene coding sequence or a promoter to not be expressed or to have a reduced level of mRNA expression, or those that reduce the activity, expression, or stability of the encoded protein. In some embodiments, the methods of the present disclosure comprise introducing the mutation by mutagenesis or genome editing.

As used herein, a “loss of function” mutation is a mutation that reduces or eliminates PILS6 gene expression. The terms “modify”, “modifying,” and the like used herein encompass modifying PILS6 to reduce or eliminate gene expression by, e.g., mutation or genome editing or by using an inhibitory polynucleotide. In some embodiments, the PILS6 mutation may be due to an insertion of all or part of one or more transposable element(s) into the gene or coding sequence.

A mutation may be, without limitation, a “substitution mutation”, which is a change in the nucleic acid sequence that results in the substitution of an amino acid for another amino acid; a “nonsense mutation” or “stop codon mutation”, which is a change in the nucleic acid sequence that results in the introduction of a premature stop codon and thus the termination of translation (resulting in a truncated protein); an “insertion mutation” of one or more amino acids, due to one or more codons having been added in the coding sequence of the nucleic acid; a “deletion mutation” of one or more amino acids, due to one or more codons or parts of codons having been deleted in the coding sequence of the nucleic acid; and a “frameshift mutation”, resulting in the nucleic acid sequence being translated in a different frame downstream of the mutation, or any combination of mutation types. A frameshift mutation can have various causes, such as the insertion, deletion, substitution, and/or duplication of one or more nucleotides. Mutations that affect pre-mRNA splicing (splice junction mutations) can also result in frameshifts. A “splice junction mutation”, which alters or abolishes the correct splicing of the pre-mRNA sequence, resulting in a protein of different amino acid sequence than the wild type. For example, one or more exons may be skipped during RNA splicing, resulting in a protein lacking the amino acids encoded by the skipped exons. Alternatively, the reading frame may be altered through incorrect splicing, or one or more introns may be retained, or alternate splice donors or acceptors may be generated, or splicing may be initiated at an alternate position (e.g., within an intron), or alternate polyadenylation signals may be generated. Accordingly, in some embodiments, the mutation is an insertion, a deletion, a substitution mutation, or any combination thereof. In some embodiments, the mutation is a “knock-out” mutation.

As used herein, a “knock-out” mutation is a mutant allele that eliminates functional PILS6 expression, i.e., the mutant allele produces no PILS6 protein having the ability to transport auxin, in a plant cell in vivo. Knock-out mutant PILS6 alleles include, for instance, insertion and/or deletion mutations that disrupt the coding region, delete a portion or the entire coding region, and frameshift or stop-codon mutations that lead to a substantial or entire deletion of the protein.

In some embodiments, the mutation in PILS6 is non-transgenic. Accordingly, it is contemplated that non-transgenic mutations may be introduced via gene editing techniques or induced mutation techniques. In some embodiments, a mutation may be introduced by mutagenesis such as by treatment with a mutagenic agent.

Any suitable mutagenic agent can be used for embodiments of the present disclosure For example, mutagens creating point mutations, deletions, insertions, rearrangements, transversions, transitions, or any combination thereof may be used. Suitable radiation mutagens include, without limitation, ultraviolet light, x-rays, gamma rays, and fast neutrons. Suitable chemical mutagens include, but are not limited to, ethyl methanesulfonate (EMS), methylmethane sulfonate (MMS), N-ethyl-N-nitrosourea (ENU), triethylmelamine (TEM), N-methyl-N-nitrosourca (MNU), procarbazine, chlorambucil, cyclophosphamide, diethyl sulfate, acrylamide monomer, melphalan, nitrogen mustard, vincristine, dimethylnitrosamine, N-methyl-N′-nitro-nitrosoguanidine 25 (MNNG), nitrosoguanidine, 2-aminopurine, 7,12 dimethyl-benz (a) anthracene (DMBA), ethylene oxide, hexamethylphosphoramide, bisulfan, diepoxyalkanes (diepoxyoctane (DEO), diepoxybutane (DEB), 2-methoxy-6-chloro-9 [3-(ethyl-2-chloro-ethyl) aminopropylamino]acridine dihydrochloride (ICR-170), sodium azide, formaldehyde, or combinations thereof.

In some embodiments, a mutation may be introduced by genome editing. Genome editing is a type of genetic engineering in which DNA is inserted, replaced, or removed, or any combination thereof, from a genome using artificially engineered nucleases or “molecular scissors.” The nucleases typically create double-stranded breaks (“DSBs”) at desired locations in the genome and harness the cell's endogenous mechanisms to repair the induced break by processes of homology dependent repair (“HDR”) or nonhomologous end-joining (“NHEJ”). Four main families of engineered nucleases include: Zinc finger nucleases (“ZFNs”), Transcription Activator-Like Effector Nucleases (“TALENs”), the Clustered Regularly Interspaced Short Palindromic Repeats (“CRISPR”) system, and engineered meganuclease with re-engineered homing endonucleases. Any method of genome editing may be used in the embodiments described herein.

Use of systems for gene editing has been widely described. For example, the use of CRISPR guide RNA in conjunction with CRISPR/Cas technology to target RNA is described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), which are hereby incorporated by reference in their entirety.

CRISPR/Cas type RNA-guided endonucleases provide an efficient system for inducing genetic modifications in genomes of many organisms and can be used in the methods described herein to introduce one or more genetic modifications in a plant genome. Non-limiting examples of genome editing nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, or homologs, modified versions, and endonuclease inactive versions thereof. An example of a fusion protein to Cas9 is a cytidine deaminase-Cas9 fusion protein used in cytidine base editing to mutate nucleotides in target genes without generating double-strand breaks as described in Komor et al., “Programmable Editing of a Target Base in Genomic DNA without Double-Stranded DNA Cleavage,” Nature 533:420-424 (2016), which is hereby incorporated by reference in its entirety.

There are two distinct components to a CRISPR/Cas system, a guide RNA and an endonuclease, such as Cas9. The guide RNA is a combination of the endogenous bacterial CRISPR RNA (“crRNA”) and trans-activating crRNA (“tracrRNA”) into a single chimeric guide RNA (“gRNA”) transcript. The gRNA combines the targeting specificity of the crRNA with the scaffolding properties of the tracrRNA into a single transcript. When the gRNA and Cas9 are expressed in the cell, the genomic target sequence can be modified or permanently disrupted. The gRNA/Cas9 complex is recruited to the target sequence by the base-pairing between the gRNA sequence which has a region of the complementarity to the target sequence in the genomic DNA. For successful binding of Cas9, the genomic target sequence must also contain the correct Protospacer Adjacent Motif (“PAM”) sequence immediately following the target sequence. The binding of the gRNA/Cas9 complex localizes the Cas9 to the genomic target sequence so that the wild type Cas9 can cut both strands of DNA causing a DSB. Cas9 generates DSBs through the combined activity of two nuclease domains, RuvC and HNH. Cas9 will cut 3-4 nucleotides upstream of the PAM sequence. A DSB can be repaired through one of two general repair pathways: (1) NHEJ DNA repair pathway or (2) the HDR pathway. The NHEJ repair pathway often results in insertions/deletions (InDels) at the DSB site that can lead to frameshifts and/or premature stop codons, effectively disrupting the open reading frame (ORF) of the targeted gene. The HDR pathway requires the presence of a repair template, which is used to fix the DSB. HDR faithfully copies the sequence of the repair template to the cut target sequence. Specific nucleotide changes can be introduced into a targeted gene by the use of HDR with a repair template. CRISPR specificity can be controlled by level of homology and binding strength of the specific gRNA for a given gene target, or by modification of the Cas endonuclease itself. For example, a D10A mutant of the RuvC domain, retains only the HNH domain and generates a DNA nick rather than a DSB.

When the guide RNA and the gene editing endonuclease are expressed in the cell, the genomic target sequence can be modified and/or permanently disrupted. The guide RNA/gene editing endonuclease complex is recruited to the target sequence by the base-pairing between the guide RNA sequence and the complementary sequence of the target sequence in the genomic DNA.

Exemplary guide RNAs for genome editing of PILS6 in Zea mays include, without limitation, CGGCGCAAGGAGGGACCGAGGGG (SEQ ID NO:29); GTCAACATTCTCCAGCCCAACGG (SEQ ID NO:30); and CTATCGAGAAGATGATACAATGG (SEQ ID NO:31).

Exemplary guide RNAs for genome editing of PILS6 in Sorghum bicolor include, without limitation, CCGTTGCTATAATTTTTGCACGG (SEQ ID NO:32); GTATATCTCATTTGGCCAATGGG (SEQ ID NO:33); GTCAACATTCTCCAGCCCAACGG (SEQ ID NO:34); and CTGCGCAAGGAGGGACCGAGGGG (SEQ ID NO:35).

Exemplary guide RNAs for genome editing of PILS6-1 in Oryza sativa include, without limitation, GTCAATATCCTCCAGCCGAACGG (SEQ ID NO:36); AGTTGGGAAAATATAAGGCAAGG (SEQ ID NO:37); GACGGTAAACACCTTGGCAATGG (SEQ ID NO:38); and GATGGAGAGGTCGCTGATGGAGG (SEQ ID NO:39).

In some embodiments, CRISPR gene editing is used to generate an PILS6 gene mutation by causing nucleotide insertions or deletions (indels) at the DSB site. In some embodiments, a point mutation, insertions, deletions, or any combination thereof, can be generated in a PILS6 gene. In some embodiments, the guide RNA targets ZmPILS6 (SEQ ID NO:1), SbPILS6 (SEQ ID NO:7), OsPILS6-1 (SEQ ID NO:13), OsPILS6-2 (SEQ ID NO:18), SiPILS6 (SEQ ID NO:22), or BdPILS6 (SEQ ID NO:26).

In some embodiments, the mutated PILS6 gene is obtained by inserting an inactivating nucleic acid molecule into the gene encoding the functional PILS6 gene or its promoter under conditions effective to inactivate the gene. Suitable inactivating nucleic acid molecules can include, for example, a transposable element. Examples of such transposable elements include, but are not limited to, an Activator (Ac) transposon, a Dissociator (Ds) transposon, or a Mutator (Mu) transposon.

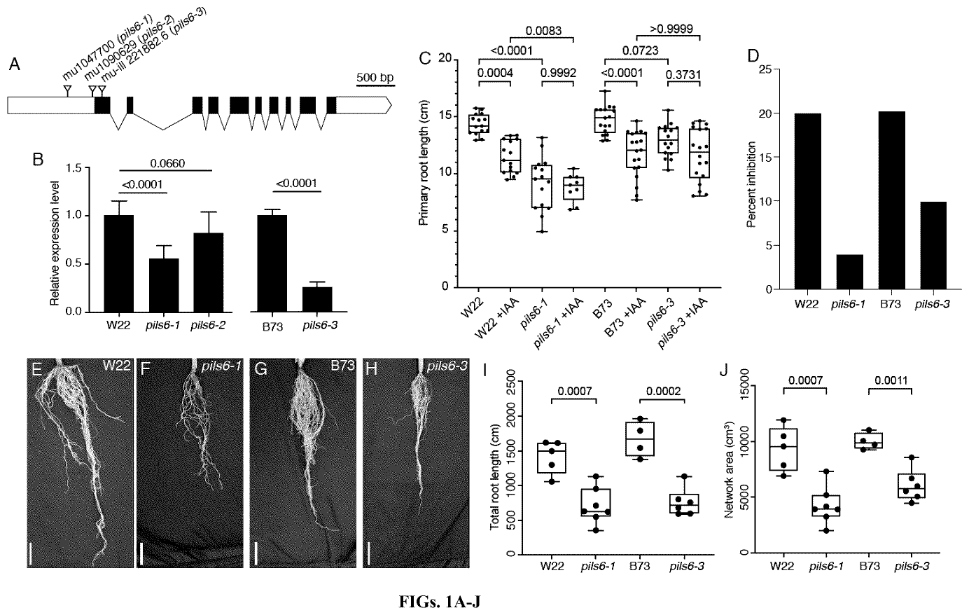

An exemplary Mu mutation in Zea mays PILS6 causing a reduction or elimination of the expression and/or activity of ZmPILS6 gene or protein, which is effective in altering crown root architecture, reducing lateral root formation, and reducing stalk height, comprises a Mu insertion event between nucleotide positions 777 and 778 of SEQ ID NO:1 (Mu1047700). This mutation is called pils6-1 and results in an interruption of the non-coding region of ZmPILS6 (FIG. 1A). The sequence of pils6-1 is set forth in Table 5 infra as SEQ ID NO:64. The sequence of Mu1047700 is CTTTCTGAT. Another exemplary Mu mutation in Zea mays PILS6 causing a reduction or elimination of the expression and/or activity of ZmPILS6 gene or protein, which is effective in altering crown root architecture, reducing lateral root formation, and reducing stalk height, comprises a Mu insertion event between nucleotide positions 1227 and 1228 of SEQ ID NO: 1 (Mu_ill_221882.6). This mutation is called pils6-3 and results in an interruption of the coding region of ZmPILS6 (FIG. 1A). The sequence of pils6-3 is set forth in Table 5 infra as SEQ ID NO:66. The sequence of Mu_ill_221882.6 is CTTCATGGGG (SEQ ID NO: 67). The Mu insertion event between nucleotide positions 1102 and 1103 of SEQ ID NO: 1 (Mu1090629), called pils6-2, results in an interruption of the non-coding region of ZmPILS6 (FIG. 1A). The sequence of pils6-2 is set forth in Table 5 infra as SEQ ID NO: 65. The sequence of Mu1090629 is GGCTTCAGT. This mutation was not effective in altering crown root architecture, reducing lateral root formation, or reducing stalk height. In some embodiments, the loss of function mutation in ZmPILS6 comprises an insertion between nucleotides 777 and 778 of SEQ ID NO: 1 or an insertion between nucleotides 1227 and 1228 of SEQ ID NO:1.

In some embodiments, the mutated PILS6 gene is obtained by subjecting at least one cell of a plant to Agrobacterium transformation under conditions effective to insert an Agrobacterium T-DNA sequence into the gene, thereby inactivating the gene. Suitable Agrobacterium T-DNA sequences can include, for example, those sequences that are carried on a binary transformation vector of pAC106, pAC161, pGABI1, pADIS1, pCSA110, pDAP101, derivatives of pBIN19, or pCAMBIA plasmid series.

In some embodiments, one or more mutations reduce PILS6 gene expression by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any number or range therein, as compared to the amount of PILS6 gene expression in a control. In some embodiments, expression of the PILS6 gene is reduced by at least 35% compared to a control. In some embodiments, one or more mutations reduce PILS6 gene expression in roots by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any number or range therein, as compared to the amount of PILS6 gene expression in wild type roots. In some embodiments, expression of the PILS6 gene in roots is reduced by at least 35% compared to wild type roots.

In some embodiments, the expression of the mutated PILS6 is 0%-10%, 15%-20%, 20%-30%, 25%-35%, 30%-40%, 35%-45%, 40%-50%, 45%-55%, 50%-60%, 55%-65%, 60%-70%, 65%-75%, 70%-80%, 75%-85%, 80%-90%, or any number or range therein, of the PILS6 gene expression in a control.

In some embodiments, one or more mutations reduce the amount of functional PILS6 protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., 100% means no PILS6 protein is detectable), or any number or range therein, as compared to the amount of the functional PILS6 protein produced by the cell not comprising the mutant PILS6 allele. The amount of PILS6 protein can be measured by any method known in the art such as by antibody detection or mass spectrometry.

Also encompassed is (i) the production of a “non-functional” PILS6 protein (e.g., a truncated PILS6 protein) having no detectable biological activity in vivo; (ii) the reduction in the absolute amount of the functional PILS6 protein (e.g., reduced amount of PILS6 protein being made due to the mutation in the PILS6 gene); or (iii) the production of an PILS6 protein with significantly reduced detectable biological activity, each as compared to the activity of a functional wild type (or unmodified) PILS6 protein.

### Inhibitory Polynucleotides

Yet another aspect of the present disclosure relates to a Poaceae plant cell comprising an inhibitory polynucleotide targeting a PIN-likes 6 (PILS6) gene encoding a PILS6 protein, where expression and/or activity of the PILS6 gene is reduced or eliminated in the plant cell compared to a plant cell without the inhibitory polynucleotide.

This aspect (and any aspect) of the present disclosure can be carried out with any of the embodiments disclosed herein.

In some embodiments, the methods of the present disclosure further comprise introducing into the plant or plant cell an inhibitory polynucleotide targeting the PILS6 gene encoding the PILS6 protein. A plant or plant cell of this and other aspects of the present disclosure may comprise an inhibitory polynucleotide targeting one or more PILS6 genes to reduce their expression. Methods for reducing gene expression involving the expression of “inhibitory polynucleotide” sequences in plants are known in the art, and include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro-RNA (miRNA) interference, as non-limiting examples.

For example, an inhibitory polynucleotide may be an antisense polynucleotide or a double stranded RNA inhibitor (RNAi) polynucleotide capable of down-regulating gene expression of PILS6.

In some embodiments, RNA interference (RNAi) occurs when an organism recognizes double-stranded RNA molecules and hydrolyzes them. The resulting hydrolysis products are small RNA fragments of, typically, 19-24 nucleotides in length, called small interfering RNAs (siRNAs) or microRNAs (miRNAs). The siRNAs then hybridize to mRNAs (or other RNAs) and cause hydrolysis of the RNA. Most plant miRNAs show extensive base pairing to, and guide cleavage of their target mRNAs.

The RNAi pathway can be exploited in plants by using recombinant technology, which entails transforming a plant with a vector comprising DNA that when expressed produces a dsRNA homologous or nearly homologous to a gene target. The gene target can be homologous to an endogenous plant RNA. RNA interference in plants can also be referred to as post-transcriptional gene silencing or RNA silencing and can be triggered by expression of an antisense strand of a target sequence of interest. In general, a plant or plant cell is transformed with DNA that is incorporated into the plant genome, and when expressed produces a dsRNA that is complementary to a gene of interest, which can be an endogenous plant gene (e.g., PILS6), leading to the reduction or elimination of expression of that gene.

In some embodiments, the inhibitory polynucleotide is an RNAi polynucleotide comprising a sense polynucleotide strand comprising at least 21 contiguous nucleotides from an PILS6 gene sequence, and an antisense polynucleotide strand that hybridizes to the sense polynucleotide strand, where the sense and the antisense polynucleotide strand form a duplex. In some embodiments, the RNAi polynucleotide comprises a sense polynucleotide strand comprising at least 21, 22, 23, 24, 25, 30, 40, 50, 60, 70, 80, 90, 100, 200, 300, 400 or more than 400 contiguous nucleotides from an PILS6 sequence, and an antisense polynucleotide strand that is complementary to the sense polynucleotide strand, wherein the sense and the antisense polynucleotide strand form a duplex. In some embodiments, the RNAi polynucleotide comprises a sense polynucleotide strand 21-400 contiguous nucleotides from an PILS6 sequence, and an antisense polynucleotide strand that is complementary to the sense polynucleotide strand, wherein the sense and the antisense polynucleotide strand form a duplex.

RNAi constructs may also include a polynucleotide sequence that separates the sense and antisense strands to facilitate duplex formation. In some embodiments, the RNAi polynucleotide further comprises a polynucleotide sequence heterologous to the sense and antisense polynucleotides. In some embodiments, the RNAi polynucleotide comprises an intron.

In some embodiments, the inhibitory polynucleotide comprises an antisense polynucleotide. In some embodiments, the inhibitory polynucleotide comprises an antisense polynucleotide of a PILS6 coding sequence of at least 21 base pairs (bp), 22 bp, 23 bp, 24 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 125 bp, 150 bp, 175 bp, 200 bp, 300 bp, 400 bp, 500 bp, 600 bp, 700 bp, 800 bp, 900 bp, or greater than 1000 bp, or any number of base pairs or range of base pairs between 20 bp and 1000 bp.

Exemplary RNAi polynucleotides targeting PILS6 in Zea mays include, without limitation, SEQ ID NOs: 40-41 as follows:

SEQ ID NO: 40:
AAGGAGGGACCGAGGGGACGTCAGTGCTGAGCATGCTCAAGTACGCCGTGCTGCCCATC
GCCAAGGTGTTCACTGTCTGCTTCATGGGGTTCCTTATGGCCTCCAAGTACGTCAACAT
TCTCCAGCCCAACGGCCGCAAGCTTCTCAATGGGCTTGTGTTTTCCCTTCTGCTTCCAT
GCCTTATATTTTCCCAATTGGGTAGAGCAATCACTATCGAGAAGATGATACAATGGTGG
TATATTCCAGTAAATATTGTTGTTGGCGCAGTATCGGGCTCTTTGATTGGATTTGTTGT
GGCATttcaagagaATGCCACAACAAATCCAATCAAAGAGCCCGATACTGCGCCAACAA
CAATATTTACTGGAATATACCACCATTGTATCATCTTCTCGATAGTGATTGCTCTACCC
AATTGGGAAAATATAAGGCATGGAAGCAGAAGGGAAAACACAAGCCCATTGAGAAGCTT
GCGGCCGTTGGGCTGGAGAATGTTGACGTACTTGGAGGCCATAAGGAACCCCATGAAGC
AGACAGTGAACACCTTGGCGATGGGCAGCACGGCGTACTTGAGCATGCTCAGCACTGAC
GTCCCCTCGGTCCCTCCTT
SEQ ID NO: 41:
GCGTTATGTCGTGATCCTTCTAACCCATTTGGCGACTCTGATAAATGCAATCAAGATGG
GAATGCATATATCTCATTTGGCCAATGGGTTGGTGCAATTATTGTTTACACATATGTAT
TCAAAATGCTTGCTCCACCACCAGGACAGACCTTTGATGGTTCTGAAGAGGATGGAATC
CCAATCAAGGCATCTGGAGAGAATACAGTGCCCCAAGTAGGAAAATATCCTATGAACAC
TAACAGTAGTACTGTACCAGAGAATGAACCTTTGTTATCTGCTGGGGAAGTTCAAAAGG
AGCGTttcaagagaACGCTCCTTTTGAACTTCCCCAGCAGATAACAAAGGTTCATTCTC
TGGTACAGTACTACTGTTAGTGTTCATAGGATATTTTCCTACTTGGGGCACTGTATTCT
CTCCAGATGCCTTGATTGGGATTCCATCCTCTTCAGAACCATCAAAGGTCTGTCCTGGT
GGTGGAGCAAGCATTTTGAATACATATGTGTAAACAATAATTGCACCAACCCATTGGCC
AAATGAGATATATGCATTCCCATCTTGATTGCATTTATCAGAGTCGCCAAATGGGTTAG
AAGGATCACGACATAACGC.

Exemplary RNAi polynucleotides targeting PILS6 in Sorghum bicolor include, without limitation, SEQ ID NOs: 42-43 as follows:

SEQ ID NO: 42:
ACGGCTGCGCAAGGAGGGACCGAGGGGACGTCGGTGCTGAGCATGCTCAAGTATGCCGT
GCTTCCCATCGCCAAGGTGTTCACTGTCTGCTTCATGGGGTTCCTCATGGCCTCCAAGT
ACGTCAACATTCTCCAGCCCAACGGCCGCAAGCTTCTCAATGGGCTTGTGTTTTCCCTT
CTGCTTCCATGCCTTATATTTTCCCAATTGGGTAGAGCAATCACTATCGAGAAGATGAT
ACAATGGTGGTATATTCCAGTAAATATTGTTGTAGGTGCAGTATCCGGCTCTTTGATTG
GATTTttcaagagaAAATCCAATCAAAGAGCCGGATACTGCACCTACAACAATATTTAC
TGGAATATACCACCATTGTATCATCTTCTCGATAGTGATTGCTCTACCCAATTGGGAAA
ATATAAGGCATGGAAGCAGAAGGGAAAACACAAGCCCATTGAGAAGCTTGCGGCCGTTG
GGCTGGAGAATGTTGACGTACTTGGAGGCCATGAGGAACCCCATGAAGCAGACAGTGAA
CACCTTGGCGATGGGAAGCACGGCATACTTGAGCATGCTCAGCACCGACGTCCCCTCGG
TCCCTCCTTGCGCAGCCGT
SEQ ID NO: 43:
TTGATGGTTCTGAAGAGGATGAACTCCCAATCAAGGCATCTGGAGAGAATACAGTGCCC
CAAATAGGAAATTATCCTATGAACACTCACACTAGTACTGTACCAGAGAATGAACCATT
GTTATCTGCTGGGGATGTTCAAAAGGAACGTGCCACTTCTGTAGGAACAAAGATAATGG
GCTTTGTTAAATGTGTGGTTAAGTTCCTGAAAGACAAGCAGCTTCTCCAGCCACCGATT
ATTGCATCTGCGTTTGCAATTGTAATTGGTGTTATCCCATTCTTGAAGAATTTTGTCCT
TACAGttcaagagaCTGTAAGGACAAAATTCTTCAAGAATGGGATAACACCAATTACAA
TTGCAAACGCAGATGCAATAATCGGTGGCTGGAGAAGCTGCTTGTCTTTCAGGAACTTA
ACCACACATTTAACAAAGCCCATTATCTTTGTTCCTACAGAAGTGGCACGTTCCTTTTG
AACATCCCCAGCAGATAACAATGGTTCATTCTCTGGTACAGTACTAGTGTGAGTGTTCA
TAGGATAATTTCCTATTTGGGGCACTGTATTCTCTCCAGATGCCTTGATTGGGAGTTCA
TCCTCTTCAGAACCATCAA.

Exemplary RNAi polynucleotides targeting PILS6-1 in Oryza sativa include, without limitation. SEQ ID NOs: 44-45 as follows:

SEQ ID NO: 44:
GGGAGGCACCGTGGGGACGTCGGTTTTCGACATGCTCAAGTACGCCGTGCTGCCCATTG
CCAAGGTGTTTACCGTCTGTTTCATGGGGTTCCTCATGGCCTCTAAGTATGTCAATATC
CTCCAGCCGAACGGCCGCAAGCTTCTCAATGGGCTTGTGTTCTCGCTTCTACTICCTTG
CCTTATATTTTCCCAACTGGGTAGAGCAATCACAATCGAAAAGATGCTGCAATGGTGGT
ATATTCCAGTAAATATTGTTGTAGGTGCAGTGTCAGGCTCTTTGATTGGCTTTGTGGTG
GCTTCttcaagagaGAAGCCACCACAAAGCCAATCAAAGAGCCTGACACTGCACCTACA
ACAATATTTACTGGAATATACCACCATTGCAGCATCTTTTCGATTGTGATTGCTCTACC
CAGTTGGGAAAATATAAGGCAAGGAAGTAGAAGCGAGAACACAAGCCCATTGAGAAGCT
TGCGGCCGTTCGGCTGGAGGATATTGACATACTTAGAGGCCATGAGGAACCCCATGAAA
CAGACGGTAAACACCTTGGCAATGGGCAGCACGGCGTACTTGAGCATGTCGAAAACCGA
CGTCCCCACGGTGCCTCCC
SEQ ID NO: 45:
TTGCTCCACCACCTGGTGAATCCTTTGATAGCGCTGAAGAAGATATTCTTCCAATTAAG
GCATCTGGAGATAATGTGGTGCCTGAAAAAGGGAAATATCCAACAAGCACTCGCACTAG
TACTGTACCTGAAAATGAGCCTTTGTTATCTTCTGAAGGTGATAAAAATGTTTCTACTT
CTCTAGGATCGAAGATAATGGGCATTGTTAGAAGCATGGTTAAGTTCCTAAAAGACAAG
CAGCTTCTTCAGCCACCAATTATTGCCTCTGTTTTTGCAATTGCCATTGGTGTTGTTCC
AGTCTttcaagagaAGACTGGAACAACACCAATGGCAATTGCAAAAACAGAGGCAATAA
TTGGTGGCTGAAGAAGCTGCTTGTCTTTTAGGAACTTAACCATGCTTCTAACAATGCCC
ATTATCTTCGATCCTAGAGAAGTAGAAACATTTTTATCACCTTCAGAAGATAACAAAGG
CTCATTTTCAGGTACAGTACTAGTGCGAGTGCTTGTTGGATATTTCCCTTTTTCAGGCA
CCACATTATCTCCAGATGCCTTAATTGGAAGAATATCTTCTTCAGCGCTATCAAAGGAT
TCACCAGGTGGTGGAGCAA.

In some embodiments, the inhibitory polynucleotide reduces PILS6 gene expression by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any amount or range therein, as compared to the amount of PILS6 gene expression in a control.

In some embodiments, the inhibitory polynucleotide reduces the amount of functional PILS6 protein by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional protein is produced by the cell) as compared to the amount of the functional PILS6 protein produced by the cell not comprising the inhibitory polynucleotide.

In some embodiments, a plant comprises the plant cell of any of the embodiments of the present disclosure. Also provided are the seeds and/or fruit of a plant of any of the embodiments disclosed herein.

In some embodiments, the seed, fruit, plant part, plant, or plant cell is from the Poaceae family. In some embodiments, the seed, fruit, plant part, plant, or plant cell is maize (Zea mays). In some embodiments, the plant or plant cell is selected from the group consisting of Zea mays (Maize or Corn), Oryza sativa (Rice), Triticum spp. (Wheat), Hordeum vulgare (Barley), Saccharum officinarum (Sugarcane), Sorghum bicolor (Sorghum), Setaria italica (Foxtail millet), Brachypodium distachyon (Brachypodium), Panicum virgatum (Switchgrass), Secale cereale (Rye), Avena sativa (Oats), Bambusa spp. (Bamboo), Cynodon dactylon (Bermuda Grass), Festuca spp. (Fescue), Pennisetum purpureum (Elephant Grass or Napier Grass), Lolium spp. (Ryegrass), and Poa pratensis (Kentucky Bluegrass). In some embodiments, the plant or plant cell is Zea mays. In some embodiments, the seed, fruit, plant part, plant, or plant cell is from maize, rice, wheat, barley, sugarcane, sorghum, millet, switchgrass, rye, oats, bamboo, Bermuda grass, fescue, elephant grass, ryegrass, or Kentucky bluegrass.

### Crown Root Architecture, Lateral Root Formation, & Stalk Height

One aspect of the present disclosure relates to a method of altering crown root architecture, lateral root formation, and/or stalk height in a Poaceae plant. This method involves reducing or eliminating expression of a PIN-likes 6 (PILS6) gene in a plant or a plant cell of the Poaceae family, where said reducing or eliminating is effective to reduce crown root architecture, lateral root formation, and/or stalk height in the plant or a plant produced from the plant cell, as compared to a wild type plant.

This aspect of the present disclosure can be carried out with any of the embodiments disclosed herein.

In some embodiments, the method involves altering crown root architecture in the plant.

A “crown root” is the area of the plant where the root system connects to the basal part of the shoot system and where the root system extends laterally into the ground (see e.g., FIGS. 1E-H). In some embodiments, the crown root architecture has a reduced size in a plant with reduced as compared to a crown root from a plant without a PILS6 modification. A plant without the modification as described herein may be, e.g., a wildtype plant. In some embodiments, crown root size is reduced by at least 5%, 10%, 15%, 20%, 25%, 30%, 35%, 40%, 45%, 50%, 55%, 60%, 65%, 70%, 75%, 80%, 85%, 90%, or 100% or any number or range therein, as compared to the crown root size in a plant without the modification. In some embodiments, crown root total root length is reduced by at least 5%, 10%, 15%, 20%, 25%, 30%, 35%, 40%, 45%, 50%, 55%, 60%, 65%, 70%, 75%, 80%, 85%, 90%, or 100% or any number or range therein, as compared to the crown root total root length in a plant without the modification. In some embodiments, crown root total root length is reduced by at least 10%, 20%, 25%, 30%, 35%, 40%, 45%, 50%, 55%, 60%, 65%, 70%, 75%, 80%, 85%, 90%, or 100% or any number or range therein, as compared to the crown root total root length in a plant without the modification. In some embodiments, crown root total root length is reduced by at least 30% as compared to the crown root total root length in a plant without the modification. In some embodiments, crown root total root length is reduced by 40-70% as compared to the crown root total root length in a plant without the modification. In some embodiments, the crown root total root length is reduced by 50-60% as compared to the crown root total root length in a plant without the modification. In some embodiments, the root network area is reduced by at least 10%, 20%, 25%, 30%, 35%, 40%, 45%, 50%, 55%, 60%, 65%, 70%, 75%, 80%, 85%, 90%, or 100% or any number or range therein, as compared to the root network area in a plant without the modification. In some embodiments, the root network area is reduced by 40-70% as compared to the root network area in a plant without the modification. In some embodiments, the root network area is reduced by 50-60% as compared to the root network area in a plant without the modification.

In some embodiments, the method involves altering lateral root formation in the plant.

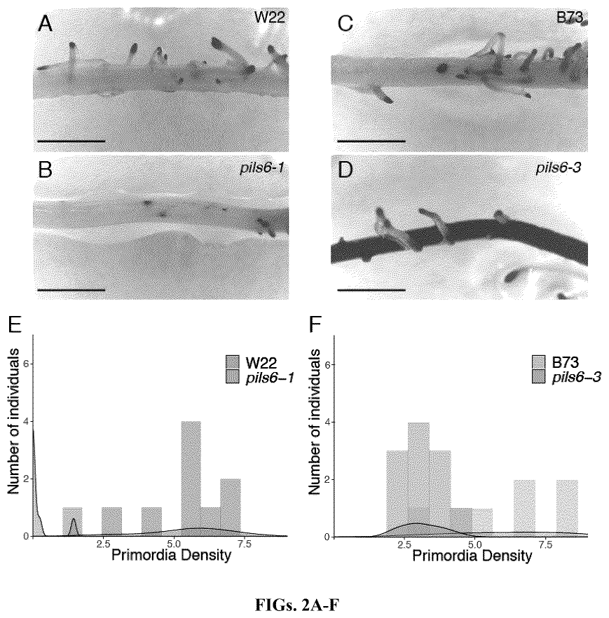

A “lateral root” is a root which branches off of another root (see e.g., FIGS. 2A-F). In some embodiments, the root system has reduced lateral root formation as compared to a root system from a plant without a PILS6 modification. In some embodiments, lateral root formation is measured from the primary root. A plant without a PILS6 modification as described herein may be, e.g., a wildtype plant. In some embodiments, lateral root formation is reduced by at least 5%, 10%, 15%, 20%, 25%, 30%, 35%, 40%, 45%, 50%, 55%, 60%, 65%, 70%, 75%, 80%, 85%, 90%, 95%, or 100%, or any number or range therein, as compared to the lateral root formation in a plant without the modification. In some embodiments, lateral root primordia density is reduced by at least 5%, 10%, 15%, 20%, 25%, 30%, 35%, 40%, 45%, 50%, 55%, 60%, 65%, 70%, 75%, 80%, 85%, 90%, 95%, or 100%, or any number or range therein, as compared to the lateral root primordia density in a plant without the modification. In some embodiments, lateral root primordia density is reduced by at least 30% compared to the lateral root primordia density in a plant without the modification.

In some embodiments, the length of the meristematic zone in the root is decreased in plants with a PILS6 modification as compared to the meristematic zone from a plant without a PILS6 modification. A plant without a PILS6 modification as described herein may be, e.g., a wildtype plant. In some embodiments, the meristematic zone is reduced by at least 2%, 5%, 10%, 15%, 20%, 25%, 30%, 35%, 40%, 45%, 50%, 55%, 60%, 65%, 70%, 75%, 80%, 85%, 90%, 95% or any number or range therein, as compared to the meristematic zone of a plant without the modification.

In some embodiments, the method involves altering stalk height in the plant.

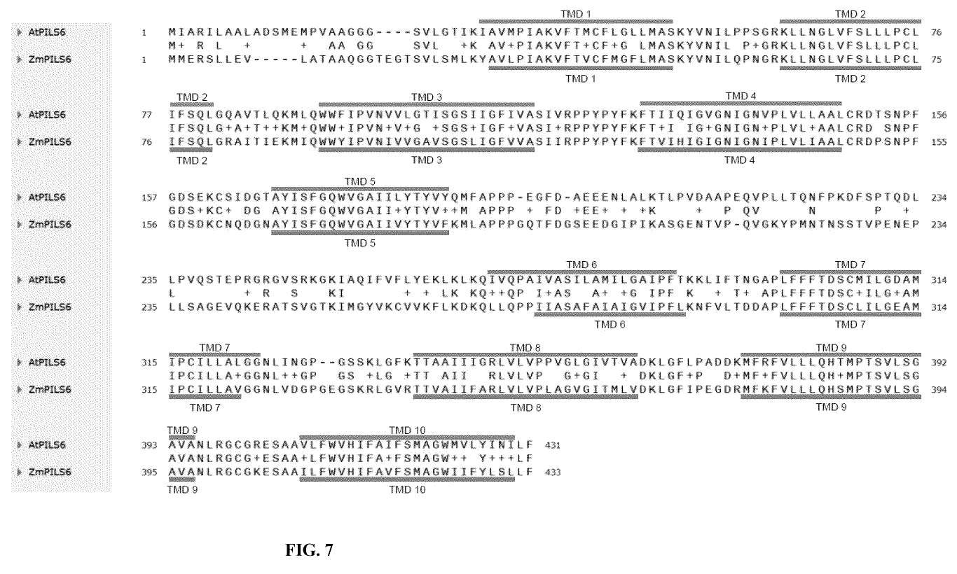

A “stalk height” or “shoot height” refers to the height of the main above ground shoot of a plant (see e.g., FIGS. 9A-E). In some embodiments, the stalk height is reduced as compared to stalk height from a plant without a PILS6 modification. A plant without a PILS6 modification as described herein may be, e.g., a wildtype plant. In some embodiments, stalk height is reduced by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500% or any number or range therein, as compared to the stalk height of a plant without the modification. In some embodiments, the stalk height is reduced by at least 10% compared to stalk height of a plant without the modification.

In some embodiments, a plant with a PILS6 modification has a similar stalk circumference as compared to stalk circumference from a plant without a PILS6 modification.

In some embodiments, a plant with a PILS6 modification has an increased ear height as compared to ear height from a plant without a PILS6 modification. In some embodiments, a plant with a PILS6 modification has an increased ear placement ratio as compared to a plant without a PILS6 modification A plant without a PILS6 modification as described herein may be, e.g., a wildtype plant. In some embodiments, ear height is increased by at least 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500% or any number or range therein, as compared to the ear height of a plant without the modification. In some embodiments, the ear height is increased by at least 10% compared to ear height of a plant without the modification.

Another aspect of the present disclosure relates to a method of increasing salt tolerance in a Poaceae plant. This method involves reducing or eliminating expression of a PIN-likes 6 (PILS6) gene in a plant or a plant cell of the Poaceae family, where said reducing or eliminating is effective to increase salt tolerance in the plant or a plant produced from the plant cell, as compared to a wild type plant.

This aspect of the present disclosure can be carried out with any of the embodiments disclosed herein.

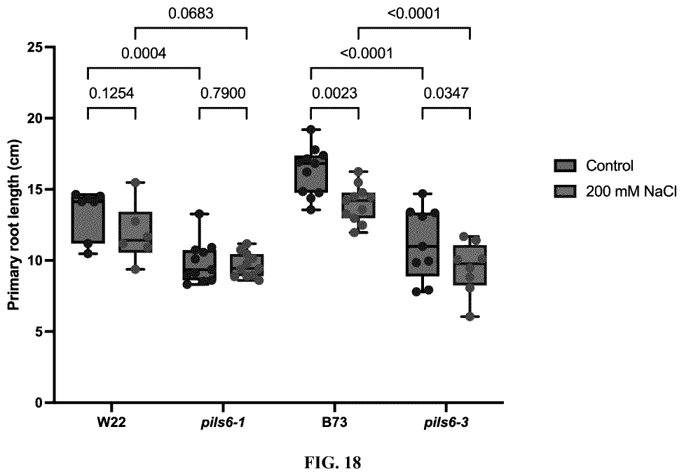

Salt tolerance in plants refers to the ability of certain plant species to endure and thrive in environments with high salinity levels. Salt stress occurs when plants are exposed to excessive salt concentrations, leading to detrimental effects on their growth and development. High salinity may disrupt a plant's water uptake, causes ion toxicity, and induces oxidative stress, which can damage cellular structures and impair metabolic functions. Increased salt tolerance would allow planting and growth in suboptimal soils with increased salt concentrations. In some embodiments, a plant with a PILS6 modification has an increased salt tolerance as compared to ear height from a plant without a PILS6 modification. A plant without a PILS6 modification as described herein may be, e.g., a wildtype plant. In some embodiments, salt tolerance may be measured by growth in the presence of salt ions, e.g., at 100 mM-200 mM NaCl as non-limiting examples (see e.g., Example 8 and FIGS. 18-21). In some embodiments, a plant with a PILS6 modification has an increased relative growth to a plant without a PILS6 modification by at least 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500% or any number or range therein.

### Nucleic Acid Constructs

A further aspect of the present disclosure relates to a nucleic acid construct comprising a nucleotide sequence targeting a PILS6 gene comprising: (i) a guide RNA or (ii) an inhibitory polynucleotide; a 5′ heterologous DNA promoter sequence; and a 3′ terminator sequence.

A “nucleic acid construct” or “vector” is a nucleic acid, plasmid, or virus used to transfer coding information to a host cell. Typically, the vector contains sequences directing transcription and translation of the relevant gene, a selectable marker, and sequences allowing autonomous replication or chromosomal integration. Suitable “expression vectors” comprise a region 5′ of the gene which harbors transcriptional initiation controls and a region 3′ of the DNA fragment which controls transcriptional termination.

The DNA sequence in the expression vector is operably linked to appropriate expression control sequences, including a promoter, to direct RNA synthesis and protein expression. “Operably linked” means an association between polynucleotide (e.g., nucleic acid) sequences on a single nucleic acid molecule such that the function of one is affected by the other. For example, a promoter is operably linked to a coding sequence when the coding sequence is under the transcriptional control of the promoter. Coding sequences can be operably linked to regulatory sequences in sense or antisense orientation. The expression vector can contain one or more selectable marker genes to provide a phenotypic trait for selection of a transformed host cell. Useful selectable markers include dihydrofolate reductase or neomycin resistance for eukaryotic cell culture, or tetracycline or ampicillin resistance in E. coli. The vector may be introduced into the host cell(s) using any of a variety of techniques, including transformation, transfection, transduction, viral infection, gene guns, Ti-mediated gene transfer, calcium phosphate transfection, DEAE-Dextran-mediated transfection, lipofection, or electroporation. Examples of vectors include, but are not limited to, viral particles, baculovirus, phage, plasmids, phagemids, cosmids, fosmids, bacterial artificial chromosomes, viral DNA, such as vaccinia and adenovirus, Ti vectors, P1-based artificial chromosomes, yeast plasmids, Bacillus vectors, and Aspergillus vectors. Examples of bacterial vectors include, but are not limited to, pQE vectors, pBluescript plasmids, pNH vectors, and lambda-ZAP vectors. Examples of eukaryotic vectors include pXT1, pSG5, pSVK3, pBPV, pMSG, and pSVLSV40 vectors.

Depending upon the targeted expression profile in a cell or tissue type, or developmental timeframe, any one of a number of suitable promoters may be used. A “promoter” refers to a nucleic acid fragment capable of controlling transcription of another nucleic acid fragment. A promoter is a non-coding genomic DNA sequence, usually upstream (5′) to and operably linked to the relevant coding sequence, and its primary function is to act as a binding site for RNA polymerase to initiate transcription by the RNA polymerase. A promoter may also include distal enhancer or repressor elements, which can be located as much as several thousand base pairs from the start site of transcription. The terms “capable of controlling expression” or “initiating transcription”, refer to the primary function of a promoter. Additionally, there is “expression” of RNA, including functional RNA, or the expression of polypeptide for operably linked encoding nucleotide sequences, as the transcribed RNA ultimately may be translated into the corresponding polypeptide. Promoters vary in their “strength” (i.e., their ability to promote transcription). The nucleotide sequence of the promoter determines the nature of the RNA polymerase binding and other related protein factors that attach to the RNA polymerase and/or promoter, and the rate of RNA synthesis.

A “constitutive” promoter is a promoter that is active under most environmental and developmental conditions. An “inducible” promoter is a promoter that is active under environmental or developmental regulation. Suitable constitutive promoters that are functional in a plant cell include, but are not limited to, the cauliflower mosaic virus 35S (CaMV35S) promoter, a tandem 35S promoter, a cauliflower mosaic virus 19S promoter, a figwart mosaic virus 35S (FMV35S) promoter, a nopaline synthase gene promoter, an octopine synthase gene promoter, a potato or tomato protease inhibitor I or II gene promoter, and a ubiquitin promoter. Suitable inducible promoters that are functional in a plant cell may include, but are not limited to, a phenylalanine ammonia-lyase gene promoter, a chalcone synthase gene promoter, a pathogenesis-related protein gene promoter, a copper-inducible regulatory element, tetracycline, and chlor-tetracycline-inducible regulatory elements. A “tissue-specific” promoter is a promoter that primarily are functional in a specific tissue type. For example, tissue specific promoters can be employed which regulate expression in only one or some tissues or organs, such as leaves, roots, fruit, seeds, anthers, ovaries, pollen, meristem, stems or flowers, or parts thereof. In some embodiments, the promoter is a root-specific promoter.

The protein-encoding nucleic acid, a promoter molecule of choice, a suitable 3′ regulatory region and, if desired, polyadenylation signals and/or a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

Single or multiple nucleic acids may be ligated into an appropriate vector, under the control of one or more suitable promoters, to prepare a nucleic acid construct. In some embodiments, the expression vector comprises the nucleic acid construct. In some embodiments, the expression vector further comprises a genome editing nuclease.

Once the nucleic acid molecule has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. “Transformation” or “transforming” refers to the introduction of a nucleic acid into a host organism. Host organisms containing a transformed nucleic acid construct or DNA fragment may be referred to as “transgenic” or “recombinant” organisms.

In some embodiments, a cell is transformed with the nucleic acid construct. In some embodiments, the cell is a bacterial cell or a plant cell. A transformed plant cell may be regenerated into a plant by any method known in the art. In some embodiments, a plant is transformed with the nucleic acid construct. Plant parts from the plant or a plant grown from the plant cell of any of the embodiments of the present disclosure are also contemplated. In some embodiments, a plant seed produced from the plant of any of the embodiments of the present disclosure is contemplated.

Once the nucleic acid construct has been prepared, it is ready to be incorporated into a host cell. Basically, this method is carried out by transforming a host cell with the nucleic acid construct under conditions effective to achieve transcription of the nucleic acid molecule in the host cell. This is achieved with standard cloning procedures known in the art, such as described by Sambrook et al., Molecular Cloning: A Laboratory Manual, Second Edition, Cold Spring Harbor Laboratory, Cold Spring Harbor, New York (1989), which is hereby incorporated by reference in its entirety. Suitable host cells are plant cells. Suitable host cells also include bacterial cells. Methods of transformation may result in transient or stable expression of the nucleic acid under control of the promoter. In one embodiment, the nucleic acid construct of the present disclosure is stably inserted into the genome of the recombinant plant cell as a result of the transformation, although transient expression can serve an important purpose, particularly when the plant under investigation is slow-growing.

Plant tissue suitable for transformation includes leaf tissue, root tissue, meristems, zygotic and somatic embryos, callus, protoplasts, tassels, pollen, embryos, anthers, and the like. The means of transformation chosen is that most suited to the tissue to be transformed.

Transient expression in plant tissue can be achieved by particle bombardment (Klein et al., “High-Velocity Microprojectiles for Delivering Nucleic Acids Into Living Cells,” Nature 327:70-73 (1987), which is hereby incorporated by reference in its entirety), also known as biolistic transformation of the host cell, as disclosed in U.S. Pat. Nos. 4,945,050; 5,036,006; and 5,100,792, all to Sanford et al., and in Emerschad et al., “Somatic Embryogenesis and Plant Development from Immature Zygotic Embryos of Seedless Grapes (Vitis vinifera),” Plant Cell Reports 14:6-12 (1995), which are hereby incorporated by reference in their entirety.

In particle bombardment, tungsten or gold microparticles (1 to 2 μm in diameter) are coated with the DNA of interest and then bombarded at the tissue using high pressure gas. In this way, it is possible to deliver foreign DNA into the nucleus and obtain a temporal expression of the gene under the current conditions of the tissue. Biologically active particles (e.g., dried bacterial cells containing the vector and heterologous DNA) can also be propelled into plant cells. Other variations of particle bombardment, now known or hereafter developed, can also be used.

An appropriate method of stably introducing a nucleic acid construct into plant cells is to infect a plant cell with Agrobacterium tumefaciens or Agrobacterium rhizogenes previously transformed with the nucleic acid construct. As described supra, the Ti (or RI) plasmid of Agrobacterium enables the highly successful transfer of a foreign nucleic acid molecule into plant cells. A variation of Agrobacterium transformation uses vacuum infiltration in which whole plants are used (Senior, “Uses of Plant Gene Silencing,” Biotechnology and Genetic Engineering Reviews 15:79-119 (1998), which is hereby incorporated by reference in its entirety).

Yet another method of introduction is fusion of protoplasts with other entities, either minicells, cells, lysosomes, or other fusible lipid-surfaced bodies (Fraley et al., “Liposome-mediated Delivery of Tobacco Mosaic Virus RNA into Tobacco Protoplasts: A Sensitive Assay for Monitoring Liposome-protoplast Interactions,” Proc. Natl. Acad. Sci. U.S.A. 79:1859-63 (1982), which is hereby incorporated by reference in its entirety). The nucleic acid molecule may also be introduced into the plant cells by electroporation (Fromm et al., “Expression of Genes Transferred into Monocot and Dicot Plant Cells by Electroporation,” Proc. Natl. Acad. Sci. USA 82:5824 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate. Other methods of transformation include polyethylene-mediated plant transformation, micro-injection, physical abrasives, and laser beams (Senior, “Uses of Plant Gene Silencing,” Biotechnology and Genetic Engineering Reviews 15:79-119 (1998), which is hereby incorporated by reference in its entirety). The precise method of transformation is not critical to the practice of the present disclosure. Any method that results in efficient transformation of the host cell of choice is appropriate for practicing the present disclosure.

In certain embodiments, transformation described herein is carried out by Agrobacterium-mediated transformation, whisker method transformation, vacuum infiltration, biolistic transformation, electroporation, micro-injection, polyethylene-mediated transformation, or laser-beam transformation.

After transformation, the transformed plant cells must be regenerated. Plant regeneration from cultured protoplasts is described in Evans et al., Handbook of Plant Cell Cultures, Vol. 1, New York, New York: MacMillan Publishing Co. (1983); Vasil, ed., Cell Culture and Somatic Cell Genetics of Plants, Vol. I (1984) and Vol. III (1986), Orlando: Acad. Press; and Fitch et al., “Somatic Embryogenesis and Plant Regeneration from Immature Zygotic Embryos of Papaya (Carica papaya L.),” Plant Cell Rep. 9:320 (1990), which are hereby incorporated by reference in their entirety.

Means for regeneration vary from species to species of plants, but generally a suspension of transformed protoplasts or a petri plate containing explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. Efficient regeneration will depend on the medium, on the genotype, and on the history of the culture. If these three variables are controlled, then regeneration is usually reproducible and repeatable.

In one embodiment, transformed cells are first identified using a selection marker simultaneously introduced into the host cells along with the nucleic acid construct of the present disclosure. Suitable selection markers include, without limitation, markers encoding for antibiotic resistance, such as the neomycin phosphotransferae II (“nptII”) gene which confers kanamycin resistance (Fraley et al., “Expression of Bacterial Genes in Plant Cells,” Proc. Natl. Acad. Sci. U.S.A. 80:4803-4807 (1983), which is hereby incorporated by reference in its entirety), and the genes which confer resistance to gentamycin, G418, hygromycin, streptomycin, spectinomycin, tetracycline, chloramphenicol, and the like. Cells or tissues are grown on a selection medium containing the appropriate antibiotic, whereby generally only those transformants expressing the antibiotic resistance marker continue to grow. The selection marker employed will depend on the target species; for certain target species, different antibiotics, herbicide, or biosynthesis selection markers are preferred.

Plant cells and tissues selected by means of an inhibitory agent or other selection marker are then tested for the acquisition of the transgene (Sambrook et al., Molecular Cloning: A Laboratory Manual, Cold Spring Harbor, New York: Cold Spring Harbor Press (1989), which is hereby incorporated by reference in its entirety).

### Plant Breeding

A further aspect of the present disclosure relates to a method of breeding for reduced crown root architecture, reduced lateral root formation, and/or reduced stalk height in a Poaceae plant. This method involves providing a candidate plant or plant part having a non-naturally occurring loss of function mutation in a PIN-likes 6 (PILS6) gene, or an inhibitory polynucleotide reducing or eliminating expression of a PIN-likes 6 (PILS6) gene, analyzing the candidate plant or plant part for the presence, in its genome, of the mutation in the PILS6 gene or the inhibitory polynucleotide. The method further involves identifying, based on said analyzing, a candidate plant suitable for breeding, where the candidate plant comprises the mutation in the PILS6 gene or the inhibitory polynucleotide, and breeding the candidate plant with at least one other plant.

This aspect of the present disclosure can be carried out with any of the embodiments disclosed herein.

In some embodiments, identifying the presence of a modified PILS6 polynucleotide sequence is achieved based on sequencing the sequences set forth herein, by identifying a sequence based on sequence identity to the sequences set forth herein. Sequence identity can be determined by any method known in the art, such as BLAST sequence alignments to the nucleotide or amino acid sequences described herein. In some embodiments, a molecular marker is developed to identify a modified PILS6 polynucleotide sequence. Non-limiting examples of sequencing can be accomplished, for example, using next generation sequencing, Sanger sequencing, TaqMan assays, UniTaq assays, real-time PCR assays, digital PCR, microarray, hybridization or other detection methods.

In some embodiments, analyzing the candidate plant for the presence, in its genome, of a modified PILS6 polynucleotide encoding an PILS6 protein, wherein the expression and/or activity of said PILS6 gene or PILS6 gene or protein is reduced or eliminated as compared to a plant without the modification involves isolating nucleic acids from the plant or plant part, analyzing nucleic acids from the plant, or plant parts for the presence of the modified PILS6 polynucleotide, and detecting the modified PILS6 polynucleotide.

In some embodiments, identifying comprises sequencing the nucleic acids of the PILS6 gene, mRNA, or cDNA from the plant, or plant part. In some embodiments, identifying comprises detecting an insertion mutation corresponding to the Mu insertion event at nucleotide position 777 of SEQ ID NO:1 (Mu1047700). In some embodiments, identifying comprises detecting an insertion mutation corresponding to the Mu insertion event at nucleotide position 1227 of SEQ ID NO:1 (Mu_ill_221882.6). In some embodiments, identifying comprises detecting an insertion at or near position 777 of SEQ ID NO: 1. In some embodiments, identifying comprises detecting an insertion at or near position 1277 of SEQ ID NO:1. In some embodiments, identifying involves a molecular marker developed to facilitate identification of the modified PILS6 polynucleotide.

In some embodiments, analyzing the candidate plant for a modified PILS6 polynucleotide involves measure crown root architecture, crown root size, crown root total root length, root network area, lateral root formation, lateral root primordia density, and/or stalk height in comparison to a plant without a modified PILS6 polynucleotide. Exemplary methods of such measurements are described herein (see e.g., Examples 1-6).

In some embodiments, the breeding involves crossing, making hybrids, backcrossing, self-crossing, double haploid breeding, and/or combinations thereof. The modified PILS6 gene can be transferred to other plants by sexual crossing. Any of a number of standard breeding techniques can be used, depending upon the species to be crossed. The plants themselves can be cultivated in accordance with conventional procedure so that the nucleic acid construct is present in the resulting plants.

Alternatively, seeds are recovered from the plants. These seeds can then be planted in the soil and cultivated using conventional procedures to produce additional plants.

The following examples are intended to exemplify the practice of embodiments of the disclosure but are by no means intended to limit the scope thereof.

### Examples

### Example 1—Materials and Methods for Examples 2-6

Plant materials. UniformMu (UFMu-06513 and UFMu-13871) and mu-illumina (mu-ill 221882.6) seed stocks were obtained from the Maize Genetics Cooperation Stock Center and genotyped using gene specific primers for ZmPILS6 (Zm00001eb149720) (Table 1).

TABLE 1
Gene Specific Primers for ZmPILS6

| | | SEQ | |
|---|---|---|---|
| Primer | | ID | |
| Name | Sequence | NO: | Purpose |
| CMS9 | CTGGAAGAAAGCTCGTCGTT | 46 | pils6-1 genotyping F |
| CMS10 | TTGATATGGATGTGCCGTGT | 47 | pils6-1 genotyping R |
| CMS11 | TAAGGAACCCCATGAAGCAG | 48 | pils6-2 genotyping F |
| CMS12 | AACGACGAGCTTTCTTCCAG | 49 | pils6-2 genotyping R |
| DK1699 | TGCCTGCCGTATTGTGTTAC | 50 | pils6-3 genotyping F |
| DK1700 | TTGTCCATTACACAACCTTCCA | 51 | pils6-3 genotyping R |
| TIR6 | AGAGAAGCCAACGCCAWCGCCTCYATTTCGTC | 52 | UFMu genotyping |
| | | | primer |
| DK1308 | CACCATGATGGAGAGATCGCTGCT | 53 | FL ZmPILS6 cDNA |
| | | | cloning F |
| DK1309 | TTAGAAGAGCAAACTCAGATAGAATATAA | 54 | FL ZmPILS6 cDNA |
| | | | cloning R |
| DK1310 | GAAGAGCAAACTCAGATAGAATATAATCC | 55 | ZmPILS6 w/o STOP |
| | | | cDNA cloning R |
| DK1691 | ACCTCTGGTCCTCATTGCAG | 56 | PILS6 RT PCR F |
| DK1692 | CTTTTGAACTTCCCCAGCAG | 57 | PILS6 RT PCR R |
| DK1695 | ATCTCGTTGGGGATGTCTTG | 58 | FPGS RT PCR F |
| DK1695 | CCCACGCGATCTTATTTTGT | 59 | FPGS RT PCR R |
| DK1570 | CTGCTGGGGAAGTTCAAAAG | 60 | PILS6 qPCR F |
| DK1571 | TAATTGGTGGCTGGAGAAGC | 61 | PILS6 qPCR R |
| N582 | ATCTCGTTGGGGATGTCTTG | 62 | FPGS qPCR F |
| N583 | AGCACCGTTCAAATGTCTCC | 63 | FPGS qPCR R |

Alleles were designated as pils6-1 (mu1047700), pils6-2 (mu1090629), and pils6-3 (mu-ill 221882.6). Both pils6-1 and pils6-2 originated from the UniformMu collection and were subsequently backcrossed into the inbred line W22 bz1-mum9 for two generations prior to characterization. The pils6-3 allele originated from the mu-illumina collection and was subsequently backcrossed into B73 once before characterization. W22 bz1-mum9 was used as control for pils6-1 and pils6-2, while B73 was used as control for pils6-3.

Auxin response assays were performed using the rolled towel assay as previously described (Draves, “Maize Seedling Growth and Hormone Response Assays Using the Rolled Towel Method,” Current Protocols 2: e562 (2022), which is hereby incorporated by reference in its entirety) using 20-40 biological replicates per genotype and treatment. Maize kernels were surface sterilized with 6% bleach and planted on germination paper and placed vertically in a 2-L beaker with 600 mL of 0.5×Linsmaier and Skoog (LS) media. Seedlings were grown for 3 days after germination (DAG) and then treated with 10 μM indole-3-acetic acid (IAA) or an equal volume 95% ethanol for 7 additional days.

For proteomics analysis, W22 and pils6-1 kernels were surface sterilized and planted in rolled towels as described above. Two days after planting, ungerminated kernels were removed from the assay. Five-day-old seedlings were removed from the rolled towels and floated on 0.5×LS media supplemented with 10 μM IAA in 0.5×LS or an equal volume of 95% ethanol in 0.5×LS for 1 hour. For each biological replicate, total root tissue from 5 seedlings was excised using a scalpel and pooled prior to flash freezing in liquid nitrogen. Three biological replicates were generated per genotype and treatment.

Plant phenotyping. Maize seedlings were grown in greenhouse conditions to the V7 stage in 1-gallon pots with soil. Seedlings were removed from the pots and root systems were rinsed with tap water to remove all soil. After drying at room temperature, crown root systems were photographed using a Cannon DSLR camera. Crown root images were analyzed using Rhizhovision Explorer (Seethepalli et al., “Rhizo Vision Explorer: Open-Source Software for Root Image Analysis and Measurement Standardization,” AoB PLANTS 13: plab056 (2021), which is hereby incorporated by reference in its entirety). Statistical analysis of crown root traits was performed using a t-test in R using the t.test function.

Plant height was measured from the base of the plant to the tip of the newly formed leaf using ImageJ. Statistical significance was performed using a t test.

Lateral root primordia staining. Kernels were sterilized and planted as described above. Six days after germination (DAG), primary roots were fixed overnight in 70% ethanol and acetic acid. Primary roots were gradually rehydrated before hydrolysis in 1 N HCl. Roots were subsequently incubated in Schiff's solution for 1 hour in the dark (Hoecker et al., “Manifestation of Heterosis During Early Maize (Zea mays L.) Root Development,” Theor. Appl. Genet. 112:421-429 (2006), which is hereby incorporated by reference in its entirety). For each genotype, 7-11 biological replicates were stained. Lateral roots and lateral root primordia were counted by eye and statistical analysis was performed using the t.test function in R.

RNA Extraction. Maize kernels (W22, B73, pils6-1, pils6-2, and pils6-3) were surface sterilized and grown in 0.5×LS via the rolled towel method as described above. For each biological replicate, primary root tissue from five independent five-day-old seedlings was harvested and pooled together, flash frozen in liquid nitrogen, and ground to a homogenous fine powder using a mortar and pestle. Total RNA was extracted from 3 of biological replicates per genotype. Total RNA was extracted as previously described using TRIzol reagent followed by column clean-up with Zymo Quick-RNA Plant Miniprep following the manufacturer's instructions (McReynolds et al., “Temporal and Spatial Auxin Responsive Networks in Maize Primary Root,” Quantitative Plant Biology 3: e21 (2022), which is hereby incorporated by reference in its entirety). The purified RNA concentration and purity was measured by using a NanoDrop One. 1 μg of RNA was used to synthesize cDNA using LunaScript® RT SuperMix Kit (New England Biolabs, catalog number E3010). All cDNA samples were diluted to 5 ng/μL prior to polymerase chain reaction (PCR) and stored at −20° C.

RT-qPCR Assay. Quantitative polymerase chain reactions (qPCR) were set up using Luna Universal qPCR Master Mix (New England Biolabs) on a CFX Connect Real-Time system (Bio Rad). For each qPCR, 15 ng of cDNA template and 10 uM of each gene-specific primer was used. A list of all gene-specific primers used is in Table 1. All primer pairs were tested for single band amplification optimal annealing temperature prior to qPCR. Thermal cycling consisted of an initial denaturation step at 95° C. for 5 min, followed by 40 cycles at 95° C. for 10 s, 53° C. for 10 s, and 72° C. for 30 s. Brown midrib4/FPGS (Zm00001eb404110) was used as a control reference gene. Data analysis was done on 3 biological replicates with two technical replicates using the delta delta Ct method as previously described (Manoli et al., “Evaluation of Candidate Reference Genes for qPCR in Maize,” Journal of Plant Physiology 169:807-815 (2012), which is hereby incorporated by reference in its entirety).

Cloning. All plasmids were verified using restriction enzyme digestions and whole-plasmid sequencing. All primers used in cloning are listed in Table 1. To create pUBII: PILS6-GS^{yellow} for transient expression, full-length minus the stop codon ZmPILS6 was amplified from maize root cDNA and cloned into pENTR/D-TOPO to generate pML2 a Gateway-compatible entry clone. pUBIL in pEN-L4-UBIL-R1 was obtained from (Karimi et al., “Building Blocks for Plant Gene Assembly,” Plant Physiology 145:1183-1191 (2007), which is hereby incorporated by reference in its entirety). GSyellow in pEN-R2L3-GSyellow was obtained from (Besbrugge et al., “GS^{yellow}, a Multifaceted Tag for Functional Protein Analysis in Monocot and Dicot Plants” Plant Physiol. 177:447-464 (2018), which is hereby incorporated by reference in its entirety). pML2 was recombined with pEN-L4-UBIL-R1, pEN-R2L3-GSyellow and pBb7m34GW (Besbrugge et al., “GS^{yellow}, a Multifaceted Tag for Functional Protein Analysis in Monocot and Dicot Plants” Plant Physiol. 177:447-464 (2018), which is hereby incorporated by reference in its entirety) using LR Clonase II Plus (Invitrogen) to generate pLD19. pLD19 was transformed into One Shot TOP10 E coli and screened on LB plates containing spectinomycin (100 μg/mL). Full length ZmPILS6 was amplified from maize root cDNA and cloned into pENTR/D-TOPO to generate pML1. pML1 was recombined into pAG426GPD-ccdb using LR Clonase to create pCC13 for yeast transport assays.

Transient expression and confocal microscopy. Agrobacterium tumefaciens strain GV3101 was transformed with pLD19 via the freeze-thaw method. Bacteria was grown in liquid LB medium containing spectinomycin (100 μg/mL) or kanamycin (100 μg/mL) overnight at 30° C. Cells were resuspended in MES Buffer (10 mM MES, 10 mM MgCl₂, PH 5.7) and cell density was adjusted to an OD600 of 0.3. Plasmid containing bacteria was mixed with P19 bacteria in a 1:1 ratio with a final OD600 of 0.4 (Zamore, “Plant RNAi: How a Viral Silencing Suppressor Inactivates siRNA. Current Biology 14: R198-R200 (2004), which is hereby incorporated by reference in its entirety). Bacteria was infiltrated into 4-week-old Nicotiana benthiamiana plants using a needleless syringe. Transient expression was observed 3 days post inoculation using a Zeiss LSM 700 confocal microscope. CFP fluorescent signals were captured at 485 nm after excitation of 405 nm. GS^{yellow} signals were captured between 525-575 nm after excitation of 514 nm. Transient assays were repeated in triplicate.

Auxin transport assays in yeast. Transport assays of radiolabeled indole-3-acetic acid (³H-IAA) were performed as previously described (Michniewicz, et al., “Transporter of IBA1 Links Auxin and Cytokinin to Influence Root Architecture,” Developmental Cell 50:599-609.e4 (2019), which is hereby incorporated by reference in its entirety). Briefly, pCC13 was transformed into Saccharomyces cerevisiae strain JBY575. Single yeast colonies were grown in liquid-URA selective media overnight at 30° C. Yeast were resuspended in 0.1M MES (pH 4.6) with 2% dextrose to an OD600 of 5. 100 μL of resuspended cells were added to a 1.5 mL Eppendorf tube and added 100 μL of 50 nM 3H-IAA and incubated at RT for 30 minutes. Yeast cells were centrifuged for 15 seconds before removing the supernatant and washing 3 times with MES solution. Cells were resuspended in 100 μL of MES buffer and ³H-IAA was measured using a scintillation counter. For each plasmid expressing yeast, 12 biological replicates were statistically analyzed using a t test.

In vivo auxin transport assays. Auxin accumulation assays were performed as previously described (Strader and Bartel, “The Arabidopsis PLEIOTROPIC DRUG RESISTANCE8/ABCG36 ATP Binding Cassette Transporter Modulates Sensitivity to the Auxin Precursor Indole-3-Butyric Acid,” The Plant Cell 21:1992-2007 (2009), which is hereby incorporated by reference in its entirety). Maize seedlings were grown for 6 days and dissected into four regions as previously described (McReynolds, et al., “Temporal and Spatial Auxin Responsive Networks in Maize Primary Root,” Quantitative Plant Biology 3: e21 (2022); Marcon et al., “A High-Resolution Tissue-Specific Proteome and Phosphoproteome Atlas of Maize Primary Roots Reveals Functional Gradients Along the Root Axes,” Plant Physiol. 168:233-246 (2015), each of which is hereby incorporated by reference in its entirety), designated the meristematic zone (“MZ”), elongation zone (“EZ”), cortical parenchyma and epidermis (“cortex”), and vasculature (“stele”). Sections were equilibrated in 40 μL of uptake buffer (20 mM MES, 10 mM sucrose, and 0.5 mM CaSO4, pH 5.6) for 30 minutes at room temperature (RT). 40 μL of 50 nM 3H-IAA was added to each sample and incubated at RT for 1 hour. Samples were rinsed 3 times with 80 L of uptake buffer. 3H-IAA in tissue samples was measured using a scintillation counter as previously described (Strader and Bartel, “The Arabidopsis PLEIOTROPIC DRUG RESISTANCE8/ABCG36 ATP Binding Cassette Transporter Modulates Sensitivity to the Auxin Precursor Indole-3-Butyric Acid,” The Plant Cell 21:1992-2007 (2009), which is hereby incorporated by reference in its entirety). Samples were moved to scintillation vials containing CytoScint (MP Biomedicals) and disintegrations per minute (DPM) were measured using a scintillation counter (Beckman Coulter LS-6500).

Indole-3-acetic acid measurements. Five-day-old B73 primary roots were hand dissected into meristematic zone (MZ), elongation zone (EZ), cortex, and stele and pooled to reach 200 mg per root region and replicate. The tissue was flash frozen and ground into a uniform powder using a mortar and pestle under liquid nitrogen. Extraction and measurements were done as previously described using isopropanol: water: HCl (2:1:2) as the extraction buffer (Müller and Munné-Bosch, “Rapid and Sensitive Hormonal Profiling of Complex Plant Samples by Liquid Chromatography Coupled to Electrospray Ionization Tandem Mass Spectrometry,” Plant Methods 7:37 (2011), which is hereby incorporated by reference in its entirety) with 50 mg of ground root tissue per replicate. Extracts were analyzed via liquid-chromatography mass spectrometry (LC-MS) on a Thermo Dioned Ultimate 3000+Q-Exactive Focus run with an internal indole-3-acetic acid (IAA) sample. For each sample, 5-6 biological replicates were analyzed (Dataset S2 in Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety).

Proteomics. Protein extraction and peptide generation. Protein extraction and peptide digestion was performed as previously described (Song et al., “Heterotrimeric G-Protein-Dependent Proteome and Phosphoproteome in Unstimulated Arabidopsis Roots,” Proteomics 18: e1800323 (2018), which is hereby incorporated by reference in its entirety). Briefly, total protein was extracted from 1 μg of 5-day-old primary root tissue (W22 and pils6-1) using the phenol-FASP method as previously detailed (Song et al., “Assessment and Refinement of Sample Preparation Methods for Deep and Quantitative Plant Proteome Profiling,” Proteomics 18: e1800220 (2018); Song et al., “Quantitative Profiling of Protein Abundance and Phosphorylation State in Plant Tissues Using Tandem Mass Tags” in Plant Proteomics: Methods and Protocols, Methods in Molecular Biology., J. V. Jorrin-Novo, L. Valledor, M. A. Castillejo, M.-D. Rey, Eds., pp. 147-156 (Springer US, 2020), each of which is hereby incorporated by reference in its entirety). Peptide digestion was done with trypsin and Lys-C as previously described (Song et al., “Assessment and Refinement of Sample Preparation Methods for Deep and Quantitative Plant Proteome Profiling,” Proteomics 18: e1800220 (2018); Song et al., “Quantitative Profiling of Protein Abundance and Phosphorylation State in Plant Tissues Using Tandem Mass Tags” in Plant Proteomics: Methods and Protocols, Methods in Molecular Biology, J. V. Jorrin-Novo, L. Valledor, M. A. Castillejo, M.-D. Rey, Eds., pp. 147-156 (Springer US, 2020), each of which is hereby incorporated by reference in its entirety. Resulting peptides were desalted using 50 mg Sep-Pak C18 cartridges (Waters), dried using a vacuum centrifuge, and resuspended in 0.1% formic acid. Peptide amounts were quantified using the Pierce BCA Protein assay kit

Tandem Mass Tag (TMT) Labeling. TMTpro™ 16plex labeling reagents (ThermoFisher, Lot VB294909) were used to label at a TMT: peptide ratio of 0.2:1 as described in (Song et al., “Quantitative Profiling of Protein Abundance and Phosphorylation State in Plant Tissues Using Tandem Mass Tags” in Plant Proteomics: Methods and Protocols, Methods in Molecular Biology., J. V. Jorrin-Novo, L. Valledor, M. A. Castillejo, M.-D. Rey, Eds., pp. 147-156 (Springer US, 2020), which is hereby incorporated by reference in its entirety). After 2 hours incubation at room temperature the labeling reaction was quenched with hydroxylamine. Next, the 16 samples were mixed together and stored at −80° C.

LC-MS/MS. An Agilent 1260 quaternary HPLC was used to deliver a flow rate of ˜600 nL min-1 via a splitter. All columns were packed in house using a Next Advance pressure cell, and the nanospray tips were fabricated using a fused silica capillary that was pulled to a sharp tip using a laser puller (Sutter P-2000). 10 μg of TMT-labeled peptides were loaded onto 10 cm capillary columns packed with 5 μM Zorbax SB-C18 (Agilent), which was connected using a zero dead volume 1 μm filter (Upchurch, M548) to a 5 cm long strong cation exchange (SCX) column packed with 5 μm PolySulfoethyl (PolyLC). The SCX column was then connected to a 20 cm nanospray tip packed with 2.5 μM C18 (Waters). The 3 sections were joined and mounted on a Nanospray Flex ion source (Thermo) for on-line nested peptide elution. A new set of columns was used for every sample. Peptides were eluted from the loading column onto the SCX column using a 0 to 80% acetonitrile gradient over 60 minutes. Peptides were then fractionated from the SCX column using a series of 1 8 salt steps (ammonium acetate) for the non-modified proteome and phosphoproteome analysis, respectively. For these analyses, buffers A (99.9% H₂O, 0.1% formic acid), B (99.9% ACN, 0.1% formic acid), C (100 mM ammonium acetate, 2% formic acid), and D (2 M ammonium acetate, 2% formic acid) were utilized. For each salt step, a 150-minute gradient program comprised of a 0-5 minute increase to the specified ammonium acetate concentration, 5-10 minutes hold, 10-14 minutes at 100% buffer A, 15-100 minutes 15-30% buffer B, 100-121 minutes 30-45% buffer B, 120-140 minutes 45-80% buffer B, 140-144 minutes 80% buffer B, and 145-150 minutes buffer A was employed. minutes buffer A was employed.

Eluted peptides were analyzed using a Thermo Scientific Q-Exactive Plus high-resolution quadrupole Orbitrap mass spectrometer, which was directly coupled to the HPLC. Data dependent acquisition was obtained using Xcalibur 4.0 software in positive ion mode with a spray voltage of 2.20 kV and a capillary temperature of 275° C. and an RF of 60. MS1 spectra were measured at a resolution of 70,000, an automatic gain control (AGC) of 3e6 with a maximum ion time of 50 ms and a mass range of 350-1500 m/z. Up to 15 MS2 were triggered at a resolution of 35,000 with a fixed first mass of 110 m/z. An AGC of 2e5 with a maximum ion time of 120 ms, an isolation window of 1.3 m/z, and a normalized collision energy of 31. Charge exclusion was set to unassigned, 1, 5-8, and >8. MS1 that triggered MS2 scans were dynamically excluded for 30 s.

Proteomics Data Analysis. The raw spectra were analyzed using MaxQuant version 1.6.14.0 (Tyanova et al., “The MaxQuant Computational Platform for Mass Spectrometry-Based Shotgun Proteomics,” Nat Protoc 11:2301-2319 (2016), which is hereby incorporated by reference in its entirety). Spectra were searched using the Andromeda search engine in MaxQuant (Cox, et al., “Andromeda: A Peptide Search Engine Integrated into the MaxQuant Environment,” J. Proteome Res. 10:1794-1805 (2011), which is hereby incorporated by reference in its entirety) against the Zea mays B73 protcome file entitled “Zea_mays.AGPv4.pep.all” and was complemented with reverse decoy sequences and common contaminants by MaxQuant.

Carbamidomethyl cysteine was set as a fixed modification while methionine oxidation and protein N-terminal acetylation were set as variable modifications. The sample type was set to “Reporter Ion MS2” with “16plex TMT selected for both lysine and N-termini”. Digestion parameters were set to “specific” and “Trypsin/P; LysC”. Up to two missed cleavages were allowed. A false discovery rate, calculated in MaxQuant using a target-decoy strategy (Elias and Gygi, “Target-Decoy Search Strategy for Increased Confidence in Large-Scale Protein Identifications by Mass Spectrometry,” Nat Methods 4:207-214 (2007), which is hereby incorporated by reference in its entirety), less than 0.01 at both the peptide spectral match and protein identification level was required. The ‘second peptide’ option identify co-fragmented peptides was not used. The match between runs feature of MaxQuant was not utilized.

Statistical analysis on the MaxQuant output was performed using the TMT-NEAT Analysis Pipeline (Clark et al., “Integrated Omics Networks Reveal the Temporal Signaling Events of Brassinosteroid Response in Arabidopsis,” Nat. Commun. 12:5858 (2021), which is hereby incorporated by reference in its entirety). Differential expression (DE) was determined using a q-value <or =0.05 (Dataset S3 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety). Upset plots were generated in R as previously described (McReynolds et al., “Temporal and Spatial Auxin Responsive Networks in Maize Primary Root,” Quantitative Plant Biology 3: e21 (2022), which is hereby incorporated by reference in its entirety). Proteins with altered abundance were hierarchically clustered based on their protein abundance under mock or IAA treatments and plotted as heatmaps using the Morpheus software from the Broad Institute.

Gene ontology analysis. Gene ontology (GO) enrichment analysis was performed at PANTHER using the Zea mays B73 inbred reference genome as previously described (McReynolds, et al., “Temporal and Spatial Auxin Responsive Networks in Maize Primary Root,” Quantitative Plant Biology 3: e21 (2022), which is hereby incorporated by reference in its entirety). The DE proteins were tested for statistical over-representation of GO terms using Fisher's exact test and an FDR correction using the Benjamini-Hochberg method with a cutoff of 0.05 (Dataset S4 of Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety). Significant GO Biological Process terms were plotted against the genotype/treatments using a multidimensional dot plot in R (Bonnot et al., “A Simple Protocol for Informative Visualization of Enriched Gene Ontology Terms,” BIO-PROTOCOL 9 (2019), which is hereby incorporated by reference in its entirety).

Weighted Gene Co-expression Network Analysis (WGCNA). A ZmPILS6 co-expression network was created using protein expression from mock and IAA treated samples. 12,265 globally detected protein abundances (Dataset S3 of Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety) were used as input to the signed WGCNA network construction using the WGCNA v1.70-3 package in R (Langfelder and Horvath, “WGCNA: An R Package for Weighted Correlation Network Analysis,” BMC Bioinformatics 9:559 (2008), which is hereby incorporated by reference in its entirety). In WGCNA networks, power was set to 9, minModuleSize was set to 100, and initial clusters were merged on eigengenes (Dataset S5 of Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety). The mergeCutHeight value was set to 0.20 across all networks. Visualization of the ZmPILS6 co-expression network (Dataset S6 of Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety) was visualized in Cytoscape version 3.9.1 using the organic layout.

Phylogenetic analysis. Sequences for Arabidopsis PILS proteins and maize PILS proteins were obtained from PLAZA (Van Bel et al., “PLAZA 5.0: Extending the Scope and Power of Comparative and Functional Genomics in Plants,” Nucleic Acids Research 50: D1468-D1474 (2022), which is hereby incorporated by reference in its entirety) to construct the phylogenetic tree.

Data availability. Raw proteomics data (Dataset S3 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety) have been deposited on MassIVE with accession number MSV000092438, which is hereby incorporated by reference in its entirety.

### Example 2—Loss of ZmPILS6 Negatively Impacts Crown Root Architecture

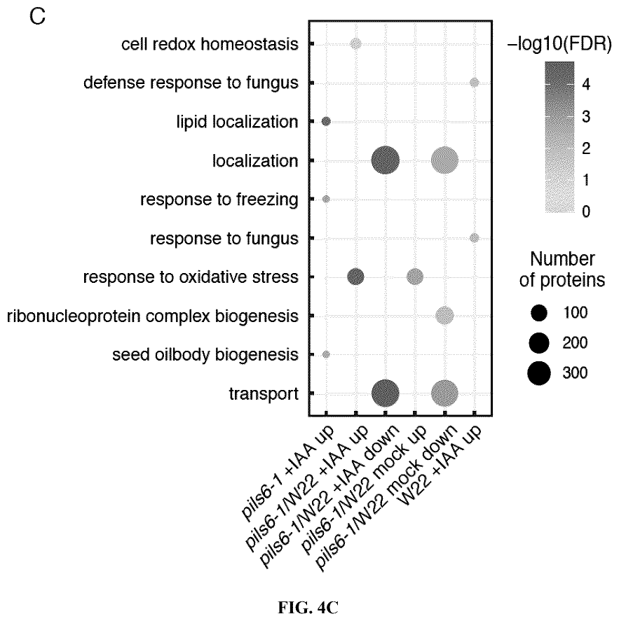

To identify candidate genes for a reverse genetics screen on auxin transporters in maize roots, we utilized available quantitative proteomics data for maize (Walley et al., “Integration of Omic Networks in a Developmental Atlas of Maize,” Science 353:814-818 (2016), which is hereby incorporated by reference in its entirety). A query of the maize expression atlas identified ZmPILS6 (Zm00001eb149720, also known as GRMZM2G050089 and Zm00001d043083) as an annotated auxin efflux carrier with enriched expression in unpollinated silks, internodes, and primary roots (FIG. 5 and Supp Table 2 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety) and as set forth in Table 2 below.

TABLE 2
ZmPILS Expression Data
Gene Number for Table 2

| Gene | Gene ID_V3 | Gene ID_V5 |
|---|---|---|
| 1 | GRMZM2G030125 | Zm00001d051044 |
| 2 | GRMZM2G050089 | Zm00001d043083 |
| 3 | GRMZM2G070563 | Zm00001d007109 |
| 4 | GRMZM2G072632 | Zm00001d024842 |
| 5 | GRMZM2G331322 | Zm00001d007499 |
| 6 | GRMZM2G475148 | Zm00001d020488 |
| 7 | GRMZM2G112598 | Zm00001d040149 |
| 8 | GRMZM2G043254 | Zm00001d020953 |

| | | | | | Leaf | | | | | Root - | | | Root - |
|---|---|---|---|---|---|---|---|---|---|---|---|---|---|
| | Vegetative | 6-7 | 7-8 | Juvenile | Zone 1 | Leaf | Leaf | | Primary | Meristem | Root | Root - | Elonga- |
| | Meristem | inter- | inter- | Leaf | (Symmet- | Zone 2 | Zone 3 | Mature | Root | Zone | Cortex | Stelle | tion Zone |
| Gene | 16-19 Day | node | node | Blade 3 | rical) | (Stomatal) | (Growth) | Leaf 8 | 5 Days | 5 Days | 5 Days | 5 Days | 5 Days |
| 1 | 0.00 | 32.01 | 0.00 | 0.00 | 26.69 | 27.86 | 46.05 | 0.00 | 11.63 | 21.89 | 30.57 | 26.85 | 104.54 |
| 2 | 7.79 | 36.01 | 17.70 | 0.00 | 0.00 | 0.00 | 9.69 | 0.00 | 25.56 | 0.00 | 4.93 | 0.00 | 4.98 |

| | | | | | | | | Ear | Ear | Ear | | |
|---|---|---|---|---|---|---|---|---|---|---|---|---|
| Secondary | | | | | B73 | B73 | | Primor- | Primor- | Primor- | | |
| Root | | Anther | Anther | W23 | Mature | Germ | Female | dium | dium | dium | Silk | Endosperm |
| 7-8 Days | Tassel | 1.0 mm | 2.0 mm | Pollen | Pollen | Pollen | Spikelet | 1 mm | 2-4 mm | 6-8 mm | (unpollinated) | 8 DAP |
| 7.10 | 9.97 | 3.80 | 0.00 | 19.06 | 13.22 | 12.93 | 120.57 | 0.00 | 26.08 | 20.17 | 26.08 | 42.01 |
| 0.00 | 0.00 | 0.00 | 0.00 | 19.76 | 0.00 | 0.00 | 0.00 | 0.00 | 0.00 | 0.00 | 64.15 | 8.95 |

| | | | Endo- | | | | | | | |
|---|---|---|---|---|---|---|---|---|---|---|
| | Endo- | Endo- | sperm | Pericarp\| | | | Germ | | | |
| | sperm | sperm | Crown | Aleurone | Embryo | Embryo | Kernels | | Uni- | |
| | 10 | 12 | 27 | 27 | 20 | GS | 2 | | quely | gene_ |
| Gene | DAP | DAP | DAP | DAP | DAP | DAP | DAI | Peptides | Ided | biotype |
| 1 | 70.88 | 61.20 | 0.00 | 13.41 | 0.00 | 0.00 | 0.00 | SLLHEAEWPGMVDK | TRUE | protein_ |
| | | | | | | | | | | coding |
| 2 | 0.00 | 0.00 | 0.00 | 13.88 | 0.00 | 0.00 | 0.00 | YPMNTNSSTVPENEPLLSAGEV | TRUE | protein_ |
| | | | | | | | | QK\|MLAPPPGQTFDGSEEDGIP | | coding |
| | | | | | | | | IK\|ASGENTVPQVGKYPMNTNS | | |
| | | | | | | | | STVPENEPLLSAGEVQK | | |

| | | MapMan_{—} | MapMan_{—} | | MapMan_Closest_{—} | Name - | |
|---|---|---|---|---|---|---|---|
| Gene | gene_set | BinCode | BinName | MapMan_Description | Arab_Match | Source | Syntany? |
| 1 | filtered_{—} | 34.99 | transport.misc | highly similar to (575) AT1G71090 \| Symbols: \| | AT1G71090 | | NA |
| | set | | | auxin efflux carrier family protein \| | | | |
| | | | | chr1: 26812551-26813924 FORWARDhighly | | | |
| | | | | similar to (784) loc_os08g09190 12008.m05054 | | | |
| | | | | protein auxin Efflux Carrier family protein, | | | |
| | | | | expressed seq = cds; | | | |
| | | | | coord = 4:135312049 . . . 135314646:−1; | | | |
| | | | | parent gene = GRMZM2G030125 | | | |
| 2 | filtered_{—} | 34.99 | transport.misc | highly similar to (508) AT5G01990 \| Symbols: \| | AT5G01990 | pin14 - | Syntenic |
| | set | | | auxin efflux carrier family protein \| chr5: | | MaizeGDB | |
| | | | | 377373-379600 REVERSEhighly similar to | | curated | |
| | | | | (729) loc_os01g60230 12001.m150756 protein | | | |
| | | | | auxin hydrogen symporter, putative, expressed | | | |
| | | | | seq = cds; | | | |
| | | | | coord = 3:185756306 . . . 185761091:−1; | | | |
| | | | | parent gene = GRMZM2G050089\| | | | |

| | | | | Female | | | | | | | | | |
|---|---|---|---|---|---|---|---|---|---|---|---|---|---|
| | | | | Spikelet | | Ear | Ear | | Endo- | | | | |
| | | | | Collected | | Primor- | Primor- | Endo- | sperm | Pericarp/ | | | Germinatin |
| | | Gene | | on day | | dium | dium | sperm | Crown | Aleurone | Embryo | Embryo | Kernels |
| Gene | Gene Set | biotype | Syntany? | as silk | Silk | 2-4 mm | 6-8 mm | 12 DAP | 27 DAP | 27 DAP | 20 DAP | 38 DAP | 2 DAI |
| 1 | filtered_{—} | protein_{—} | NA | 61.26 | 33.63 | 49.05 | 52.11 | 17.07 | 8.96 | 30.29 | 39.27 | 14.98 | 24.97 |
| | set | coding | | | | | | | | | | | |
| 2 | filtered_{—} | protein_{—} | Syntenic | 12.19 | 26.53 | 16.28 | 18.36 | 8.78 | 2.01 | 9.08 | 13.66 | 13.54 | 22.96 |
| | set | coding | | | | | | | | | | | |
| 3 | filtered_{—} | protein_{—} | Non- | 0.05 | 0.21 | 0.22 | 0.25 | 0.05 | 0.00 | 0.10 | 0.30 | 0.39 | 0.10 |
| | set | coding | Syntenic | | | | | | | | | | |
| 4 | filtered_{—} | protein_{—} | Non- | 0.15 | 0.21 | 0.13 | 0.10 | 0.02 | 0.03 | 0.05 | 0.16 | 0.12 | 0.05 |
| | set | coding | Syntenic | | | | | | | | | | |
| 5 | working_{—} | protein_{—} | Non- | 0.00 | 0.18 | 0.19 | 0.13 | 0.01 | 0.03 | 0.06 | 0.07 | 0.15 | 0.04 |
| | set | coding | Syntenic | | | | | | | | | | |
| 6 | filtered_{—} | protein_{—} | Non- | 0.03 | 0.07 | 0.17 | 0.06 | 0.01 | 0.00 | 0.00 | 0.09 | 0.10 | 0.08 |
| | set | coding | Syntenic | | | | | | | | | | |
| 7 | filtered_{—} | protein_{—} | Non- | 0.00 | 0.06 | 0.05 | 0.10 | 0.02 | 0.00 | 0.01 | 0.13 | 0.03 | 0.04 |
| | set | coding | Syntenic | | | | | | | | | | |
| 8 | | | | | | | | | | | | | |

| | | | Leaf | | | | | | Root - | Root - | | | |
|---|---|---|---|---|---|---|---|---|---|---|---|---|---|
| | 6-7 | 7-8 | Zone 1 | Leaf | Leaf | | Vegetative | B73 | Meristem | Elonga- | Root - | Primary | Secondary |
| | inter- | inter- | (Symmet- | Zone 2 | Zone 3 | Mature | Meristem | Mature | Zone | tion Zone | Cortex | Root | Root |
| Gene | node | node | rical) | (Stomatal) | (Growth) | Leaf 8 | 16-19 Day | Pollen | 5 Days | 5 Days | 5 Days | 5 Days | 7-8 Days |
| 1 | 82.64 | 78.49 | 51.01 | 50.70 | 70.05 | 24.71 | 30.18 | 1.07 | 29.24 | 49.50 | 15.62 | 44.39 | 41.92 |
| 2 | 26.72 | 16.83 | 21.00 | 25.60 | 42.09 | 12.48 | 15.00 | 2.18 | 20.34 | 36.63 | 7.35 | 30.22 | 29.34 |
| 3 | 0.00 | 0.02 | 0.27 | 0.00 | 0.09 | 1.39 | 0.32 | 0.02 | 0.10 | 0.09 | 0.57 | 0.07 | 0.13 |
| 4 | 0.08 | 0.12 | 0.22 | 0.26 | 0.10 | 1.11 | 0.19 | 0.01 | 0.05 | 0.16 | 0.02 | 0.06 | 0.09 |
| 5 | 0.03 | 0.00 | 0.12 | 0.16 | 0.04 | 0.86 | 0.09 | 0.00 | 0.04 | 0.04 | 0.12 | 0.06 | 0.03 |
| 6 | 0.06 | 0.04 | 0.14 | 0.09 | 0.08 | 0.72 | 0.38 | 0.00 | 0.00 | 0.05 | 0.23 | 0.05 | 0.10 |
| 7 | 0.03 | 0.08 | 0.08 | 0.05 | 0.06 | 0.59 | 0.17 | 0.02 | 0.01 | 0.01 | 0.00 | 0.02 | 0.02 |
| 8 | | | | | | | | | | | | | |

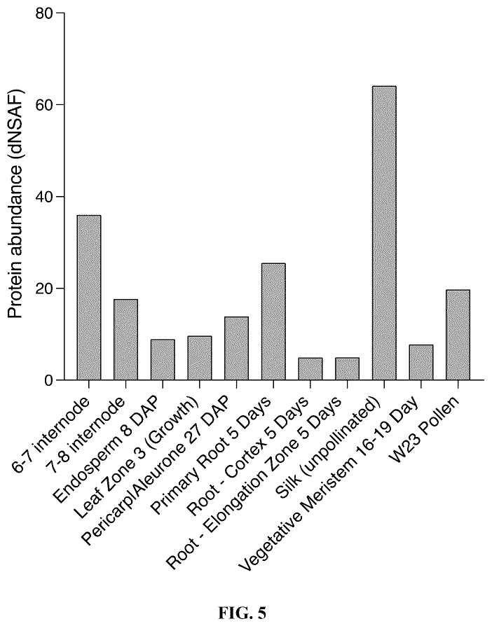

ZmPILS6 is the ortholog of Arabidopsis PILS6 based on a phylogenetic analysis of full-length PILS proteins sequences from TAIR and MaizeGDB (FIG. 6). An alignment of PILS6 proteins from Arabidopsis and maize indicates that there are 10 conserved transmembrane domains, a divergent amino acid sequences in the N-terminus and middle region (FIG. 7).

To study the function of ZmPILS6, three Mu transposon insertion alleles were identified from maize stock center and designated as pils6-1 (mu1047700), pils6-2 (mu1090629), and pils6-3 (mu-ill 221882.6) (FIG. 1A). Both pils6-1 and pils6-2 are W22 Uniform Mu lines with transposon insertions in the 5′ UTR, while pils6-3 is a B73 Mu Illumina line with a transposon insertion in the first exon (FIG. 1A). ZmPILS6 expression was examined in all three alleles and their respective inbred background using RT-qPCR and found to be significantly reduced in both pils6-1 and pils6-3 alleles, but not significantly changed in the pils6-2 allele (FIG. 1B). Based on this expression data, the pils6-2 allele was not used further in this study.

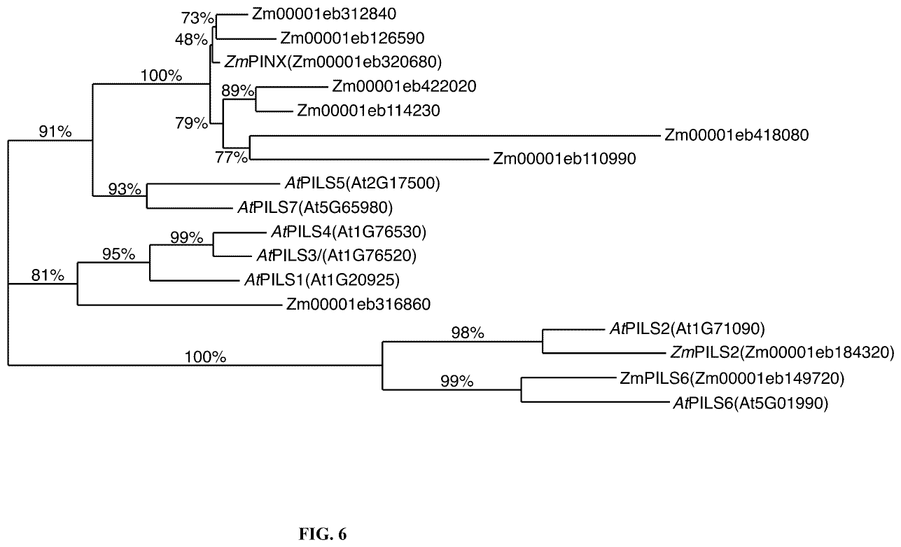

A phenotypic analysis of pils6 loss of function alleles indicates a role in maize root and shoot development (FIGS. 1C-J and FIG. 7). Compared to their inbred controls, both pils6-1 and pils6-3 primary roots are short (FIG. 1C). Exogenous auxin treatment can inhibit primary root growth (Draves, “Maize Seedling Growth and Hormone Response Assays Using the Rolled Towel Method,” Current Protocols 2: e562 (2022), which is hereby incorporated by reference in its entirety). To determine if auxin response is altered in pils6 roots, three-day-old seedlings were treated with mock solution or 10 μM indole-3-acetic acid (IAA) for four days and primary root lengths were measured (1C). Compared to W22 and B73, pils6-1 and pils6-3 roots were less sensitive to exogenous IAA treatment (FIG. 1D). The V7 stage crown root systems of pils6-1 and pils6-3 were reduced compared to their respective inbred controls, W22 and B73 (FIGS. 1E-H). Quantification of pils6 crown root traits indicated that both total root length (FIG. 1I) and root network area (FIG. 1J) were significantly reduced compared to the inbred controls. Loss of ZmPILS6 also led to a reduction in branch points and fewer root tips (FIG. 8, Dataset S1 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety).

TABLE 3
Statistics
T test statistics with unequal variance

| Average Value | W22 | pils6-1 | P-value | B73 | pils6-3 | P-value |
|---|---|---|---|---|---|---|
| Network Area | 9332.69 | 4254.28 | 0.00183 | 10005.36 | 6052.69 | 0.000525 |
| Average Diameter | 0.254 | 0.213 | 0.0577 | 0.275 | 0.33 | 0.0876 |
| Surface Area | 42023.81 | 17996.85 | 0.00360 | 5453.72 | 29188.29 | 0.000428 |
| Total Root Length | 1416.49 | 707.76 | 0.00082 | 1667.74 | 760.29 | 0.0015 |
| Perimeter | 2094.96 | 1140.42 | 0.00109 | 2141.55 | 1080.33 | 0.00397 |
| Volume | 142619.06 | 51529.28 | 0.0192 | 221012.38 | 157948.77 | 0.165 |
| Number of Root Tips | 627 | 346 | 0.0469 | 556 | 382 | 0.0400 |
| Number of Branch | 1799 | 780 | 0.003617 | 2755 | 827 | 0.0155 |
| Points | | | | | | |
| Median Diameter | 0.214 | 0.186 | 0.0664 | 0.205 | 0.228 | 0.388 |
| Maximum Diameter | 1.186 | 1.146 | 0.832 | 1.515 | 1.775 | 0.225 |

TABLE 4
Phenotypes of pils6-3 Mutant vs Wild Type Control

| | Total | | | | | | | | | |
|---|---|---|---|---|---|---|---|---|---|---|
| | Root | Average | Surface | Network | | | Number | Number | Median | Maximum |
| | Length | Diameter | Area | Area | Perimeter | Volume | of Root | of Branch | Diameter | Diameter |
| Genotype | (cm) | (cm) | (cm²) | (cm²) | (cm) | (cm³) | Tips | Points | (cm) | (cm) |
| B73 | 1790.14 | 0.25 | 7088.49 | 9712.77 | 2352.71 | 221779.12 | 595 | 3089 | 0.17 | 1.86 |
| B73 | 1960.35 | 0.24 | 7629.58 | 10041.33 | 2536.06 | 195255.58 | 691 | 3704 | 0.17 | 1.47 |
| B73 | 1378.71 | 0.27 | 3447.55 | 9261.92 | 1928.67 | 154150.5 | 459 | 1802 | 0.22 | 1.16 |
| B73 | 1541.76 | 0.34 | 3649.25 | 11005.4 | 1748.74 | 312864.31 | 479 | 2424 | 0.26 | 1.57 |
| pils6-3 | 593.65 | 0.31 | 21239.83 | 4486.04 | 856.62 | 104103.38 | 312 | 618 | 0.22 | 1.43 |
| pils6-3 | 1132.16 | 0.32 | 42591.16 | 8560.86 | 1564.86 | 218236.16 | 373 | 1265 | 0.22 | 2.09 |
| pils6-3 | 596.6 | 0.37 | 25786.63 | 5537.41 | 862.99 | 152126.79 | 321 | 583 | 0.26 | 2.19 |
| pils6-3 | 756.96 | 0.33 | 28912.64 | 5988.26 | 1090.96 | 165628.6 | 402 | 798 | 0.22 | 1.83 |
| pils6-3 | 802.17 | 0.36 | 33853.43 | 6681.95 | 1079.47 | 200075.11 | 458 | 893 | 0.26 | 1.69 |
| pils6-3 | 680.21 | 0.29 | 22746.02 | 5061.61 | 1027.06 | 107522.58 | 426 | 806 | 0.19 | 1.42 |
| W22 | 1613.45 | 0.29 | 54465.08 | 11899.69 | 2301.1 | 208187.68 | 607 | 1953 | 0.24 | 1.32 |
| W22 | 1297.69 | 0.22 | 32860.19 | 7865.64 | 2077.18 | 89230.31 | 626 | 1523 | 0.19 | 0.92 |
| W22 | 1618.82 | 0.22 | 41955.01 | 9546.64 | 2502.24 | 121488.82 | 976 | 2084 | 0.19 | 1.04 |
| W22 | 1055.92 | 0.25 | 30202.11 | 6900.41 | 1595.36 | 94991.07 | 343 | 1219 | 0.21 | 1.35 |
| W22 | 1496.56 | 0.29 | 50636.67 | 10451.07 | 1998.94 | 199197.41 | 584 | 2214 | 0.24 | 1.3 |
| pils6-1 | 549.78 | 0.21 | 13527.98 | 3267.13 | 897.61 | 38303.7 | 280 | 584 | 0.19 | 1.61 |
| pils6-1 | 953.89 | 0.23 | 25089.66 | 5194.51 | 1341.11 | 81272.95 | 363 | 1651 | 0.19 | 1.42 |
| pils6-1 | 356.04 | 0.18 | 7531.26 | 2007.84 | 648.8 | 15436.66 | 168 | 223 | 0.16 | 0.72 |
| pils6-1 | 1130.92 | 0.24 | 32152.89 | 7307.74 | 1720.27 | 100974.01 | 466 | 1317 | 0.21 | 1.75 |
| pils6-1 | 630.26 | 0.22 | 16126.68 | 4172.4 | 1106.82 | 44169.94 | 390 | 470 | 0.19 | 0.82 |
| pils6-1 | 711.83 | 0.19 | 15559.35 | 3922.24 | 1233.16 | 35617.17 | 436 | 666 | 0.17 | 0.86 |
| pils6-1 | 621.58 | 0.22 | 15990.13 | 3908.08 | 1035.17 | 44930.52 | 320 | 547 | 0.19 | 0.84 |

These root characteristics are important traits of agricultural relevance (Seethepalli et al., “RhizoVision Explorer: Open-Source Software for Root Image Analysis and Measurement Standardization,” AoB PLANTS 13: plab056 (2021); Lynch, “Root Phenotypes for Improved Nutrient Capture: an Underexploited Opportunity for Global Agriculture,” New Phytologist 223:548-564 (2019); Lynch, “Rightsizing Root Phenotypes for Drought Resistance,” J. Exp. Bot. 69:3279-3292 (2018); Postma et al., “The Optimal Lateral Root Branching Density for Maize Depends on Nitrogen and Phosphorus Availability,” Plant Physiology 166:590-602 (2014); Zhan et al., “Reduced Lateral Root Branching Density Improves Drought Tolerance in Maize,” Plant Physiology 168:1603-1615 (2015); Zhan and Lynch, “Reduced Frequency of Lateral Root Branching Improves N Capture from Low-N Soils in Maize,” J. Exp. Bot. 66:2055-2065 (2015), each of which is hereby incorporated by reference in its entirety). Reducing PILS6 expression in maize also negatively impacts shoot height

(FIGS. 9A-E). Greenhouse grown V7 stage pils6-1 and pils6-3 plants exhibit short plant stature compared to their inbred controls (FIGS. 9A-E). Altogether these data indicate that ZmPILS6 is a positive regulator of plant growth and development in maize. This finding contrasts with the known roles of the PILS6 ortholog in Arabidopsis, which is a negative regulator of root and shoot morphogenesis (Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019), which is hereby incorporated by reference in its entirety). This may be due to divergences PILS6 sequences between these two orthologs (FIG. 7) or differences in downstream consequences of reducing PILS6 levels.

### Example 3—ZmPILS6 is Required for Lateral Root Formation

In Arabidopsis, PILS2 and PILS5 are known to regulate lateral root formation (Barbez et al., “A novel Putative Auxin Carrier Family Regulates Intracellular Auxin Homeostasis in Plants,” Nature 485:119-122 (2012), which is hereby incorporated by reference in its entirety). Both pils2 and pils5 in Arabidopsis have increased lateral root density (Barbez et al., “A novel Putative Auxin Carrier Family Regulates Intracellular Auxin Homeostasis in Plants,” Nature 485:119-122 (2012), which is hereby incorporated by reference in its entirety). In order to determine if ZmPILS6 plays a role in this same process, lateral root formation was examined in six-day-old roots of pils6-1 and pils6-3 using an established Feulgen staining protocol (Hoecker et al., “Manifestation of Heterosis During Early Maize (Zea mays L.) Root Development,” Theor. Appl. Genet. 112:421-429 (2006), which is hereby incorporated by reference in its entirety). Compared to wild-type W22 (FIG. 2A), the mature zone of pils6-1 roots exhibited fewer lateral roots (FIG. 2B). This same phenotype was also observed in pils6-3 (FIG. 2D) compared to B73 (FIG. 2C). Both knock-down alleles had a reduction in lateral root primordia density, which was determined as the number of primordia divided by the length of the primary root (FIG. 2E,F). These results demonstrate that ZmPILS6 is a positive regulator of lateral initiation in maize, which contrasts with Arabidopsis PILS2 and PILS5.

### Example 4—ZmPILS6 can Transport Indole-3-Acetic Acid

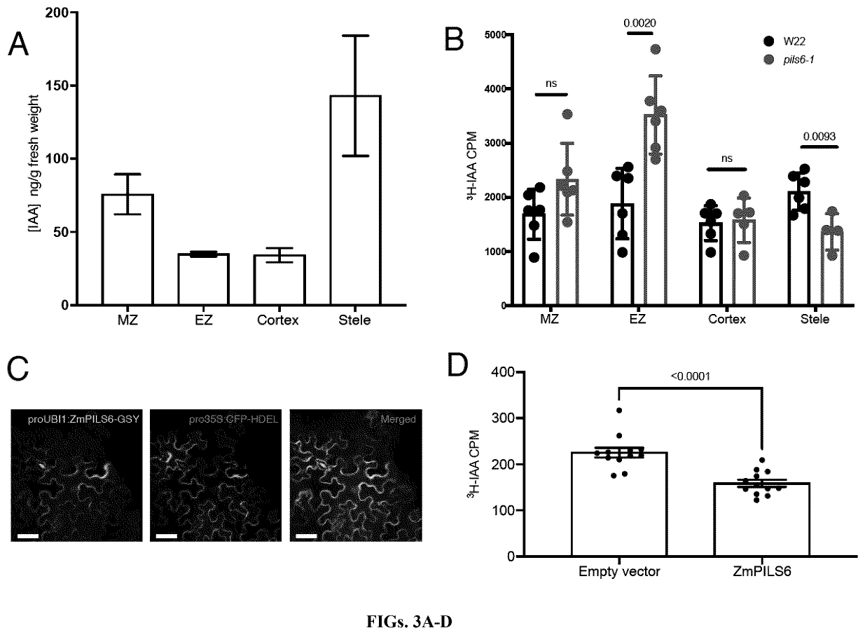

Auxin distribution and response is non-uniform across the maize primary root (McReynolds et al., “Temporal and Spatial Auxin Responsive Networks in Maize Primary Root,” Quantitative Plant Biology 3: e21 (2022); Nishimura, et al., “Immunohistochemical Observation of Indole-3-Acetic Acid at the IAA Synthetic Maize Coleoptile Tips,” Plant Signaling & Behavior 6:2013 (2011); Jansen et al., “Phloem-Associated Auxin Response Maxima Determine Radial Positioning of Lateral Roots in Maize,” Philos. Trans. R. Soc. Lond. B. Biol. Sci. 367:1525-1533 (2012), each of which is hereby incorporated by reference in its entirety). To further investigate this pattern endogenous levels of IAA were quantified using LC-MS on five-day-old primary roots hand dissected into four regions: the meristematic zone (MZ), elongation zone (EZ), cortex, and stele (which were obtained from the differentiation zone) (FIG. 3A, Dataset S2 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety). Endogenous IAA levels were highest in the stele and meristematic zone, while the elongation zone and cortex contained comparable amounts of IAA (FIG. 3A). This asymmetric pattern could be established due to tissue specific auxin biosynthesis, transport and/or metabolism. To determine if ZmPILS6 can contribute to IAA transport in primary roots, a modified radiolabeled 3H-IAA assay was performed using wild-type and pils6-1 dissected roots. In the absence of ZmPILS6, 3H-IAA hyperaccumulates in the elongation zone (FIG. 3B). Conversely, 3H-IAA hypo-accumulates in the stele of pils6-1 roots compared to W22 (FIG. 3B). These data suggest that ZmPILS6 is required for normal auxin transport properties in primary maize roots.

ZmPILS6 is localized to the endoplasmic reticulum in Nicotiana benthimiana cells (FIG. 3C), which is consistent with the known subcellular localization of Arabidopsis PILS orthologs. To establish if ZmPILS6 is capable of directly transporting 3H-IAA, full-length ZmPILS6 protein was expressed in yeast. In these assays we observed a significant decrease in 3H-IAA accumulation in yeast cells expressing ZmPILS6 compared to the empty vector control, which is consistent with ZmPILS6 efflux activity in yeast (FIG. 3D). Altogether these data suggest that ZmPILS6 is capable of directly effluxing IAA within root tissues, likely across the ER membrane. Furthermore, the spatial dynamics of IAA efflux in maize primary roots is in part conferred by ZmPILS6. In pils6-1 roots, auxin efflux is specifically altered within the elongation zone and stele of the primary root, but normal within the meristematic zone.

### Example 5—Loss of ZmPILS6 Impacts Auxin Responsive Proteome Remodeling

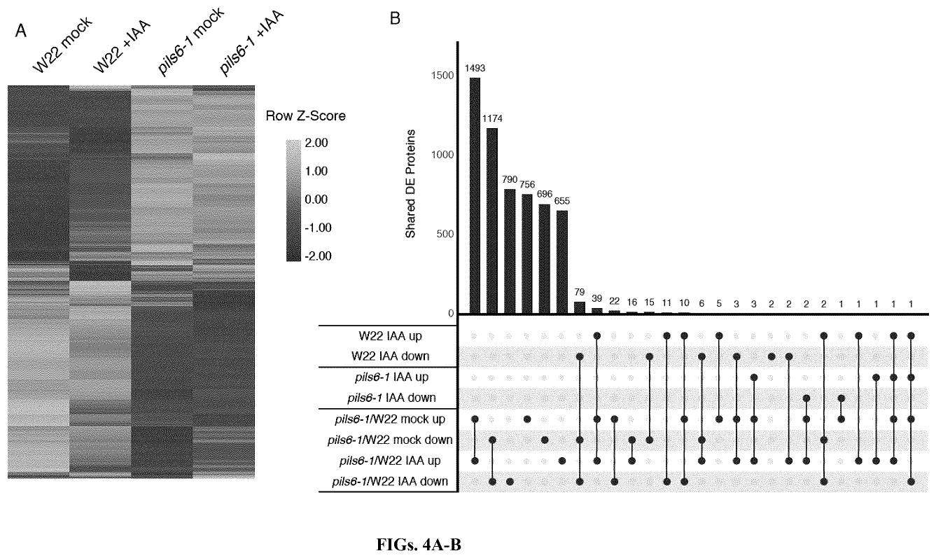

Auxin signaling can rapidly and extensively impact proteome dynamics in numerous angiosperm organs (Pu et al., “Quantitative Early Auxin Root Proteomics Identifies GAUT10, a Galacturonosyltransferase, as a Novel Regulator of Root Meristem Maintenance,” Mol Cell Proteomics 18:1157-1170 (2019); Clark et al., “Auxin Induces Widespread Proteome Remodeling in Arabidopsis Seedlings,” Proteomics 19: e1900199 (2019); Friml et al., “ABP1-TMK Auxin Perception for Global Phosphorylation and Auxin Canalization,” Nature 609:575-581 (2022); Singh et al., “Auxin-Responsive (Phospho) Protcome Analysis Reveals Key Biological Processes and Signaling Associated with Shoot-Borne Crown Root Development in Rice,” Plant Cell Physiol. 63:1968-1979 (2023), each of which is hereby incorporated by reference in its entirety). Proteomic profiling of pils6-1 and W22 roots was performed in the presence and absence of auxin treatment (10 μM IAA) to identify protein expression patterns in the mutant (Dataset S3 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety). In W22, 178 proteins exhibited altered abundance in response to IAA treatment. In comparison, auxin responsiveness in pils6-1 is reduced with only 9 auxin-responsive proteins. Overall, greater changes in proteome composition were observed between pils6-7 and W22 in the absence of auxin, whereby thousands of proteins were increased or decreased compared to W22 (FIG. 4A). The protein abundance of both PILS6 and its ortholog, PILS2, were down in pils6-1 compared to W22 (Dataset S3 of Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety). Notably, a comparison of the auxin-responsive proteins between the genotypes uncovered a greater influence on the proteome. Specifically, 1493 proteins are constitutively increased in pils6-1 compared to W22 following auxin treatment, while 1174 proteins remained repressed in pils6-7 roots in the presence of auxin (FIG. 4B). In addition, discordant protein abundance changes were observed between auxin-responsive proteins in pils6-1 and W22 (FIG. 4B). For example, 11 proteins which are auxin-induced in W22 are auxin-repressed in pils6-1. The observed decrease in auxin signaling proteins in pils6-1 roots compared to W22 indicates that ZmPILS6 is a positive regulator of auxin responses in maize.

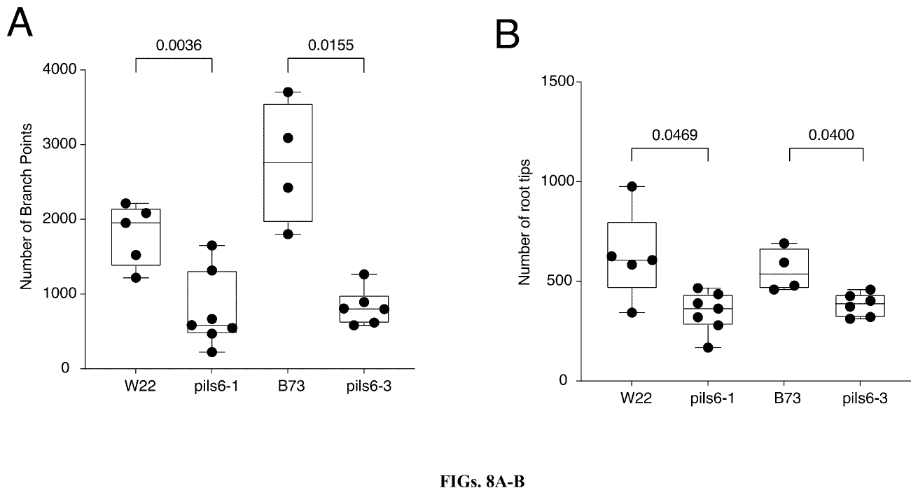

Proteins involved in transport and localization are overrepresented among DE proteins in pils6-1 roots (FIG. 4C, Dataset S4 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety). In addition to the enriched GO terms, several hormone related proteins were impacted in pils6-1 including auxin biosynthesis and transport, gibberellic acid response, abscisic acid responsive transcription factors, brassinosteroid related kinases, and jasmonic acid proteins associated with plant development (Dataset S3 of Cowling et al., “ZmPILS6 is an Auxin Efflux Carrier Required for Maize Root Morphogenesis,” Proc. Natl. Acad. Sci. USA 121.22: c2313216121 (2024), which is hereby incorporated by reference in its entirety). Using these proteomics data, a weighted gene co-expression network analysis was performed to predict and identify proteins that may be linked to ZmPILS6, which identified 19 modules (FIG. 10). The ‘greenyellow’ module contains 1299 highly correlated proteins, including ZmPILS6 and its paralog ZmPILS2 (Dataset S5 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety). In addition, there were 415 proteins that were highly correlated to ZmPILS6 based on our proteomics data (FIG. 4D). These include vesicle coat proteins, SNAP receptors (SNARE) proteins, kinases, phosphatases, and ABC transporters, which are color-coded in the network (FIG. 4D, Dataset S6 of Cowling et al., “ZmPILS6 is an Auxin Efflux Carrier Required for Maize Root Morphogenesis,” Proc. Natl. Acad. Sci. USA 121.22: e2313216121 (2024), which is hereby incorporated by reference in its entirety). The regulation of polar auxin transport by AGC kinases and several distinct LRR-RLKs (Armengot et al., “Regulation of Polar Auxin Transport by Protein and Lipid Kinases,” J. Exp. Bot. 67:4015-4037 (2016); Sun, et al., “PIN-LIKES Coordinate Brassinosteroid Signaling with Nuclear Auxin Input in Arabidopsis thaliana,” Current Biology 30:1579-1588.c6 (2020); DeGennaro et al., “Initiation of Aboveground Organ Primordia Depends on Combined Action of Auxin, ERECTA Family Genes, and PINOID,” Plant Physiol. 190:794-812 (2022), which is hereby incorporated by reference in its entirety) and ZmPILS6 is known to be phosphorylated in vivo (Walley et al., “Integration of Omic Networks in a Developmental Atlas of Maize,” Science 353:814-818 (2016), which is hereby incorporated by reference in its entirety). In addition, interactions between Arabidopsis PINs and ABCBs influence protein complex stability and function (Blakeslee et al., “Interactions Among PIN-FORMED and P-Glycoprotein Auxin Transporters in Arabidopsis,” Plant Cell 19:131-147 (2007); Titapiwatanakun et al., “ABCB19/PGP19 Stabilises PIN1 in Membrane Microdomains in Arabidopsis,” The Plant Journal 57:27-44 (2009), each of which is hereby incorporated by reference in its entirety). The identification of SNARE and vesicle coat proteins in the ZmPILS6 network is consistent with several reports on polarization of PIN proteins by SNAREs (Shirakawa et al., “Vacuolar SNAREs Function in the Formation of the Leaf Vascular Network by Regulating Auxin Distribution,” Plant Cell Physiol 50:1319-1328 (2009); Gu, et al., “The Arabidopsis R-SNARE VAMP714 is Essential for Polarisation of PIN Proteins and Auxin Responses,” New Phytol 230:550-566 (2021); Zhang et al., “SNARE Proteins VAMP721 and VAMP722 Mediate the Post-Golgi Trafficking Required for Auxin-Mediated Development in Arabidopsis,” Plant J. 108:426-440 (2021), each of which is hereby incorporated by reference in its entirety), and suggests that ZmPILS6 may also require SNAREs for proper subcellular localization.

### Example 6—Discussion of Examples 2-5

PILS proteins are evolutionarily conserved among land plants (Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019); Bogaert et al., “Auxin's Origin: do PILS Hold the Key?,” Trends in Plant Science 27:227-236 (2022), each of which is hereby incorporated by reference in its entirety). Most land plants contain several PILS orthologs, while sister algal groups contain only 1-2 annotated PILS genes (Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019), which is hereby incorporated by reference in its entirety). This radiation of the PILS gene family during land plant evolution may have been accompanied by neo- or sub-functionalization, which will require future investigation into the individual roles of PILS proteins across different land plants. The ER-localization and ability to contribute to auxin transport appears to be conserved between maize and Arabidopsis, suggesting evolutionarily conserved properties of these PILS. However, while this study supports a role for Clade II (PILS2/6 clade) proteins in regulating root formation, there are apparent diverged functions between Arabidopsis (a core eudicot) and maize (a monocot). PILS proteins in Arabidopsis are negative regulators of root and shoot growth but ZmPILS6 is a positive driver of organogenesis in maize. The short root phenotype and reduced stature of pils6 plants may be linked to decreased expression of numerous cell cycle proteins compared to W22. Additional investigation into the cellular properties underlying the maize pils6 phenotypes will be required to determine if organ sizes are smaller due to reduced cell division and/or cell elongation.

Lateral root formation in maize can occur from endodermal or pericycle cells within the elongation zone (Hochholdinger et al., “Genetic Dissection of Root Formation in Maize (Zea mays) Reveals Root-type Specific Developmental Programmes,” Annals of Botany 93:359-368 (2004), which is hereby incorporated by reference in its entirety). Low concentrations of auxin have been shown to positively influence lateral root formation in maize via pericycle cell elongation while high doses were inhibitory (Alarcón et al., “Auxin Modulated Initiation of Lateral Roots Is Linked to Pericycle Cell Length in Maize,” Frontiers in Plant Science 10 (2019), which is hereby incorporated by reference in its entirety). The observed defect in lateral root formation in pils6 roots could be explained by the altered auxin transport within the elongation zone compared to W22. Additional investigation into the in vivo levels of auxin in different regions of pils6 roots as well as characterization of lateral root founder cell properties will be required to better understand the role of ZmPILS6 in this process.

The maize and Arabidopsis PILS6 proteins have notable differences in their N-termini and middle regions that may contribute to the functional differences between these two orthologs. Future investigation into PILS interacting proteins and their associated downstream processes will be required to determine the mechanisms underpinning these functional differences in planta. The apparent reduction in auxin signaling based in pils6 roots at the proteome level is in contrast to increased auxin signaling observed in Arabidopsis pils mutants (Barbez et al., “A novel Putative Auxin Carrier Family Regulates Intracellular Auxin Homeostasis in Plants,” Nature 485:119-122 (2012); Feraru et al., “PILS6 is a Temperature-Sensitive Regulator of Nuclear Auxin Input and Organ Growth in Arabidopsis thaliana,” Proc. Natl. Acad. Sci. U.S.A. 116:3893-3898 (2019), each of which is hereby incorporated by reference in its entirety). However, additional investigation into the ER localization of ZmPILS6 in maize root cells will be required to determine if it is present in ER adjacent to the nucleus or in other regions of the cell that may influence nuclear auxin signaling. In addition, the identification of ZmPILS6 several co-expressed kinases, ABC transporters, and SNAREs will enable continued studies into protein complexes that influence auxin transport in maize.

### Example 7—Further Phenotypic Evaluation of Pils-6 Mutants

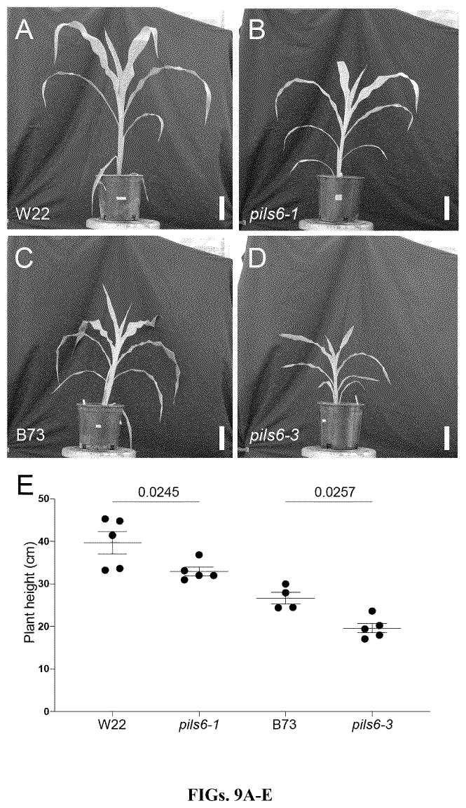

Further characterization of pils6 adult plants was performed to evaluate plant height, car height and stalk circumference of maize pils6-1 and pils-6 plants and their respective wild type controls (W22 and B73, respectively) under field conditions (e.g., see FIG. 11). Kernels were mechanically planted in mid-May 2024 at the Iowa State University Ag Engineering and Agronomy Research Farm in Boone, Iowa. A single line of seeds was planted in rows of ˜10 feet with 15 kernels per row. Plants grew until mid-August, when the plants were at full maturity. No additional water or nutrients were applied to the field. Measurements were taken using a tailor's measuring tape.

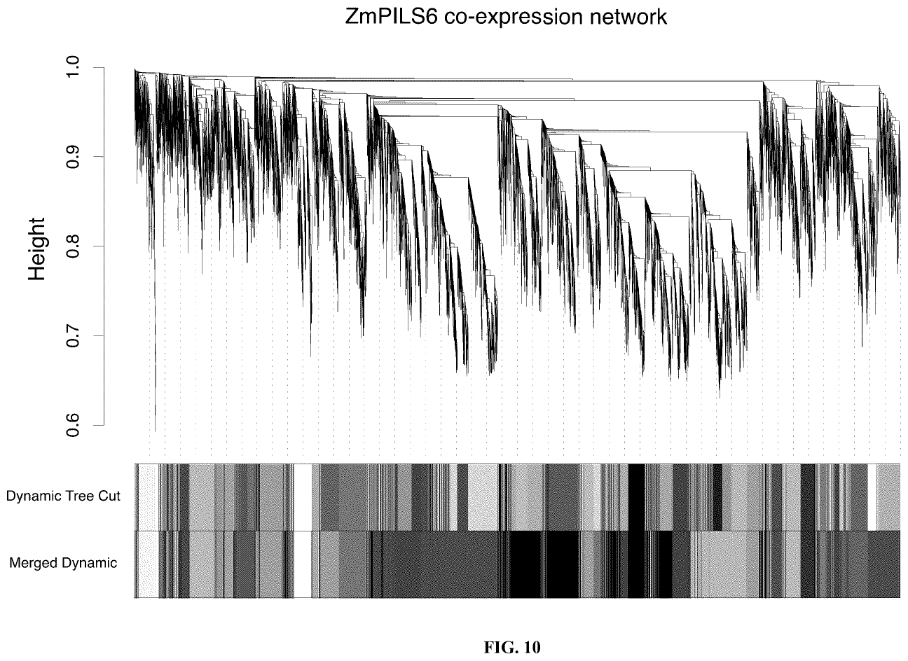

Method for measuring: Plant height was measured from the base of the plant at ground level to the lowest formed tassel branch. Due to tassel damage from manual pollination and smut infection on most plants, the tassel was excluded from the height measurement. Ear height was measured from the base of the plant at ground level to the internode of the first developing ear. The ear placement ratio was calculated as (ear height/Plant height)×100%. Stalk circumference was measured at the lowest internode of the base of the plant. FIG. 12A is a schematic diagram showing how the measurements of plant height, ear height, and stalk circumference were taken. Statistical analysis was done using one-way ANOVA followed by Tukey's multiple comparisons test.

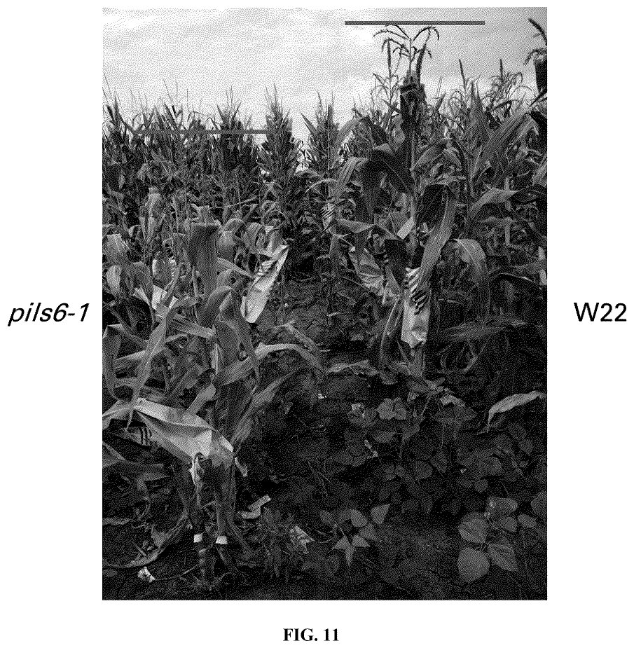

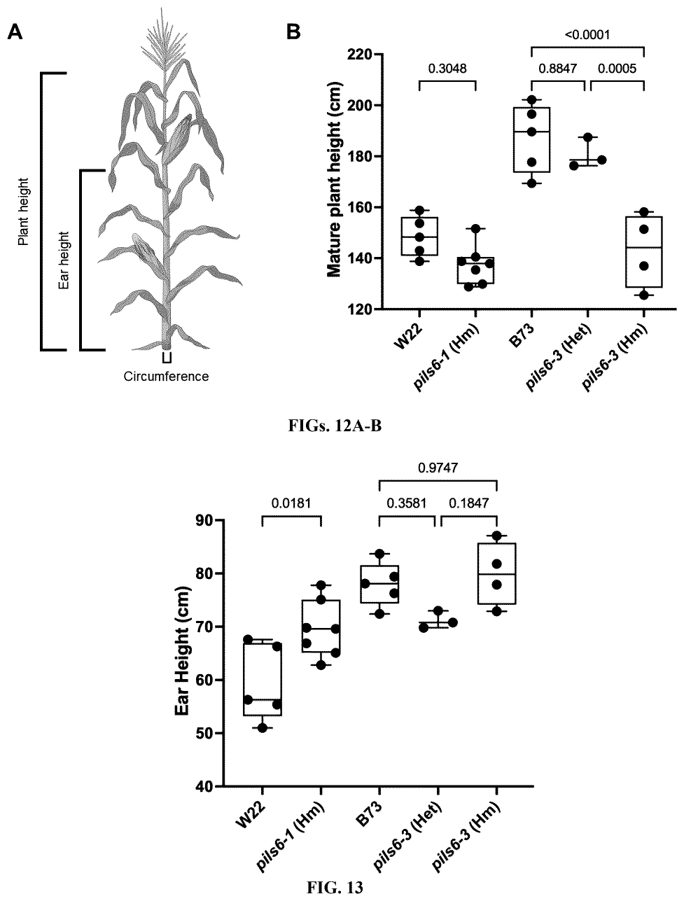

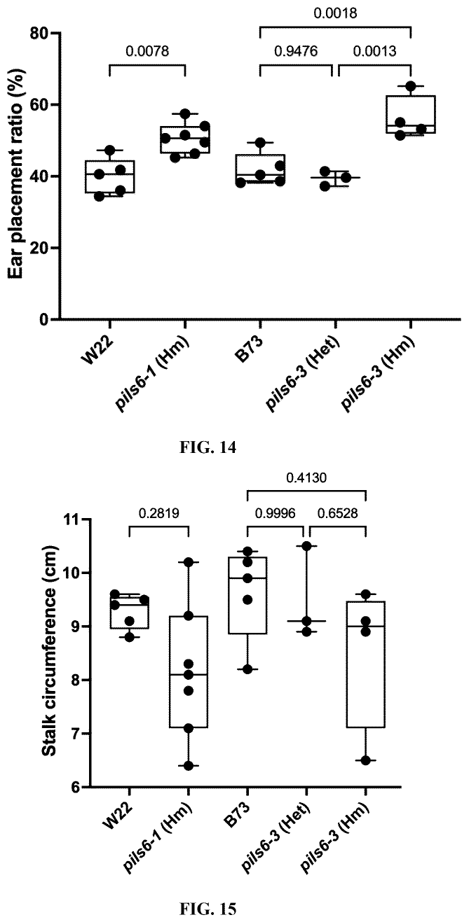

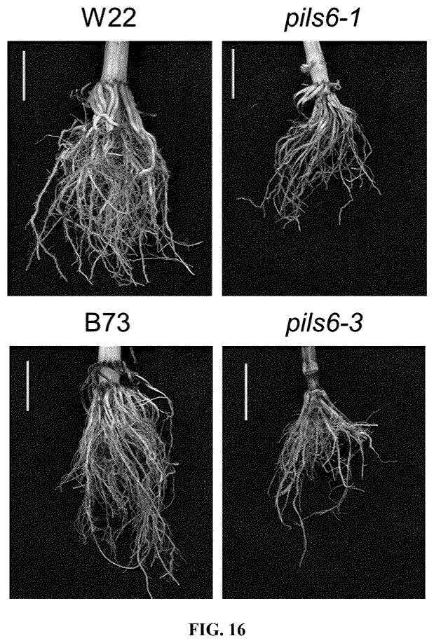

Mature plant height was reduced for homozygous pils6 mutants in comparison to their wild type controls (FIG. 12B). Ear height was increased in homozygous pils6 mutants in comparison to their wild type controls (FIG. 13). As shown in FIG. 14, mature pils6 plants had higher ear placement than their respective controls. Stalk circumference was not significantly smaller for mature pils6 stalks than their respective controls (FIG. 15). And, as shown in FIG. 16, pils6 mutants have reduced crown root architecture compared to their respective controls in plants grown to V7 stage (˜37 days) under field conditions.

In summary, maize plants deficient in PILS6 were shorter than the wild-type plants. The height of the ear on pils6-3 plants was the same as the wild-type plants. However, the ear height to plant height ratio was significantly different in both alleles of pils6. Higher ear placement could provide benefits for reducing exposure to soil-borne pathogens that may affect ear quality. Higher ear placement relative to stalk height on shorter stature plants ensures that harvesting can still be accomplished with modern corn heads. A slight, uniform increase in ear height could benefit harvesting without negatively impacting stalk susceptibility to storm damage.

### Example 8—Pils-6 Mutants are Less Sensitive to Salt Stress

Soil salinity is a major abiotic stress that adversely affects crop productivity, particularly in arid and semi-arid regions where irrigation practices can lead to the accumulation of salts in the soil. High salinity levels disrupt the ionic balance and osmotic potential of plants, leading to reduced water uptake, ion toxicity, and oxidative stress, which collectively impair plant growth and yield.

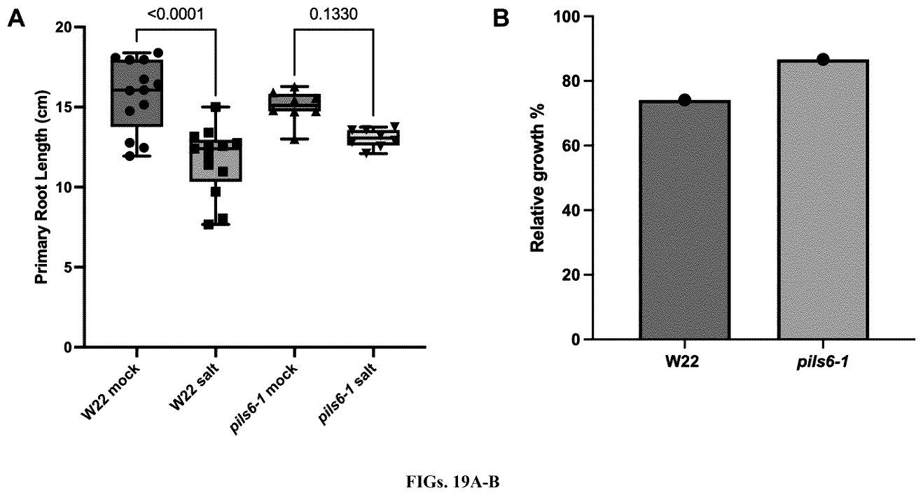

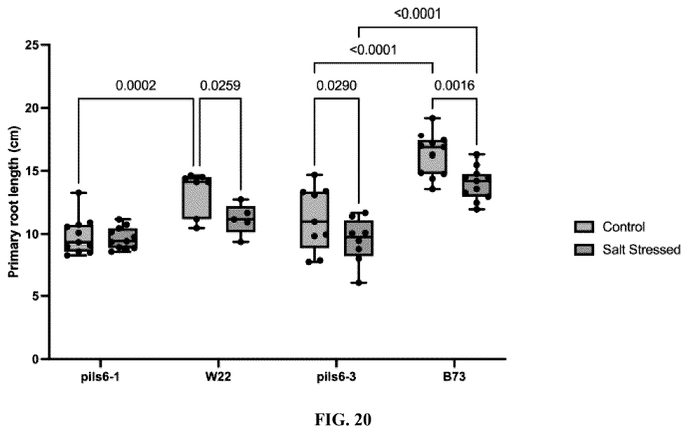

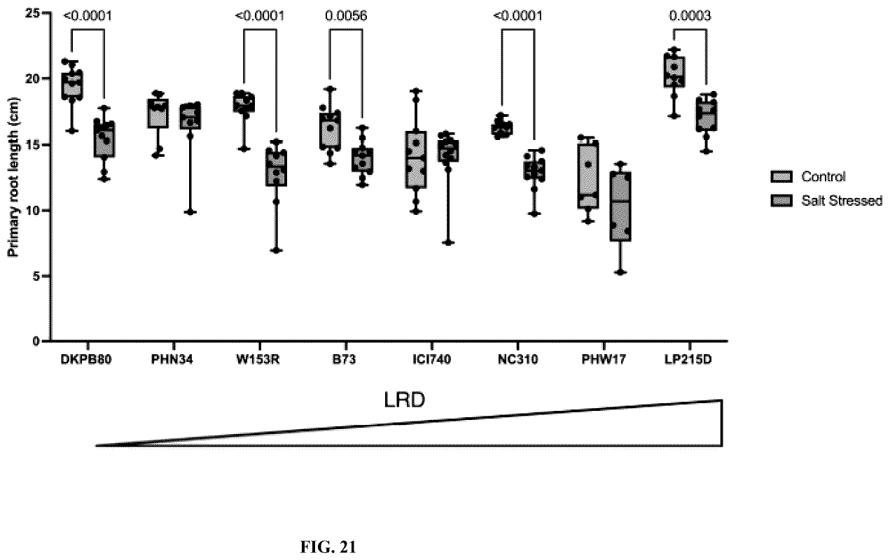

In order to test for ability of pils6 mutant plants to grow under environmental stress conditions such as salt, pils6-1 and pils-6 seedlings and their respective wild type controls (W22 and B73, respectively) were grown under control (0.5×LS media, no extra NaCl) conditions for 5 days, then grown with control or salt-stressed (0.5×LS media, plus 200 mM NaCl) conditions for an additional 5 days of growth. Statistical analysis was done using a two-way ANOVA. As shown in FIG. 18 pils6-1 plants were less sensitive to salt stress, showing less of an impact on growth under salt conditions than the wild type controls. Further results are shown in FIGS. 19A-B, FIG. 20, and FIG. 21. Maize is a salt sensitive (“glycophyte”) crop and exhibits stunted growth at levels of 100 mM NaCl. Glycophytes can leverage multiple mechanisms to cope with salt stress, including altered hormone biology. Fertilizer sources can vary in their ability to cause salt-damage to crops, and this is quantified with a “salt index”. For example, the range in salt index is ˜8 to 120 for CaSO4 to KCl, respectively. With KCl, or potash, being the most common form of K₂O for maize production. Too much fertilizer combined with low buffering-capacity soils and environmental conditions can create salt stress for maize. A reduction in PILS6 levels in pils6-1 and pils6-3 led to increased salt tolerance at 200 mM NaCl in 10-day-old seedlings. This could be beneficial to early seedling establishment during the first fertilizer treatment. Salt tolerant maize could also allow planting and growth in suboptimal soils with increased salt concentrations.

### Example 9—Meristematic Zone of Pils-6 Mutant Roots

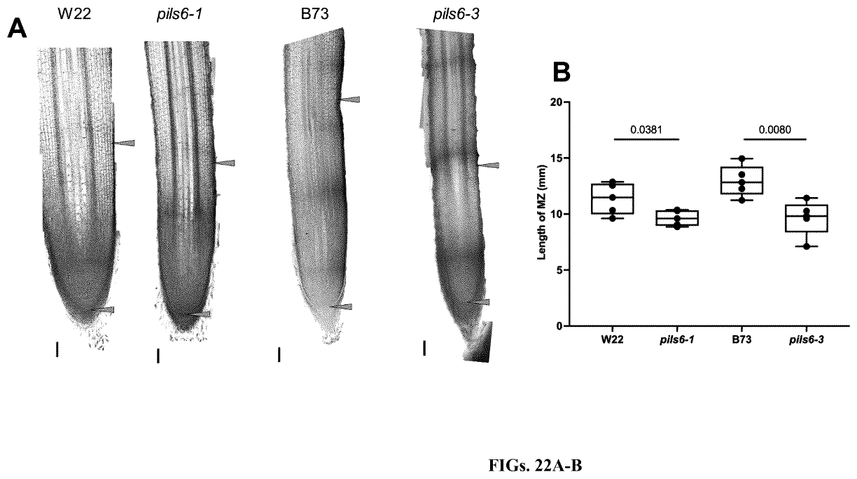

The length of the meristematic zone in pils6-1 and pils-6-3 mutants was measured and compared to their respective wild type varieties as shown in FIGS. 22A-B. To make these measurements, kernels were grown on rolled towels until the plants were 5 days old. Primary roots were cut from the plant and fixed in 70% ethanol at 4° C. until processed. A vibrating microtome was used to cut longitudinal sections that were 90 μm in thickness. Samples were stained with 0.025% Toluidine blue. Individual images photo merged using Adobe Photoshop. The root meristem size was reduced in size in pils6 mutants compared to their wild type controls.

### Example 10—Introgression of Pils-6 Mutants

The pils6-1 allele was introgressed into maize genotypes B104, PHZ51, P207, and LH244. After crossing into each inbred, the F1 plants and subsequent generations were genotyped for the wild-type PILS6 and pils6-1 alleles using polymerase chain reaction (PCR) and allele specific primers (Cowling et al., “ZmPILS6 is an Auxin Efflux Carrier Required for Maize Root Morphogenesis,” PNAS 121 (22) e2313216121 (2024), which is hereby incorporated by reference in its entirety). B104 is an excellent inbred for performing research because it is easily transformable. In addition, B104 can be useful for generating suitable hybrid lines (Feys et al., “Growth Rate Rather Than Growth Duration Drives Growth Heterosis in Maize B104 Hybrids,” Plant Cell Environ. 41 (2): 374-382 (2018), which is hereby incorporated by reference in its entirety). PHZ51 is a non-Stiff Stalk inbred developed in Iowa that has been used for hybrid trials (Perkins et al., Performance and Phenotypic Stability of Maize Hybrids Containing Exotic Introgressions in Multi-Environment Trials,” Crop Science 64 (2): 756-771 (2024), which is hereby incorporated by reference in its entirety). PH207 is an elite inbred line with an available genome that is used for developing hybrids (Hirsch et al., “Draft Assembly of Elite Inbred Line PH207 Provides Insights into Genomic and Transcriptome Diversity in Maize,” The Plant Cell 28 (11); 2700-2714 (2016), which is hereby incorporated by reference in its entirety). LH244 is an inbred corn line developed for breeding programs that has a draft genome (NLM-NCBI BioProject PRJEB40019, which is hereby incorporated by reference in its entirety). In addition, LH244 is transformable and is used by the Wisconsin Crop Innovation Center. The introgression of pils6-1 into these desirable inbred lines enables breeding strategies for incorporating desirable traits into hybrid lines. controls.

### Example 11—PILS6 Sequences

Exemplary PILS6 sequences as described herein are set forth in Table 5.

TABLE 5
PILS6 Sequences

SEQ ID NO: 4 DNA Arabidopsis thaliana PIN-LIKES 6 (At5G01990) AtPILS6 gene
ACTTGTAAACTTGAGTATTTTAATTAAATGAAAATAAACCATAAATAAATGAAGTTCTAAACTTGAGTAA
TAATAAAAACAGAATAAAAAAAAAGGTATGTGGGGAAGTTTTGAAGGGGCTTTTGGGCTTAGAAAAGGAA
AGAAGTAAAGGAAGAAGCGGCGCACGTGGCAGGCAGTCACGAGAGTCACGAGAGATCGAGAGAAGTCTTT
GATTCTCTTCTCTCATCAAATCAAAATATCATCTCAGAGACTCCGATTCTCTCTGTTTCTCCTCCACTTT
TCAACCCAAGTCCACCCTCAAAATTCTCAGAATGATTGCTCGGATCCTTGCCGCCTTAGCCGATTCCATG
GAGATGCCGGTGGCTGCCGGTGGTGGATCAGTGCTCGGTACCATCAAAATCGCTGTGATGCCAATTGCAA
AGGTATTCACCATGTGCTTCTTGGGTCTTCTCATGGCCTCCAAGTACGTTAACATCTTGCCTCCCTCTGG
CCGCAAACTCTTGAACGGGGTAATTAATTCTCCACACTAGACTTTTTCACTTTCTCTATAAATCCAAAAA
AAGCAAACTTTGAATGAAACTGTCTCTCCTTTTCACACAGTTGGTCTTCTCGCTTTTGCTTCCCTGCTTG
ATCTTTTCCCAGCTCGGACAAGCCGTCACTCTCCAAAAAATGCTTCAGTGGTATTTGTTTCCTTCTTTGT
CCCCCCTAGTCTCTCTCTAGTCCTTATATAGATCATCATATTCTTTTCCATCTTTCAGGTGGTTCATTCC
CGTTAACGTCGTTCTTGGCACCATCTCTGGCTCCATAATCGGTTTCATCGTTGCTTCCATCGTTCGTCCG
CCTTACCCTTACTTCAAGTTTACAATCATACAAATTGGAGTTGGTTAGACCCCTTTTTTTCCCTACCCTC
GCTTTTCTGCTCTGACACTAGTCTCACTTTATGTGCCTTGTTTGTTTCAGGCAACATTGGCAATGTGCCT
CTTGTTTTACTTGCGGCTCTTTGTAGGGACACTTCCAACCCTTTTGGTGACTCCGAAAAATGTAGCATTG
ATGGCACTGCTTATATCTCCTTTGGTCAATGGGTACTCTTTTTTCTCTTTTTAACTGCTTCTATGCTGTA
ACTATTATTATACCTACTAGAATCAGTCCATTAGCTGCTTCAGTTTCTGTCTCTTATGTAACAGTGATAC
AGTTTCTTGTCTTCTGCTTTTTTTTTCTGTTTGGATGAAGAGGAGCTTGATGTATGAGGAGTATCCTCAT
TGAAACTATTTTGTATAGGATCTTTAGTTCCAATGTACTGTTTGTCATCAATGTCTTATGGGAATGGATA
TTATTTCTCTCTGTGGGACTTCAATTAACCACAAGGTTTACTTTTTATATCAGGTTGGTGCCATCATCCT
CTATACATATGTGTACCAGATGTTTGCTCCTCCTCCAGAAGGGTTTGATGCTGAAGAAGAAAATCTTGCT
TTAAAAACCCTTCCAGTAGATGCTGCTCCAGAGCAAGTCCCCTTGCTTACTCAGAATTTTCCCAAGGACT
TTTCGCCGACTCAAGATCTTTTGCCTGTACAGAGCACCGAACCTAGGGGAAGAGGGGTTTCAAGGAAAGG
CAAGGTGGGACTGCAATTATGGAATCTTTATTCTATGTTTCAGGAGTATTTTACCTGATATAAGCTGGTT
CTTACGAAACTTTTTATCTTTTGCTCCGTGATGTAGATTGCACAGATCTTTGTTTTCCTCTATGAGAAGC
TGAAACTGAAGCAAATTGTCCAGCCTGCAATTGTTGCTTCGGTAAGTATCTCGTCTTTTCTATTCCCCTT
TTTTATCTGACAATCTAAAGATCAAGAGACCGTGCGATTGCCTTTACCATGTAACTAAACTAGACGTGTT
TTTGTTAACTTCAGATCCTTGCCATGATACTCGGAGCAATACCTTTCACAAAGAAGTTGATATTCACAAA
TGGTGCACCTCTATTCTTCTTCACAGACAGCTGCATGATTCTTGGGTAAGCACACTCTTTATTTGTCTTC
TGTGAAGCAGCTGCAAATTAATTAGTTAATATATGTAAAACCTTTGGCTATCAGGGACGCGATGATACCG
TGTATCTTACTGGCACTGGGGGGAAATCTCATTAACGGTAGGTTTGGTGGTTGCTTTCTTTTCATACTGA
CTTAGTTTTTAGCCCCATCCATCACATGGATCTTATTGGTTCCTTGGGTAAATTTTAGGACCAGGAAGTT
CAAAACTTGGTTTCAAGACAACAGCAGCTATTATAATTGGACGGTTGGTGTTAGTGCCACCAGTAGGACT
AGGAATCGTGACAGTAGCAGACAAACTTGGGTTCCTTCCTGCTGATGATAAGATGTTCCGATTTGTGTTA
CTCCTTCAGCACACAATGCCTACATCAGTGCTCTCAGGAGCTGTTGCCAACCTTAGAGGCTGTGGAAGGG
AATCAGCTGCTGTGCTCTTCTGGGTCCACATTTTCGCCATCTTCTCAATGGCTGGATGGATGGTACTCTA
CATTAACATACTCTTCTGAATTCTCTTCTTATTGACACATTCATCTGCTACTATACAATACTTCTGTGTA
AATACAGTGGCATCTCCTATGTGCCTGAACCAACCAAAATTCTGATTTTTTTTTTTTCCTTTCCTGTGTA
AATGTTAGTTTATAACTCTTT
SEQ ID NO: 5 Arabidopsis thaliana PIN-LIKES 6 (At5G01990) AtPILS6 coding
sequence
ATGATTGCTCGGATCCTTGCCGCCTTAGCCGATTCCATGGAGATGCCGGTGGCTGCCGGTGGTGGATCAG
TGCTCGGTACCATCAAAATCGCTGTGATGCCAATTGCAAAGGTATTCACCATGTGCTTCTTGGGTCTTCT
CATGGCCTCCAAGTACGTTAACATCTTGCCTCCCTCTGGCCGCAAACTCTTGAACGGGTTGGTCTTCTCG
CTTTTGCTTCCCTGCTTGATCTTTTCCCAGCTCGGACAAGCCGTCACTCTCCAAAAAATGCTTCAGTGGT
GGTTCATTCCCGTTAACGTCGTTCTTGGCACCATCTCTGGCTCCATAATCGGTTTCATCGTTGCTTCCAT
CGTTCGTCCGCCTTACCCTTACTTCAAGTTTACAATCATACAAATTGGAGTTGGCAACATTGGCAATGTG
CCTCTTGTTTTACTTGCGGCTCTTTGTAGGGACACTTCCAACCCTTTTGGTGACTCCGAAAAATGTAGCA
TTGATGGCACTGCTTATATCTCCTTTGGTCAATGGGTTGGTGCCATCATCCTCTATACATATGTGTACCA
GATGTTTGCTCCTCCTCCAGAAGGGTTTGATGCTGAAGAAGAAAATCTTGCTTTAAAAACCCTTCCAGTA
GATGCTGCTCCAGAGCAAGTCCCCTTGCTTACTCAGAATTTTCCCAAGGACTTTTCGCCGACTCAAGATC
TTTTGCCTGTACAGAGCACCGAACCTAGGGGAAGAGGGGTTTCAAGGAAAGGCAAGATTGCACAGATCTT
TGTTTTCCTCTATGAGAAGCTGAAACTGAAGCAAATTGTCCAGCCTGCAATTGTTGCTTCGATCCTTGCC
ATGATACTCGGAGCAATACCTTTCACAAAGAAGTTGATATTCACAAATGGTGCACCTCTATTCTTCTTCA
CAGACAGCTGCATGATTCTTGGGGACGCGATGATACCGTGTATCTTACTGGCACTGGGGGGAAATCTCAT
TAACGGACCAGGAAGTTCAAAACTTGGTTTCAAGACAACAGCAGCTATTATAATTGGACGGTTGGTGTTA
GTGCCACCAGTAGGACTAGGAATCGTGACAGTAGCAGACAAACTTGGGTTCCTTCCTGCTGATGATAAGA
TGTTCCGATTTGTGTTACTCCTTCAGCACACAATGCCTACATCAGTGCTCTCAGGAGCTGTTGCCAACCT
TAGAGGCTGTGGAAGGGAATCAGCTGCTGTGCTCTTCTGGGTCCACATTTTCGCCATCTTCTCAATGGCT
GGATGGATGGTACTCTACATTAACATACTCTTCTGA
SEQ ID NO: 6 Arabidopsis thaliana PIN-LIKES 6 (At5G01990) AtPILS6
amino acid sequence
MIARILAALADSMEMPVAAGGGSVLGTIKIAVMPIAKVFTMCFLGLLMASKYVNILPPSGRKLLNGLVES
LLLPCLIFSQLGQAVTLQKMLQWWFIPVNVVLGTISGSIIGFIVASIVRPPYPYFKFTIIQIGVGNIGNV
PLVLLAALCRDTSNPFGDSEKCSIDGTAYISFGQWVGAIILYTYVYQMFAPPPEGFDAEEENLALKTLPV
DAAPEQVPLLTQNFPKDFSPTQDLLPVQSTEPRGRGVSRKGKIAQIFVFLYEKLKLKQIVQPAIVASILA
MILGAIPFTKKLIFTNGAPLFFFTDSCMILGDAMIPCILLALGGNLINGPGSSKLGFKTTAAIIIGRLVL
VPPVGLGIVTVADKLGFLPADDKMFRFVLLLQHTMPTSVLSGAVANLRGCGRESAAVLFWVHIFAIFSMA
GWMVLYINILF
SEQ ID NO: 7 Sorghum bicolor PIN-LIKES 6 (LOC8055297) SbPILS6 gene
(Accession No. NC_012872.2: 65937697-65942524)
CTAGCAGTCCAACCAAACCCGCGCGTCGTCTCGCGAGCCGCGACTCCCCATCCCCATCGTCTCGTCGTTT
CCCCCGAGCGCCCAGATCCCATCCCTACCCGCTCCGCGCGACGCGAATCACAAGCCCGCAACTCTTGGGA
CTTTGGGAGGCGCCCCCCACCCCTGCCTGCCTGACCTCGGATCAAAGCCTCCTCCGTCCGCCTTAATCGC
GGCAGGTCGTTCGGGCCCGGGCCGTCACGCGGGAGGGGTTCATCACCCGGGAGAGCTCGGGGCTCGGCTC
CCTTCTTCTCGGGACCCCCGCGCGCGCACCGGCTGGGTCACCTCGACGCGGCGGCCACGCGGTGAGTCGA
GTTCTTCGCTTCTCGGGCCGCCAGTCCGCCTCTCGCTTGGTTGGGGCTGCTAGCGGGGTTCAGGATTCGC
GGGTGGCGTGGCGTGGCGTCGTCCGCGATCGCTCTCGCTCGCATTGAGCGATTATTCGCGACTGCTTGTT
CGTTTGCTTTCTCTCTGTTTTTTTTCTCTTGTGGGGATGTAGATTGTGATATGTGGCGTTGGGATTCTGA
GCTCGGTTGCTTCCTAAGCTTCCGCGGAGTGGTTTAACGCGTACGTATATGGTAATCGGTTTTGAAGCTG
CTAATATGGATGTGCCGTGTGCGTGACGAATCGAATCGGCCGCAGGATGCCTACGCCCTACGCAGCTCGG
CAGCTGGGGGAATTGGATCCCCCGACCATGCGAATTTGCAACCCAGAGTGCTCCGGGTTCTCGGTGTCGT
CATCTTTTACTAGTATAAGTAGTAGTCGTTGTTTGGAATCTAGTTTGTCATAATTTCTCTTCCCGATTTG
ATTGGTTTGTCGATGCGTCTGTTTTCGTCGAACAAGAAGTTTGTTATTGCTAAGCTTCCCGTTTCGGGGG
GAAGCTGCCTGCCGTACTGTGTTACGCGAATCCGGTGAGTTGGGCTCTGCGGTTCCTCTCGACGCGCTCT
GCGCCAACGGAAGACGAGCTCTGTTCCAGCTGGGTGGCCCCTGAACAACCAGCGCCAAAAGCAGTTCCAG
AATTTTCCTGATGTTGCCTCTATATTTTGTCTTCTAAGTCATGCTTAAATAATGGCAGAGTCCTGGTTGC
GTGGGTCACTGGATCCTGAGGCAAGAAAAAGAAGGCTTCAGTAGTCAGGTCTGGGTCAAGCCATGATGGA
GAGGTCGCTGCTGGAGGTGCTGGCCACGGCTGCGCAAGGAGGGACCGAGGGGACGTCGGTGCTGAGCATG
CTCAAGTATGCCGTGCTTCCCATCGCCAAGGTGTTCACTGTCTGCTTCATGGGGTTCCTCATGGCCTCCA
AGTACGTCAACATTCTCCAGCCCAACGGCCGCAAGCTTCTCAATGGGGTGAGCTTTGTCTCTTGTAATTA
ATAAAAGCTAGCTTTTGCGCAATGGACAATTGTAGCTGGATTTTGTGATTTTGTTCAGAGTAGGAATATG
TGTAGTAGTTGGGGACTTTTGTGCTCATATTAGACATCACATTTTCACTGTTTGCTGCAGCTTGTGTTTT
CCCTTCTGCTTCCATGCCTTATATTTTCCCAATTGGGTAGAGCAATCACTATCGAGAAGATGATACAATG
GTAAGGATAATTTCTATTGTTATCGTACTTAAGTTTTGTTATTATAAACCTAGTGTTATGCTTCGTCCAG
GTGTGCTATATGGCTCTTGATTTCTTATGTGTGAGCTATTGACTGAGATGTTGTTTTCAACTAATAATCT
CTATATTTGTATATGCTGCTGGCCCTCTATTAGCAATACATCCTGCAAGTATTAGATTACTGAAAATGAC
AAACCAATGGTGTGCTATTGTTGATCTTTCTGGTTGGGCCAACCTTTAAGTAATTAAGTTCCATATGGAA
CAATTTAGCTTTGGAAGCTCTAGCTTTCTACAAAAGGACAAAGAAAAGACCAACTGAAGGTGAAGGAAAT
CAGCACTCAAGCATTAATTTGGTTCCAGTTGAATTTATGGATCATATGGATTGACTCCACATTCCATGAT
TAATTTGTACCGCTACTTTATTTTTAACATACTGAAATACTGCAGTTAGTAAAGATTGGAGAAACTATTC
CAAGTAGTTTGTTTCCATATAATTTTTTTCTTACTAGCATACATATCTTGTTGAAAGTGATAGAAATTTA
TGTAATGGTCTATTCACTATCGTGTCCTACATTTTGACATACTCATGGATTCCTTTCCCCTTCTTGTGTG
TATATGTATAGCAAGTGTCCACTTTTATGCATAAGCTTCTTTAGGAGTATGTGTCCTCGAAATTTCCTTT
GTCAAATAACTGTTCTGAACTTCTGTTCTGCTTTTACTTCTGTTTTTTAATATCATCTATACTTGCATGT
TTGCAGGTGGTATATTCCAGTAAATATTGTTGTAGGTGCAGTATCCGGCTCTTTGATTGGATTTGTTGTG
GCATCTATCATCCGACCTCCATATCCATACTTTAAGTTCACTATTATCCATATAGGAATAGGTACCACTT
TTGTGTTTTGAAAATAAGTTTTATTTTCTATTTACTATTCGTTATCCAATACCATTGTGGTCCTTTTTTT
TGCAGGAAATATTGGAAATATACCTCTGGTCCTCATTGCAGCGTTATGTCGGGATCCTTCTAACCCATTT
GGTGACTCTGATAAATGCAATCAAGATGGGAATGCGTATATCTCATTTGGCCAATGGGTACGCCATCTCA
GCTTTTACATGCTCATTTTTACTCAAGTCAGCATAGTGACCTTGTTGTTTAAAGATATCTGTTTATCATC
TAGACTATGCATACAATTGGTTATTCACTCAGTTAGTCCTCTATATGTTGCAGGTTGGTGCAATTATTGT
TTACACATATGTATTCAAGATGCTTGCTCCACCACCAGGACAGACCTTTGATGGTTCTGAAGAGGATGAA
CTCCCAATCAAGGCATCTGGAGAGAATACAGTGCCCCAAATAGGAAATTATCCTATGAACACTCACACTA
GTACTGTACCAGAGAATGAACCATTGTTATCTGCTGGGGATGTTCAAAAGGAACGTGCCACTTCTGTAGG
AACAAAGGTAAGAAATTCGAATAGTGGAGAGCTGATTTCGATATCAGTTTGGTGCGCGTACTTCCAACTG
CAAAACTAAATGATTTTGTATATTAATGCAGATAATGGGCTTTGTTAAATGTGTGGTTAAGTTCCTGAAA
GACAAGCAGCTTCTCCAGCCACCGATTATTGCATCTGTATGTATTGCTGCTGTAGTGTTCTACTAACAAT
TTTTAAACCGAATTCTACTAGCAATTATTTATATACTAATTCTACAACTTTTTCCCCTTGACCATTGTTT
AACGCAGGCGTTTGCAATTGTAATTGGTGTTATCCCATTCTTGAAGAATTTTGTCCTTACAGATGATGCC
CCTCTGTTCTTTTTCACAGACAGCTGCCTCATTCTTGGGTATCTACATGTTTCATTTCTTGTTTTGTTCT
CTTGCCAATGAAGTTATCAAACTGAGATGAATGTATCTATCATCTATCACCTAATTCTGTGGCATACTTT
TCAGAGAAGCTATGATCCCATGCATTTTACTTGCTGTTGGGGGCAATCTTGTCGATGGTAAGTAATTATT
TTAGTTAAAACTTTGGAGAAATTGCATGGGGTGCAGTCACCCTTACATGGTTTAGAGGTATTTCTCATGT
TTTTGTCTTTATCCATGTTTATTTATGGAATCAGGCCCTGGTGAAGGAAGTAAGAGGCTTGGCGTCCGTA
CCACCGTTGCTATAATTTTTGCACGGTTGGTCTTGGTCCCTCTTGCTGGGGTTGGCATTATCATATTAGT
TGATAAACTTGGTTTCATTCCCAAAGATGATAAAATGTTCAAGTTTGTCCTGCTACTGCAGCATTCTATG
CCCACATCAGTGCTGTCAGGTATGTGAAAAGGGATGTGTCTTATCAGCCAACACTTATCTTCATGTGCTC
ATGTGTACAGATTCTTAACAATTTTGTGTGTTAGTTGAGATCTGTAAGGCATTGGGTGGTGCCTCGCATG
TTCTCCATGTTAGGAAACCACATTATGGAATCTGAAGATAGAATTGTTTGTTGCAGGTGCTGTTGCAAAT
CTGAGAGGGTGTGGAAAAGAATCAGCTGCAATTTTGTTCTGGGTTCACATTTTTGCTGTGTTCTCCATGG
CAGGATGGATTATTTTCTATCTGAGCTTGCTCTTCTAAGTATGTCACACCCTACTACCGCAACAATCTGC
AGTTTATTCTTGCATCCCCGCCTGTCAGATGTAACGCAATTTTTTGCTAGATAATAATGTTAGTTGATCT
GATTCCAATCAGTTGATTGCAGATACCTTACTGCACAGGAATATCTTTGAGATCAAATATTGTCCAAAAT
ATTGACGAAGAAACAAGTACCATGAACTGTACAGTAGCCACTAAATCGAAGTTGGAGAGGTACAATTGTA
TCAGGACAGATATATGGATATGATCACCCCCTATAATACCGTGGTCTCCTGCTGCTGCAAGCAGTGAGCG
ATGTGAGGACAGTGTTTCCCTGCACGTATCATCAGGTCACGGAGCCTAGATTGGGATGATGCCCTTCTTA
TCAGAACCCTAGACTATGCAGTGCTCCACGATATTCATTCATTTGTACTTCCGCGTATTGTAAATGTGAA
CCTTGAGGCTTTGTTCAAATAGTGTCCTTCTGGGCGAGGGCTGAAATAGTATAGATGTATTTAATACTAC
CTTAGGCCTGTCTATTTGGGTGTTGGCTTTGCAGGAAATGATGCTTTATCATGCAGAACTTGTGCCCA
SEQ ID NO: 8 Sorghum bicolor PIN-LIKES 6 (LOC8055297) SbPILS6 transcript
variant X1
TAGCAGTCCAACCAAACCCGCGCGTCGTCTCGCGAGCCGCGACTCCCCATCCCCATCGTCTCGTCGTTTC
CCCCGAGCGCCCAGATCCCATCCCTACCCGCTCCGCGCGACGCGAATCACAAGCCCGCAACTCTTGGGAC
TTTGGGAGGCGCCCCCCACCCCTGCCTGCCTGACCTCGGATCAAAGCCTCCTCCGTCCGCCTTAATCGCG
GCAGGTCGTTCGGGCCCGGGCCGTCACGCGGGAGGGGTTCATCACCCGGGAGAGCTCGGGGCTCGGCTCC
CTTCTTCTCGGGACCCCCGCGCGCGCACCGGCTGGGTCACCTCGACGCGGCGGCCACGCGAGTCCTGGTT
GCGTGGGTCACTGGATCCTGAGGCAAGAAAAAGAAGGCTTCAGTAGTCAGGTCTGGGTCAAGCCATGATG
GAGAGGTCGCTGCTGGAGGTGCTGGCCACGGCTGCGCAAGGAGGGACCGAGGGGACGTCGGTGCTGAGCA
TGCTCAAGTATGCCGTGCTTCCCATCGCCAAGGTGTTCACTGTCTGCTTCATGGGGTTCCTCATGGCCTC
CAAGTACGTCAACATTCTCCAGCCCAACGGCCGCAAGCTTCTCAATGGGCTTGTGTTTTCCCTTCTGCTT
CCATGCCTTATATTTTCCCAATTGGGTAGAGCAATCACTATCGAGAAGATGATACAATGGTGGTATATTC
CAGTAAATATTGTTGTAGGTGCAGTATCCGGCTCTTTGATTGGATTTGTTGTGGCATCTATCATCCGACC
TCCATATCCATACTTTAAGTTCACTATTATCCATATAGGAATAGGAAATATTGGAAATATACCTCTGGTC
CTCATTGCAGCGTTATGTCGGGATCCTTCTAACCCATTTGGTGACTCTGATAAATGCAATCAAGATGGGA
ATGCGTATATCTCATTTGGCCAATGGGTTGGTGCAATTATTGTTTACACATATGTATTCAAGATGCTTGC
TCCACCACCAGGACAGACCTTTGATGGTTCTGAAGAGGATGAACTCCCAATCAAGGCATCTGGAGAGAAT
ACAGTGCCCCAAATAGGAAATTATCCTATGAACACTCACACTAGTACTGTACCAGAGAATGAACCATTGT
TATCTGCTGGGGATGTTCAAAAGGAACGTGCCACTTCTGTAGGAACAAAGATAATGGGCTTTGTTAAATG
TGTGGTTAAGTTCCTGAAAGACAAGCAGCTTCTCCAGCCACCGATTATTGCATCTGCGTTTGCAATTGTA
ATTGGTGTTATCCCATTCTTGAAGAATTTTGTCCTTACAGATGATGCCCCTCTGTTCTTTTTCACAGACA
GCTGCCTCATTCTTGGAGAAGCTATGATCCCATGCATTTTACTTGCTGTTGGGGGCAATCTTGTCGATGG
CCCTGGTGAAGGAAGTAAGAGGCTTGGCGTCCGTACCACCGTTGCTATAATTTTTGCACGGTTGGTCTTG
GTCCCTCTTGCTGGGGTTGGCATTATCATATTAGTTGATAAACTTGGTTTCATTCCCAAAGATGATAAAA
TGTTCAAGTTTGTCCTGCTACTGCAGCATTCTATGCCCACATCAGTGCTGTCAGGTGCTGTTGCAAATCT
GAGAGGGTGTGGAAAAGAATCAGCTGCAATTTTGTTCTGGGTTCACATTTTTGCTGTGTTCTCCATGGCA
GGATGGATTATTTTCTATCTGAGCTTGCTCTTCTAAATACCTTACTGCACAGGAATATCTTTGAGATCAA
ATATTGTCCAAAATATTGACGAAGAAACAAGTACCATGAACTGTACAGTAGCCACTAAATCGAAGTTGGA
GAGGTACAATTGTATCAGGACAGATATATGGATATGATCACCCCCTATAATACCGTGGTCTCCTGCTGCT
GCAAGCAGTGAGCGATGTGAGGACAGTGTTTCCCTGCACGTATCATCAGGTCACGGAGCCTAGATTGGGA
TGATGCCCTTCTTATCAGAACCCTAGACTATGCAGTGCTCCACGATATTCATTCATTTGTACTTCCGCGT
ATTGTAAATGTGAACCTTGAGGCTTTGTTCAAATAGTGTCCTTCTGGGCGAGGGCTGAAATAGTATAGAT
GTATTTAATACTACCTTAGGCCTGTCTATTTGGGTGTTGGCTTTGCAGGAAATGATGCTTTATCATGCAG
AACTTGTGCCCA
SEQ ID NO: 9 Sorghum bicolor PIN-LIKES 6 (LOC8055297) transcript variant X2
GGGTTCAGGATTCGCGGGTGGCGTGGCGTGGCGTCGTCCGCGATCGCTCTCGCTCGCATGAGCGATTATT
CGCGACTGCTTGTTCGTTTGCTTTCTCTCTGTTTTTTTTCTCTTGTGGGGATGTAGATTGTGATATGTGG
CGTTGGGATTCTGAGCTCGGTTGCTTCCTAAGCTTCCGGGAGTGGTTTAACGCGTACGTATATGGTAATC
GGTTTTGAAGCTGCTAATATGGATGTGCGTGTGCGTGACGAATCGAATCGGCCGCAGGATGCCTACGCCC
TACGCAGCTCGGCAGCGGGGGAATTGGATCCCCCGACCATGCGAATTTGCAACCCAGAGTGCTCCGGGTT
CTCGGGTCGTCATCTTTTACTAGTATAAGTAGTAGTCGTTGTTTGGAATCTAGTTTGTCATAATTCTCTT
CCCGATTTGATTGGTTTGTCGATGCGTCTGTTTTCGTCGAACAAGAAGTTTGTATTGCTAAGCTTCCCGT
TTCGGGGGGAAGCTGCCTGCCGTACTGTGTTACGCGAATCCGGTCCTGGTTGCGTGGGTCACTGGATCCT
GAGGCAAGAAAAAGAAGGCTTCAGTAGTCAGGTCTGGGTCAAGCCATGATGGAGAGGTCGCTGCTGGAGG
TGCTGGCCACGGCTGCGCAAGGAGGGACCGAGGGGACGTCGGTGCTGAGCATGCTCAAGTATGCCGTGCT
TCCCATCGCCAGGTGTTCACTGTCTGCTTCATGGGGTTCCTCATGGCCTCCAAGTACGTCAACATTCTCC
GCCCAACGGCCGCAAGCTTCTCAATGGGCTTGTGTTTTCCCTTCTGCTTCCATGCCTTAATTTTCCCAAT
TGGGTAGAGCAATCACTATCGAGAAGATGATACAATGGTGGTATATTCAGTAAATATTGTTGTAGGTGCA
GTATCCGGCTCTTTGATTGGATTTGTTGTGGCATCTACATCCGACCTCCATATCCATACTTTAAGTTCAC
TATTATCCATATAGGAATAGGAAATATTGGAAATATACCTCTGGTCCTCATTGCAGCGTTATGTCGGGAT
CCTTCTAACCCATTTGGTGACTCTGATAAATGCAATCAAGATGGGAATGCGTATATCTCATTTGGCCAAT
GGGTTGGTGCAATTATTGTTTACACATATGTATTCAAGATGCTTGCTCCACCACCAGGACAGACCTTTGA
TGGTTCTGAAGAGGATGAACTCCCAATCAAGGCATCTGGAGAGAATACAGTGCCCCAAATAGGAAATTAT
CCTATGAACACTCACACTAGTACTGTACCAGAGAATGAACCATTGTTATCTGCTGGGGATGTTCAAAAGG
AACGTGCCACTTCTGTAGGAACAAAGATAATGGGCTTTGTTAAATGTGTGGTTAAGTTCCTGAAAGACAA
GCAGCTTCTCCAGCCACCGATTATTGCATCTGCGTTTGCAATTGTAATTGGTGTTATCCCATTCTTGAAG
AATTTTGTCCTTACAGATGATGCCCCTCTGTTCTTTTTCACAGACAGCTGCCTCATTCTTGGAGAAGCTA
TGATCCCATGCATTTTACTTGCTGTTGGGGGCAATCTTGTCGATGGCCCTGGTGAAGGAAGTAAGAGGCT
TGGCGTCCGTACCACCGTTGCTATAATTTTTGCACGGTTGGTCTTGGTCCCTCTTGCTGGGGTTGGCATT
ATCATATTAGTTGATAAACTTGGTTTCATTCCCAAAGATGATAAAATGTTCAAGTTTGTCCTGCTACTGC
AGCATTCTATGCCCACATCAGTGCTGTCAGGTGCTGTTGCAAATCTGAGAGGGTGTGGAAAAGAATCAGC
TGCAATTTTGTTCTGGGTTCACATTTTTGCTGTGTTCTCCATGGCAGGATGGATTATTTTCTATCTGAGC
TTGCTCTTCTAAATACCTTACTGCACAGGAATATCTTTGAGATCAAATATTGTCCAAAATATTGACGAAG
AAACAAGTACCATGAACTGTACAGTAGCCACTAAATCGAAGTTGGAGAGGTACAATTGTATCAGGACAGA
TATATGGATATGATCACCCCCTATAATACCGTGGTCTCCTGCTGCTGCAAGCAGTGAGCGATGTGAGGAC
AGTGTTTCCCTGCACGTATCATCAGGTCACGGAGCCTAGATTGGGATGATGCCCTTCTTATCAGAACCCT
AGACTATGCAGTGCTCCACGATATTCATTCATTTGTACTTCCGCGTATTGTAAATGTGAACCTTGAGGCT
TTGTTCAAATAGTGTCCTTCTGGGCGAGGGCTGAAATAGTATAGATGTATTTAATACTACCTTAGGCCTG
TCTATTTGGGTGTTGGCTTTGCAGGAAATGATGCTTTATCATGCAGAACTTGTGCCCA
SEQ ID NO: 10 Sorghum bicolor PIN-LIKES 6 (LOC8055297) SbPILS6 transcript
variant X3
CTAGCAGTCCAACCAAACCCGCGCGTCGTCTCGCGAGCCGCGACTCCCCATCCCCATCGTCTCGTCGTTT
CCCCCGAGCGCCCAGATCCCATCCCTACCCGCTCCGCGCGACGCGAATCACAAGCCCGCAACTCTTGGGA
CTTTGGGAGGCGCCCCCCACCCCTGCCTGCCTGACCTCGGATCAAAGCCTCCTCCGTCCGCCTTAATCGC
GGCAGGTCGTTCGGGCCCGGGCCGTCACGCGGGAGGGGTTCATCACCCGGGAGAGCTCGGGGCTCGGCTC
CCTTCTTCTCGGGACCCCCGCGCGCGCACCGGCTGGGTCACCTCGACGCGGCGGCCACGCGAGTCCTGGT
TGCGTGGGTCACTGGATCCTGAGGCAAGAAAAAGAAGGCTTCAGTAGTCAGGTCTGGGTCAAGCCATGAT
GGAGAGGTCGCTGCTGGAGGTGCTGGCCACGGCTGCGCAAGGAGGGACCGAGGGGACGTCGGTGCTGAGC
ATGCTCAAGTATGCCGTGCTTCCCATCGCCAAGGTGTTCACTGTCTGCTTCATGGGGTTCCTCATGGCCT
CCAAGTACGTCAACATTCTCCAGCCCAACGGCCGCAAGCTTCTCAATGGGCTTGTGTTTTCCCTTCTGCT
TCCATGCCTTATATTTTCCCAATTGGGTAGAGCAATCACTATCGAGAAGATGATACAATGGTGGTATATT
CCAGTAAATATTGTTGTAGGTGCAGTATCCGGCTCTTTGATTGGATTTGTTGTGGCATCTATCATCCGAC
CTCCATATCCATACTTTAAGTTCACTATTATCCATATAGGAATAGGAAATATTGGAAATATACCTCTGGT
CCTCATTGCAGCGTTATGTCGGGATCCTTCTAACCCATTTGGTGACTCTGATAAATGCAATCAAGATGGG
AATGCGTATATCTCATTTGGCCAATGGGTTGGTGCAATTATTGTTTACACATATGTATTCAAGATGCTTG
CTCCACCACCAGGACAGACCTTTGATGGTTCTGAAGAGGATGAACTCCCAATCAAGGCATCTGGAGAGAA
TACAGTGCCCCAAATAGGAAATTATCCTATGAACACTCACACTAGTACTGTACCAGAGAATGAACCATTG
TTATCTGCTGGGGATGTTCAAAAGGAACGTGCCACTTCTGTAGGAACAAAGATAATGGGCTTTGTTAAAT
GTGTGGTTAAGTTCCTGAAAGACAAGCAGCTTCTCCAGCCACCGATTATTGCATCTGCGTTTGCAATTGT
AATTGGTGTTATCCCATTCTTGAAGAATTTTGTCCTTACAGATGATGCCCCTCTGTTCTTTTTCACAGAC
AGCTGCCTCATTCTTGGAGAAGCTATGATCCCATGCATTTTACTTGCTGTTGGGGGCAATCTTGTCGATG
GCCCTGGTGAAGGAAGTAAGAGGCTTGGCGTCCGTACCACCGTTGCTATAATTTTTGCACGGTTGGTCTT
GGTCCCTCTTGCTGGGGTTGGCATTATCATATTAGTTGATAAACTTGGTTTCATTCCCAAAGATGATAAA
ATGTTCAAGTTTGTCCTGCTACTGCAGCATTCTATGCCCACATCAGTGCTGTCAGGTGCTGTTGCAAATC
TGAGAGGGTGTGGAAAAGAATCAGCTGCAATTTTGTTCTGGGTTCACATTTTTGCTGTGTTCTCCATGGC
AGGATGGATTATTTTCTATCTGAGCTTGCTCTTCTAATTGATTGCAGATACCTTACTGCACAGGAATATC
TTTGAGATCAAATATTGTCCAAAATATTGACGAAGAAACAAGTACCATGAACTGTACAGTAGCCACTAAA
TCGAAGTTGGAGAGGTACAATTGTATCAGGACAGATATATGGATATGATCACCCCCTATAATACCGTGGT
CTCCTGCTGCTGCAAGCAGTGAGCGATGTGAGGACAGTGTTTCCCTGCACGTATCATCAGGTCACGGAGC
CTAGATTGGGATGATGCCCTTCTTATCAGAACCCTAGACTATGCAGTGCTCCACGATATTCATTCATTTG
TACTTCCGCGTATTGTAAATGTGAACCTTGAGGCTTTGTTCAAATAGTGTCCTTCTGGGCGAGGGCTGAA
ATAGTATAGATGTATTTAATACTACCTTAGGCCTGTCTATTTGGGTGTTGGCTTTGCAGGAAATGATGCT
TTATCATGCAGAACTTGTGCCCA
SEQ ID NO: 11 Sorghum bicolor PIN-LIKES 6 (LOC8055297) SbPILS6 coding
sequence
ATGATGGAGAGGTCGCTGCTGGAGGTGCTGGCCACGGCTGCGCAAGGAGGGACCGAGGGGACGTCGGTGC
TGAGCATGCTCAAGTATGCCGTGCTTCCCATCGCCAAGGTGTTCACTGTCTGCTTCATGGGGTTCCTCAT
GGCCTCCAAGTACGTCAACATTCTCCAGCCCAACGGCCGCAAGCTTCTCAATGGGCTTGTGTTTTCCCTT
CTGCTTCCATGCCTTATATTTTCCCAATTGGGTAGAGCAATCACTATCGAGAAGATGATACAATGGTGGT
ATATTCCAGTAAATATTGTTGTAGGTGCAGTATCCGGCTCTTTGATTGGATTTGTTGTGGCATCTATCAT
CCGACCTCCATATCCATACTTTAAGTTCACTATTATCCATATAGGAATAGGAAATATTGGAAATATACCT
CTGGTCCTCATTGCAGCGTTATGTCGGGATCCTTCTAACCCATTTGGTGACTCTGATAAATGCAATCAAG
ATGGGAATGCGTATATCTCATTTGGCCAATGGGTTGGTGCAATTATTGTTTACACATATGTATTCAAGAT
GCTTGCTCCACCACCAGGACAGACCTTTGATGGTTCTGAAGAGGATGAACTCCCAATCAAGGCATCTGGA
GAGAATACAGTGCCCCAAATAGGAAATTATCCTATGAACACTCACACTAGTACTGTACCAGAGAATGAAC
CATTGTTATCTGCTGGGGATGTTCAAAAGGAACGTGCCACTTCTGTAGGAACAAAGATAATGGGCTTTGT
TAAATGTGTGGTTAAGTTCCTGAAAGACAAGCAGCTTCTCCAGCCACCGATTATTGCATCTGCGTTTGCA
ATTGTAATTGGTGTTATCCCATTCTTGAAGAATTTTGTCCTTACAGATGATGCCCCTCTGTTCTTTTTCA
CAGACAGCTGCCTCATTCTTGGAGAAGCTATGATCCCATGCATTTTACTTGCTGTTGGGGGCAATCTTGT
CGATGGCCCTGGTGAAGGAAGTAAGAGGCTTGGCGTCCGTACCACCGTTGCTATAATTTTTGCACGGTTG
GTCTTGGTCCCTCTTGCTGGGGTTGGCATTATCATATTAGTTGATAAACTTGGTTTCATTCCCAAAGATG
ATAAAATGTTCAAGTTTGTCCTGCTACTGCAGCATTCTATGCCCACATCAGTGCTGTCAGGTGCTGTTGC
AAATCTGAGAGGGTGTGGAAAAGAATCAGCTGCAATTTTGTTCTGGGTTCACATTTTTGCTGTGTTCTCC
ATGGCAGGATGGATTATTTTCTATCTGAGCTTGCTCTTCTAA
SEQ ID NO: 12 Sorghum bicolor PIN-LIKES 6 (LOC8055297) SbPILS6 amino acid
sequence
MMERSLLEVLATAAQGGTEGTSVLSMLKYAVLPIAKVFTVCFMGFLMASKYVNILQPNGRKLLNGLVFSL
LLPCLIFSQLGRAITIEKMIQWWYIPVNIVVGAVSGSLIGFVVASIIRPPYPYFKFTIIHIGIGNIGNIP
LVLIAALCRDPSNPFGDSDKCNQDGNAYISFGQWVGAIIVYTYVFKMLAPPPGQTFDGSEEDELPIKASG
ENTVPQIGNYPMNTHTSTVPENEPLLSAGDVQKERATSVGTKIMGFVKCVVKFLKDKQLLOPPIIASAFA
IVIGVIPFLKNFVLTDDAPLFFFTDSCLILGEAMIPCILLAVGGNLVDGPGEGSKRLGVRTTVAIIFARL
VLVPLAGVGIIILVDKLGFIPKDDKMFKFVLLLQHSMPTSVLSGAVANLRGCGKESAAILFWVHIFAVES
MAGWIIFYLSLLF
SEQ ID NO: 13 Oryza sativa PIN-LIKES 6-1 (LOC4327495) Gene NC_089035
REGION: 35441596 . . . 35446895
GATAGCTTAATCTCGAGCGCTTCGAGTTCGATCTCTCGTGAAGGGGACGACGAGGGCATTGTCTCCCTCC
TCGCGCGCGCGGAGTCGCCGCCGCCGCCGCCTCGTCCTCGGCCACGCGGTGAGTCCTCGCGCCGACCTCG
CCGTTTTGGTTGCGTTTTTTTTTAGTTTTCTCTGCAGCGGGATTTGGAGACGATGGGTGTCGCTGTGTGT
GCTGTGAAATGGGGATTTGTGGTTTTTTTTTTTGGTTTGTGGAGTACGAGTACGCGGGGGAAGTTTCGAG
GTCGGTTGCATCCGGAGCTCCCGCTGGAGTGGTCTACTCTAGTGCGGATTTGGAAGCGGAGAAGTTGGAG
TTGGAGGTGGCGTGACGGATCAAGCGGAGTCGCGATCGCTCGCCGGAGCCGTGCGGATTCCGGTGCTAGC
GAACAACGGCGGCACCGTCGGCGTTTTGTAGCGGTCAAGCTGCTGCTGCTGCTCGGAATCTAGCTAGGTT
TTTGTTCTGTCCTAATCCGGGTTTCCCGATTGGATTTGATGGTCTTCTCCCCACGCATGAGAGTTGGTTA
GCACGGAAGCTTTGGGGATTGTTCCGTTTAATTCGGCGCGCTTGCTGAGTTGTGCGCGCTCACGCTGCGC
TCCAGTTGGCTAGGCCTGCGGGGGAGGCGATCTCTGTTCCGCTTTGGCGCTCTCTGGACAACCGACTTCA
AAAGCGGCTCCTGATTTTTTTCCTGCTGTTGCTTGTATATTTTGCCTTCTAAGTCACTCATGTTGGCAGG
AATTGCGTGGGTCCTGGTTGTGGAAGTCAGGAAGTGAAGAAGAAGAAGAAGAAGTGAACTAGTGAAGCCT
GGGGCAAGCCATGATGGAGAGGTCGCTGATGGAGGCGCTGGCTACGGCGGCGCAGGGAGGCACCGTGGGG
ACGTCGGTTTTCGACATGCTCAAGTACGCCGTGCTGCCCATTGCCAAGGTGTTTACCGTCTGTTTCATGG
GGTTCCTCATGGCCTCTAAGTATGTCAATATCCTCCAGCCGAACGGCCGCAAGCTTCTCAATGGGGTGAG
CCTTGTTCATTCTCTTGCAATATAATTTTATCTGTAAGATTTATGAGTTTTTGCTGTGACTATGGAGAAT
GGTAAGTTGGCTGCTGTCTAGAATAGGAAGATGTGGGGGCAGTTGCTTGTGCTTACTTGCATTCCTGATG
TTCTCGATTATGCTTACATTTTGGTCGTTTGGTGCAGCTTGTGTTCTCGCTTCTACTTCCTTGCCTTATA
TTTTCCCAACTGGGTAGAGCAATCACAATCGAAAAGATGCTGCAATGGTAATGACAGATTCTAGTTAATA
TGCTTTGTTAAGTTTGCTGCATGCTTTTCGCATTTCATTGTGTAGTCTATTGATCATGGTTCTTACCATA
ACTAGATCGTTCTGGTTGCGGGAATTTTAAGTCATCATCCAAATGGAGTAAATTAGCTCCCAGAATTTTC
TGCAAAAAGATCTTAAGTGTACCAATCGAAAGCGAAGGAAGTGTTGCATTATCTGAAAGTTTCAGGACTA
ATTTAAGACGTCAAAATACTTAAATTACCATATTGTTTGTTACTGGCAAAATCCATCTGGTTGGAATTGG
TTGGACTTCATCTATAAGGCCATCCAATCCTTGTTATTATTTATGATGACATAAGGCCTTTTTCTTTGCA
AAAGATAATCGCATTGTGCCTATGTTTTGCTTTTACTGTTTTTAACATTCTTACAGTAGCAATATTTTGT
TGTTTGCAGGTGGTATATTCCAGTAAATATTGTTGTAGGTGCAGTGTCAGGCTCTTTGATTGGCTTTGTG
GTGGCTTCTATCATCCGACCACCTTATCCATACTTCAAGTTCACTGTGATTCACATTGGAATAGGTACCT
GGTTTTCTTTATTAAATAAAGTTGCCTTCTCATTTATCATTCTTCTATTTTATATTGATCCGATCCCTTA
TTTCGATTCAGGGAATATTGGAAATATACCTCTGGTTCTCATTGCAGCATTATGTCGAGATCCCACCAAT
CCTTTTGGTGACTCTGATAAATGTAATGAAGATGGGAATGCGTATATCTCATTTGGTCAATGGGTACATG
TCTTTTCTTTTCTGAAGTTGCATGAGTTCATGCTTTATTCAGGACCTCTACTATGCAAAATAAATGTAAC
AGGTTGAATTTAGGTTAGGTATGCATTGAGATCATTCACTCAGTGAATCCTTTAAATGTTTCAGGTTGGT
GCAATTATTGTTTACACATATGTGTTCAAGATGCTTGCTCCACCACCTGGTGAATCCTTTGATAGCGCTG
AAGAAGATATTCTTCCAATTAAGGCATCTGGAGATAATGTGGTGCCTGAAAAAGGGAAATATCCAACAAG
CACTCGCACTAGTACTGTACCTGAAAATGAGCCTTTGTTATCTTCTGAAGGTGATAAAAATGTTTCTACT
TCTCTAGGATCGAAGGTGAGAAATACCATCAGAAATTTGGTATGTTTACTTCTTATATCAATTTGATTTC
TAACATGAGCTAAAAATCATGCTCTGTTGATGCAGATAATGGGCATTGTTAGAAGCATGGTTAAGTTCCT
AAAAGACAAGCAGCTTCTTCAGCCACCAATTATTGCCTCTGTATGTATCTACACTACAATATTATGCTAG
CAATAAACACAGAACATTTCTGTATACTGTGGTTTGACAGCAACTATTTTACACTATGCACAGGTTTTTG
CAATTGCCATTGGTGTTGTTCCAGTCTTGAAGAATTTTGTCCTCACTGATGATGCACCTCTGTTCTTCTT
CACAGACAGCTGTCTCATTCTAGGGTATTTACATGACTTTCAGTTCCCTTTTTGTAAAATAAAGTACACT
TTCCTATAAAATTACAAAAGCACGATGAGCCACCTTGGTTCCTAGTTTCTAATTCTGTCATTCCTTTTTC
AGGGAAGCAATGATCCCTTGCATTTTGCTTGCAGTGGGGGGCAATCTTGTCGATGGTAAGCACCTAGTTC
TGTTGAAACTATTGCGCGGCTCCTACCATTCTATAGTACTAGGAGCCTAGGATGCATCTCCAGATCTTAT
CCTAGCTCATGTAACCATGTTTATTGCATCAGGTCCTGGTGAAGGAAGTAACAGGCTTGGTGTGCGGACA
ACCGTTGCTATTATTTTTGCACGGCTCATCTTGGTTCCTCTTGCTGGGGTTGGCATCATTGTGTTGGTTG
ATAAACTTGGTTTCATTCCTAAAGATGACAAAATGTTCAAGTTTGTCTTGCTACTGCAGCATTCTATGCC
CACATCTGTGCTGTCAGGTACTAAGAAATGAATAAGGGACACTACTTATCACTTATTATCATGTGAGCAT
GTTCAAAGATAGATTCCTTCTAATAGTTTTATGTGTTGGTTGAGATCTGTAAGGCATTGGTGGTGGAACA
CCATGTGCTTTATGTTAGCGACCAAATAATGGAATATGATCTGAGTTTTTTTTTTTTTTTTGGCAGGTGC
TGTTGCAAACCTGAGGGGCTGTGGAAAAGAATCTGCTGCTATCTTGTTCTGGGTGCACATCTTTGCTGTG
TTCTCCATGGCAGGATGGATTATTTTGTATTTGAGCTTACTCTTCTAAGTACGTCATACCCTGGTAATGG
CAGTTAATCTGTGGCATTTGTGCTCAAGTTGCAGCTCTACAGATTAACTACTATACGAGTCCTTGAATAC
TTAGAGCCACTCTCTGATTTGATTTGCAGATGCTATACTGCACAGGAACGTCCATAAGATCAAAATATCA
CCAGATATACCAGAAAAAGATAGTACCATGACATGTACATTAGCCAGTAAACCATGGCGACAGCCTGAAG
GTACGAGTTGTATCAGTATTCAGTATAGATATGGTTCTCCTGCCCAGTGCCCAATCGCTTAAGCGCCACT
GCTATGTTAATTTTGCTAATCGCTCGCTGCTACAAGCAACTGAGCAATGAGAAAATGAGGGCATAGCGGT
ATTGCCGATGCCCAGATCATCAGGTCCTCATGACCTTTAGGTCGGTTTCCTATATAAGCCTCTTTCCTTT
ATTAATGTAGCCCCCCCCACCCCCCCACCCCCCCCCCCCCCCCCCACAATGATCTTCATTGAATGAACTA
CAAGCTTCAAGTGTGTTTTTGTGGTGCAGTTATATTTGAAAGAGTAAGAGTAAACTACAAATTGGACCAC
TGTTTATTGGCAAAGTTTTGCTTTGGGCCTCCCTTTAACCAATGTTTTCACTTTGGACTAGATAATTATA
CTTGTGTCACTTTGGATCACCCCTAACTCTTATGTTAAGTACATCAATAATCTCGATTATACCGGCTTTT
GCATGCCGATGAACTCGTGAAATTGTATGTAGACCACACCTTTGGCACAGAATGAGTTGGATGTAAATGG
ATAAGAGTTGAGAGTTGTCCAAGTGACACAAATAAAAAATTATCTAGTCCAAAGTGAAAATATTGGTTAA
AAGGCAAGCCCAAACCGAAACTTTGATTATAAAAGGTGGCCCAAAGCATGAACTTCGGTTGAAACTTTGA
TTATATATCTTGATTATATGGATACAATCTCTCATGCATGAACTGATGCGTGCACATGCATAATGTTTAT
GTGATCACATGATTCGCATCATGCAGGATGGGTGCCCGACATCCACCCACGCACATACATGAATGCCAAA
ATCAAAATATTTGCCTGCAAGCTCATGCCAGCGGTTCCTAGTATGCATGTGGGTCATGTAGCCTTAACTA
GCATTGGTACTTCCAGATCCAGTGATATCAGAGTACATATGTGATCTCTCCATGTGCTTATAGATCTGGG
AACCTGCAGATCTGAAGTTCTGAACCCACAGTTTTCATTCACTTTTAATCTTTTGAATCTGAAGGAGAGG
GAGATTCATGATTCATCACCGTACCAAAAAAAAAGGTGAGAGATTTCTATAGTGAACTTTAACCAAGCAG
ACAGCTCAATATGCCTCTTGGTGTGCGTATTGGTTGGGGTCTCAAAATCAGATGCAAATCCGTCAAACGG
AATATACTTGGAAGATACGTATCGCGTCAGCACTCAGCATGGAGTCAGACAGGTACTGACATTCAGACAC
GCTAGGGTCGGACAAAAAACAGATACTGCCAATATTCGTTCATCGGAAATAGAACAAAAAAACATGTATT
GACAATAAATCAGTGTACAGAACAGTACCGTATCGATATGGCGAGCAGATAATAACGCAAGGATACGTAT
GAGCCGTCTCTCATCAGCAGAAATGGCTGCATGTATTCGCTTCGCACCCA
SEQ ID NO: 14 Oryza sativa PIN-LIKES 6-1 (LOC4327495) GenBank Accession No.
XM_015765602.2 transcript
GATAGCTTAATCTCGAGCGCTTCGAGTTCGATCTCTCGTGAAGGGGACGACGAGGGCATTGTCTCCCTCC
TCGCGCGCGCGGAGTCGCCGCCGCCGCCGCCTCGTCCTCGGCCACGCGGAATTGCGTGGGTCCTGGTTGT
GGAAGTCAGGAAGTGAAGAAGAAGAAGAAGAAGTGAACTAGTGAAGCCTGGGGCAAGCCATGATGGAGAG
GTCGCTGATGGAGGCGCTGGCTACGGCGGCGCAGGGAGGCACCGTGGGGACGTCGGTTTTCGACATGCTC
AAGTACGCCGTGCTGCCCATTGCCAAGGTGTTTACCGTCTGTTTCATGGGGTTCCTCATGGCCTCTAAGT
ATGTCAATATCCTCCAGCCGAACGGCCGCAAGCTTCTCAATGGGCTTGTGTTCTCGCTTCTACTTCCTTG
CCTTATATTTTCCCAACTGGGTAGAGCAATCACAATCGAAAAGATGCTGCAATGGTGGTATATTCCAGTA
AATATTGTTGTAGGTGCAGTGTCAGGCTCTTTGATTGGCTTTGTGGTGGCTTCTATCATCCGACCACCTT
ATCCATACTTCAAGTTCACTGTGATTCACATTGGAATAGGGAATATTGGAAATATACCTCTGGTTCTCAT
TGCAGCATTATGTCGAGATCCCACCAATCCTTTTGGTGACTCTGATAAATGTAATGAAGATGGGAATGCG
TATATCTCATTTGGTCAATGGGTTGGTGCAATTATTGTTTACACATATGTGTTCAAGATGCTTGCTCCAC
CACCTGGTGAATCCTTTGATAGCGCTGAAGAAGATATTCTTCCAATTAAGGCATCTGGAGATAATGTGGT
GCCTGAAAAAGGGAAATATCCAACAAGCACTCGCACTAGTACTGTACCTGAAAATGAGCCTTTGTTATCT
TCTGAAGGTGATAAAAATGTTTCTACTTCTCTAGGATCGAAGATAATGGGCATTGTTAGAAGCATGGTTA
AGTTCCTAAAAGACAAGCAGCTTCTTCAGCCACCAATTATTGCCTCTGTTTTTGCAATTGCCATTGGTGT
TGTTCCAGTCTTGAAGAATTTTGTCCTCACTGATGATGCACCTCTGTTCTTCTTCACAGACAGCTGTCTC
ATTCTAGGGGAAGCAATGATCCCTTGCATTTTGCTTGCAGTGGGGGGCAATCTTGTCGATGGTCCTGGTG
AAGGAAGTAACAGGCTTGGTGTGCGGACAACCGTTGCTATTATTTTTGCACGGCTCATCTTGGTTCCTCT
TGCTGGGGTTGGCATCATTGTGTTGGTTGATAAACTTGGTTTCATTCCTAAAGATGACAAAATGTTCAAG
TTTGTCTTGCTACTGCAGCATTCTATGCCCACATCTGTGCTGTCAGGTGCTGTTGCAAACCTGAGGGGCT
GTGGAAAAGAATCTGCTGCTATCTTGTTCTGGGTGCACATCTTTGCTGTGTTCTCCATGGCAGGATGGAT
TATTTTGTATTTGAGCTTACTCTTCTAAGTACGTCATACCCTGATGCTATACTGCACAGGAACGTCCATA
AGATCAAAATATCACCAGATATACCAGAAAAAGATAGTACCATGACATGTACATTAGCCAGTAAACCATG
GCGACAGCCTGAAGGTACGAGTTGTATCAGTATTCAGTATAGATATGGTTCTCCTGCCCAGTGCCCAATC
GCTTAAGCGCCACTGCTATGTTAATTTTGCTAATCGCTCGCTGCTACAAGCAACTGAGCAATGAGAAAAT
GAGGGCATAGCGGTATTGCCGATGCCCAGATCATCAGGTCCTCATGACCTTTAGGTCGGTTTCCTATATA
AGCCTCTTTCCTTTATTAATGTAGCCCCCCCCACCCCCCCACCCCCCCCCCCCCCCCCCACAATGATCTT
CATTGAATGAACTACAAGCTTCAAGTGTGTTTTTGTGGTGCAGTTA
SEQ ID NO: 15 Oryza sativa PIN-LIKES 6-1 (LOC4327495) GenBank Accession No.
XR_003239989 transcript variant X2
GATAGCTTAATCTCGAGCGCTTCGAGTTCGATCTCTCGTGAAGGGGACGACGAGGGCATTGTCTCCCTCC
TCGCGCGCGCGGAGTCGCCGCCGCCGCCGCCTCGTCCTCGGCCACGCGGAATTGCGTGGGTCCTGGTTGT
GGAAGTCAGGAAGTGAAGAAGAAGAAGAAGAAGTGAACTAGTGAAGCCTGGGGCAAGCCATGATGGAGAG
GTCGCTGATGGAGGCGCTGGCTACGGCGGCGCAGGGAGGCACCGTGGGGACGTCGGTTTTCGACATGCTC
AAGTACGCCGTGCTGCCCATTGCCAAGGTGTTTACCGTCTGTTTCATGGGGTTCCTCATGGCCTCTAAGT
ATGTCAATATCCTCCAGCCGAACGGCCGCAAGCTTCTCAATGGGCTTGTGTTCTCGCTTCTACTTCCTTG
CCTTATATTTTCCCAACTGGGTAGAGCAATCACAATCGAAAAGATGCTGCAATGGTGGTATATTCCAGTA
AATATTGTTGTAGGTGCAGTGTCAGGCTCTTTGATTGGCTTTGTGGTGGCTTCTATCATCCGACCACCTT
ATCCATACTTCAAGTTCACTGTGATTCACATTGGAATAGGGAATATTGGAAATATACCTCTGGTTCTCAT
TGCAGCATTATGTCGAGATCCCACCAATCCTTTTGGTGACTCTGATAAATGTAATGAAGATGGGAATGCG
TATATCTCATTTGGTCAATGGGTTGGTGCAATTATTGTTTACACATATGTGTTCAAGATGCTTGCTCCAC
CACCTGGTGAATCCTTTGATAGCGCTGAAGAAGATATTCTTCCAATTAAGGCATCTGGAGATAATGTGGT
GCCTGAAAAAGGGAAATATCCAACAAGCACTCGCACTAGTACTGTACCTGAAAATGAGCCTTTGTTATCT
TCTGAAGGTGATAAAAATGTTTCTACTTCTCTAGGATCGAAGATAATGGGCATTGTTAGAAGCATGGTTA
AGTTCCTAAAAGACAAGCAGCTTCTTCAGCCACCAATTATTGCCTCTGTTTTTGCAATTGCCATTGGTGT
TGTTCCAGTCTTGAAGAATTTTGTCCTCACTGATGATGCACCTCTGTTCTTCTTCACAGACAGCTGTCTC
ATTCTAGGGGAAGCAATGATCCCTTGCATTTTGCTTGCAGTGGGGGGCAATCTTGTCGATGGTCCTGGTG
AAGGAAGTAACAGGCTTGGTGTGCGGACAACCGTTGCTATTATTTTTGCACGGCTCATCTTGGTTCCTCT
TGCTGGGGTTGGCATCATTGTGTTGGTTGATAAACTTGGTTTCATTCCTAAAGATGACAAAATGTTCAAG
TTTGTCTTGCTACTGCAGCATTCTATGCCCACATCTGTGCTGTCAGGTGCTGTTGCAAACCTGAGGGGCT
GTGGAAAAGAATCTGCTGCTATCTTGTTCTGGGTGCACATCTTTGCTGTGTTCTCCATGGCAGGATGGAT
TATTTTGTATTTGAGCTTACTCTTCTAAGTACGTCATACCCTGATGCTATACTGCACAGGAACGTCCATA
AGATCAAAATATCACCAGATATACCAGAAAAAGATAGTACCATGACATGTACATTAGCCAGTAAACCATG
GCGACAGCCTGAAGGATGGGTGCCCGACATCCACCCACGCACATACATGAATGCCAAAATCAAAATATTT
GCCTGCAAGCTCATGCCAGCGGTTCCTAGTATGCATGTGGGTCATGTAGCCTTAACTAGCATTGGTACTT
CCAGATCCAGTGATATCAGAGTACATATGTGATCTCTCCATGTGCTTATAGATCTGGGAACCTGCAGATC
TGAAGTTCTGAACCCACAGTTTTCATTCACTTTTAATCTTTTGAATCTGAAGGAGAGGGAGATTCATGAT
TCATCACCGTACCAAAAAAAAAGGTGAGAGATTTCTATAGTGAACTTTAACCAAGCAGACAGCTCAATAT
GCCTCTTGGTGTGCGTATTGGTTGGGGTCTCAAAATCAGATGCAAATCCGTCAAACGGAATATACTTGGA
AGATACGTATCGCGTCAGCACTCAGCATGGAGTCAGACAGGTACTGACATTCAGACACGCTAGGGTCGGA
CAAAAAACAGATACTGCCAATATTCGTTCATCGGAAATAGAACAAAAAAACATGTATTGACAATAAATCA
GTGTACAGAACAGTACCGTATCGATATGGCGAGCAGATAATAACGCAAGGATACGTATGAGCCGTCTCTC
ATCAGCAGAAATGGCTGCATGTATTCGCTTCGCACCCA
SEQ ID NO: 16 Oryza sativa PIN-LIKES 6-1 (LOC4327495) coding sequence
ATGATGGAGAGGTCGCTGATGGAGGCGCTGGCTACGGCGGCGCAGGGAGGCACCGTGGGGACGTCGGTTT
TCGACATGCTCAAGTACGCCGTGCTGCCCATTGCCAAGGTGTTTACCGTCTGTTTCATGGGGTTCCTCAT
GGCCTCTAAGTATGTCAATATCCTCCAGCCGAACGGCCGCAAGCTTCTCAATGGGCTTGTGTTCTCGCTT
CTACTTCCTTGCCTTATATTTTCCCAACTGGGTAGAGCAATCACAATCGAAAAGATGCTGCAATGGTGGT
ATATTCCAGTAAATATTGTTGTAGGTGCAGTGTCAGGCTCTTTGATTGGCTTTGTGGTGGCTTCTATCAT
CCGACCACCTTATCCATACTTCAAGTTCACTGTGATTCACATTGGAATAGGGAATATTGGAAATATACCT
CTGGTTCTCATTGCAGCATTATGTCGAGATCCCACCAATCCTTTTGGTGACTCTGATAAATGTAATGAAG
ATGGGAATGCGTATATCTCATTTGGTCAATGGGTTGGTGCAATTATTGTTTACACATATGTGTTCAAGAT
GCTTGCTCCACCACCTGGTGAATCCTTTGATAGCGCTGAAGAAGATATTCTTCCAATTAAGGCATCTGGA
GATAATGTGGTGCCTGAAAAAGGGAAATATCCAACAAGCACTCGCACTAGTACTGTACCTGAAAATGAGC
CTTTGTTATCTTCTGAAGGTGATAAAAATGTTTCTACTTCTCTAGGATCGAAGATAATGGGCATTGTTAG
AAGCATGGTTAAGTTCCTAAAAGACAAGCAGCTTCTTCAGCCACCAATTATTGCCTCTGTTTTTGCAATT
GCCATTGGTGTTGTTCCAGTCTTGAAGAATTTTGTCCTCACTGATGATGCACCTCTGTTCTTCTTCACAG
ACAGCTGTCTCATTCTAGGGGAAGCAATGATCCCTTGCATTTTGCTTGCAGTGGGGGGCAATCTTGTCGA
TGGTCCTGGTGAAGGAAGTAACAGGCTTGGTGTGCGGACAACCGTTGCTATTATTTTTGCACGGCTCATC
TTGGTTCCTCTTGCTGGGGTTGGCATCATTGTGTTGGTTGATAAACTTGGTTTCATTCCTAAAGATGACA
AAATGTTCAAGTTTGTCTTGCTACTGCAGCATTCTATGCCCACATCTGTGCTGTCAGGTGCTGTTGCAAA
CCTGAGGGGCTGTGGAAAAGAATCTGCTGCTATCTTGTTCTGGGTGCACATCTTTGCTGTGTTCTCCATG
GCAGGATGGATTATTTTGTATTTGAGCTTACTCTTCTAA
SEQ ID NO: 17 Oryza sativa PIN-LIKES 6-1 (LOC4327495) amino acid sequence
MMERSLMEALATAAQGGTVGTSVFDMLKYAVLPIAKVFTVCFMGFLMASKYVNILQPNGRKLLNGLVFSL
LLPCLIFSQLGRAITIEKMLQWWYIPVNIVVGAVSGSLIGFVVASIIRPPYPYFKFTVIHIGIGNIGNIP
LVLIAALCRDPTNPFGDSDKCNEDGNAYISFGQWVGAIIVYTYVFKMLAPPPGESFDSAEEDILPIKASG
DNVVPEKGKYPTSTRTSTVPENEPLLSSEGDKNVSTSLGSKIMGIVRSMVKFLKDKOLLQPPIIASVFAI
AIGVVPVLKNFVLTDDAPLFFFTDSCLILGEAMIPCILLAVGGNLVDGPGEGSNRLGVRTTVAIIFARLI
LVPLAGVGIIVLVDKLGFIPKDDKMFKFVLLLQHSMPTSVLSGAVANLRGCGKESAAILFWVHIFAVESM
AGWIILYLSLLF
SEQ ID NO: 18 Oryza sativa PIN-LIKES 6-2 (LOC4339130) Gene NC_089039
REGION: complement (23811988 . . . 23815523)
GTGGCACGCTTCTCCTCTCGTCGCCGCCTCGTCTTCTCAACTTCCCTCGGCTCGCGGCGGCGTCGGCCGT
GAATCCTCCGCCACCGCCGCCGCCGCCGCCGCCGCCGACGAGCCCTACCACGATCGCCTCGCCGCATTCC
CGGGGAGATTCCGCCGCCGTCGGCTTCTTCCCCGTGCTCCCGCGGCGGCGTTTTGAGCTCTCTCTCCCCT
GCGGGTTTTCCGCCCTTTTTGGGCACGGATGAGTGGGATCACATTGCGCGATCTCTAGCTCAAGTCTCGG
ATTTCCGGCGGTTGGTAGGCTCGTACTAAGCTGCCTAAATCTCGGATATCCTTTTTTTTTCTTTCTAATA
ATGCTGGATTAACCCTAAATTTCGGCGAGTTGGTATCCGATTCCTACGCTGATTGAGTGATTGGGTTGGA
TCCTGACACGGAAGGGGAAAGTGGTTGTACAAGTCGGGAGGAGGAGTTGGCCGTTTCGGGAACTGATTCC
TTGCTATTTTTGGCTACAAGTTCGCCGCCCCGGGCAGCTGCTTGGCCGTCGCGACGCAGCCGCGTCTGTT
CGATCAGTTCAGCTCGCGCGGTTTTAGGTGGGGAGGAATCCTGTTCGATTTCGCCGGTTGCTTGTGGTTT
CGTGTCGGAGGCGGGGGCGGCGATGATGGGGAGGTCGGTGCTGGAGATGGTGGTGGCGGCGGCGCAGGGC
GGCGGCGGCGCGGCGGGGGAGTCGGTGTTGGGCATGTTCAGGTACGCCGTGCTGCCCATCGCCAAGGTGT
TCGTCGTCTGCTTCATGGGGTTCCTCATGGCGTCCAAGCGCGTCGGCGTGCTCAAGCCCAGCGGCCGCAA
GCTTCTCAATGCGGTAAGGCGCCTAATTCAGTAGTTCTCACAACCATCATGGTGGCTCGCTAATTTTCAG
AATATAATGGAATTCAGACTAGCTATGGGAACATCAGCAGAATTCAGGCTAGCTATGTTGATTTATCTTG
TCTTTTTTTTTTTTGTTATTTTTGCAGCTTGTGTTCTCTCTGCTCCTTCCTTGCCTAATATTTGCGCAGC
TCGGGAGATCAATCACAATTGACAAGATCATGGAGTGGTAAGAAAGAACATATTAGTACTCATTTAGATG
ATTAGCCACAAGTTGTATCTTTCCCTTGGCGATATTTGGGGTCTTCTTGTGTCAAAGCATTTCAACATTA
CTCATGATGTTGCATTGCAGGTGGTTCATTCCAGCAAACATTGCTCTGGGTGCAGTCTCTGCATCTTTGG
TTGGACTTATAGTGGCATTGATTGTACGGCCGCCTTATCCATACTTCAAGTTCACCATCACTCACATTGG
AATAGGTTTGTACATTCCTCACGCCCTTATGAATATCGCTAGCTTCTGATTCATCACGCATTCTGCATTA
TATTTTTAACATTTCACTTAATGCAGGGAACATTGGAAATATACCACTTGTCCTTATCTCGGCATTATGT
CGCGACCAATTAAATCCGTTTGGTGACTCGAACAAATGTACTCAAGATGGAAATGCATACTTATCATTTG
GTCAATGGGTATGTCCAAATCCGTTTGTTTATGGAGTTGCACATCCCTTTTTAAAATGCTACTCCAAATA
CTTATCATATCTATTGGATTGTTTACCGGTGCAGGTTGGTGCCATTATTGTTTACACATATGTGTTCAAG
ATGCTTGCTCCACCCCCCGGGCAGACCTTTGACAGCTGCGATGAGGAGAGAGATAAACTTCCAATTAAGG
CTCCTAATACCATGTCAAGTGTAGCGAAATATCCATCAAGTGCTCATGGCAATACACATGAGGAAGAGCC
CCTGCTATCTATAGAGGAGGAGGAGGAGGAGGAGGGGCAGGACGTTCATTCTTTAGGTTCAAAAGTAAGA
AGTGGGCACAACATAATGCTAGTTCAGCATATTGGAAAAAAAAAATAGAGGCACTTCAGTAGTTGTTACC
AAAATAACTAATAATACTCCATTTATGGCTTAATGCAGATAATGATTCCTATCAAGGGCATGGTTAGATT
CCTACAAAAGAAGCAGCTCCTCCAACCACCAATCATTGCATCTGTAAGTGCTCGGTATATTTGGTTTTTG
TCTCTTATCACTGCCGTAAAACAATACTTGCATTTTTCTCAATTATCTGAATGTTCCTTTGCGTCATGTA
ACAGGTTCTTGCAATTACTCTCGGTGTTGTGCCATTCTTGAAGAATTTGATCCTCACAGATGACGCGCCT
CTATTCTTTTTAACAGATAGCTGTCTCATTCTTGGGTATTGACCTGTCTCATGACTTGTAGTTTCTTCTT
GCAATATGCCTATAGTGTCTTGTAATAACAAATATGATTCGCATTTAAAAACCATCTCTTGCTAACAATG
CTGTTTTATATATTTCAGGGAAGCTATGATTCCGTGCATTTTGCTTGCCGTTGGTGGCAATCTTGTTGAC
GGTAAGCTCATCTTGTATTCTCAAATCTCATTAGAACCGTGCTGCAATATGAAGTGTGCTTTTAGTACAT
TTATACTTCTGCAATTTATATGTTTATCTGAAGTCCATACTTGTGGTTTATTATGGTTTTAGGCCCTGGT
GAAGGAAGTAGGAGACTTGGGGTTCGGACCACCGTCGCGATTATATTTGCACGCTTGATATTGGTTCCAA
TTGCTGGTATTGGCATCGTCTCATTTGCTGACAAGCTTGGATTTATTCCAAAAGGAGACAAGATGTTCAA
GTTCGTCCTGTTACTGCAGCACTCTATGCCCACATCGGTACTATCAGGTACGAAAAAGTATAAGGATGAC
TGAGACATCATATTTGGCATGTTAATCAACCTTTGAGTATAATACAGTTGCAATACTAATCTGCTATTTT
TTATGGGCAGGTGCTGTTGCAAACCTAAGAGGTTGTGGCAAGGAATCAGCTGCAATCTTGTTCTGGGTGC
ACATTTTTGCTGTCTTCTCCATGGCAGGATGGATTATCTTATATCTTACCATGCTGTTCTAGGTATTTGA
TTTTGAGCCCCCTCTGGGTGTGTGTGCCTGAATATGATGCTCCTCCTGGGTTAGCTGCTGTCGGCAGTAC
TGTGTCTGGTTGTACATTTTTCCGCTGTCCCTCGAGTTATTGTAGTTTATAATGTTTCATCTGATTCTTT
TGTAGATACTAACTGCACTATGACATAATATGATATGTTCATGAAATACATGTTGCTGCAAAGTGCAAGG
TGATTGGAAGTGCGACCCTGTTGGATCTGCGGGTTGCATTTTCCGGGCCGTGGACAATTGCGAGAGGCCT
GCTATTGTATGTTTTGTTTGGTTAGCTGCTGAAAACAATAGATGAGGCAAAGGAATTGTGTCAGCACCTC
TTGTACATTTGATCGTAAAATACAGGATATCTCCAACTAATAGAGAGCCTTGTATCCCCAAATTCTTGCT
TTTGGTTTAGTAGGACCAGTAGATTGAAGTGCATTATGTAAGAGGAGATTTTTTTTTTCTAGAGAAGCGT
ACTGTGAATTTGAAATCCATGGCATATTTCTACAGA
SEQ ID NO: 19 Oryza sativa PIN-LIKES 6-2 (LOC4339130) GenBank Accession No.
XM_015782757.3 transcript
GTGGCACGCTTCTCCTCTCGTCGCCGCCTCGTCTTCTCAACTTCCCTCGGCTCGCGGCGGCGTCGGCCGT
GAATCCTCCGCCACCGCCGCCGCCGCCGCCGCCGCCGACGAGCCCTACCACGATCGCCTCGCCGCATTCC
CGGGGAGATTCCGCCGCCGTCGGCTTCTTCCCCGTGCTCCCGCGGCGGCGTTTTGAGCTCTCTCTCCCCT
GCGGGTTTTCCGCCCTTTTTGGGCACGGATGAGTGGGATCACATTGCGCGATCTCTAGCTCAAGTCTCGG
ATTTCCGGCGGTTGGTAGGCTCGTACTAAGCTGCCTAAATCTCGGATATCCTTTTTTTTTCTTTCTAATA
ATGCTGGATTAACCCTAAATTTCGGCGAGTTGGTATCCGATTCCTACGCTGATTGAGTGATTGGGTTGGA
TCCTGACACGGAAGGGGAAAGTGGTTGTACAAGTCGGGAGGAGGAGTTGGCCGTTTCGGGAACTGATTCC
TTGCTATTTTTGGCTACAAGTTCGCCGCCCCGGGCAGCTGCTTGGCCGTCGCGACGCAGCCGCGTCTGTT
CGATCAGTTCAGCTCGCGCGGTTTTAGGTGGGGAGGAATCCTGTTCGATTTCGCCGGTTGCTTGTGGTTT
CGTGTCGGAGGCGGGGGCGGCGATGATGGGGAGGTCGGTGCTGGAGATGGTGGTGGCGGCGGCGCAGGGC
GGCGGCGGCGCGGCGGGGGAGTCGGTGTTGGGCATGTTCAGGTACGCCGTGCTGCCCATCGCCAAGGTGT
TCGTCGTCTGCTTCATGGGGTTCCTCATGGCGTCCAAGCGCGTCGGCGTGCTCAAGCCCAGCGGCCGCAA
GCTTCTCAATGCGCTTGTGTTCTCTCTGCTCCTTCCTTGCCTAATATTTGCGCAGCTCGGGAGATCAATC
ACAATTGACAAGATCATGGAGTGGTGGTTCATTCCAGCAAACATTGCTCTGGGTGCAGTCTCTGCATCTT
TGGTTGGACTTATAGTGGCATTGATTGTACGGCCGCCTTATCCATACTTCAAGTTCACCATCACTCACAT
TGGAATAGGGAACATTGGAAATATACCACTTGTCCTTATCTCGGCATTATGTCGCGACCAATTAAATCCG
TTTGGTGACTCGAACAAATGTACTCAAGATGGAAATGCATACTTATCATTTGGTCAATGGGTTGGTGCCA
TTATTGTTTACACATATGTGTTCAAGATGCTTGCTCCACCCCCCGGGCAGACCTTTGACAGCTGCGATGA
GGAGAGAGATAAACTTCCAATTAAGGCTCCTAATACCATGTCAAGTGTAGCGAAATATCCATCAAGTGCT
CATGGCAATACACATGAGGAAGAGCCCCTGCTATCTATAGAGGAGGAGGAGGAGGAGGAGGGGCAGGACG
TTCATTCTTTAGGTTCAAAAATAATGATTCCTATCAAGGGCATGGTTAGATTCCTACAAAAGAAGCAGCT
CCTCCAACCACCAATCATTGCATCTGTTCTTGCAATTACTCTCGGTGTTGTGCCATTCTTGAAGAATTTG
ATCCTCACAGATGACGCGCCTCTATTCTTTTTAACAGATAGCTGTCTCATTCTTGGGGAAGCTATGATTC
CGTGCATTTTGCTTGCCGTTGGTGGCAATCTTGTTGACGGCCCTGGTGAAGGAAGTAGGAGACTTGGGGT
TCGGACCACCGTCGCGATTATATTTGCACGCTTGATATTGGTTCCAATTGCTGGTATTGGCATCGTCTCA
TTTGCTGACAAGCTTGGATTTATTCCAAAAGGAGACAAGATGTTCAAGTTCGTCCTGTTACTGCAGCACT
CTATGCCCACATCGGTACTATCAGGTGCTGTTGCAAACCTAAGAGGTTGTGGCAAGGAATCAGCTGCAAT
CTTGTTCTGGGTGCACATTTTTGCTGTCTTCTCCATGGCAGGATGGATTATCTTATATCTTACCATGCTG
TTCTAGATACTAACTGCACTATGACATAATATGATATGTTCATGAAATACATGTTGCTGCAAAGTGCAAG
GTGATTGGAAGTGCGACCCTGTTGGATCTGCGGGTTGCATTTTCCGGGCCGTGGACAATTGCGAGAGGCC
TGCTATTGTATGTTTTGTTTGGTTAGCTGCTGAAAACAATAGATGAGGCAAAGGAATTGTGTCAGCACCT
CTTGTACATTTGATCGTAAAATACAGGATATCTCCAACTAATAGAGAGCCTTGTATCCCCAAATTCTTGC
TTTTGGTTTAGTAGGACCAGTAGATTGAAGTGCATTATGTAAGAGGAGATTTTTTTTTTCTAGAGAAGCG
TACTGTGAATTTGAAATCCATGGCATATTTCTACAGA
SEQ ID NO: 20 Oryza sativa PIN-LIKES 6-2 (LOC4339130) GenBank Accession No.
XM_015782757.3 coding sequence
ATGATGGGGAGGTCGGTGCTGGAGATGGTGGTGGCGGCGGCGCAGGGCGGCGGCGGCGCGGCGGGGGAGT
CGGTGTTGGGCATGTTCAGGTACGCCGTGCTGCCCATCGCCAAGGTGTTCGTCGTCTGCTTCATGGGGTT
CCTCATGGCGTCCAAGCGCGTCGGCGTGCTCAAGCCCAGCGGCCGCAAGCTTCTCAATGCGCTTGTGTTC
TCTCTGCTCCTTCCTTGCCTAATATTTGCGCAGCTCGGGAGATCAATCACAATTGACAAGATCATGGAGT
GGTGGTTCATTCCAGCAAACATTGCTCTGGGTGCAGTCTCTGCATCTTTGGTTGGACTTATAGTGGCATT
GATTGTACGGCCGCCTTATCCATACTTCAAGTTCACCATCACTCACATTGGAATAGGGAACATTGGAAAT
ATACCACTTGTCCTTATCTCGGCATTATGTCGCGACCAATTAAATCCGTTTGGTGACTCGAACAAATGTA
CTCAAGATGGAAATGCATACTTATCATTTGGTCAATGGGTTGGTGCCATTATTGTTTACACATATGTGTT
CAAGATGCTTGCTCCACCCCCCGGGCAGACCTTTGACAGCTGCGATGAGGAGAGAGATAAACTTCCAATT
AAGGCTCCTAATACCATGTCAAGTGTAGCGAAATATCCATCAAGTGCTCATGGCAATACACATGAGGAAG
AGCCCCTGCTATCTATAGAGGAGGAGGAGGAGGAGGAGGGGCAGGACGTTCATTCTTTAGGTTCAAAAAT
AATGATTCCTATCAAGGGCATGGTTAGATTCCTACAAAAGAAGCAGCTCCTCCAACCACCAATCATTGCA
TCTGTTCTTGCAATTACTCTCGGTGTTGTGCCATTCTTGAAGAATTTGATCCTCACAGATGACGCGCCTC
TATTCTTTTTAACAGATAGCTGTCTCATTCTTGGGGAAGCTATGATTCCGTGCATTTTGCTTGCCGTTGG
TGGCAATCTTGTTGACGGCCCTGGTGAAGGAAGTAGGAGACTTGGGGTTCGGACCACCGTCGCGATTATA
TTTGCACGCTTGATATTGGTTCCAATTGCTGGTATTGGCATCGTCTCATTTGCTGACAAGCTTGGATTTA
TTCCAAAAGGAGACAAGATGTTCAAGTTCGTCCTGTTACTGCAGCACTCTATGCCCACATCGGTACTATC
AGGTGCTGTTGCAAACCTAAGAGGTTGTGGCAAGGAATCAGCTGCAATCTTGTTCTGGGTGCACATTTTT
GCTGTCTTCTCCATGGCAGGATGGATTATCTTATATCTTACCATGCTGTTCTAG
SEQ ID NO: 21 Oryza sativa PIN-LIKES 6-2 (LOC4339130) GenBank Accession
No. XM_015782757.3 amino acid sequence
MMGRSVLEMVVAAAQGGGGAAGESVLGMFRYAVLPIAKVFVVCFMGFLMASKRVGVLKPSGRKLLNALVF
SLLLPCLIFAQLGRSITIDKIMEWWFIPANIALGAVSASLVGLIVALIVRPPYPYFKFTITHIGIGNIGN
IPLVLISALCRDQLNPFGDSNKCTQDGNAYLSFGQWVGAIIVYTYVFKMLAPPPGQTFDSCDEERDKLPI
KAPNTMSSVAKYPSSAHGNTHEEEPLLSIEEEEEEEGODVHSLGSKIMIPIKGMVRFLOKKQLLOPPIIA
SVLAITLGVVPFLKNLILTDDAPLFFLTDSCLILGEAMIPCILLAVGGNLVDGPGEGSRRLGVRTTVAII
FARLILVPIAGIGIVSFADKLGFIPKGDKMFKFVLLLQHSMPTSVLSGAVANLRGCGKESAAILFWVHIF
AVFSMAGWIILYLTMLF
SEQ ID NO: 22 Setaria italica PIN-LIKES 6 (SETIT_001507mg) Gene NC_028454
REGION: 39919686 . . . 39924250
GACCCCGCGTCGTCTCGCGGAGTCGTCTCGTCCTTTCCGTTCTCCCCCCTTCCCCCCCGCGGGAGTCGGA
GCCCAACCCATCCCACCCCAGATCCCACCCGCTCCCCGAATCGCCGCCACTTTTTCCATTAGCAGCCTTC
CCGCAATCCCGCGGCCAGTTCGAGCCCTTCGCGGGAGGGACGGGCTCACTGATTACTCGGGCTCAGCCCC
CCTCCCTCCCAGCAGCGCGGGGTCACCTCGGCCGTCTCGCGGTGAGTTGAAATGTTGAATGGAGTCCTCG
GTCCAATCTTCCCTTCCGCTTGGGATTGTTTTCGGTGTTCTGAATTTGCGTGGCGTACTGGCGTGGCGTG
TGGGGGTCATGGCGTGCCGTCGTCGCGATCGCTCTCCCTCGCATTGCGCGATTCGATTTTTTTTTTTTTT
TACTTTGCTTGCTTGTTTGCCTTTTGGGATGTAATGGTGATTTCTACTGCTGGCGATTCTGAGCTCGGTT
GCGTCTGAGCTTCCGCAGAGTGGTTTAGTGCGGATTTGGTAAAACAGTTTGAGTTGGTATGGCGTGACGA
ATCTAACCTGAGCAGATACGCAGCTGCGGGGAATTGGATCCCCCAACCATGCGAATTTGGAACGGAGGAG
TAGTGGAGTGTTCGGGAATCGGTGTCGTCATTTTTCTACGAGTCGAGTTAGTTGGAATCTAGTTAGGTCA
TGGTTCTCGTTCCGAGATGATTAAATCTTCTATGCGTGGGTTTTCATCAAACTAGAAGTTGGTTGCTGCT
AAGCTTCCCGCTTCGGAAGCTCCATGCCGGATTTGGTTACGCGTACCGGTGAGCTGAGCGAACGGCGGGC
TCTCTTCCAGTTGGGTTGCCCCTGAACAACCAGAGCCAAAAAGCAGTTCCAGAATTTTCCAGACGCTGCC
TCTATATTTTGCCTCCTAACTTCTAAGTCATTCATAATGGCAGACTCCTGATTGCGTTGGACACGGGTCC
TAAGCTGTGAAGGCGGAAAGGAAAGGAAAGGAAAAGGAAGGCTTCAGTCCCAGGTCAAGCCATGATGGAG
AGGTCGCTGCTGCAGGCGCTGGCCACGGCGGCGCAGGGAGGCACCTCGGGGACTTCGGTACTGAGCATGC
TCAAGTATGCTGTGCTGCCCATCGCCAAGGTGTTCACGGTCTGCTTCATGGGGTTCCTCATGGCCTGCAA
GTACGTCAACATCCTCCAGCCCAACGGCCGCAAGCTTCTCAACGGGGTGAGCTTTGTCTCCTTGTAATAA
AAGCTAGCTATGGGAAGGCTGTCTAATGGATTTTGTTCAAAGTAGGAAGACGTGGAGCATTAGAGGCCAT
GATTGCACTAATGCGCTTTCCCACTATCAAATTAGACTGACATTTTCATTGTTTGCTGCAGCTTGTGTTT
TCGCTTCTGCTTCCTTGCCTTATATTTTCCCAACTGGGTAGAGCAATCACTATTGAGAAGATGTTACAGT
GGTAAGGAATATGGATTGATATCGTACATCAATTTTGTTTTATCTAGTAACATAATACTGTGTTACTTCA
TCTAAGTGTGCTGTATGGTTGTTGAATTCTGATGTGAGAGCTATTGACCAAGATGTTGTTTTCTGCTAAT
ATTCTGTCTACTTGTATGTGCCTCTAGTCCTCTATTGTCTATAATCAGAAACAACCATTACTATATTACT
TAAAAGTACAACGAAATAGTCTACATGTTTCTGGTTGGGTCAACTTTTAAATCATGAACTTCCAAACGGA
ACAAATTAGCTTGCTGAGCTCAAAATTCCTTCAGAAGGGACAGAAAAGAAATGGGCGTTCAAGTGTTAAT
TTGGTTCCAGTTGAATCTATGGGTCATATGGTTTTGACTACTTTAGTTATTTAATATACTGAAATATTGC
AATTGTTGGAGTTTGGAGAAACTAGTCCATGTCATTTATCTGCATATGTATGTAATAATCTAAATTGTCA
TAATTTTTCTTACTGGGAATGTTTATCTTGTTGTAATTTAATATATTGAACTTGATGTTATGCTGTTATG
GACTATTCAGTCCTCGTTTGCATTGACACATGCCTCAATTTTCTTCCACTCCCCGCATGTATATGCATTA
CAAGTGTCAATTTTCTTTTTTCTTTTGCATAAACTACTTTAGGAGTATGTTCTGGAGTTCAGCTTTGCCA
AATAAGCTGTGCACTCGTGTTCTGCCTCTCCTGTTATATTTCTAATAAGGTCATATATGCATGTTTGCAG
GTGGTATATTCCAGTAAATATTGTTGTAGGTGCAGTATCTGGCTCTTTGATTGGCTTTGTTGTGGCATCT
ATCATCCGACCCCCATATCCGTACTTCAAGTTCACCATTATCCATATAGGAATAGGTACCACCTTTTGTT
TCTTGAAAATAACTGCATTTGTATTTTTTTTTTCATTCAAATATCCAATATCAATACAGCTCCTTGTGTT
TTATGCAGGAAATATTGGAAATATACCTCTGGTCCTCATTGCAGCGTTATGTCGGGATCCATCTAATCCG
TTTGGTGACTCTGATAAATGCAACCAAGATGGAAATGCATATATCTCATTTGGTCAATGGGTATGTCTAT
CCTCTTCTCTTTTCCCAGCTCATAAATGTCCATGTTTTACTCAGAAGTCTGAATACCAACTCTGTAGATC
AAAGACAATCGCTTAGCATCTATATAGGCTACAAATAGCTGTTGGGATCGGTCACTCAGTGAATCCTTTT
CGTGTTGCAGGTTGGTGCTATTATTGTTTACACATATGTGTTCAGGATGCTTGCTCCACCACCAGGACGC
ACCTTTGATGGCTCTGATGACGATGAACTTCCAGTCAAGGCATCTGGAGAGAATGCGGTGCCCGAACTCA
GCAAATATCCAATTCCAACAAGCACTCACACTAGCACTGTACCAGAGGATGAACCATTGTTAGCTGCTGA
GAAAGTTCAAAAAGAATGTGCCACTTCTGTAGGATCAAAGGTAAGAAAATGTAGTCATCGACTTGGGTTT
CTAGTTATCGTTGTGATGCTCTTACTTCCCGCTAAAAAAAATAACCATGTTCTTACTAATGCAGATAATG
GGCCATGTTAAATGTGTGATTAAATTCCTGAAAGACAAGCAGCTTCTCCAGCCACCGATTATTGCATCTG
TATGTATTGCTGTTGCACTTTTTAGTAGTGTTTATATAAATCATTATGCATTCTGCGTCTTGACTTTATT
TTTAAAAATGCACAGGCTTTTGCAATTGTCATTGGTATTGTCCCATTCCTGAAGAATTTTGTACTCACAG
ATGAAGCGCCTCTCTTCTTTTTCACAGACAGCTGCCTGATTCTTGGGTATTTACATGTCTCATTCTCTGT
TTCCTTATGAATTATACAGATTATAGCCAATAAAGTTAGCAAACTTTGACAATCTATAATAGTATCTAGT
GTCTAATCCGTAAAACACTTTCAGGGAAGCTATGATCCCATGCATTTTACTTGCTGTTGGGGGCAATCTC
GTTGACGGTAAGCATCTTTTTTGTTTGAAACTCTGGGAAAATTGCATGGGGCGAGATCAACCTTAGCAGT
ACCATCTTATAGGGTTTAGAGGCATTTTTCATGATTTTGTCTTTACCTATGTTTATGGCATCAGGTCCTG
GTGAAGGAAGTAAGAGGCTTGGTGTGCGTACAACCGTTGCTATAATTTTTGCACGTTTGGTCTTGGTCCC
TCTTGCTGGGGTTGGCATCTTCATGGTAGTTGATAAACTTGGTTTCATTCCCAAAGACGACAAAATGTTC
AAGTTTGTCGTGCTACTGCAGCATTCTATGCCCACATCAGTGCTGTCAGGTACGATAAGACACCAAGGAC
TACTACTTATCACTCATCATGTGCTTAGGGAGAACAGATTCCTCTTATAATGCTGTGTTATATGAGATCT
GTTAGGTAATGGGGGTGGGGTGCCATGTTCCACATGGTAGCAAACCATGTTATGGAATCTGATTATACAT
TTTTTTTTGCAGGTGCTGTTGCGAACCTGAGAGGGTGTGGTAAAGAATCAGCTGCAATCTTGTTCTGGGT
TCACATTTTTGCTGTGTTCTCCATGGCAGGATGGATCATTGTATATTTGAGCTTGCTCTTCTAAGTATGT
CGCTCCCTTATTTTAGCTGCTTTTTGCTCCATTTGTCATCGCATTTGCACCTGTCTGCTGTAACAGAAAA
CCTTGATATTTACGTTGATCTGATATTTGGTCAACTGATCTGCAGACACCTTACTGCACACGAAGATCTT
TGAAAGAACATATCATCCAAAGTATCACGGCGAAAATAGTGACATGAATATGTACAGTAGCTAGTAAATC
ATGGTGAGGTATGAGTTGTATCAGCATTGATATACGGTCCTTCCCAATGCTATGGTAATTGTGGTTCTCT
TCCCACTGCTGCAAGCAGTGAGCGAAGATGGCACAGCGGTGTTGCCCTGCTCGGTCACACCCGAACCACT
TAGATGGGGATGCCACGTTGTCCCAATCCATCCATTCGCTTTGTTCATTCCATCTCCTCACCAAAATATT
AGCTTAATTTACTCC
SEQ ID NO: 23 Setaria italica PIN-LIKES 6 (SETIT_001507mg) GenBank Accession
No. XM_004970317.4 transcript
GACCCCGCGTCGTCTCGCGGAGTCGTCTCGTCCTTTCCGTTCTCCCCCCTTCCCCCCCGCGGGAGTCGGA
GCCCAACCCATCCCACCCCAGATCCCACCCGCTCCCCGAATCGCCGCCACTTTTTCCATTAGCAGCCTTC
CCGCAATCCCGCGGCCAGTTCGAGCCCTTCGCGGGAGGGACGGGCTCACTGATTACTCGGGCTCAGCCCC
CCTCCCTCCCAGCAGCGCGGGGTCACCTCGGCCGTCTCGCGACTCCTGATTGCGTTGGACACGGGTCCTA
AGCTGTGAAGGCGGAAAGGAAAGGAAAGGAAAAGGAAGGCTTCAGTCCCAGGTCAAGCCATGATGGAGAG
GTCGCTGCTGCAGGCGCTGGCCACGGCGGCGCAGGGAGGCACCTCGGGGACTTCGGTACTGAGCATGCTC
AAGTATGCTGTGCTGCCCATCGCCAAGGTGTTCACGGTCTGCTTCATGGGGTTCCTCATGGCCTGCAAGT
ACGTCAACATCCTCCAGCCCAACGGCCGCAAGCTTCTCAACGGGCTTGTGTTTTCGCTTCTGCTTCCTTG
CCTTATATTTTCCCAACTGGGTAGAGCAATCACTATTGAGAAGATGTTACAGTGGTGGTATATTCCAGTA
AATATTGTTGTAGGTGCAGTATCTGGCTCTTTGATTGGCTTTGTTGTGGCATCTATCATCCGACCCCCAT
ATCCGTACTTCAAGTTCACCATTATCCATATAGGAATAGGAAATATTGGAAATATACCTCTGGTCCTCAT
TGCAGCGTTATGTCGGGATCCATCTAATCCGTTTGGTGACTCTGATAAATGCAACCAAGATGGAAATGCA
TATATCTCATTTGGTCAATGGGTTGGTGCTATTATTGTTTACACATATGTGTTCAGGATGCTTGCTCCAC
CACCAGGACGCACCTTTGATGGCTCTGATGACGATGAACTTCCAGTCAAGGCATCTGGAGAGAATGCGGT
GCCCGAACTCAGCAAATATCCAATTCCAACAAGCACTCACACTAGCACTGTACCAGAGGATGAACCATTG
TTAGCTGCTGAGAAAGTTCAAAAAGAATGTGCCACTTCTGTAGGATCAAAGATAATGGGCCATGTTAAAT
GTGTGATTAAATTCCTGAAAGACAAGCAGCTTCTCCAGCCACCGATTATTGCATCTGCTTTTGCAATTGT
CATTGGTATTGTCCCATTCCTGAAGAATTTTGTACTCACAGATGAAGCGCCTCTCTTCTTTTTCACAGAC
AGCTGCCTGATTCTTGGGGAAGCTATGATCCCATGCATTTTACTTGCTGTTGGGGGCAATCTCGTTGACG
GTCCTGGTGAAGGAAGTAAGAGGCTTGGTGTGCGTACAACCGTTGCTATAATTTTTGCACGTTTGGTCTT
GGTCCCTCTTGCTGGGGTTGGCATCTTCATGGTAGTTGATAAACTTGGTTTCATTCCCAAAGACGACAAA
ATGTTCAAGTTTGTCGTGCTACTGCAGCATTCTATGCCCACATCAGTGCTGTCAGGTGCTGTTGCGAACC
TGAGAGGGTGTGGTAAAGAATCAGCTGCAATCTTGTTCTGGGTTCACATTTTTGCTGTGTTCTCCATGGC
AGGATGGATCATTGTATATTTGAGCTTGCTCTTCTAAGTATGTCGCTCCCTTATTTTAGCTGCTTTTTGC
TCCATTTGTCATCGCATTTGCACCTGTCTGCTGTAACAGAAAACCTTGATATTTACGTTGATCTGATATT
TGGTCAACTGATCTGCAGACACCTTACTGCACACGAAGATCTTTGAAAGAACATATCATCCAAAGTATCA
CGGCGAAAATAGTGACATGAATATGTACAGTAGCTAGTAAATCATGGTGAGGTATGAGTTGTATCAGCAT
TGATATACGGTCCTTCCCAATGCTATGGTAATTGTGGTTCTCTTCCCACTGCTGCAAGCAGTGAGCGAAG
ATGGCACAGCGGTGTTGCCCTGCTCGGTCACACCCGAACCACTTAGATGGGGATGCCACGTTGTCCCAAT
CCATCCATTCGCTTTGTTCATTCCATCTCCTCACCAAAATATTAGCTTAATTTA
SEQ ID NO: 24 Setaria italica PIN-LIKES 6 (SETIT_001507mg) coding sequence
ATGATGGAGAGGTCGCTGCTGCAGGCGCTGGCCACGGCGGCGCAGGGAGGCACCTCGGGGACTTCGGTAC
TGAGCATGCTCAAGTATGCTGTGCTGCCCATCGCCAAGGTGTTCACGGTCTGCTTCATGGGGTTCCTCAT
GGCCTGCAAGTACGTCAACATCCTCCAGCCCAACGGCCGCAAGCTTCTCAACGGGCTTGTGTTTTCGCTT
CTGCTTCCTTGCCTTATATTTTCCCAACTGGGTAGAGCAATCACTATTGAGAAGATGTTACAGTGGTGGT
ATATTCCAGTAAATATTGTTGTAGGTGCAGTATCTGGCTCTTTGATTGGCTTTGTTGTGGCATCTATCAT
CCGACCCCCATATCCGTACTTCAAGTTCACCATTATCCATATAGGAATAGGAAATATTGGAAATATACCT
CTGGTCCTCATTGCAGCGTTATGTCGGGATCCATCTAATCCGTTTGGTGACTCTGATAAATGCAACCAAG
ATGGAAATGCATATATCTCATTTGGTCAATGGGTTGGTGCTATTATTGTTTACACATATGTGTTCAGGAT
GCTTGCTCCACCACCAGGACGCACCTTTGATGGCTCTGATGACGATGAACTTCCAGTCAAGGCATCTGGA
GAGAATGCGGTGCCCGAACTCAGCAAATATCCAATTCCAACAAGCACTCACACTAGCACTGTACCAGAGG
ATGAACCATTGTTAGCTGCTGAGAAAGTTCAAAAAGAATGTGCCACTTCTGTAGGATCAAAGATAATGGG
CCATGTTAAATGTGTGATTAAATTCCTGAAAGACAAGCAGCTTCTCCAGCCACCGATTATTGCATCTGCT
TTTGCAATTGTCATTGGTATTGTCCCATTCCTGAAGAATTTTGTACTCACAGATGAAGCGCCTCTCTTCT
TTTTCACAGACAGCTGCCTGATTCTTGGGGAAGCTATGATCCCATGCATTTTACTTGCTGTTGGGGGCAA
TCTCGTTGACGGTCCTGGTGAAGGAAGTAAGAGGCTTGGTGTGCGTACAACCGTTGCTATAATTTTTGCA
CGTTTGGTCTTGGTCCCTCTTGCTGGGGTTGGCATCTTCATGGTAGTTGATAAACTTGGTTTCATTCCCA
AAGACGACAAAATGTTCAAGTTTGTCGTGCTACTGCAGCATTCTATGCCCACATCAGTGCTGTCAGGTGC
TGTTGCGAACCTGAGAGGGTGTGGTAAAGAATCAGCTGCAATCTTGTTCTGGGTTCACATTTTTGCTGTG
TTCTCCATGGCAGGATGGATCATTGTATATTTGAGCTTGCTCTTCTAA
SEQ ID NO: 25 Setaria italica PIN-LIKES 6 (SETIT_001507mg) amino acid
sequence
MMERSLLQALATAAQGGTSGTSVLSMLKYAVLPIAKVFTVCFMGFLMACKYVNILQPNGRKLLNGLVESL
LLPCLIFSQLGRAITIEKMLQWWYIPVNIVVGAVSGSLIGFVVASIIRPPYPYFKFTIIHIGIGNIGNIP
LVLIAALCRDPSNPFGDSDKCNQDGNAYISFGQWVGAIIVYTYVERMLAPPPGRTFDGSDDDELPVKASG
ENAVPELSKYPIPTSTHTSTVPEDEPLLAAEKVQKECATSVGSKIMGHVKCVIKFLKDKQLLOPPIIASA
FAIVIGIVPFLKNFVLTDEAPLFFFTDSCLILGEAMIPCILLAVGGNLVDGPGEGSKRLGVRTTVAIIFA
RLVLVPLAGVGIFMVVDKLGFIPKDDKMFKFVVLLQHSMPTSVLSGAVANLRGCGKESAAILFWVHIFAV
FSMAGWIIVYLSLLF
SEQ ID NO: 26 Brachypodium distachyon PIN-LIKES 6 gene (BRADI_2g53360v3)
gene NC_016132 REGION: 52289841 . . . 52295181
AGAAACCAAATCACACCCACCCTGGCCACGCCTCGACGCGACTCATCTCGTCGTTTCCCCTTCGGAGGCC
TGCCTGCCAGATTCATCCCTCCCTCCCCTCCCGGTCTCCCCCACCCGACTTGCCTCCAAAAGCAAAGCCC
CCCGCCATTCACTACTCCGGCTTCACCTTAATCACGCTCTTAATCCTCGAACCCTCACGGAGCGGGGGTT
TGGTCGGCAGGACTGCCTCCCGCGGGCTCGGCTACGCTCACTCCGCCCCTCCCCGACCCCCGAGCAGGCG
CAAGTCCCCTCCTCGACTTCGCGGTGGGTTCTCTCGCTGATCCCCGTCTCGTTTGTCTGACTTGCTCTTT
GTTTTTCGGCATTGGGAGGCGCAGAGCTCGGGGGATGGAGGTCGTCGTCGTGGACCCCGCTCTCTCGCAT
TGGATTATCCTTATCTTAAGTGTCGATTTGTGGTTTTTAGTTGCGTCTGAGCTCTGAAGTTCCCTCGGGG
TGGGTTTCCGTGCGGTCGAAACTGTTGAGTTGGTCGTGGCGTGACGAATCGAACGGAGCAGGCGGATGCT
TACGTAGCTCTAGGGGAATCGCCCTACGCGATTATTCGAATTTGGGGGTAGTGAACACCGACGCTCTTCA
GAATCTGGTACCGTCATTTGTAACTGTCGAGCTGTTTGGAATCTAGTTGGGTTTCTTCTCCCCCTAGCTT
TTTTCCCCAATTCGATCTGAAACTCTTCTCCTCATGCATCAGTGGACGCGAGGTTTTGCGCGAACGGGAA
GTTAGTTAGTGCAAGTTTCGTCGTGGCTACTTTGGTTAATTCGTCACGCTGGGTTGTGGGTATTCCAGTT
GGGCTATCTTTAGAACAACCAACACCAAAAGCTCTCCTCCGATTTTCCTGCTGTTGCTCCTATATTTTGC
CTTCTAAGTCGTTTTTAATCGCAGAATTGAGGGAAGGAGAGAGAAGACTTAAGTCTAGTCCGGGTCAGCC
ATGATGGAGAGGAGGTCGCTGCTGGAGGCGCTGGTCACAGCGGCGCAGGGAGGTTCCTCACAGACGTCGG
TGCTGTCCATGCTCAAGTACGCCGTGATGCCCATCGCCAAGGTATTCACCGTCTGCTTCATGGGTTTCCT
CATGGCCACCAAGTACGTCAACATCCTCCAGCCCAACGGCCGCAAGCTTCTCAACGGGGTGAGCTTTCTT
TATTCTCTTTGTGATAAATGCCTTGCTAATGAGACCTATAGCCGCACTGTGAGCTTGAAATATGGTGTTT
GTCCAGAGTAGGAAGGTATAAGCTACTTGACAGTGTGCTTACATTCTTACTGTTTGGTGCAGCTTGTGTT
CTCACTTCTGCTTCCTTGCCTTATATTTTCCCAACTGGGTAGCGCAATCACGATCGAGAAGTTGCTGCAG
TGGTGAGGATTGCTTTTGGATGATTTATTACTCTCTTTTCTAATGATTTAGTAATTCAGTCTTCTCCTAT
GCCTTGTTGAATGCCCTGTCTATATTATGTCTTTAATTTTGTCGTGTGAGCTACTGACTGAGTTGTTGTT
AATATTAATTAGATTTTTATTACTCAAAGTATCATTGTGTAATTTTACTATTGCTGAATTTCCTGACAAA
ATTAAGCCTGGAACTTCCAAATATAACAAAGTAACTCCCATGACCTAAGTTTCAAGAAAGGCAAACAGGG
TACCATCCAAGAGTGAAGGAAATATGTAGTAGGGATTAATTGGTTCCAGTTGGTTTTTTAGGGTTGGAAT
GGAATTGAATCAAGAGTTTCTGGACTATTTTTATAGCAGCACTTCTTTCTAATAATATACTGAAATATTT
CTATAACTAGAGACATTATTTGTCATCTACTAATAGTACATGGCCCACCCAACACCAAGTTAGGGAGAGT
ATCCCCGTGATCTCTTAGTTAAGAAGTTTATCATTTATGTTAGGAAGATATCTTTTAGCTTGAGCCTTAA
GAAAATTAGGGCATCAACAGTAGTACAGTCAGTATTGACTCTCCACGTCATTTAGAGTTGACGAGATAAT
AATATATACAATGGGTTGTGTGACTTGTGTTTTTAGACTTACCATAGATGATCTGTTAGTATTAAATGCG
ATGACGTACAATGGTGAATATGTTGACTCATAGCTGACTCTTAGTCGATTCTTGCCTTAAGAGTCTGACT
CTTCACGAAGAGTCAATTCTCTTCTCTCTCCTATTTCTCTTTCTTTCACACCATCAAATTTCAATAAGAG
TCAATCAAGAGTCAGCCTTTGTGGATGGCCTTGGAGATAAAATCTACCATTTGTTACTTAGATTGGACTG
TAAGTCCATCATAAATCAATATAAAGAGAACCATCTATGAGGAATTAATCAAGAAAGTATTAGAAGAACA
ACTCTTCTATCTTGGCCTAGGGCAAGAACGTCCCTCGACCCCCTCCCATCCCCTCCTCAACCCTAGCCGA
TTCCCTACCGCCCCAAACACGGGCCATAGCACTATTCCAGTGCATTAATTTACCTAGAGGTCTTTACCAT
ATTTGTTTGTTACCGGCAATATTCATCTCGTTAGTTGAACTTCATCTTTCATGTCATCCAATCTTGTCGT
AGCTGTTTGTGTACGATGACATAATGCCTTTTGTGATTGTGTTCTTCCTATTTATAACATTTCTCCCTTT
TTTGTACTTTGTAGGTGGTATATTCCAGTAAATATCGTTGTAGGTGCTGTGTCCGGTTCTCTCATTGGCT
TTGTGGTGGCGTCTATCATTCGGCCTCCTTATCCGTACTTCAAGTTCACTGTTATACACATTGGAATAGG
TGCTTGTTTTTCTTCCTGAAGAAAGGTTGCATTTCATTTATGATCTTCTAGCTGATACTGATATTACCCC
TTATTTTTTTGACACAGGAAATATTGGAAATATACCTCTGGTTCTCATTGCAGCATTATGTCGCGATCCA
TCCAATCCTTTTGGTGATTCTGATAAATGCAGCCAAGACGGAAATGCATATATCTCATTTGGTCAATGGG
TATGTGTTTTTTCTTTTCCATGGGTGCATGTGTTCAGTGTTTTACTTACAAGCCACTAAGTCAGAATGAC
TAGCATGCAATATAAAGAGAATAGCTCAGCATTTTAGAAGAATATGAATAACAACTGAGATCATTTGCTG
AATGAATTCTTCACGTGAGGCAGGTTGGTGCGATTATTGTTTACACGTATGTGTTCAAGATGCTTTCTCC
ACCACCAGGAGAGACCTTTGATGGTGAAGAGGAGAAACTTCCAGTTATGGCATCTGGAGAGAATACGTTG
CCTGAACTAGGTAAATATCCAACAAGCACTCGGAATAGTACTGTACCTGAGAATGAGCCTTTGTTATCTG
TTGAGGGGGACAAAAAAGGTGCTACTTCTCTAGGATCAAAGGTAAGAAATGTTAGCAGTGATTTCTATTT
TAATTTGTTGCACTTACATCCCCATGTGAAATTAAATAATTGTGTTCTATTGTCACAGATAATTGGCTAT
GTTAGATGCGTGGTTAAATTCCTAAAAGACAAGCAGCTTCTTCAGCCACCAATTATTGCCTCTGTATGTA
CTCATGTGAATGTATTCTACTGGCAATTATTACAGAAAAATATCTCTTCCTGATGTGCCATGAAAATTAT
TTTACACTATGGAACAGGTTTTTGCAATTGGCATCGGTGTTGTTCCATTCTTGAAGGGTTTGATATTCAC
GGACGATGCACCTCTATTCTTCTTCACAGACAGCTGTCTCATTCTTGGGTATTTGCATCTTCCTGTCAGA
TTCAATTTCCAAAACATAGACCTCATGCCAGTTAAATTATAGAAGTAAACATAGGGACAATGATTTCGTT
TTTCTAAATTCTGTCACACATTTTCAGGGAAGCTATGATCCCATGCATTTTGCTTGCTGTGGGAGGCAAC
CTTGTTGATGGTAAGTATCTATTTCCATCGAAACTGCTGCCAGATCGAACCCTGTAATGGTCTTGAGATG
CATTTATCCAGATCTCAAACACTAGCCGTTGTTTATTCACCAGGTCCTGGTGAAGGAAGTAAGAGACTTG
GTATGCGAACCACCATTGCAATTATTTTTGCACGGTTGGTCTTGGTTCCTATTGCTGGGGTTGGCATTGT
CTTGCTAGTTGATAAACTCGGTTTCATTCCCAAAGATGACAAAATGTTCAAGTTTGTCCTACTACTGCAG
CATTCTATGCCCACATCAGTTTTGTCAGGTATGAGATAATAAGGGCCGCTGCTTATCACTTACCATCATG
TGCTCATGTTTCAAGATTCCTCTCATAATGTTTTGTTTGTTGAGATCTGTAAAACAGTGGGTGTGGGGGC
GCCATGTGCTGCATGTTAGCAAACCGCATTATGAGATCTGATAATAAGATTGTTCTGTTTACAGGTGCTG
TTGCAAACCTGAGAGGGTGTGGAAAAGAATCGGCCGCCATCTTGTTCTGGGTGCACATTTTTGCAGTGTT
TTCCATGGCAGCATGGATTATTTTCTATTTGACCTTGCTCTTCTAAGTACGTGGCTTCCAGTATGCAGTT
CCTTTTCTGTTTTGGGCTTACTTCTATGCATATTAACTGCGGTTTCTTGATTCTCAAATTATTCTGGTTT
GATTTACAGAGACTACCGAACGGCAATGCCTGCAAAAATTAGTTACTGTCAAAAAAAAATATCGAAGAAG
ATAGTGTCATGACTTGTACAATAGCTACTAAATCCTTGTGGCAGCTCTGGAGGTACGAGTTGTATCGGTG
CATATATGGTTCATCATGCCCGCCTGTGCAAATGCCACTGTTATGTTAAATTTGCCTCCTGGCTGCTGCA
AGTATGGAGGGATGAGAATCGCAGCAGCTAGTGCCCTGCCCATATATACCATCAGGCTACCTTAACCTCT
TAGACTGGGTTTGCTGCCCTTATTATTGTCACCTGTATTTAAAATATTTCGATCCATTTGCCTACTTGGT
TCAGGTGTAATTTGGGTGGCGTATCTGTGAATTTTGATGGCCCAGTAGTCAGTTCTTGAATTCAGATAGC
AACTATATAAGCATATCATCGGGCGCATTGGACAGCAAACCATGATGCATGATTCTGTGCCCACTGTCTA
CCGATACATGGATCTGATTATCACAAGCGATGATGTGCAACAGTGCAAGCCTCATGCCAGCAGGGTGTCA
AAGCATGCGTGCATGTCCCTGGTGTAGCCAAACCGACAGCAAAACATTATGGAGAAGTTCTGAGATGAAC
TGCCGCGCCTGATCTCTCCATGTGCTTGTATGTAGATCTGGGACTCGGCAGAGCTTGAATCATTAATTTT
GACAACCATGATACTTTTGAA
SEQ ID NO: 27 Brachypodium distachyon PIN-LIKES 6 coding sequence
ATGATGGAGAGGAGGTCGCTGCTGGAGGCGCTGGTCACAGCGGCGCAGGGAGGTTCCTCACAGACGTCGG
TGCTGTCCATGCTCAAGTACGCCGTGATGCCCATCGCCAAGGTATTCACCGTCTGCTTCATGGGTTTCCT
CATGGCCACCAAGTACGTCAACATCCTCCAGCCCAACGGCCGCAAGCTTCTCAACGGGCTTGTGTTCTCA
CTTCTGCTTCCTTGCCTTATATTTTCCCAACTGGGTAGCGCAATCACGATCGAGAAGTTGCTGCAGTGGT
GGTATATTCCAGTAAATATCGTTGTAGGTGCTGTGTCCGGTTCTCTCATTGGCTTTGTGGTGGCGTCTAT
CATTCGGCCTCCTTATCCGTACTTCAAGTTCACTGTTATACACATTGGAATAGGAAATATTGGAAATATA
CCTCTGGTTCTCATTGCAGCATTATGTCGCGATCCATCCAATCCTTTTGGTGATTCTGATAAATGCAGCC
AAGACGGAAATGCATATATCTCATTTGGTCAATGGGTTGGTGCGATTATTGTTTACACGTATGTGTTCAA
GATGCTTTCTCCACCACCAGGAGAGACCTTTGATGGTGAAGAGGAGAAACTTCCAGTTATGGCATCTGGA
GAGAATACGTTGCCTGAACTAGGTAAATATCCAACAAGCACTCGGAATAGTACTGTACCTGAGAATGAGC
CTTTGTTATCTGTTGAGGGGGACAAAAAAGGTGCTACTTCTCTAGGATCAAAGATAATTGGCTATGTTAG
ATGCGTGGTTAAATTCCTAAAAGACAAGCAGCTTCTTCAGCCACCAATTATTGCCTCTGTTTTTGCAATT
GGCATCGGTGTTGTTCCATTCTTGAAGGGTTTGATATTCACGGACGATGCACCTCTATTCTTCTTCACAG
ACAGCTGTCTCATTCTTGGGGAAGCTATGATCCCATGCATTTTGCTTGCTGTGGGAGGCAACCTTGTTGA
TGGTCCTGGTGAAGGAAGTAAGAGACTTGGTATGCGAACCACCATTGCAATTATTTTTGCACGGTTGGTC
TTGGTTCCTATTGCTGGGGTTGGCATTGTCTTGCTAGTTGATAAACTCGGTTTCATTCCCAAAGATGACA
AAATGTTCAAGTTTGTCCTACTACTGCAGCATTCTATGCCCACATCAGTTTTGTCAGGTGCTGTTGCAAA
CCTGAGAGGGTGTGGAAAAGAATCGGCCGCCATCTTGTTCTGGGTGCACATTTTTGCAGTGTTTTCCATG
GCAGCATGGATTATTTTCTATTTGACCTTGCTCTTCTAA
SEQ ID NO: 28 Brachypodium distachyon PIN-LIKES 6 amino acid sequence
MMERRSLLEALVTAAQGGSSQTSVLSMLKYAVMPIAKVFTVCFMGFLMATKYVNILQPNGRKLINGLVES
LLLPCLIFSQLGSAITIEKLLOWWYIPVNIVVGAVSGSLIGFVVASIIRPPYPYFKFTVIHIGIGNIGNI
PLVLIAALCRDPSNPFGDSDKCSQDGNAYISFGQWVGAIIVYTYVEKMLSPPPGETEDGEEEKLPVMASG
ENTLPELGKYPTSTRNSTVPENEPLLSVEGDKKGATSLGSKIIGYVRCVVKFLKDKQLLQPPIIASVFAI
GIGVVPFLKGLIFTDDAPLFFFTDSCLILGEAMIPCILLAVGGNLVDGPGEGSKRLGMRTTIAIIFARLV
LVPIAGVGIVLLVDKLGFIPKDDKMFKFVLLLQHSMPTSVLSGAVANLRGCGKESAAILFWVHIFAVESM
AAWIIFYLTLLF
SEQ ID NO: 64 Zmpils6-1 (Mu1047700 insertion)
GGATATAAAATGCGCGTGTATTATCTAGTCCAGTCCAACCGACCCGGCATCGTCTCGC
GAGCCGCGACTCGTCTCGTCATTTCCATCGAACGCCCAGATCCCACATTCCCACCTAG
CTCTGCGCGATCCATCCCGGCCGCCACGCCAAGGAGGCCCTCGCCTGACCTCGGATCA
AAGCCTCCTCCATCCGCCTTCATCCCGGCCGGTGGTTCGGGCCGTCACGGGAGGGGTC
CATCACTGGTTACTCGGTGGAGAGCTCGGGGCTCGGCTCCCATCTCACGATTCTCACC
CCACACGCGCACCGGGTCACCTCGATTCGGCAGCCTCGCGGTGAGTTGAGTTCTTCGC
TTGTCGGGCCAGTCCGCCTCTCGCTTGGCGCGGTTATGGATTCGCGGGTGGCGTGGCG
TCGTCCGCGATCGCTCTAGCTCGCATTGAGCGATTCGCGAGTTGCGACTCCTTGTTTT
GTTTGCTCTCTGTTTTTTTTTATGTCGATGGTGATATGTGGCGGCGGGATTCTGAGCT
CGGTTGCTTCTAACCTTCCGCGGAGTGGTTTAGCTAATCGGTTTTGAAGCTGTTGATA
TGGATGTGCCGTGTGCGTGACGAATCGAATGGGCCGCAGCAGGATTCCTACGCAGCTC
GGCAGCAGGGGGAATTGGATCCTGCGACCATGCGAATTTGGAACCCTGGAGCAGTGGA
GTGCTAGGAGGTCTCGGTGTCGTCATTTTTTTCTAGGGGCGTAGGAGTCGACTTGGTT
GGAATCTAGTTAGTCATAATTTC[CTTTCTGAT]TCTTTCTGATTTCATTGGTCTGTC
CATGCGTCTGTTTTCGTCAAACAAGAAGTTTGTTATTGCCAAGCATCCCGCTATGGAG
GAAGCTGCCTGCCGTATTGTGTTACGCGAATCCGGTGAGCTGGGCTCTGCGCGTTCCT
CTTGACGCCCTTTGCGCGAACGACGAGCTTTCTTCCAGCTCGGTTGCCCCTGAACAAC
CAACGCCAAAAGCAGCTCCAGAATTTTCCTGATGTCGTCTCTATATTTTTGCCTTCTA
AGTCATGCTTAATGGCAGAGTCCTTGTTGCGTGGGTCACTGCCTCACTGGGTTCTGAG
GCAAGGAAAAGAAGGCTTCAGTAGTCAGGTCTGGGTCAAGCCATGATGGAGAGATCGC
TGCTGGAGGTGCTGGCCACGGCGGCGCAAGGAGGGACCGAGGGGACGTCAGTGCTGAG
CATGCTCAAGTACGCCGTGCTGCCCATCGCCAAGGTGTTCACTGTCTGCTTCATGGGG
TTCCTTATGGCCTCCAAGTACGTCAACATTCTCCAGCCCAACGGCCGCAAGCTTCTCA
ATGGGgtgagctttgtctcttgtaataactaataagtgctagctaatggaaggttgtg
taatggacaattgtttttctattgaggaattattttattaagtcaagatgattaccca
tcttggattttgtttaaagtaggaatatgtgaagcagtagaggagttgattgtctcat
ttgttttctcgctgtatcaaattagatataacattttcactgtttgttgcagCTTGTG
TTTTCCCTTCTGCTTCCATGCCTTATATTTTCCCAATTGGGTAGAGCAATCACTATCG
AGAAGATGATACAATGgtaaagataattttgattgttatcgtacttcagttttgttat
tataaacctagtgttatgcttcatccaagcgtgctatatttgctcttgattttttgat
gtgtgagttattgaccgagatgttgttttcagctaatcatctatttgtatatgctgct
gaccctctataagtaatacatcctacaaatattatattactgaccacacttgattggt
gtgctattgtttatctttctatttggatcaatctttgagtaattaggttccatatgga
acaaattagttttagaagctctagctttctacaaaaggacaaagaaaagactacctga
aggtgaagaaaatcagcgctcaagcattaatttggttccagttgaatttatggatcaa
tatgggattgacttcagattccaggattaatttagtagcactactttatgtttaactt
tttgaatactgcagtaaagattggagacactattccaagtagtttgtttccatatgat
atagtaacctatatttttttcttactggcaatttatatcttgttgaaaatgatagaaa
tttatgtaatggactattcactattctgtcctatattttgacatacccctggattctt
tcccccttctggtgtgtatatgtataacaagtgtccacttttgtgcataagcttcttt
aggagtgtgtgtcctggaaattttcctttgtcaaataactgttctgcacttctgttct
gcttttacttttttatatcatctatacttgcatgtttgcagGTGGTATATTCCAGTAA
ATATTGTTGTTGGCGCAGTATCGGGCTCTTTGATTGGATTTGTTGTGGCATCTATCAT
CAGACCTCCATATCCATACTTCAAGTTCACTGTTATCCATATAGGAATAGgtaccact
tttctgttttgaaaataagttttatttttatctatcatttgtttatccaataccaatc
caggttctttttttgcagGAAATATTGGAAATATACCTCTGGTCCTCATTGCAGCGTT
ATGTCGTGATCCTTCTAACCCATTTGGCGACTCTGATAAATGCAATCAAGATGGGAAT
GCATATATCTCATTTGGCCAATGGgtacgccatacttttctcttttctcggcttgtac
atgctcatttttactcatgtcagcatagtgaccttgttgtttaaagattgccacttat
cacctagactatgcatacaattagctgttgaggttattcactcggtgagtcctctata
tgttgcagGTTGGTGCAATTATTGTTTACACATATGTATTCAAAATGCTTGCTCCACC
ACCAGGACAGACCTTTGATGGTTCTGAAGAGGATGGAATCCCAATCAAGGCATCTGGA
GAGAATACAGTGCCCCAAGTAGGAAAATATCCTATGAACACTAACAGTAGTACTGTAC
CAGAGAATGAACCTTTGTTATCTGCTGGGGAAGTTCAAAAGGAGCGTGCCACTTCTGT
AGGAACAAAGgtaagaaattctaatagtggagctgatttcgaatatccatttggtgcg
tttacttcccactgcaaaactaaatgattttgttgttttatgcagATAATGGGCTATG
TTAAATGTGTGGTTAAGTTTCTGAAAGACAAGCAGCTTCTCCAGCCACCAATTATTGC
ATCTgtatgtattgctgctgtagtgttctactagcattttttaaaccaatttctttga
gtaattatttatatactaactctacgtctttttttcttttggccattgtttaacacag
GCTTTTGCAATTGCAATCGGTGTTATCCCATTCTTGAAGAATTTTGTACTTACGGATG
ATGCTCCTCTGTTCTTTTTCACAGACAGCTGCCTCATTCTTGGgtatctacatgtttc
atttctattttgtttttgcgaaaacatctctcttgccaataaagttatcaaactgaga
tgaatgtatctatcacctaattctgtggcatacttttcagAGAAGCTATGATCCCATG
CATTTTACTTGCTGTTGGGGGCAATCTTGTCGATGgtaagtagctattttagttaaaa
ctttggagaattgcatggggtgcgataatccttagaatgaccatctttattggtttag
agatatttctaatgtttttgcctttacccatgtttttttatggaatcagGCCCTGGTG
AAGGAAGTAAGAGGCTTGGCGTGCGTACCACTGTTGCTATAATTTTTGCACGGTTGGT
CTTGGTCCCTCTTGCTGGGGTTGGCATTACCATGTTAGTTGATAAACTTGGTTTCATT
CCCGAAGGTGATAGAATGTTCAAGTTTGTCCTGCTACTGCAGCATTCTATGCCCACAT
CAGTGTTGTCAGgtatgtgaagccaacaagggatgtatcttatcagctatcacttgtc
ttcatgtgctcatgcgtacagattcttgttaacaattttgtgtgttgagatctgtaag
gaaaccacattatggactctgacgataggattgtttgttgcagGTGCTGTTGCAAATC
TGAGAGGTTGTGGAAAAGAATCAGCTGCAATTTTGTTCTGGGTTCACATTTTTGCTGT
GTTCTCCATGGCGGGATGGATTATATTCTATCTGAGTTTGCTCTTCTAAGTATGTCAC
GCCCTGCTACCACAACAATCTGCAATTTGTGCCCGCATTCCAGCCTGTCAGATGTAAC
TGCATTTTCTTGCTAGATAATGTTACTTGATCTGATTTCAGTCCGCTGGTTCAGATGC
CTTACTGCGCAGGAATATCTTTGAGATCAAATATGGTCCAAAATGTTGAGGAAGAAAC
CAGTATCATGAACTGTACAGATATATCGTTGCAGAGGTACAAGTGTATCTGTACAGAT
ATATGGATACGGTCCCTCCTATAATCCCATATTACCGTGGTCTCCTGCTGCTACAAGC
CGTGAGCGATGGGAACACTTTCCCTTTCCCTCCACGTATCAACAGGTTCACACCCAAC
CCTAGATTGGGATGCTGGCCTTCTTAGTTCTCCATAATATTATTTCATTTGTACTTCC
TGTATATTGTAAACCTTGAGATGGCTGTGTTCAAATAGTTTCTTATTGGGCGAGGGCT
GAAGTAGTAGTATAGATGTATTATACTACCTATTTGAGTGTTCGGTTATGCCGCAAAT
GATGCTTTATTATGCAGAACTTGTGCCCGCTGCTGGCCCGTCGGTCGTACTAACTAAA
ATTGCCAATCGCGGAGACTTGCATGTTGCAAGCTCAGGCCGGCGCCCAGCGGTTTGCA
GCAAATTTTTTTTTTTGACTCGGCTATCTCCAATAGAATACTCAACTCAATATTTATA
TTTAAACTCTACTCTATC
SEQ ID NO: 65 Zmpils6-2 (Mu1090629 insertion)
GGATATAAAATGCGCGTGTATTATCTAGTCCAGTCCAACCGACCCGGCATCGTCTCGC
GAGCCGCGACTCGTCTCGTCATTTCCATCGAACGCCCAGATCCCACATTCCCACCTAG
CTCTGCGCGATCCATCCCGGCCGCCACGCCAAGGAGGCCCTCGCCTGACCTCGGATCA
AAGCCTCCTCCATCCGCCTTCATCCCGGCCGGTGGTTCGGGCCGTCACGGGAGGGGTC
CATCACTGGTTACTCGGTGGAGAGCTCGGGGCTCGGCTCCCATCTCACGATTCTCACC
CCACACGCGCACCGGGTCACCTCGATTCGGCAGCCTCGCGGTGAGTTGAGTTCTTCGC
TTGTCGGGCCAGTCCGCCTCTCGCTTGGCGCGGTTATGGATTCGCGGGTGGCGTGGCG
TCGTCCGCGATCGCTCTAGCTCGCATTGAGCGATTCGCGAGTTGCGACTCCTTGTTTT
GTTTGCTCTCTGTTTTTTTTTATGTCGATGGTGATATGTGGCGGCGGGATTCTGAGCT
CGGTTGCTTCTAACCTTCCGCGGAGTGGTTTAGCTAATCGGTTTTGAAGCTGTTGATA
TGGATGTGCCGTGTGCGTGACGAATCGAATGGGCCGCAGCAGGATTCCTACGCAGCTC
GGCAGCAGGGGGAATTGGATCCTGCGACCATGCGAATTTGGAACCCTGGAGCAGTGGA
GTGCTAGGAGGTCTCGGTGTCGTCATTTTTTTCTAGGGGCGTAGGAGTCGACTTGGTT
GGAATCTAGTTAGTCATAATTTCTCTTTCTGATTTCATTGGTCTGTCCATGCGTCTGT
TTTCGTCAAACAAGAAGTTTGTTATTGCCAAGCATCCCGCTATGGAGGAAGCTGCCTG
CCGTATTGTGTTACGCGAATCCGGTGAGCTGGGCTCTGCGCGTTCCTCTTGACGCCCT
TTGCGCGAACGACGAGCTTTCTTCCAGCTCGGTTGCCCCTGAACAACCAACGCCAAAA
GCAGCTCCAGAATTTTCCTGATGTCGTCTCTATATTTTTGCCTTCTAAGTCATGCTTA
ATGGCAGAGTCCTTGTTGCGTGGGTCACTGCCTCACTGGGTTCTGAGGCAAGGAAAAG
[GGCTTCAGT]AAGGCTTCAGTAGTCAGGTCTGGGTCAAGCCATGATGGAGAGATCGC
TGCTGGAGGTGCTGGCCACGGCGGCGCAAGGAGGGACCGAGGGGACGTCAGTGCTGAG
CATGCTCAAGTACGCCGTGCTGCCCATCGCCAAGGTGTTCACTGTCTGCTTCATGGGG
TTCCTTATGGCCTCCAAGTACGTCAACATTCTCCAGCCCAACGGCCGCAAGCTTCTCA
ATGGGgtgagctttgtctcttgtaataactaataagtgctagctaatggaaggttgtg
taatggacaattgtttttctattgaggaattattttattaagtcaagatgattaccca
tcttggattttgtttaaagtaggaatatgtgaagcagtagaggagttgattgtctcat
ttgttttctcgctgtatcaaattagatataacattttcactgtttgttgcagCTTGTG
TTTTCCCTTCTGCTTCCATGCCTTATATTTTCCCAATTGGGTAGAGCAATCACTATCG
AGAAGATGATACAATGgtaaagataattttgattgttatcgtacttcagttttgttat
tataaacctagtgttatgcttcatccaagcgtgctatatttgctcttgattttttgat
gtgtgagttattgaccgagatgttgttttcagctaatcatctatttgtatatgctgct
gaccctctataagtaatacatcctacaaatattatattactgaccacacttgattggt
gtgctattgtttatctttctatttggatcaatctttgagtaattaggttccatatgga
acaaattagttttagaagctctagctttctacaaaaggacaaagaaaagactacctga
aggtgaagaaaatcagcgctcaagcattaatttggttccagttgaatttatggatcaa
tatgggattgacttcagattccaggattaatttagtagcactactttatgtttaactt
tttgaatactgcagtaaagattggagacactattccaagtagtttgtttccatatgat
atagtaacctatatttttttcttactggcaatttatatcttgttgaaaatgatagaaa
tttatgtaatggactattcactattctgtcctatattttgacatacccctggattctt
tcccccttctggtgtgtatatgtataacaagtgtccacttttgtgcataagcttcttt
aggagtgtgtgtcctggaaattttcctttgtcaaataactgttctgcacttctgttct
gcttttacttttttatatcatctatacttgcatgtttgcagGTGGTATATTCCAGTAA
ATATTGTTGTTGGCGCAGTATCGGGCTCTTTGATTGGATTTGTTGTGGCATCTATCAT
CAGACCTCCATATCCATACTTCAAGTTCACTGTTATCCATATAGGAATAGgtaccact
tttctgttttgaaaataagttttatttttatctatcatttgtttatccaataccaatc
caggttctttttttgcagGAAATATTGGAAATATACCTCTGGTCCTCATTGCAGCGTT
ATGTCGTGATCCTTCTAACCCATTTGGCGACTCTGATAAATGCAATCAAGATGGGAAT
GCATATATCTCATTTGGCCAATGGgtacgccatacttttctcttttctcggcttgtac
atgctcatttttactcatgtcagcatagtgaccttgttgtttaaagattgccacttat
cacctagactatgcatacaattagctgttgaggttattcactcggtgagtcctctata
tgttgcagGTTGGTGCAATTATTGTTTACACATATGTATTCAAAATGCTTGCTCCACC
ACCAGGACAGACCTTTGATGGTTCTGAAGAGGATGGAATCCCAATCAAGGCATCTGGA
GAGAATACAGTGCCCCAAGTAGGAAAATATCCTATGAACACTAACAGTAGTACTGTAC
CAGAGAATGAACCTTTGTTATCTGCTGGGGAAGTTCAAAAGGAGCGTGCCACTTCTGT
AGGAACAAAGgtaagaaattctaatagtggagctgatttcgaatatccatttggtgcg
tttacttcccactgcaaaactaaatgattttgttgttttatgcagATAATGGGCTATG
TTAAATGTGTGGTTAAGTTTCTGAAAGACAAGCAGCTTCTCCAGCCACCAATTATTGC
ATCTgtatgtattgctgctgtagtgttctactagcattttttaaaccaatttctttga
gtaattatttatatactaactctacgtctttttttcttttggccattgtttaacacag
GCTTTTGCAATTGCAATCGGTGTTATCCCATTCTTGAAGAATTTTGTACTTACGGATG
ATGCTCCTCTGTTCTTTTTCACAGACAGCTGCCTCATTCTTGGgtatctacatgtttc
atttctattttgtttttgcgaaaacatctctcttgccaataaagttatcaaactgaga
tgaatgtatctatcacctaattctgtggcatacttttcagAGAAGCTATGATCCCATG
CATTTTACTTGCTGTTGGGGGCAATCTTGTCGATGgtaagtagctattttagttaaaa
ctttggagaattgcatggggtgcgataatccttagaatgaccatctttattggtttag
agatatttctaatgtttttgcctttacccatgtttttttatggaatcagGCCCTGGTG
AAGGAAGTAAGAGGCTTGGCGTGCGTACCACTGTTGCTATAATTTTTGCACGGTTGGT
CTTGGTCCCTCTTGCTGGGGTTGGCATTACCATGTTAGTTGATAAACTTGGTTTCATT
CCCGAAGGTGATAGAATGTTCAAGTTTGTCCTGCTACTGCAGCATTCTATGCCCACAT
CAGTGTTGTCAGgtatgtgaagccaacaagggatgtatcttatcagctatcacttgtc
ttcatgtgctcatgcgtacagattcttgttaacaattttgtgtgttgagatctgtaag
gaaaccacattatggactctgacgataggattgtttgttgcagGTGCTGTTGCAAATC
TGAGAGGTTGTGGAAAAGAATCAGCTGCAATTTTGTTCTGGGTTCACATTTTTGCTGT
GTTCTCCATGGCGGGATGGATTATATTCTATCTGAGTTTGCTCTTCTAAGTATGTCAC
GCCCTGCTACCACAACAATCTGCAATTTGTGCCCGCATTCCAGCCTGTCAGATGTAAC
TGCATTTTCTTGCTAGATAATGTTACTTGATCTGATTTCAGTCCGCTGGTTCAGATGC
CTTACTGCGCAGGAATATCTTTGAGATCAAATATGGTCCAAAATGTTGAGGAAGAAAC
CAGTATCATGAACTGTACAGATATATCGTTGCAGAGGTACAAGTGTATCTGTACAGAT
ATATGGATACGGTCCCTCCTATAATCCCATATTACCGTGGTCTCCTGCTGCTACAAGC
CGTGAGCGATGGGAACACTTTCCCTTTCCCTCCACGTATCAACAGGTTCACACCCAAC
CCTAGATTGGGATGCTGGCCTTCTTAGTTCTCCATAATATTATTTCATTTGTACTTCC
TGTATATTGTAAACCTTGAGATGGCTGTGTTCAAATAGTTTCTTATTGGGCGAGGGCT
GAAGTAGTAGTATAGATGTATTATACTACCTATTTGAGTGTTCGGTTATGCCGCAAAT
GATGCTTTATTATGCAGAACTTGTGCCCGCTGCTGGCCCGTCGGTCGTACTAACTAAA
ATTGCCAATCGCGGAGACTTGCATGTTGCAAGCTCAGGCCGGCGCCCAGCGGTTTGCA
GCAAATTTTTTTTTTTGACTCGGCTATCTCCAATAGAATACTCAACTCAATATTTATA
TTTAAACTCTACTCTATC
SEQ ID NO: 66 Zmpils6-3 (Mu_ill_221882.6 insertion)
GGATATAAAATGCGCGTGTATTATCTAGTCCAGTCCAACCGACCCGGCATCGTCTCGC
GAGCCGCGACTCGTCTCGTCATTTCCATCGAACGCCCAGATCCCACATTCCCACCTAG
CTCTGCGCGATCCATCCCGGCCGCCACGCCAAGGAGGCCCTCGCCTGACCTCGGATCA
AAGCCTCCTCCATCCGCCTTCATCCCGGCCGGTGGTTCGGGCCGTCACGGGAGGGGTC
CATCACTGGTTACTCGGTGGAGAGCTCGGGGCTCGGCTCCCATCTCACGATTCTCACC
CCACACGCGCACCGGGTCACCTCGATTCGGCAGCCTCGCGGTGAGTTGAGTTCTTCGC
TTGTCGGGCCAGTCCGCCTCTCGCTTGGCGCGGTTATGGATTCGCGGGTGGCGTGGCG
TCGTCCGCGATCGCTCTAGCTCGCATTGAGCGATTCGCGAGTTGCGACTCCTTGTTTT
GTTTGCTCTCTGTTTTTTTTTATGTCGATGGTGATATGTGGCGGCGGGATTCTGAGCT
CGGTTGCTTCTAACCTTCCGCGGAGTGGTTTAGCTAATCGGTTTTGAAGCTGTTGATA
TGGATGTGCCGTGTGCGTGACGAATCGAATGGGCCGCAGCAGGATTCCTACGCAGCTC
GGCAGCAGGGGGAATTGGATCCTGCGACCATGCGAATTTGGAACCCTGGAGCAGTGGA
GTGCTAGGAGGTCTCGGTGTCGTCATTTTTTTCTAGGGGCGTAGGAGTCGACTTGGTT
GGAATCTAGTTAGTCATAATTTCTCTTTCTGATTTCATTGGTCTGTCCATGCGTCTGT
TTTCGTCAAACAAGAAGTTTGTTATTGCCAAGCATCCCGCTATGGAGGAAGCTGCCTG
CCGTATTGTGTTACGCGAATCCGGTGAGCTGGGCTCTGCGCGTTCCTCTTGACGCCCT
TTGCGCGAACGACGAGCTTTCTTCCAGCTCGGTTGCCCCTGAACAACCAACGCCAAAA
GCAGCTCCAGAATTTTCCTGATGTCGTCTCTATATTTTTGCCTTCTAAGTCATGCTTA
ATGGCAGAGTCCTTGTTGCGTGGGTCACTGCCTCACTGGGTTCTGAGGCAAGGAAAAG
AAGGCTTCAGTAGTCAGGTCTGGGTCAAGCCATGATGGAGAGATCGCTGCTGGAGGTG
CTGGCCACGGCGGCGCAAGGAGGGACCGAGGGGACGTCAGTGCTGAGCATGCTCAAGT
ACGCCGTGC[CTTCATGGGG]TGCCCATCGCCAAGGTGTTCACTGTCTGCTTCATGGG
GTTCCTTATGGCCTCCAAGTACGTCAACATTCTCCAGCCCAACGGCCGCAAGCTTCTC
AATGGGgtgagctttgtctcttgtaataactaataagtgctagctaatggaaggttgt
gtaatggacaattgtttttctattgaggaattattttattaagtcaagatgattaccc
atcttggattttgtttaaagtaggaatatgtgaagcagtagaggagttgattgtctca
tttgttttctcgctgtatcaaattagatataacattttcactgtttgttgcagCTTGT
GTTTTCCCTTCTGCTTCCATGCCTTATATTTTCCCAATTGGGTAGAGCAATCACTATC
GAGAAGATGATACAATGgtaaagataattttgattgttatcgtacttcagttttgtta
ttataaacctagtgttatgcttcatccaagcgtgctatatttgctcttgattttttga
tgtgtgagttattgaccgagatgttgttttcagctaatcatctatttgtatatgctgc
tgaccctctataagtaatacatcctacaaatattatattactgaccacacttgattgg
tgtgctattgtttatctttctatttggatcaatctttgagtaattaggttccatatgg
aacaaattagttttagaagctctagctttctacaaaaggacaaagaaaagactacctg
aaggtgaagaaaatcagcgctcaagcattaatttggttccagttgaatttatggatca
atatgggattgacttcagattccaggattaatttagtagcactactttatgtttaact
ttttgaatactgcagtaaagattggagacactattccaagtagtttgtttccatatga
tatagtaacctatatttttttcttactggcaatttatatcttgttgaaaatgatagaa
atttatgtaatggactattcactattctgtcctatattttgacatacccctggattct
ttcccccttctggtgtgtatatgtataacaagtgtccacttttgtgcataagcttctt
taggagtgtgtgtcctggaaattttcctttgtcaaataactgttctgcacttctgttc
tgcttttacttttttatatcatctatacttgcatgtttgcagGTGGTATATTCCAGTA
AATATTGTTGTTGGCGCAGTATCGGGCTCTTTGATTGGATTTGTTGTGGCATCTATCA
TCAGACCTCCATATCCATACTTCAAGTTCACTGTTATCCATATAGGAATAGgtaccac
ttttctgttttgaaaataagttttatttttatctatcatttgtttatccaataccaat
ccaggttctttttttgcagGAAATATTGGAAATATACCTCTGGTCCTCATTGCAGCGT
TATGTCGTGATCCTTCTAACCCATTTGGCGACTCTGATAAATGCAATCAAGATGGGAA
TGCATATATCTCATTTGGCCAATGGgtacgccatacttttctcttttctcggcttgta
catgctcatttttactcatgtcagcatagtgaccttgttgtttaaagattgccactta
tcacctagactatgcatacaattagctgttgaggttattcactcggtgagtcctctat
atgttgcagGTTGGTGCAATTATTGTTTACACATATGTATTCAAAATGCTTGCTCCAC
CACCAGGACAGACCTTTGATGGTTCTGAAGAGGATGGAATCCCAATCAAGGCATCTGG
AGAGAATACAGTGCCCCAAGTAGGAAAATATCCTATGAACACTAACAGTAGTACTGTA
CCAGAGAATGAACCTTTGTTATCTGCTGGGGAAGTTCAAAAGGAGCGTGCCACTTCTG
TAGGAACAAAGgtaagaaattctaatagtggagctgatttcgaatatccatttggtgc
gtttacttcccactgcaaaactaaatgattttgttgttttatgcagATAATGGGCTAT
GTTAAATGTGTGGTTAAGTTTCTGAAAGACAAGCAGCTTCTCCAGCCACCAATTATTG
CATCTgtatgtattgctgctgtagtgttctactagcattttttaaaccaatttctttg
agtaattatttatatactaactctacgtctttttttcttttggccattgtttaacaca
gGCTTTTGCAATTGCAATCGGTGTTATCCCATTCTTGAAGAATTTTGTACTTACGGAT
GATGCTCCTCTGTTCTTTTTCACAGACAGCTGCCTCATTCTTGGgtatctacatgttt
catttctattttgtttttgcgaaaacatctctcttgccaataaagttatcaaactgag
atgaatgtatctatcacctaattctgtggcatacttttcagAGAAGCTATGATCCCAT
GCATTTTACTTGCTGTTGGGGGCAATCTTGTCGATGgtaagtagctattttagttaaa
actttggagaattgcatggggtgcgataatccttagaatgaccatctttattggttta
gagatatttctaatgtttttgcctttacccatgtttttttatggaatcagGCCCTGGT
GAAGGAAGTAAGAGGCTTGGCGTGCGTACCACTGTTGCTATAATTTTTGCACGGTTGG
TCTTGGTCCCTCTTGCTGGGGTTGGCATTACCATGTTAGTTGATAAACTTGGTTTCAT
TCCCGAAGGTGATAGAATGTTCAAGTTTGTCCTGCTACTGCAGCATTCTATGCCCACA
TCAGTGTTGTCAGgtatgtgaagccaacaagggatgtatcttatcagctatcacttgt
cttcatgtgctcatgcgtacagattcttgttaacaattttgtgtgttgagatctgtaa
ggaaaccacattatggactctgacgataggattgtttgttgcagGTGCTGTTGCAAAT
CTGAGAGGTTGTGGAAAAGAATCAGCTGCAATTTTGTTCTGGGTTCACATTTTTGCTG
TGTTCTCCATGGCGGGATGGATTATATTCTATCTGAGTTTGCTCTTCTAAGTATGTCA
CGCCCTGCTACCACAACAATCTGCAATTTGTGCCCGCATTCCAGCCTGTCAGATGTAA
CTGCATTTTCTTGCTAGATAATGTTACTTGATCTGATTTCAGTCCGCTGGTTCAGATG
CCTTACTGCGCAGGAATATCTTTGAGATCAAATATGGTCCAAAATGTTGAGGAAGAAA
CCAGTATCATGAACTGTACAGATATATCGTTGCAGAGGTACAAGTGTATCTGTACAGA
TATATGGATACGGTCCCTCCTATAATCCCATATTACCGTGGTCTCCTGCTGCTACAAG
CCGTGAGCGATGGGAACACTTTCCCTTTCCCTCCACGTATCAACAGGTTCACACCCAA
CCCTAGATTGGGATGCTGGCCTTCTTAGTTCTCCATAATATTATTTCATTTGTACTTC
CTGTATATTGTAAACCTTGAGATGGCTGTGTTCAAATAGTTTCTTATTGGGCGAGGGC
TGAAGTAGTAGTATAGATGTATTATACTACCTATTTGAGTGTTCGGTTATGCCGCAAA
TGATGCTTTATTATGCAGAACTTGTGCCCGCTGCTGGCCCGTCGGTCGTACTAACTAA
AATTGCCAATCGCGGAGACTTGCATGTTGCAAGCTCAGGCCGGCGCCCAGCGGTTTGC
AGCAAATTTTTTTTTTTGACTCGGCTATCTCCAATAGAATACTCAACTCAATATTTAT
ATTTAAACTCTACTCTATC

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of altering crown root architecture, lateral root formation, and/or stalk height in a Poaceae plant, said method comprising:
reducing or eliminating expression of a PIN-likes 6 (PILS6) gene in a plant or a plant cell of the Poaceae family, wherein said reducing or eliminating is effective to reduce crown root architecture, lateral root formation, and/or stalk height in the plant or a plant produced from the plant cell, as compared to a wild type plant.

2. The method of claim 1, wherein said reducing or eliminating expression of a PIN-likes 6 (PILS6) gene comprises:
(i) introducing a loss-of-function mutation into a coding sequence and/or a regulatory sequence of a PILS6 gene; or
(ii) introducing into the plant or the plant cell an inhibitory polynucleotide targeting the PILS6 gene, thereby decreasing expression of the PILS6 gene.

3.-5. (canceled)

6. The method of claim 2, wherein said reducing or eliminating expression of a PIN-likes 6 (PILS6) gene comprises introducing a loss-of-function mutation into a coding sequence and/or a regulatory sequence of a PILS6 gene, and wherein the loss-of-function mutation is an insertion between nucleotides 777 and 778 of SEQ ID NO: 1 or an insertion between nucleotides 1227 and 1228 of SEQ ID NO:1.

7. (canceled)

8. The method of claim 1, wherein the plant or the plant cell is from maize, rice, wheat, barley, sugarcane, sorghum, millet, switchgrass, rye, oats, bamboo, Bermuda grass, fescue, elephant grass, ryegrass, or Kentucky bluegrass.

9.-10. (canceled)

11. The method of claim 1, wherein the PILS6 gene is ZmPILS6 and comprises a nucleotide sequence encoding an amino acid sequence of SEQ ID NO: 3 or an amino acid sequence having at least 95% sequence identity to the amino acid sequence of SEQ ID NO:3.

12. The method of claim 1, wherein the PILS6 gene expression in roots is reduced by at least 35% compared to PIL6 gene expression in roots of a wild type plant.

13. The method of claim 1, wherein the crown root total root length is reduced by at least 30% compared to the crown root total root length of a wild type plant, the lateral root primordia density is reduced by at least 30% compared to the lateral root primordia density of a wild type plant, and/or the stalk height is reduced by at least 10% compared to stalk height of a wild type plant.

14.-15. (canceled)

16. A Poaceae plant cell comprising:
a non-naturally occurring loss-of-function mutation in a PIN-likes 6 (PILS6) gene encoding a PILS6 protein or fragment thereof, wherein expression and/or activity of the PILS6 gene or protein is reduced or eliminated in the plant cell as compared to a plant cell without the mutation or
an inhibitory polynucleotide targeting a PILS6 gene encoding a PILS6 protein, wherein expression and/or activity of the PILS6 gene is reduced or eliminated in the plant cell compared to a plant cell without the inhibitory polynucleotide.

17.-18. (canceled)

19. The plant cell of claim 16, wherein the PILS6 gene comprises a nucleotide sequence encoding the amino acid sequence of SEQ ID NO:3 or an amino acid sequence having at least 95% sequence identity to the amino acid sequence of SEQ ID NO:3.

20. The plant cell of claim 16, wherein the plant cell comprises a loss-of-function mutation, and wherein the loss-of-function mutation is an insertion between nucleotides 777 and 778 of SEQ ID NO: 1 or an insertion between nucleotides 1227 and 1228 of SEQ ID NO:1.

21.-27. (canceled)

28. A nucleic acid construct comprising:
a nucleotide sequence targeting a PILS6 gene comprising:
(i) a guide RNA; or
(ii) an inhibitory polynucleotide;
a 5′ heterologous DNA promoter sequence; and
a 3′ terminator sequence.

29. The nucleic acid construct of claim 28, wherein the nucleic acid construct further comprises:
a genome editing nuclease.

30. An expression vector comprising the nucleic acid construct of claim 28.

31. (canceled)

32. The nucleic acid construct of claim 28, wherein the PILS6 gene is ZmPILS6 and comprises a nucleotide sequence encoding an amino acid sequence of SEQ ID NO: 3 or an amino acid sequence having at least 95% sequence identity to the amino acid sequence of SEQ ID NO:3.

33.-35. (canceled)

36. A plant transformed with the nucleic acid construct according to claim 28.

37. A plant part from the plant of claim 36.

38. A plant seed produced from the plant of claim 36.

39. A method of breeding for reduced crown root architecture, reduced lateral root formation, or reduced stalk height in a Poaceae plant, said method comprising:
providing a candidate plant or plant part comprising a Poaceae plant cell of claim 16;
analyzing the candidate plant or plant part for the presence, in its genome, of the mutation in the PILS6 gene or the inhibitory polynucleotide;
identifying, based on said analyzing, a candidate plant suitable for breeding, wherein the candidate plant comprises the loss-of-function mutation in the PILS6 gene or the inhibitory polynucleotide; and
breeding the candidate plant with at least one other plant.

40. The method according to claim 39, wherein said analyzing comprises:
isolating nucleic acids from the plant or plant part;
analyzing nucleic acids from the plant or plant part for presence of the loss-of-function mutation in the PILS6 gene or the inhibitory polynucleotide; and
detecting the loss of function-loss-of-function mutation in the PILS6 gene or the inhibitory polynucleotide.

41.-45. (canceled)

46. A method of increasing salt tolerance in a Poaceae plant, said method comprising:
reducing or eliminating expression of a PIN-likes 6 (PILS6) gene in a plant or a plant cell of the Poaceae family, wherein said reducing or eliminating is effective to increase salt tolerance in the plant or a plant produced from the plant cell, as compared to a wild type plant.


SEQUENCE LISTING (67 sequences)

SEQ ID NO: 1 - DNA, 4995 bp, Zea mays
  source 1..4995: mol_type = genomic DNA
  misc_feature 1..4995: note = ZmPILS6 gene
  misc_difference 777^778: note = mu1047700 insertion pils6-1 allele; CTTTCTGAT
  misc_difference 1102^1103: note = mu1090629 insertion pils6-2 allele; GGCTTCAGT
  misc_difference 1227^1228: note = mu_ill_221882.6 insertion pils6-3 allele; CTTCATGGGG
ggatataaaatgcgcgtgtattatctagtccagtccaaccgacccggcatcgtctcgcgagccgcgactcgtctcgtcatttccatcgaacgcccagatcccacattcccacctagctctgcgcgatccatcccggccgccacgccaaggaggccctcgcctgacctcggatcaaagcctcctccatccgccttcatcccggccggtggttcgggccgtcacgggaggggtccatcactggttactcggtggagagctcggggctcggctcccatctcacgattctcaccccacacgcgcaccgggtcacctcgattcggcagcctcgcggtgagttgagttcttcgcttgtcgggccagtccgcctctcgcttggcgcggttatggattcgcgggtggcgtggcgtcgtccgcgatcgctctagctcgcattgagcgattcgcgagttgcgactccttgttttgtttgctctctgtttttttttatgtcgatggtgatatgtggcggcgggattctgagctcggttgcttctaaccttccgcggagtggtttagctaatcggttttgaagctgttgatatggatgtgccgtgtgcgtgacgaatcgaatgggccgcagcaggattcctacgcagctcggcagcagggggaattggatcctgcgaccatgcgaatttggaaccctggagcagtggagtgctaggaggtctcggtgtcgtcatttttttctaggggcgtaggagtcgacttggttggaatctagttagtcataatttctctttctgatttcattggtctgtccatgcgtctgttttcgtcaaacaagaagtttgttattgccaagcatcccgctatggaggaagctgcctgccgtattgtgttacgcgaatccggtgagctgggctctgcgcgttcctcttgacgccctttgcgcgaacgacgagctttcttccagctcggttgcccctgaacaaccaacgccaaaagcagctccagaattttcctgatgtcgtctctatatttttgccttctaagtcatgcttaatggcagagtccttgttgcgtgggtcactgcctcactgggttctgaggcaaggaaaagaaggcttcagtagtcaggtctgggtcaagccatgatggagagatcgctgctggaggtgctggccacggcggcgcaaggagggaccgaggggacgtcagtgctgagcatgctcaagtacgccgtgctgcccatcgccaaggtgttcactgtctgcttcatggggttccttatggcctccaagtacgtcaacattctccagcccaacggccgcaagcttctcaatggggtgagctttgtctcttgtaataactaataagtgctagctaatggaaggttgtgtaatggacaattgtttttctattgaggaattattttattaagtcaagatgattacccatcttggattttgtttaaagtaggaatatgtgaagcagtagaggagttgattgtctcatttgttttctcgctgtatcaaattagatataacattttcactgtttgttgcagcttgtgttttcccttctgcttccatgccttatattttcccaattgggtagagcaatcactatcgagaagatgatacaatggtaaagataattttgattgttatcgtacttcagttttgttattataaacctagtgttatgcttcatccaagcgtgctatatttgctcttgattttttgatgtgtgagttattgaccgagatgttgttttcagctaatcatctatttgtatatgctgctgaccctctataagtaatacatcctacaaatattatattactgaccacacttgattggtgtgctattgtttatctttctatttggatcaatctttgagtaattaggttccatatggaacaaattagttttagaagctctagctttctacaaaaggacaaagaaaagactacctgaaggtgaagaaaatcagcgctcaagcattaatttggttccagttgaatttatggatcaatatgggattgacttcagattccaggattaatttagtagcactactttatgtttaactttttgaatactgcagtaaagattggagacactattccaagtagtttgtttccatatgatatagtaacctatatttttttcttactggcaatttatatcttgttgaaaatgatagaaatttatgtaatggactattcactattctgtcctatattttgacatacccctggattctttcccccttctggtgtgtatatgtataacaagtgtccacttttgtgcataagcttctttaggagtgtgtgtcctggaaattttcctttgtcaaataactgttctgcacttctgttctgcttttacttttttatatcatctatacttgcatgtttgcaggtggtatattccagtaaatattgttgttggcgcagtatcgggctctttgattggatttgttgtggcatctatcatcagacctccatatccatacttcaagttcactgttatccatataggaataggtaccacttttctgttttgaaaataagttttatttttatctatcatttgtttatccaataccaatccaggttctttttttgcaggaaatattggaaatatacctctggtcctcattgcagcgttatgtcgtgatccttctaacccatttggcgactctgataaatgcaatcaagatgggaatgcatatatctcatttggccaatgggtacgccatacttttctcttttctcggcttgtacatgctcatttttactcatgtcagcatagtgaccttgttgtttaaagattgccacttatcacctagactatgcatacaattagctgttgaggttattcactcggtgagtcctctatatgttgcaggttggtgcaattattgtttacacatatgtattcaaaatgcttgctccaccaccaggacagacctttgatggttctgaagaggatggaatcccaatcaaggcatctggagagaatacagtgccccaagtaggaaaatatcctatgaacactaacagtagtactgtaccagagaatgaacctttgttatctgctggggaagttcaaaaggagcgtgccacttctgtaggaacaaaggtaagaaattctaatagtggagctgatttcgaatatccatttggtgcgtttacttcccactgcaaaactaaatgattttgttgttttatgcagataatgggctatgttaaatgtgtggttaagtttctgaaagacaagcagcttctccagccaccaattattgcatctgtatgtattgctgctgtagtgttctactagcattttttaaaccaatttctttgagtaattatttatatactaactctacgtctttttttcttttggccattgtttaacacaggcttttgcaattgcaatcggtgttatcccattcttgaagaattttgtacttacggatgatgctcctctgttctttttcacagacagctgcctcattcttgggtatctacatgtttcatttctattttgtttttgcgaaaacatctctcttgccaataaagttatcaaactgagatgaatgtatctatcacctaattctgtggcatacttttcagagaagctatgatcccatgcattttacttgctgttgggggcaatcttgtcgatggtaagtagctattttagttaaaactttggagaattgcatggggtgcgataatccttagaatgaccatctttattggtttagagatatttctaatgtttttgcctttacccatgtttttttatggaatcaggccctggtgaaggaagtaagaggcttggcgtgcgtaccactgttgctataatttttgcacggttggtcttggtccctcttgctggggttggcattaccatgttagttgataaacttggtttcattcccgaaggtgatagaatgttcaagtttgtcctgctactgcagcattctatgcccacatcagtgttgtcaggtatgtgaagccaacaagggatgtatcttatcagctatcacttgtcttcatgtgctcatgcgtacagattcttgttaacaattttgtgtgttgagatctgtaaggaaaccacattatggactctgacgataggattgtttgttgcaggtgctgttgcaaatctgagaggttgtggaaaagaatcagctgcaattttgttctgggttcacatttttgctgtgttctccatggcgggatggattatattctatctgagtttgctcttctaagtatgtcacgccctgctaccacaacaatctgcaatttgtgcccgcattccagcctgtcagatgtaactgcattttcttgctagataatgttacttgatctgatttcagtccgctggttcagatgccttactgcgcaggaatatctttgagatcaaatatggtccaaaatgttgaggaagaaaccagtatcatgaactgtacagatatatcgttgcagaggtacaagtgtatctgtacagatatatggatacggtccctcctataatcccatattaccgtggtctcctgctgctacaagccgtgagcgatgggaacactttccctttccctccacgtatcaacaggttcacacccaaccctagattgggatgctggccttcttagttctccataatattatttcatttgtacttcctgtatattgtaaaccttgagatggctgtgttcaaatagtttcttattgggcgagggctgaagtagtagtatagatgtattatactacctatttgagtgttcggttatgccgcaaatgatgctttattatgcagaacttgtgcccgctgctggcccgtcggtcgtactaactaaaattgccaatcgcggagacttgcatgttgcaagctcaggccggcgcccagcggtttgcagcaaatttttttttttgactcggctatctccaatagaatactcaactcaatatttatatttaaactctactctatc

SEQ ID NO: 2 - DNA, 1302 bp, Zea mays
  source 1..1302: mol_type = unassigned DNA
  misc_feature 1..1302: note = Coding sequence of ZmPILS6
atgatggagagatcgctgctggaggtgctggccacggcggcgcaaggagggaccgaggggacgtcagtgctgagcatgctcaagtacgccgtgctgcccatcgccaaggtgttcactgtctgcttcatggggttccttatggcctccaagtacgtcaacattctccagcccaacggccgcaagcttctcaatgggcttgtgttttcccttctgcttccatgccttatattttcccaattgggtagagcaatcactatcgagaagatgatacaatggtggtatattccagtaaatattgttgttggcgcagtatcgggctctttgattggatttgttgtggcatctatcatcagacctccatatccatacttcaagttcactgttatccatataggaataggaaatattggaaatatacctctggtcctcattgcagcgttatgtcgtgatccttctaacccatttggcgactctgataaatgcaatcaagatgggaatgcatatatctcatttggccaatgggttggtgcaattattgtttacacatatgtattcaaaatgcttgctccaccaccaggacagacctttgatggttctgaagaggatggaatcccaatcaaggcatctggagagaatacagtgccccaagtaggaaaatatcctatgaacactaacagtagtactgtaccagagaatgaacctttgttatctgctggggaagttcaaaaggagcgtgccacttctgtaggaacaaagataatgggctatgttaaatgtgtggttaagtttctgaaagacaagcagcttctccagccaccaattattgcatctgcttttgcaattgcaatcggtgttatcccattcttgaagaattttgtacttacggatgatgctcctctgttctttttcacagacagctgcctcattcttggagaagctatgatcccatgcattttacttgctgttgggggcaatcttgtcgatggccctggtgaaggaagtaagaggcttggcgtgcgtaccactgttgctataatttttgcacggttggtcttggtccctcttgctggggttggcattaccatgttagttgataaacttggtttcattcccgaaggtgatagaatgttcaagtttgtcctgctactgcagcattctatgcccacatcagtgttgtcaggtgctgttgcaaatctgagaggttgtggaaaagaatcagctgcaattttgttctgggttcacatttttgctgtgttctccatggcgggatggattatattctatctgagtttgctcttctaa

SEQ ID NO: 3 - protein, 433 aa, Zea mays
  REGION 1..433: note = amino acid sequence of ZmPILS6
MMERSLLEVLATAAQGGTEGTSVLSMLKYAVLPIAKVFTVCFMGFLMASKYVNILQPNGRKLLNGLVFSLLLPCLIFSQLGRAITIEKMIQWWYIPVNIVVGAVSGSLIGFVVASIIRPPYPYFKFTVIHIGIGNIGNIPLVLIAALCRDPSNPFGDSDKCNQDGNAYISFGQWVGAIIVYTYVFKMLAPPPGQTFDGSEEDGIPIKASGENTVPQVGKYPMNTNSSTVPENEPLLSAGEVQKERATSVGTKIMGYVKCVVKFLKDKQLLQPPIIASAFAIAIGVIPFLKNFVLTDDAPLFFFTDSCLILGEAMIPCILLAVGGNLVDGPGEGSKRLGVRTTVAIIFARLVLVPLAGVGITMLVDKLGFIPEGDRMFKFVLLLQHSMPTSVLSGAVANLRGCGKESAAILFWVHIFAVFSMAGWIIFYLSLLF

SEQ ID NO: 4 - DNA, 2681 bp, Arabidopsis thaliana
  source 1..2681: mol_type = genomic DNA
  misc_feature 1..2681: note = PIN-LIKES 6 (At5G01990) gene AtPILS6
acttgtaaacttgagtattttaattaaatgaaaataaaccataaataaatgaagttctaaacttgagtaataataaaaacagaataaaaaaaaaggtatgtggggaagttttgaaggggcttttgggcttagaaaaggaaagaagtaaaggaagaagcggcgcacgtggcaggcagtcacgagagtcacgagagatcgagagaagtctttgattctcttctctcatcaaatcaaaatatcatctcagagactccgattctctctgtttctcctccacttttcaacccaagtccaccctcaaaattctcagaatgattgctcggatccttgccgccttagccgattccatggagatgccggtggctgccggtggtggatcagtgctcggtaccatcaaaatcgctgtgatgccaattgcaaaggtattcaccatgtgcttcttgggtcttctcatggcctccaagtacgttaacatcttgcctccctctggccgcaaactcttgaacggggtaattaattctccacactagactttttcactttctctataaatccaaaaaaagcaaactttgaatgaaactgtctctccttttcacacagttggtcttctcgcttttgcttccctgcttgatcttttcccagctcggacaagccgtcactctccaaaaaatgcttcagtggtatttgtttccttctttgtcccccctagtctctctctagtccttatatagatcatcatattcttttccatctttcaggtggttcattcccgttaacgtcgttcttggcaccatctctggctccataatcggtttcatcgttgcttccatcgttcgtccgccttacccttacttcaagtttacaatcatacaaattggagttggttagacccctttttttccctaccctcgcttttctgctctgacactagtctcactttatgtgccttgtttgtttcaggcaacattggcaatgtgcctcttgttttacttgcggctctttgtagggacacttccaacccttttggtgactccgaaaaatgtagcattgatggcactgcttatatctcctttggtcaatgggtactcttttttctctttttaactgcttctatgctgtaactattattatacctactagaatcagtccattagctgcttcagtttctgtctcttatgtaacagtgatacagtttcttgtcttctgctttttttttctgtttggatgaagaggagcttgatgtatgaggagtatcctcattgaaactattttgtataggatctttagttccaatgtactgtttgtcatcaatgtcttatgggaatggatattatttctctctgtgggacttcaattaaccacaaggtttactttttatatcaggttggtgccatcatcctctatacatatgtgtaccagatgtttgctcctcctccagaagggtttgatgctgaagaagaaaatcttgctttaaaaacccttccagtagatgctgctccagagcaagtccccttgcttactcagaattttcccaaggacttttcgccgactcaagatcttttgcctgtacagagcaccgaacctaggggaagaggggtttcaaggaaaggcaaggtgggactgcaattatggaatctttattctatgtttcaggagtattttacctgatataagctggttcttacgaaactttttatcttttgctccgtgatgtagattgcacagatctttgttttcctctatgagaagctgaaactgaagcaaattgtccagcctgcaattgttgcttcggtaagtatctcgtcttttctattccccttttttatctgacaatctaaagatcaagagaccgtgcgattgcctttaccatgtaactaaactagacgtgtttttgttaacttcagatccttgccatgatactcggagcaatacctttcacaaagaagttgatattcacaaatggtgcacctctattcttcttcacagacagctgcatgattcttgggtaagcacactctttatttgtcttctgtgaagcagctgcaaattaattagttaatatatgtaaaacctttggctatcagggacgcgatgataccgtgtatcttactggcactggggggaaatctcattaacggtaggtttggtggttgctttcttttcatactgacttagtttttagccccatccatcacatggatcttattggttccttgggtaaattttaggaccaggaagttcaaaacttggtttcaagacaacagcagctattataattggacggttggtgttagtgccaccagtaggactaggaatcgtgacagtagcagacaaacttgggttccttcctgctgatgataagatgttccgatttgtgttactccttcagcacacaatgcctacatcagtgctctcaggagctgttgccaaccttagaggctgtggaagggaatcagctgctgtgctcttctgggtccacattttcgccatcttctcaatggctggatggatggtactctacattaacatactcttctgaattctcttcttattgacacattcatctgctactatacaatacttctgtgtaaatacagtggcatctcctatgtgcctgaaccaaccaaaattctgattttttttttttcctttcctgtgtaaatgttagtttataactcttt

SEQ ID NO: 5 - DNA, 1296 bp, Arabidopsis thaliana
  source 1..1296: mol_type = unassigned DNA
  misc_feature 1..1296: note = PIN-LIKES 6 (At5G01990) AtPILS6 coding sequence
atgattgctcggatccttgccgccttagccgattccatggagatgccggtggctgccggtggtggatcagtgctcggtaccatcaaaatcgctgtgatgccaattgcaaaggtattcaccatgtgcttcttgggtcttctcatggcctccaagtacgttaacatcttgcctccctctggccgcaaactcttgaacgggttggtcttctcgcttttgcttccctgcttgatcttttcccagctcggacaagccgtcactctccaaaaaatgcttcagtggtggttcattcccgttaacgtcgttcttggcaccatctctggctccataatcggtttcatcgttgcttccatcgttcgtccgccttacccttacttcaagtttacaatcatacaaattggagttggcaacattggcaatgtgcctcttgttttacttgcggctctttgtagggacacttccaacccttttggtgactccgaaaaatgtagcattgatggcactgcttatatctcctttggtcaatgggttggtgccatcatcctctatacatatgtgtaccagatgtttgctcctcctccagaagggtttgatgctgaagaagaaaatcttgctttaaaaacccttccagtagatgctgctccagagcaagtccccttgcttactcagaattttcccaaggacttttcgccgactcaagatcttttgcctgtacagagcaccgaacctaggggaagaggggtttcaaggaaaggcaagattgcacagatctttgttttcctctatgagaagctgaaactgaagcaaattgtccagcctgcaattgttgcttcgatccttgccatgatactcggagcaatacctttcacaaagaagttgatattcacaaatggtgcacctctattcttcttcacagacagctgcatgattcttggggacgcgatgataccgtgtatcttactggcactggggggaaatctcattaacggaccaggaagttcaaaacttggtttcaagacaacagcagctattataattggacggttggtgttagtgccaccagtaggactaggaatcgtgacagtagcagacaaacttgggttccttcctgctgatgataagatgttccgatttgtgttactccttcagcacacaatgcctacatcagtgctctcaggagctgttgccaaccttagaggctgtggaagggaatcagctgctgtgctcttctgggtccacattttcgccatcttctcaatggctggatggatggtactctacattaacatactcttctga

SEQ ID NO: 6 - protein, 431 aa, Arabidopsis thaliana
  REGION 1..431: note = PIN-LIKES 6 (At5G01990) AtPILS6 amino acid sequence
MIARILAALADSMEMPVAAGGGSVLGTIKIAVMPIAKVFTMCFLGLLMASKYVNILPPSGRKLLNGLVFSLLLPCLIFSQLGQAVTLQKMLQWWFIPVNVVLGTISGSIIGFIVASIVRPPYPYFKFTIIQIGVGNIGNVPLVLLAALCRDTSNPFGDSEKCSIDGTAYISFGQWVGAIILYTYVYQMFAPPPEGFDAEEENLALKTLPVDAAPEQVPLLTQNFPKDFSPTQDLLPVQSTEPRGRGVSRKGKIAQIFVFLYEKLKLKQIVQPAIVASILAMILGAIPFTKKLIFTNGAPLFFFTDSCMILGDAMIPCILLALGGNLINGPGSSKLGFKTTAAIIIGRLVLVPPVGLGIVTVADKLGFLPADDKMFRFVLLLQHTMPTSVLSGAVANLRGCGRESAAVLFWVHIFAIFSMAGWMVLYINILF

SEQ ID NO: 7 - DNA, 4828 bp, Sorghum bicolor
  source 1..4828: mol_type = genomic DNA
  misc_feature 1..4828: note = PIN-LIKES 6 (LOC8055297) gene SbPILS6
ctagcagtccaaccaaacccgcgcgtcgtctcgcgagccgcgactccccatccccatcgtctcgtcgtttcccccgagcgcccagatcccatccctacccgctccgcgcgacgcgaatcacaagcccgcaactcttgggactttgggaggcgccccccacccctgcctgcctgacctcggatcaaagcctcctccgtccgccttaatcgcggcaggtcgttcgggcccgggccgtcacgcgggaggggttcatcacccgggagagctcggggctcggctcccttcttctcgggacccccgcgcgcgcaccggctgggtcacctcgacgcggcggccacgcggtgagtcgagttcttcgcttctcgggccgccagtccgcctctcgcttggttggggctgctagcggggttcaggattcgcgggtggcgtggcgtggcgtcgtccgcgatcgctctcgctcgcattgagcgattattcgcgactgcttgttcgtttgctttctctctgttttttttctcttgtggggatgtagattgtgatatgtggcgttgggattctgagctcggttgcttcctaagcttccgcggagtggtttaacgcgtacgtatatggtaatcggttttgaagctgctaatatggatgtgccgtgtgcgtgacgaatcgaatcggccgcaggatgcctacgccctacgcagctcggcagctgggggaattggatcccccgaccatgcgaatttgcaacccagagtgctccgggttctcggtgtcgtcatcttttactagtataagtagtagtcgttgtttggaatctagtttgtcataatttctcttcccgatttgattggtttgtcgatgcgtctgttttcgtcgaacaagaagtttgttattgctaagcttcccgtttcggggggaagctgcctgccgtactgtgttacgcgaatccggtgagttgggctctgcggttcctctcgacgcgctctgcgccaacggaagacgagctctgttccagctgggtggcccctgaacaaccagcgccaaaagcagttccagaattttcctgatgttgcctctatattttgtcttctaagtcatgcttaaataatggcagagtcctggttgcgtgggtcactggatcctgaggcaagaaaaagaaggcttcagtagtcaggtctgggtcaagccatgatggagaggtcgctgctggaggtgctggccacggctgcgcaaggagggaccgaggggacgtcggtgctgagcatgctcaagtatgccgtgcttcccatcgccaaggtgttcactgtctgcttcatggggttcctcatggcctccaagtacgtcaacattctccagcccaacggccgcaagcttctcaatggggtgagctttgtctcttgtaattaataaaagctagcttttgcgcaatggacaattgtagctggattttgtgattttgttcagagtaggaatatgtgtagtagttggggacttttgtgctcatattagacatcacattttcactgtttgctgcagcttgtgttttcccttctgcttccatgccttatattttcccaattgggtagagcaatcactatcgagaagatgatacaatggtaaggataatttctattgttatcgtacttaagttttgttattataaacctagtgttatgcttcgtccaggtgtgctatatggctcttgatttcttatgtgtgagctattgactgagatgttgttttcaactaataatctctatatttgtatatgctgctggccctctattagcaatacatcctgcaagtattagattactgaaaatgacaaaccaatggtgtgctattgttgatctttctggttgggccaacctttaagtaattaagttccatatggaacaatttagctttggaagctctagctttctacaaaaggacaaagaaaagaccaactgaaggtgaaggaaatcagcactcaagcattaatttggttccagttgaatttatggatcatatggattgactccacattccatgattaatttgtaccgctactttatttttaacatactgaaatactgcagttagtaaagattggagaaactattccaagtagtttgtttccatataatttttttcttactagcatacatatcttgttgaaagtgatagaaatttatgtaatggtctattcactatcgtgtcctacattttgacatactcatggattcctttccccttcttgtgtgtatatgtatagcaagtgtccacttttatgcataagcttctttaggagtatgtgtcctcgaaatttcctttgtcaaataactgttctgaacttctgttctgcttttacttctgttttttaatatcatctatacttgcatgtttgcaggtggtatattccagtaaatattgttgtaggtgcagtatccggctctttgattggatttgttgtggcatctatcatccgacctccatatccatactttaagttcactattatccatataggaataggtaccacttttgtgttttgaaaataagttttattttctatttactattcgttatccaataccattgtggtccttttttttgcaggaaatattggaaatatacctctggtcctcattgcagcgttatgtcgggatccttctaacccatttggtgactctgataaatgcaatcaagatgggaatgcgtatatctcatttggccaatgggtacgccatctcagcttttacatgctcatttttactcaagtcagcatagtgaccttgttgtttaaagatatctgtttatcatctagactatgcatacaattggttattcactcagttagtcctctatatgttgcaggttggtgcaattattgtttacacatatgtattcaagatgcttgctccaccaccaggacagacctttgatggttctgaagaggatgaactcccaatcaaggcatctggagagaatacagtgccccaaataggaaattatcctatgaacactcacactagtactgtaccagagaatgaaccattgttatctgctggggatgttcaaaaggaacgtgccacttctgtaggaacaaaggtaagaaattcgaatagtggagagctgatttcgatatcagtttggtgcgcgtacttccaactgcaaaactaaatgattttgtatattaatgcagataatgggctttgttaaatgtgtggttaagttcctgaaagacaagcagcttctccagccaccgattattgcatctgtatgtattgctgctgtagtgttctactaacaatttttaaaccgaattctactagcaattatttatatactaattctacaactttttccccttgaccattgtttaacgcaggcgtttgcaattgtaattggtgttatcccattcttgaagaattttgtccttacagatgatgcccctctgttctttttcacagacagctgcctcattcttgggtatctacatgtttcatttcttgttttgttctcttgccaatgaagttatcaaactgagatgaatgtatctatcatctatcacctaattctgtggcatacttttcagagaagctatgatcccatgcattttacttgctgttgggggcaatcttgtcgatggtaagtaattattttagttaaaactttggagaaattgcatggggtgcagtcacccttacatggtttagaggtatttctcatgtttttgtctttatccatgtttatttatggaatcaggccctggtgaaggaagtaagaggcttggcgtccgtaccaccgttgctataatttttgcacggttggtcttggtccctcttgctggggttggcattatcatattagttgataaacttggtttcattcccaaagatgataaaatgttcaagtttgtcctgctactgcagcattctatgcccacatcagtgctgtcaggtatgtgaaaagggatgtgtcttatcagccaacacttatcttcatgtgctcatgtgtacagattcttaacaattttgtgtgttagttgagatctgtaaggcattgggtggtgcctcgcatgttctccatgttaggaaaccacattatggaatctgaagatagaattgtttgttgcaggtgctgttgcaaatctgagagggtgtggaaaagaatcagctgcaattttgttctgggttcacatttttgctgtgttctccatggcaggatggattattttctatctgagcttgctcttctaagtatgtcacaccctactaccgcaacaatctgcagtttattcttgcatccccgcctgtcagatgtaacgcaattttttgctagataataatgttagttgatctgattccaatcagttgattgcagataccttactgcacaggaatatctttgagatcaaatattgtccaaaatattgacgaagaaacaagtaccatgaactgtacagtagccactaaatcgaagttggagaggtacaattgtatcaggacagatatatggatatgatcaccccctataataccgtggtctcctgctgctgcaagcagtgagcgatgtgaggacagtgtttccctgcacgtatcatcaggtcacggagcctagattgggatgatgcccttcttatcagaaccctagactatgcagtgctccacgatattcattcatttgtacttccgcgtattgtaaatgtgaaccttgaggctttgttcaaatagtgtccttctgggcgagggctgaaatagtatagatgtatttaatactaccttaggcctgtctatttgggtgttggctttgcaggaaatgatgctttatcatgcagaacttgtgccca

SEQ ID NO: 8 - RNA, 2182 nt, Sorghum bicolor
  source 1..2182: mol_type = mRNA
  misc_feature 1..2182: note = PIN-LIKES 6 (LOC8055297) SbPILS6 transcript variant X1
tagcagtccaaccaaacccgcgcgtcgtctcgcgagccgcgactccccatccccatcgtctcgtcgtttcccccgagcgcccagatcccatccctacccgctccgcgcgacgcgaatcacaagcccgcaactcttgggactttgggaggcgccccccacccctgcctgcctgacctcggatcaaagcctcctccgtccgccttaatcgcggcaggtcgttcgggcccgggccgtcacgcgggaggggttcatcacccgggagagctcggggctcggctcccttcttctcgggacccccgcgcgcgcaccggctgggtcacctcgacgcggcggccacgcgagtcctggttgcgtgggtcactggatcctgaggcaagaaaaagaaggcttcagtagtcaggtctgggtcaagccatgatggagaggtcgctgctggaggtgctggccacggctgcgcaaggagggaccgaggggacgtcggtgctgagcatgctcaagtatgccgtgcttcccatcgccaaggtgttcactgtctgcttcatggggttcctcatggcctccaagtacgtcaacattctccagcccaacggccgcaagcttctcaatgggcttgtgttttcccttctgcttccatgccttatattttcccaattgggtagagcaatcactatcgagaagatgatacaatggtggtatattccagtaaatattgttgtaggtgcagtatccggctctttgattggatttgttgtggcatctatcatccgacctccatatccatactttaagttcactattatccatataggaataggaaatattggaaatatacctctggtcctcattgcagcgttatgtcgggatccttctaacccatttggtgactctgataaatgcaatcaagatgggaatgcgtatatctcatttggccaatgggttggtgcaattattgtttacacatatgtattcaagatgcttgctccaccaccaggacagacctttgatggttctgaagaggatgaactcccaatcaaggcatctggagagaatacagtgccccaaataggaaattatcctatgaacactcacactagtactgtaccagagaatgaaccattgttatctgctggggatgttcaaaaggaacgtgccacttctgtaggaacaaagataatgggctttgttaaatgtgtggttaagttcctgaaagacaagcagcttctccagccaccgattattgcatctgcgtttgcaattgtaattggtgttatcccattcttgaagaattttgtccttacagatgatgcccctctgttctttttcacagacagctgcctcattcttggagaagctatgatcccatgcattttacttgctgttgggggcaatcttgtcgatggccctggtgaaggaagtaagaggcttggcgtccgtaccaccgttgctataatttttgcacggttggtcttggtccctcttgctggggttggcattatcatattagttgataaacttggtttcattcccaaagatgataaaatgttcaagtttgtcctgctactgcagcattctatgcccacatcagtgctgtcaggtgctgttgcaaatctgagagggtgtggaaaagaatcagctgcaattttgttctgggttcacatttttgctgtgttctccatggcaggatggattattttctatctgagcttgctcttctaaataccttactgcacaggaatatctttgagatcaaatattgtccaaaatattgacgaagaaacaagtaccatgaactgtacagtagccactaaatcgaagttggagaggtacaattgtatcaggacagatatatggatatgatcaccccctataataccgtggtctcctgctgctgcaagcagtgagcgatgtgaggacagtgtttccctgcacgtatcatcaggtcacggagcctagattgggatgatgcccttcttatcagaaccctagactatgcagtgctccacgatattcattcatttgtacttccgcgtattgtaaatgtgaaccttgaggctttgttcaaatagtgtccttctgggcgagggctgaaatagtatagatgtatttaatactaccttaggcctgtctatttgggtgttggctttgcaggaaatgatgctttatcatgcagaacttgtgccca

SEQ ID NO: 9 - RNA, 2368 nt, Sorghum bicolor
  source 1..2368: mol_type = mRNA
  misc_feature 1..2368: note = PIN-LIKES 6 (LOC8055297) SbPILS6 transcript variant X2
gggttcaggattcgcgggtggcgtggcgtggcgtcgtccgcgatcgctctcgctcgcatgagcgattattcgcgactgcttgttcgtttgctttctctctgttttttttctcttgtggggatgtagattgtgatatgtggcgttgggattctgagctcggttgcttcctaagcttccgggagtggtttaacgcgtacgtatatggtaatcggttttgaagctgctaatatggatgtgcgtgtgcgtgacgaatcgaatcggccgcaggatgcctacgccctacgcagctcggcagcgggggaattggatcccccgaccatgcgaatttgcaacccagagtgctccgggttctcgggtcgtcatcttttactagtataagtagtagtcgttgtttggaatctagtttgtcataattctcttcccgatttgattggtttgtcgatgcgtctgttttcgtcgaacaagaagtttgtattgctaagcttcccgtttcggggggaagctgcctgccgtactgtgttacgcgaatccggtcctggttgcgtgggtcactggatcctgaggcaagaaaaagaaggcttcagtagtcaggtctgggtcaagccatgatggagaggtcgctgctggaggtgctggccacggctgcgcaaggagggaccgaggggacgtcggtgctgagcatgctcaagtatgccgtgcttcccatcgccaggtgttcactgtctgcttcatggggttcctcatggcctccaagtacgtcaacattctccgcccaacggccgcaagcttctcaatgggcttgtgttttcccttctgcttccatgccttaattttcccaattgggtagagcaatcactatcgagaagatgatacaatggtggtatattcagtaaatattgttgtaggtgcagtatccggctctttgattggatttgttgtggcatctacatccgacctccatatccatactttaagttcactattatccatataggaataggaaatattggaaatatacctctggtcctcattgcagcgttatgtcgggatccttctaacccatttggtgactctgataaatgcaatcaagatgggaatgcgtatatctcatttggccaatgggttggtgcaattattgtttacacatatgtattcaagatgcttgctccaccaccaggacagacctttgatggttctgaagaggatgaactcccaatcaaggcatctggagagaatacagtgccccaaataggaaattatcctatgaacactcacactagtactgtaccagagaatgaaccattgttatctgctggggatgttcaaaaggaacgtgccacttctgtaggaacaaagataatgggctttgttaaatgtgtggttaagttcctgaaagacaagcagcttctccagccaccgattattgcatctgcgtttgcaattgtaattggtgttatcccattcttgaagaattttgtccttacagatgatgcccctctgttctttttcacagacagctgcctcattcttggagaagctatgatcccatgcattttacttgctgttgggggcaatcttgtcgatggccctggtgaaggaagtaagaggcttggcgtccgtaccaccgttgctataatttttgcacggttggtcttggtccctcttgctggggttggcattatcatattagttgataaacttggtttcattcccaaagatgataaaatgttcaagtttgtcctgctactgcagcattctatgcccacatcagtgctgtcaggtgctgttgcaaatctgagagggtgtggaaaagaatcagctgcaattttgttctgggttcacatttttgctgtgttctccatggcaggatggattattttctatctgagcttgctcttctaaataccttactgcacaggaatatctttgagatcaaatattgtccaaaatattgacgaagaaacaagtaccatgaactgtacagtagccactaaatcgaagttggagaggtacaattgtatcaggacagatatatggatatgatcaccccctataataccgtggtctcctgctgctgcaagcagtgagcgatgtgaggacagtgtttccctgcacgtatcatcaggtcacggagcctagattgggatgatgcccttcttatcagaaccctagactatgcagtgctccacgatattcattcatttgtacttccgcgtattgtaaatgtgaaccttgaggctttgttcaaatagtgtccttctgggcgagggctgaaatagtatagatgtatttaatactaccttaggcctgtctatttgggtgttggctttgcaggaaatgatgctttatcatgcagaacttgtgccca

SEQ ID NO: 10 - RNA, 2193 nt, Sorghum bicolor
  source 1..2193: mol_type = mRNA
  misc_feature 1..2193: note = PIN-LIKES 6 (LOC8055297) SbPILS6 transcript variant X3
ctagcagtccaaccaaacccgcgcgtcgtctcgcgagccgcgactccccatccccatcgtctcgtcgtttcccccgagcgcccagatcccatccctacccgctccgcgcgacgcgaatcacaagcccgcaactcttgggactttgggaggcgccccccacccctgcctgcctgacctcggatcaaagcctcctccgtccgccttaatcgcggcaggtcgttcgggcccgggccgtcacgcgggaggggttcatcacccgggagagctcggggctcggctcccttcttctcgggacccccgcgcgcgcaccggctgggtcacctcgacgcggcggccacgcgagtcctggttgcgtgggtcactggatcctgaggcaagaaaaagaaggcttcagtagtcaggtctgggtcaagccatgatggagaggtcgctgctggaggtgctggccacggctgcgcaaggagggaccgaggggacgtcggtgctgagcatgctcaagtatgccgtgcttcccatcgccaaggtgttcactgtctgcttcatggggttcctcatggcctccaagtacgtcaacattctccagcccaacggccgcaagcttctcaatgggcttgtgttttcccttctgcttccatgccttatattttcccaattgggtagagcaatcactatcgagaagatgatacaatggtggtatattccagtaaatattgttgtaggtgcagtatccggctctttgattggatttgttgtggcatctatcatccgacctccatatccatactttaagttcactattatccatataggaataggaaatattggaaatatacctctggtcctcattgcagcgttatgtcgggatccttctaacccatttggtgactctgataaatgcaatcaagatgggaatgcgtatatctcatttggccaatgggttggtgcaattattgtttacacatatgtattcaagatgcttgctccaccaccaggacagacctttgatggttctgaagaggatgaactcccaatcaaggcatctggagagaatacagtgccccaaataggaaattatcctatgaacactcacactagtactgtaccagagaatgaaccattgttatctgctggggatgttcaaaaggaacgtgccacttctgtaggaacaaagataatgggctttgttaaatgtgtggttaagttcctgaaagacaagcagcttctccagccaccgattattgcatctgcgtttgcaattgtaattggtgttatcccattcttgaagaattttgtccttacagatgatgcccctctgttctttttcacagacagctgcctcattcttggagaagctatgatcccatgcattttacttgctgttgggggcaatcttgtcgatggccctggtgaaggaagtaagaggcttggcgtccgtaccaccgttgctataatttttgcacggttggtcttggtccctcttgctggggttggcattatcatattagttgataaacttggtttcattcccaaagatgataaaatgttcaagtttgtcctgctactgcagcattctatgcccacatcagtgctgtcaggtgctgttgcaaatctgagagggtgtggaaaagaatcagctgcaattttgttctgggttcacatttttgctgtgttctccatggcaggatggattattttctatctgagcttgctcttctaattgattgcagataccttactgcacaggaatatctttgagatcaaatattgtccaaaatattgacgaagaaacaagtaccatgaactgtacagtagccactaaatcgaagttggagaggtacaattgtatcaggacagatatatggatatgatcaccccctataataccgtggtctcctgctgctgcaagcagtgagcgatgtgaggacagtgtttccctgcacgtatcatcaggtcacggagcctagattgggatgatgcccttcttatcagaaccctagactatgcagtgctccacgatattcattcatttgtacttccgcgtattgtaaatgtgaaccttgaggctttgttcaaatagtgtccttctgggcgagggctgaaatagtatagatgtatttaatactaccttaggcctgtctatttgggtgttggctttgcaggaaatgatgctttatcatgcagaacttgtgccca

SEQ ID NO: 11 - DNA, 1302 bp, Sorghum bicolor
  source 1..1302: mol_type = unassigned DNA
  misc_feature 1..1302: note = PIN-LIKES 6 (LOC8055297) SbPILS6 coding sequence
atgatggagaggtcgctgctggaggtgctggccacggctgcgcaaggagggaccgaggggacgtcggtgctgagcatgctcaagtatgccgtgcttcccatcgccaaggtgttcactgtctgcttcatggggttcctcatggcctccaagtacgtcaacattctccagcccaacggccgcaagcttctcaatgggcttgtgttttcccttctgcttccatgccttatattttcccaattgggtagagcaatcactatcgagaagatgatacaatggtggtatattccagtaaatattgttgtaggtgcagtatccggctctttgattggatttgttgtggcatctatcatccgacctccatatccatactttaagttcactattatccatataggaataggaaatattggaaatatacctctggtcctcattgcagcgttatgtcgggatccttctaacccatttggtgactctgataaatgcaatcaagatgggaatgcgtatatctcatttggccaatgggttggtgcaattattgtttacacatatgtattcaagatgcttgctccaccaccaggacagacctttgatggttctgaagaggatgaactcccaatcaaggcatctggagagaatacagtgccccaaataggaaattatcctatgaacactcacactagtactgtaccagagaatgaaccattgttatctgctggggatgttcaaaaggaacgtgccacttctgtaggaacaaagataatgggctttgttaaatgtgtggttaagttcctgaaagacaagcagcttctccagccaccgattattgcatctgcgtttgcaattgtaattggtgttatcccattcttgaagaattttgtccttacagatgatgcccctctgttctttttcacagacagctgcctcattcttggagaagctatgatcccatgcattttacttgctgttgggggcaatcttgtcgatggccctggtgaaggaagtaagaggcttggcgtccgtaccaccgttgctataatttttgcacggttggtcttggtccctcttgctggggttggcattatcatattagttgataaacttggtttcattcccaaagatgataaaatgttcaagtttgtcctgctactgcagcattctatgcccacatcagtgctgtcaggtgctgttgcaaatctgagagggtgtggaaaagaatcagctgcaattttgttctgggttcacatttttgctgtgttctccatggcaggatggattattttctatctgagcttgctcttctaa

SEQ ID NO: 12 - protein, 433 aa, Sorghum bicolor
  REGION 1..433: note = PIN-LIKES 6 (LOC8055297) SbPILS6 amino acid sequence
MMERSLLEVLATAAQGGTEGTSVLSMLKYAVLPIAKVFTVCFMGFLMASKYVNILQPNGRKLLNGLVFSLLLPCLIFSQLGRAITIEKMIQWWYIPVNIVVGAVSGSLIGFVVASIIRPPYPYFKFTIIHIGIGNIGNIPLVLIAALCRDPSNPFGDSDKCNQDGNAYISFGQWVGAIIVYTYVFKMLAPPPGQTFDGSEEDELPIKASGENTVPQIGNYPMNTHTSTVPENEPLLSAGDVQKERATSVGTKIMGFVKCVVKFLKDKQLLQPPIIASAFAIVIGVIPFLKNFVLTDDAPLFFFTDSCLILGEAMIPCILLAVGGNLVDGPGEGSKRLGVRTTVAIIFARLVLVPLAGVGIIILVDKLGFIPKDDKMFKFVLLLQHSMPTSVLSGAVANLRGCGKESAAILFWVHIFAVFSMAGWIIFYLSLLF

SEQ ID NO: 13 - DNA, 5300 bp, Oryza sativa
  source 1..5300: mol_type = genomic DNA
  misc_feature 1..5300: note = PIN-LIKES 6-1 (LOC4327495) Gene NC_089035 REGION: 35441596..35446895
gatagcttaatctcgagcgcttcgagttcgatctctcgtgaaggggacgacgagggcattgtctccctcctcgcgcgcgcggagtcgccgccgccgccgcctcgtcctcggccacgcggtgagtcctcgcgccgacctcgccgttttggttgcgtttttttttagttttctctgcagcgggatttggagacgatgggtgtcgctgtgtgtgctgtgaaatggggatttgtggtttttttttttggtttgtggagtacgagtacgcgggggaagtttcgaggtcggttgcatccggagctcccgctggagtggtctactctagtgcggatttggaagcggagaagttggagttggaggtggcgtgacggatcaagcggagtcgcgatcgctcgccggagccgtgcggattccggtgctagcgaacaacggcggcaccgtcggcgttttgtagcggtcaagctgctgctgctgctcggaatctagctaggtttttgttctgtcctaatccgggtttcccgattggatttgatggtcttctccccacgcatgagagttggttagcacggaagctttggggattgttccgtttaattcggcgcgcttgctgagttgtgcgcgctcacgctgcgctccagttggctaggcctgcgggggaggcgatctctgttccgctttggcgctctctggacaaccgacttcaaaagcggctcctgatttttttcctgctgttgcttgtatattttgccttctaagtcactcatgttggcaggaattgcgtgggtcctggttgtggaagtcaggaagtgaagaagaagaagaagaagtgaactagtgaagcctggggcaagccatgatggagaggtcgctgatggaggcgctggctacggcggcgcagggaggcaccgtggggacgtcggttttcgacatgctcaagtacgccgtgctgcccattgccaaggtgtttaccgtctgtttcatggggttcctcatggcctctaagtatgtcaatatcctccagccgaacggccgcaagcttctcaatggggtgagccttgttcattctcttgcaatataattttatctgtaagatttatgagtttttgctgtgactatggagaatggtaagttggctgctgtctagaataggaagatgtgggggcagttgcttgtgcttacttgcattcctgatgttctcgattatgcttacattttggtcgtttggtgcagcttgtgttctcgcttctacttccttgccttatattttcccaactgggtagagcaatcacaatcgaaaagatgctgcaatggtaatgacagattctagttaatatgctttgttaagtttgctgcatgcttttcgcatttcattgtgtagtctattgatcatggttcttaccataactagatcgttctggttgcgggaattttaagtcatcatccaaatggagtaaattagctcccagaattttctgcaaaaagatcttaagtgtaccaatcgaaagcgaaggaagtgttgcattatctgaaagtttcaggactaatttaagacgtcaaaatacttaaattaccatattgtttgttactggcaaaatccatctggttggaattggttggacttcatctataaggccatccaatccttgttattatttatgatgacataaggcctttttctttgcaaaagataatcgcattgtgcctatgttttgcttttactgtttttaacattcttacagtagcaatattttgttgtttgcaggtggtatattccagtaaatattgttgtaggtgcagtgtcaggctctttgattggctttgtggtggcttctatcatccgaccaccttatccatacttcaagttcactgtgattcacattggaataggtacctggttttctttattaaataaagttgccttctcatttatcattcttctattttatattgatccgatcccttatttcgattcagggaatattggaaatatacctctggttctcattgcagcattatgtcgagatcccaccaatccttttggtgactctgataaatgtaatgaagatgggaatgcgtatatctcatttggtcaatgggtacatgtcttttcttttctgaagttgcatgagttcatgctttattcaggacctctactatgcaaaataaatgtaacaggttgaatttaggttaggtatgcattgagatcattcactcagtgaatcctttaaatgtttcaggttggtgcaattattgtttacacatatgtgttcaagatgcttgctccaccacctggtgaatcctttgatagcgctgaagaagatattcttccaattaaggcatctggagataatgtggtgcctgaaaaagggaaatatccaacaagcactcgcactagtactgtacctgaaaatgagcctttgttatcttctgaaggtgataaaaatgtttctacttctctaggatcgaaggtgagaaataccatcagaaatttggtatgtttacttcttatatcaatttgatttctaacatgagctaaaaatcatgctctgttgatgcagataatgggcattgttagaagcatggttaagttcctaaaagacaagcagcttcttcagccaccaattattgcctctgtatgtatctacactacaatattatgctagcaataaacacagaacatttctgtatactgtggtttgacagcaactattttacactatgcacaggtttttgcaattgccattggtgttgttccagtcttgaagaattttgtcctcactgatgatgcacctctgttcttcttcacagacagctgtctcattctagggtatttacatgactttcagttccctttttgtaaaataaagtacactttcctataaaattacaaaagcacgatgagccaccttggttcctagtttctaattctgtcattcctttttcagggaagcaatgatcccttgcattttgcttgcagtggggggcaatcttgtcgatggtaagcacctagttctgttgaaactattgcgcggctcctaccattctatagtactaggagcctaggatgcatctccagatcttatcctagctcatgtaaccatgtttattgcatcaggtcctggtgaaggaagtaacaggcttggtgtgcggacaaccgttgctattatttttgcacggctcatcttggttcctcttgctggggttggcatcattgtgttggttgataaacttggtttcattcctaaagatgacaaaatgttcaagtttgtcttgctactgcagcattctatgcccacatctgtgctgtcaggtactaagaaatgaataagggacactacttatcacttattatcatgtgagcatgttcaaagatagattccttctaatagttttatgtgttggttgagatctgtaaggcattggtggtggaacaccatgtgctttatgttagcgaccaaataatggaatatgatctgagttttttttttttttttggcaggtgctgttgcaaacctgaggggctgtggaaaagaatctgctgctatcttgttctgggtgcacatctttgctgtgttctccatggcaggatggattattttgtatttgagcttactcttctaagtacgtcataccctggtaatggcagttaatctgtggcatttgtgctcaagttgcagctctacagattaactactatacgagtccttgaatacttagagccactctctgatttgatttgcagatgctatactgcacaggaacgtccataagatcaaaatatcaccagatataccagaaaaagatagtaccatgacatgtacattagccagtaaaccatggcgacagcctgaaggtacgagttgtatcagtattcagtatagatatggttctcctgcccagtgcccaatcgcttaagcgccactgctatgttaattttgctaatcgctcgctgctacaagcaactgagcaatgagaaaatgagggcatagcggtattgccgatgcccagatcatcaggtcctcatgacctttaggtcggtttcctatataagcctctttcctttattaatgtagccccccccacccccccaccccccccccccccccccacaatgatcttcattgaatgaactacaagcttcaagtgtgtttttgtggtgcagttatatttgaaagagtaagagtaaactacaaattggaccactgtttattggcaaagttttgctttgggcctccctttaaccaatgttttcactttggactagataattatacttgtgtcactttggatcacccctaactcttatgttaagtacatcaataatctcgattataccggcttttgcatgccgatgaactcgtgaaattgtatgtagaccacacctttggcacagaatgagttggatgtaaatggataagagttgagagttgtccaagtgacacaaataaaaaattatctagtccaaagtgaaaatattggttaaaaggcaagcccaaaccgaaactttgattataaaaggtggcccaaagcatgaacttcggttgaaactttgattatatatcttgattatatggatacaatctctcatgcatgaactgatgcgtgcacatgcataatgtttatgtgatcacatgattcgcatcatgcaggatgggtgcccgacatccacccacgcacatacatgaatgccaaaatcaaaatatttgcctgcaagctcatgccagcggttcctagtatgcatgtgggtcatgtagccttaactagcattggtacttccagatccagtgatatcagagtacatatgtgatctctccatgtgcttatagatctgggaacctgcagatctgaagttctgaacccacagttttcattcacttttaatcttttgaatctgaaggagagggagattcatgattcatcaccgtaccaaaaaaaaaggtgagagatttctatagtgaactttaaccaagcagacagctcaatatgcctcttggtgtgcgtattggttggggtctcaaaatcagatgcaaatccgtcaaacggaatatacttggaagatacgtatcgcgtcagcactcagcatggagtcagacaggtactgacattcagacacgctagggtcggacaaaaaacagatactgccaatattcgttcatcggaaatagaacaaaaaaacatgtattgacaataaatcagtgtacagaacagtaccgtatcgatatggcgagcagataataacgcaaggatacgtatgagccgtctctcatcagcagaaatggctgcatgtattcgcttcgcaccca

SEQ ID NO: 14 - RNA, 1936 nt, Oryza sativa
  source 1..1936: mol_type = mRNA
  misc_feature 1..1936: note = PIN-LIKES 6-1 (LOC4327495) GenBank Accession No. XM_015765602.2 transcript
gatagcttaatctcgagcgcttcgagttcgatctctcgtgaaggggacgacgagggcattgtctccctcctcgcgcgcgcggagtcgccgccgccgccgcctcgtcctcggccacgcggaattgcgtgggtcctggttgtggaagtcaggaagtgaagaagaagaagaagaagtgaactagtgaagcctggggcaagccatgatggagaggtcgctgatggaggcgctggctacggcggcgcagggaggcaccgtggggacgtcggttttcgacatgctcaagtacgccgtgctgcccattgccaaggtgtttaccgtctgtttcatggggttcctcatggcctctaagtatgtcaatatcctccagccgaacggccgcaagcttctcaatgggcttgtgttctcgcttctacttccttgccttatattttcccaactgggtagagcaatcacaatcgaaaagatgctgcaatggtggtatattccagtaaatattgttgtaggtgcagtgtcaggctctttgattggctttgtggtggcttctatcatccgaccaccttatccatacttcaagttcactgtgattcacattggaatagggaatattggaaatatacctctggttctcattgcagcattatgtcgagatcccaccaatccttttggtgactctgataaatgtaatgaagatgggaatgcgtatatctcatttggtcaatgggttggtgcaattattgtttacacatatgtgttcaagatgcttgctccaccacctggtgaatcctttgatagcgctgaagaagatattcttccaattaaggcatctggagataatgtggtgcctgaaaaagggaaatatccaacaagcactcgcactagtactgtacctgaaaatgagcctttgttatcttctgaaggtgataaaaatgtttctacttctctaggatcgaagataatgggcattgttagaagcatggttaagttcctaaaagacaagcagcttcttcagccaccaattattgcctctgtttttgcaattgccattggtgttgttccagtcttgaagaattttgtcctcactgatgatgcacctctgttcttcttcacagacagctgtctcattctaggggaagcaatgatcccttgcattttgcttgcagtggggggcaatcttgtcgatggtcctggtgaaggaagtaacaggcttggtgtgcggacaaccgttgctattatttttgcacggctcatcttggttcctcttgctggggttggcatcattgtgttggttgataaacttggtttcattcctaaagatgacaaaatgttcaagtttgtcttgctactgcagcattctatgcccacatctgtgctgtcaggtgctgttgcaaacctgaggggctgtggaaaagaatctgctgctatcttgttctgggtgcacatctttgctgtgttctccatggcaggatggattattttgtatttgagcttactcttctaagtacgtcataccctgatgctatactgcacaggaacgtccataagatcaaaatatcaccagatataccagaaaaagatagtaccatgacatgtacattagccagtaaaccatggcgacagcctgaaggtacgagttgtatcagtattcagtatagatatggttctcctgcccagtgcccaatcgcttaagcgccactgctatgttaattttgctaatcgctcgctgctacaagcaactgagcaatgagaaaatgagggcatagcggtattgccgatgcccagatcatcaggtcctcatgacctttaggtcggtttcctatataagcctctttcctttattaatgtagccccccccacccccccaccccccccccccccccccacaatgatcttcattgaatgaactacaagcttcaagtgtgtttttgtggtgcagtta

SEQ ID NO: 15 - RNA, 2278 nt, Oryza sativa
  source 1..2278: mol_type = mRNA
  misc_feature 1..2278: note = PIN-LIKES 6-1 (LOC4327495) GenBank Accession No. XR_003239989 transcript variant X2
gatagcttaatctcgagcgcttcgagttcgatctctcgtgaaggggacgacgagggcattgtctccctcctcgcgcgcgcggagtcgccgccgccgccgcctcgtcctcggccacgcggaattgcgtgggtcctggttgtggaagtcaggaagtgaagaagaagaagaagaagtgaactagtgaagcctggggcaagccatgatggagaggtcgctgatggaggcgctggctacggcggcgcagggaggcaccgtggggacgtcggttttcgacatgctcaagtacgccgtgctgcccattgccaaggtgtttaccgtctgtttcatggggttcctcatggcctctaagtatgtcaatatcctccagccgaacggccgcaagcttctcaatgggcttgtgttctcgcttctacttccttgccttatattttcccaactgggtagagcaatcacaatcgaaaagatgctgcaatggtggtatattccagtaaatattgttgtaggtgcagtgtcaggctctttgattggctttgtggtggcttctatcatccgaccaccttatccatacttcaagttcactgtgattcacattggaatagggaatattggaaatatacctctggttctcattgcagcattatgtcgagatcccaccaatccttttggtgactctgataaatgtaatgaagatgggaatgcgtatatctcatttggtcaatgggttggtgcaattattgtttacacatatgtgttcaagatgcttgctccaccacctggtgaatcctttgatagcgctgaagaagatattcttccaattaaggcatctggagataatgtggtgcctgaaaaagggaaatatccaacaagcactcgcactagtactgtacctgaaaatgagcctttgttatcttctgaaggtgataaaaatgtttctacttctctaggatcgaagataatgggcattgttagaagcatggttaagttcctaaaagacaagcagcttcttcagccaccaattattgcctctgtttttgcaattgccattggtgttgttccagtcttgaagaattttgtcctcactgatgatgcacctctgttcttcttcacagacagctgtctcattctaggggaagcaatgatcccttgcattttgcttgcagtggggggcaatcttgtcgatggtcctggtgaaggaagtaacaggcttggtgtgcggacaaccgttgctattatttttgcacggctcatcttggttcctcttgctggggttggcatcattgtgttggttgataaacttggtttcattcctaaagatgacaaaatgttcaagtttgtcttgctactgcagcattctatgcccacatctgtgctgtcaggtgctgttgcaaacctgaggggctgtggaaaagaatctgctgctatcttgttctgggtgcacatctttgctgtgttctccatggcaggatggattattttgtatttgagcttactcttctaagtacgtcataccctgatgctatactgcacaggaacgtccataagatcaaaatatcaccagatataccagaaaaagatagtaccatgacatgtacattagccagtaaaccatggcgacagcctgaaggatgggtgcccgacatccacccacgcacatacatgaatgccaaaatcaaaatatttgcctgcaagctcatgccagcggttcctagtatgcatgtgggtcatgtagccttaactagcattggtacttccagatccagtgatatcagagtacatatgtgatctctccatgtgcttatagatctgggaacctgcagatctgaagttctgaacccacagttttcattcacttttaatcttttgaatctgaaggagagggagattcatgattcatcaccgtaccaaaaaaaaaggtgagagatttctatagtgaactttaaccaagcagacagctcaatatgcctcttggtgtgcgtattggttggggtctcaaaatcagatgcaaatccgtcaaacggaatatacttggaagatacgtatcgcgtcagcactcagcatggagtcagacaggtactgacattcagacacgctagggtcggacaaaaaacagatactgccaatattcgttcatcggaaatagaacaaaaaaacatgtattgacaataaatcagtgtacagaacagtaccgtatcgatatggcgagcagataataacgcaaggatacgtatgagccgtctctcatcagcagaaatggctgcatgtattcgcttcgcaccca

SEQ ID NO: 16 - DNA, 1299 bp, Oryza sativa
  source 1..1299: mol_type = unassigned DNA
  misc_feature 1..1299: note = PIN-LIKES 6-1 (LOC4327495) coding sequence
atgatggagaggtcgctgatggaggcgctggctacggcggcgcagggaggcaccgtggggacgtcggttttcgacatgctcaagtacgccgtgctgcccattgccaaggtgtttaccgtctgtttcatggggttcctcatggcctctaagtatgtcaatatcctccagccgaacggccgcaagcttctcaatgggcttgtgttctcgcttctacttccttgccttatattttcccaactgggtagagcaatcacaatcgaaaagatgctgcaatggtggtatattccagtaaatattgttgtaggtgcagtgtcaggctctttgattggctttgtggtggcttctatcatccgaccaccttatccatacttcaagttcactgtgattcacattggaatagggaatattggaaatatacctctggttctcattgcagcattatgtcgagatcccaccaatccttttggtgactctgataaatgtaatgaagatgggaatgcgtatatctcatttggtcaatgggttggtgcaattattgtttacacatatgtgttcaagatgcttgctccaccacctggtgaatcctttgatagcgctgaagaagatattcttccaattaaggcatctggagataatgtggtgcctgaaaaagggaaatatccaacaagcactcgcactagtactgtacctgaaaatgagcctttgttatcttctgaaggtgataaaaatgtttctacttctctaggatcgaagataatgggcattgttagaagcatggttaagttcctaaaagacaagcagcttcttcagccaccaattattgcctctgtttttgcaattgccattggtgttgttccagtcttgaagaattttgtcctcactgatgatgcacctctgttcttcttcacagacagctgtctcattctaggggaagcaatgatcccttgcattttgcttgcagtggggggcaatcttgtcgatggtcctggtgaaggaagtaacaggcttggtgtgcggacaaccgttgctattatttttgcacggctcatcttggttcctcttgctggggttggcatcattgtgttggttgataaacttggtttcattcctaaagatgacaaaatgttcaagtttgtcttgctactgcagcattctatgcccacatctgtgctgtcaggtgctgttgcaaacctgaggggctgtggaaaagaatctgctgctatcttgttctgggtgcacatctttgctgtgttctccatggcaggatggattattttgtatttgagcttactcttctaa

SEQ ID NO: 17 - protein, 432 aa, Oryza sativa
  REGION 1..432: note = PIN-LIKES 6-1 (LOC4327495) amino acid sequence
MMERSLMEALATAAQGGTVGTSVFDMLKYAVLPIAKVFTVCFMGFLMASKYVNILQPNGRKLLNGLVFSLLLPCLIFSQLGRAITIEKMLQWWYIPVNIVVGAVSGSLIGFVVASIIRPPYPYFKFTVIHIGIGNIGNIPLVLIAALCRDPTNPFGDSDKCNEDGNAYISFGQWVGAIIVYTYVFKMLAPPPGESFDSAEEDILPIKASGDNVVPEKGKYPTSTRTSTVPENEPLLSSEGDKNVSTSLGSKIMGIVRSMVKFLKDKQLLQPPIIASVFAIAIGVVPVLKNFVLTDDAPLFFFTDSCLILGEAMIPCILLAVGGNLVDGPGEGSNRLGVRTTVAIIFARLILVPLAGVGIIVLVDKLGFIPKDDKMFKFVLLLQHSMPTSVLSGAVANLRGCGKESAAILFWVHIFAVFSMAGWIILYLSLLF

SEQ ID NO: 18 - DNA, 3536 bp, Oryza sativa
  source 1..3536: mol_type = genomic DNA
  misc_feature 1..3536: note = PIN-LIKES 6-2 (LOC4339130) Gene NC_089039 REGION: complement(23811988..23815523)
gtggcacgcttctcctctcgtcgccgcctcgtcttctcaacttccctcggctcgcggcggcgtcggccgtgaatcctccgccaccgccgccgccgccgccgccgccgacgagccctaccacgatcgcctcgccgcattcccggggagattccgccgccgtcggcttcttccccgtgctcccgcggcggcgttttgagctctctctcccctgcgggttttccgccctttttgggcacggatgagtgggatcacattgcgcgatctctagctcaagtctcggatttccggcggttggtaggctcgtactaagctgcctaaatctcggatatcctttttttttctttctaataatgctggattaaccctaaatttcggcgagttggtatccgattcctacgctgattgagtgattgggttggatcctgacacggaaggggaaagtggttgtacaagtcgggaggaggagttggccgtttcgggaactgattccttgctatttttggctacaagttcgccgccccgggcagctgcttggccgtcgcgacgcagccgcgtctgttcgatcagttcagctcgcgcggttttaggtggggaggaatcctgttcgatttcgccggttgcttgtggtttcgtgtcggaggcgggggcggcgatgatggggaggtcggtgctggagatggtggtggcggcggcgcagggcggcggcggcgcggcgggggagtcggtgttgggcatgttcaggtacgccgtgctgcccatcgccaaggtgttcgtcgtctgcttcatggggttcctcatggcgtccaagcgcgtcggcgtgctcaagcccagcggccgcaagcttctcaatgcggtaaggcgcctaattcagtagttctcacaaccatcatggtggctcgctaattttcagaatataatggaattcagactagctatgggaacatcagcagaattcaggctagctatgttgatttatcttgtcttttttttttttgttatttttgcagcttgtgttctctctgctccttccttgcctaatatttgcgcagctcgggagatcaatcacaattgacaagatcatggagtggtaagaaagaacatattagtactcatttagatgattagccacaagttgtatctttcccttggcgatatttggggtcttcttgtgtcaaagcatttcaacattactcatgatgttgcattgcaggtggttcattccagcaaacattgctctgggtgcagtctctgcatctttggttggacttatagtggcattgattgtacggccgccttatccatacttcaagttcaccatcactcacattggaataggtttgtacattcctcacgcccttatgaatatcgctagcttctgattcatcacgcattctgcattatatttttaacatttcacttaatgcagggaacattggaaatataccacttgtccttatctcggcattatgtcgcgaccaattaaatccgtttggtgactcgaacaaatgtactcaagatggaaatgcatacttatcatttggtcaatgggtatgtccaaatccgtttgtttatggagttgcacatccctttttaaaatgctactccaaatacttatcatatctattggattgtttaccggtgcaggttggtgccattattgtttacacatatgtgttcaagatgcttgctccaccccccgggcagacctttgacagctgcgatgaggagagagataaacttccaattaaggctcctaataccatgtcaagtgtagcgaaatatccatcaagtgctcatggcaatacacatgaggaagagcccctgctatctatagaggaggaggaggaggaggaggggcaggacgttcattctttaggttcaaaagtaagaagtgggcacaacataatgctagttcagcatattggaaaaaaaaaatagaggcacttcagtagttgttaccaaaataactaataatactccatttatggcttaatgcagataatgattcctatcaagggcatggttagattcctacaaaagaagcagctcctccaaccaccaatcattgcatctgtaagtgctcggtatatttggtttttgtctcttatcactgccgtaaaacaatacttgcatttttctcaattatctgaatgttcctttgcgtcatgtaacaggttcttgcaattactctcggtgttgtgccattcttgaagaatttgatcctcacagatgacgcgcctctattctttttaacagatagctgtctcattcttgggtattgacctgtctcatgacttgtagtttcttcttgcaatatgcctatagtgtcttgtaataacaaatatgattcgcatttaaaaaccatctcttgctaacaatgctgttttatatatttcagggaagctatgattccgtgcattttgcttgccgttggtggcaatcttgttgacggtaagctcatcttgtattctcaaatctcattagaaccgtgctgcaatatgaagtgtgcttttagtacatttatacttctgcaatttatatgtttatctgaagtccatacttgtggtttattatggttttaggccctggtgaaggaagtaggagacttggggttcggaccaccgtcgcgattatatttgcacgcttgatattggttccaattgctggtattggcatcgtctcatttgctgacaagcttggatttattccaaaaggagacaagatgttcaagttcgtcctgttactgcagcactctatgcccacatcggtactatcaggtacgaaaaagtataaggatgactgagacatcatatttggcatgttaatcaacctttgagtataatacagttgcaatactaatctgctattttttatgggcaggtgctgttgcaaacctaagaggttgtggcaaggaatcagctgcaatcttgttctgggtgcacatttttgctgtcttctccatggcaggatggattatcttatatcttaccatgctgttctaggtatttgattttgagccccctctgggtgtgtgtgcctgaatatgatgctcctcctgggttagctgctgtcggcagtactgtgtctggttgtacatttttccgctgtccctcgagttattgtagtttataatgtttcatctgattcttttgtagatactaactgcactatgacataatatgatatgttcatgaaatacatgttgctgcaaagtgcaaggtgattggaagtgcgaccctgttggatctgcgggttgcattttccgggccgtggacaattgcgagaggcctgctattgtatgttttgtttggttagctgctgaaaacaatagatgaggcaaaggaattgtgtcagcacctcttgtacatttgatcgtaaaatacaggatatctccaactaatagagagccttgtatccccaaattcttgcttttggtttagtaggaccagtagattgaagtgcattatgtaagaggagattttttttttctagagaagcgtactgtgaatttgaaatccatggcatatttctacaga

SEQ ID NO: 19 - RNA, 2347 nt, Oryza sativa
  source 1..2347: mol_type = mRNA
  misc_feature 1..2347: note = PIN-LIKES 6-2 (LOC4339130) GenBank Accession No. XM_015782757.3 transcript
gtggcacgcttctcctctcgtcgccgcctcgtcttctcaacttccctcggctcgcggcggcgtcggccgtgaatcctccgccaccgccgccgccgccgccgccgccgacgagccctaccacgatcgcctcgccgcattcccggggagattccgccgccgtcggcttcttccccgtgctcccgcggcggcgttttgagctctctctcccctgcgggttttccgccctttttgggcacggatgagtgggatcacattgcgcgatctctagctcaagtctcggatttccggcggttggtaggctcgtactaagctgcctaaatctcggatatcctttttttttctttctaataatgctggattaaccctaaatttcggcgagttggtatccgattcctacgctgattgagtgattgggttggatcctgacacggaaggggaaagtggttgtacaagtcgggaggaggagttggccgtttcgggaactgattccttgctatttttggctacaagttcgccgccccgggcagctgcttggccgtcgcgacgcagccgcgtctgttcgatcagttcagctcgcgcggttttaggtggggaggaatcctgttcgatttcgccggttgcttgtggtttcgtgtcggaggcgggggcggcgatgatggggaggtcggtgctggagatggtggtggcggcggcgcagggcggcggcggcgcggcgggggagtcggtgttgggcatgttcaggtacgccgtgctgcccatcgccaaggtgttcgtcgtctgcttcatggggttcctcatggcgtccaagcgcgtcggcgtgctcaagcccagcggccgcaagcttctcaatgcgcttgtgttctctctgctccttccttgcctaatatttgcgcagctcgggagatcaatcacaattgacaagatcatggagtggtggttcattccagcaaacattgctctgggtgcagtctctgcatctttggttggacttatagtggcattgattgtacggccgccttatccatacttcaagttcaccatcactcacattggaatagggaacattggaaatataccacttgtccttatctcggcattatgtcgcgaccaattaaatccgtttggtgactcgaacaaatgtactcaagatggaaatgcatacttatcatttggtcaatgggttggtgccattattgtttacacatatgtgttcaagatgcttgctccaccccccgggcagacctttgacagctgcgatgaggagagagataaacttccaattaaggctcctaataccatgtcaagtgtagcgaaatatccatcaagtgctcatggcaatacacatgaggaagagcccctgctatctatagaggaggaggaggaggaggaggggcaggacgttcattctttaggttcaaaaataatgattcctatcaagggcatggttagattcctacaaaagaagcagctcctccaaccaccaatcattgcatctgttcttgcaattactctcggtgttgtgccattcttgaagaatttgatcctcacagatgacgcgcctctattctttttaacagatagctgtctcattcttggggaagctatgattccgtgcattttgcttgccgttggtggcaatcttgttgacggccctggtgaaggaagtaggagacttggggttcggaccaccgtcgcgattatatttgcacgcttgatattggttccaattgctggtattggcatcgtctcatttgctgacaagcttggatttattccaaaaggagacaagatgttcaagttcgtcctgttactgcagcactctatgcccacatcggtactatcaggtgctgttgcaaacctaagaggttgtggcaaggaatcagctgcaatcttgttctgggtgcacatttttgctgtcttctccatggcaggatggattatcttatatcttaccatgctgttctagatactaactgcactatgacataatatgatatgttcatgaaatacatgttgctgcaaagtgcaaggtgattggaagtgcgaccctgttggatctgcgggttgcattttccgggccgtggacaattgcgagaggcctgctattgtatgttttgtttggttagctgctgaaaacaatagatgaggcaaaggaattgtgtcagcacctcttgtacatttgatcgtaaaatacaggatatctccaactaatagagagccttgtatccccaaattcttgcttttggtttagtaggaccagtagattgaagtgcattatgtaagaggagattttttttttctagagaagcgtactgtgaatttgaaatccatggcatatttctacaga

SEQ ID NO: 20 - DNA, 1314 bp, Oryza sativa
  source 1..1314: mol_type = unassigned DNA
  misc_feature 1..1314: note = PIN-LIKES 6-2 (LOC4339130) GenBank Accession No. XM_015782757.3 coding sequence
atgatggggaggtcggtgctggagatggtggtggcggcggcgcagggcggcggcggcgcggcgggggagtcggtgttgggcatgttcaggtacgccgtgctgcccatcgccaaggtgttcgtcgtctgcttcatggggttcctcatggcgtccaagcgcgtcggcgtgctcaagcccagcggccgcaagcttctcaatgcgcttgtgttctctctgctccttccttgcctaatatttgcgcagctcgggagatcaatcacaattgacaagatcatggagtggtggttcattccagcaaacattgctctgggtgcagtctctgcatctttggttggacttatagtggcattgattgtacggccgccttatccatacttcaagttcaccatcactcacattggaatagggaacattggaaatataccacttgtccttatctcggcattatgtcgcgaccaattaaatccgtttggtgactcgaacaaatgtactcaagatggaaatgcatacttatcatttggtcaatgggttggtgccattattgtttacacatatgtgttcaagatgcttgctccaccccccgggcagacctttgacagctgcgatgaggagagagataaacttccaattaaggctcctaataccatgtcaagtgtagcgaaatatccatcaagtgctcatggcaatacacatgaggaagagcccctgctatctatagaggaggaggaggaggaggaggggcaggacgttcattctttaggttcaaaaataatgattcctatcaagggcatggttagattcctacaaaagaagcagctcctccaaccaccaatcattgcatctgttcttgcaattactctcggtgttgtgccattcttgaagaatttgatcctcacagatgacgcgcctctattctttttaacagatagctgtctcattcttggggaagctatgattccgtgcattttgcttgccgttggtggcaatcttgttgacggccctggtgaaggaagtaggagacttggggttcggaccaccgtcgcgattatatttgcacgcttgatattggttccaattgctggtattggcatcgtctcatttgctgacaagcttggatttattccaaaaggagacaagatgttcaagttcgtcctgttactgcagcactctatgcccacatcggtactatcaggtgctgttgcaaacctaagaggttgtggcaaggaatcagctgcaatcttgttctgggtgcacatttttgctgtcttctccatggcaggatggattatcttatatcttaccatgctgttctag

SEQ ID NO: 21 - protein, 437 aa, Oryza sativa
  REGION 1..437: note = PIN-LIKES 6-2 (LOC4339130) GenBank Accession No. XM_015782757.3 amino acid sequence
MMGRSVLEMVVAAAQGGGGAAGESVLGMFRYAVLPIAKVFVVCFMGFLMASKRVGVLKPSGRKLLNALVFSLLLPCLIFAQLGRSITIDKIMEWWFIPANIALGAVSASLVGLIVALIVRPPYPYFKFTITHIGIGNIGNIPLVLISALCRDQLNPFGDSNKCTQDGNAYLSFGQWVGAIIVYTYVFKMLAPPPGQTFDSCDEERDKLPIKAPNTMSSVAKYPSSAHGNTHEEEPLLSIEEEEEEEGQDVHSLGSKIMIPIKGMVRFLQKKQLLQPPIIASVLAITLGVVPFLKNLILTDDAPLFFLTDSCLILGEAMIPCILLAVGGNLVDGPGEGSRRLGVRTTVAIIFARLILVPIAGIGIVSFADKLGFIPKGDKMFKFVLLLQHSMPTSVLSGAVANLRGCGKESAAILFWVHIFAVFSMAGWIILYLTMLF

SEQ ID NO: 22 - DNA, 4565 bp, Setaria italica
  source 1..4565: mol_type = genomic DNA
  misc_feature 1..4565: note = PIN-LIKES 6 (SETIT_001507mg) Gene NC_028454 REGION: 39919686..39924250
gaccccgcgtcgtctcgcggagtcgtctcgtcctttccgttctccccccttcccccccgcgggagtcggagcccaacccatcccaccccagatcccacccgctccccgaatcgccgccactttttccattagcagccttcccgcaatcccgcggccagttcgagcccttcgcgggagggacgggctcactgattactcgggctcagcccccctccctcccagcagcgcggggtcacctcggccgtctcgcggtgagttgaaatgttgaatggagtcctcggtccaatcttcccttccgcttgggattgttttcggtgttctgaatttgcgtggcgtactggcgtggcgtgtgggggtcatggcgtgccgtcgtcgcgatcgctctccctcgcattgcgcgattcgatttttttttttttttactttgcttgcttgtttgccttttgggatgtaatggtgatttctactgctggcgattctgagctcggttgcgtctgagcttccgcagagtggtttagtgcggatttggtaaaacagtttgagttggtatggcgtgacgaatctaacctgagcagatacgcagctgcggggaattggatcccccaaccatgcgaatttggaacggaggagtagtggagtgttcgggaatcggtgtcgtcatttttctacgagtcgagttagttggaatctagttaggtcatggttctcgttccgagatgattaaatcttctatgcgtgggttttcatcaaactagaagttggttgctgctaagcttcccgcttcggaagctccatgccggatttggttacgcgtaccggtgagctgagcgaacggcgggctctcttccagttgggttgcccctgaacaaccagagccaaaaagcagttccagaattttccagacgctgcctctatattttgcctcctaacttctaagtcattcataatggcagactcctgattgcgttggacacgggtcctaagctgtgaaggcggaaaggaaaggaaaggaaaaggaaggcttcagtcccaggtcaagccatgatggagaggtcgctgctgcaggcgctggccacggcggcgcagggaggcacctcggggacttcggtactgagcatgctcaagtatgctgtgctgcccatcgccaaggtgttcacggtctgcttcatggggttcctcatggcctgcaagtacgtcaacatcctccagcccaacggccgcaagcttctcaacggggtgagctttgtctccttgtaataaaagctagctatgggaaggctgtctaatggattttgttcaaagtaggaagacgtggagcattagaggccatgattgcactaatgcgctttcccactatcaaattagactgacattttcattgtttgctgcagcttgtgttttcgcttctgcttccttgccttatattttcccaactgggtagagcaatcactattgagaagatgttacagtggtaaggaatatggattgatatcgtacatcaattttgttttatctagtaacataatactgtgttacttcatctaagtgtgctgtatggttgttgaattctgatgtgagagctattgaccaagatgttgttttctgctaatattctgtctacttgtatgtgcctctagtcctctattgtctataatcagaaacaaccattactatattacttaaaagtacaacgaaatagtctacatgtttctggttgggtcaacttttaaatcatgaacttccaaacggaacaaattagcttgctgagctcaaaattccttcagaagggacagaaaagaaatgggcgttcaagtgttaatttggttccagttgaatctatgggtcatatggttttgactactttagttatttaatatactgaaatattgcaattgttggagtttggagaaactagtccatgtcatttatctgcatatgtatgtaataatctaaattgtcataatttttcttactgggaatgtttatcttgttgtaatttaatatattgaacttgatgttatgctgttatggactattcagtcctcgtttgcattgacacatgcctcaattttcttccactccccgcatgtatatgcattacaagtgtcaattttcttttttcttttgcataaactactttaggagtatgttctggagttcagctttgccaaataagctgtgcactcgtgttctgcctctcctgttatatttctaataaggtcatatatgcatgtttgcaggtggtatattccagtaaatattgttgtaggtgcagtatctggctctttgattggctttgttgtggcatctatcatccgacccccatatccgtacttcaagttcaccattatccatataggaataggtaccaccttttgtttcttgaaaataactgcatttgtattttttttttcattcaaatatccaatatcaatacagctccttgtgttttatgcaggaaatattggaaatatacctctggtcctcattgcagcgttatgtcgggatccatctaatccgtttggtgactctgataaatgcaaccaagatggaaatgcatatatctcatttggtcaatgggtatgtctatcctcttctcttttcccagctcataaatgtccatgttttactcagaagtctgaataccaactctgtagatcaaagacaatcgcttagcatctatataggctacaaatagctgttgggatcggtcactcagtgaatccttttcgtgttgcaggttggtgctattattgtttacacatatgtgttcaggatgcttgctccaccaccaggacgcacctttgatggctctgatgacgatgaacttccagtcaaggcatctggagagaatgcggtgcccgaactcagcaaatatccaattccaacaagcactcacactagcactgtaccagaggatgaaccattgttagctgctgagaaagttcaaaaagaatgtgccacttctgtaggatcaaaggtaagaaaatgtagtcatcgacttgggtttctagttatcgttgtgatgctcttacttcccgctaaaaaaaataaccatgttcttactaatgcagataatgggccatgttaaatgtgtgattaaattcctgaaagacaagcagcttctccagccaccgattattgcatctgtatgtattgctgttgcactttttagtagtgtttatataaatcattatgcattctgcgtcttgactttatttttaaaaatgcacaggcttttgcaattgtcattggtattgtcccattcctgaagaattttgtactcacagatgaagcgcctctcttctttttcacagacagctgcctgattcttgggtatttacatgtctcattctctgtttccttatgaattatacagattatagccaataaagttagcaaactttgacaatctataatagtatctagtgtctaatccgtaaaacactttcagggaagctatgatcccatgcattttacttgctgttgggggcaatctcgttgacggtaagcatcttttttgtttgaaactctgggaaaattgcatggggcgagatcaaccttagcagtaccatcttatagggtttagaggcatttttcatgattttgtctttacctatgtttatggcatcaggtcctggtgaaggaagtaagaggcttggtgtgcgtacaaccgttgctataatttttgcacgtttggtcttggtccctcttgctggggttggcatcttcatggtagttgataaacttggtttcattcccaaagacgacaaaatgttcaagtttgtcgtgctactgcagcattctatgcccacatcagtgctgtcaggtacgataagacaccaaggactactacttatcactcatcatgtgcttagggagaacagattcctcttataatgctgtgttatatgagatctgttaggtaatgggggtggggtgccatgttccacatggtagcaaaccatgttatggaatctgattatacatttttttttgcaggtgctgttgcgaacctgagagggtgtggtaaagaatcagctgcaatcttgttctgggttcacatttttgctgtgttctccatggcaggatggatcattgtatatttgagcttgctcttctaagtatgtcgctcccttattttagctgctttttgctccatttgtcatcgcatttgcacctgtctgctgtaacagaaaaccttgatatttacgttgatctgatatttggtcaactgatctgcagacaccttactgcacacgaagatctttgaaagaacatatcatccaaagtatcacggcgaaaatagtgacatgaatatgtacagtagctagtaaatcatggtgaggtatgagttgtatcagcattgatatacggtccttcccaatgctatggtaattgtggttctcttcccactgctgcaagcagtgagcgaagatggcacagcggtgttgccctgctcggtcacacccgaaccacttagatggggatgccacgttgtcccaatccatccattcgctttgttcattccatctcctcaccaaaatattagcttaatttactcc

SEQ ID NO: 23 - RNA, 2084 nt, Setaria italica
  source 1..2084: mol_type = mRNA
  misc_feature 1..2084: note = PIN-LIKES 6 (SETIT_001507mg) GenBank Accession No. XM_004970317.4 transcript
gaccccgcgtcgtctcgcggagtcgtctcgtcctttccgttctccccccttcccccccgcgggagtcggagcccaacccatcccaccccagatcccacccgctccccgaatcgccgccactttttccattagcagccttcccgcaatcccgcggccagttcgagcccttcgcgggagggacgggctcactgattactcgggctcagcccccctccctcccagcagcgcggggtcacctcggccgtctcgcgactcctgattgcgttggacacgggtcctaagctgtgaaggcggaaaggaaaggaaaggaaaaggaaggcttcagtcccaggtcaagccatgatggagaggtcgctgctgcaggcgctggccacggcggcgcagggaggcacctcggggacttcggtactgagcatgctcaagtatgctgtgctgcccatcgccaaggtgttcacggtctgcttcatggggttcctcatggcctgcaagtacgtcaacatcctccagcccaacggccgcaagcttctcaacgggcttgtgttttcgcttctgcttccttgccttatattttcccaactgggtagagcaatcactattgagaagatgttacagtggtggtatattccagtaaatattgttgtaggtgcagtatctggctctttgattggctttgttgtggcatctatcatccgacccccatatccgtacttcaagttcaccattatccatataggaataggaaatattggaaatatacctctggtcctcattgcagcgttatgtcgggatccatctaatccgtttggtgactctgataaatgcaaccaagatggaaatgcatatatctcatttggtcaatgggttggtgctattattgtttacacatatgtgttcaggatgcttgctccaccaccaggacgcacctttgatggctctgatgacgatgaacttccagtcaaggcatctggagagaatgcggtgcccgaactcagcaaatatccaattccaacaagcactcacactagcactgtaccagaggatgaaccattgttagctgctgagaaagttcaaaaagaatgtgccacttctgtaggatcaaagataatgggccatgttaaatgtgtgattaaattcctgaaagacaagcagcttctccagccaccgattattgcatctgcttttgcaattgtcattggtattgtcccattcctgaagaattttgtactcacagatgaagcgcctctcttctttttcacagacagctgcctgattcttggggaagctatgatcccatgcattttacttgctgttgggggcaatctcgttgacggtcctggtgaaggaagtaagaggcttggtgtgcgtacaaccgttgctataatttttgcacgtttggtcttggtccctcttgctggggttggcatcttcatggtagttgataaacttggtttcattcccaaagacgacaaaatgttcaagtttgtcgtgctactgcagcattctatgcccacatcagtgctgtcaggtgctgttgcgaacctgagagggtgtggtaaagaatcagctgcaatcttgttctgggttcacatttttgctgtgttctccatggcaggatggatcattgtatatttgagcttgctcttctaagtatgtcgctcccttattttagctgctttttgctccatttgtcatcgcatttgcacctgtctgctgtaacagaaaaccttgatatttacgttgatctgatatttggtcaactgatctgcagacaccttactgcacacgaagatctttgaaagaacatatcatccaaagtatcacggcgaaaatagtgacatgaatatgtacagtagctagtaaatcatggtgaggtatgagttgtatcagcattgatatacggtccttcccaatgctatggtaattgtggttctcttcccactgctgcaagcagtgagcgaagatggcacagcggtgttgccctgctcggtcacacccgaaccacttagatggggatgccacgttgtcccaatccatccattcgctttgttcattccatctcctcaccaaaatattagcttaattta

SEQ ID NO: 24 - DNA, 1308 bp, Setaria italica
  source 1..1308: mol_type = unassigned DNA
  misc_feature 1..1308: note = PIN-LIKES 6 (SETIT_001507mg) coding sequence
atgatggagaggtcgctgctgcaggcgctggccacggcggcgcagggaggcacctcggggacttcggtactgagcatgctcaagtatgctgtgctgcccatcgccaaggtgttcacggtctgcttcatggggttcctcatggcctgcaagtacgtcaacatcctccagcccaacggccgcaagcttctcaacgggcttgtgttttcgcttctgcttccttgccttatattttcccaactgggtagagcaatcactattgagaagatgttacagtggtggtatattccagtaaatattgttgtaggtgcagtatctggctctttgattggctttgttgtggcatctatcatccgacccccatatccgtacttcaagttcaccattatccatataggaataggaaatattggaaatatacctctggtcctcattgcagcgttatgtcgggatccatctaatccgtttggtgactctgataaatgcaaccaagatggaaatgcatatatctcatttggtcaatgggttggtgctattattgtttacacatatgtgttcaggatgcttgctccaccaccaggacgcacctttgatggctctgatgacgatgaacttccagtcaaggcatctggagagaatgcggtgcccgaactcagcaaatatccaattccaacaagcactcacactagcactgtaccagaggatgaaccattgttagctgctgagaaagttcaaaaagaatgtgccacttctgtaggatcaaagataatgggccatgttaaatgtgtgattaaattcctgaaagacaagcagcttctccagccaccgattattgcatctgcttttgcaattgtcattggtattgtcccattcctgaagaattttgtactcacagatgaagcgcctctcttctttttcacagacagctgcctgattcttggggaagctatgatcccatgcattttacttgctgttgggggcaatctcgttgacggtcctggtgaaggaagtaagaggcttggtgtgcgtacaaccgttgctataatttttgcacgtttggtcttggtccctcttgctggggttggcatcttcatggtagttgataaacttggtttcattcccaaagacgacaaaatgttcaagtttgtcgtgctactgcagcattctatgcccacatcagtgctgtcaggtgctgttgcgaacctgagagggtgtggtaaagaatcagctgcaatcttgttctgggttcacatttttgctgtgttctccatggcaggatggatcattgtatatttgagcttgctcttctaa

SEQ ID NO: 25 - protein, 435 aa, Setaria italica
  REGION 1..435: note = PIN-LIKES 6 (SETIT_001507mg) amino acid sequence
MMERSLLQALATAAQGGTSGTSVLSMLKYAVLPIAKVFTVCFMGFLMACKYVNILQPNGRKLLNGLVFSLLLPCLIFSQLGRAITIEKMLQWWYIPVNIVVGAVSGSLIGFVVASIIRPPYPYFKFTIIHIGIGNIGNIPLVLIAALCRDPSNPFGDSDKCNQDGNAYISFGQWVGAIIVYTYVFRMLAPPPGRTFDGSDDDELPVKASGENAVPELSKYPIPTSTHTSTVPEDEPLLAAEKVQKECATSVGSKIMGHVKCVIKFLKDKQLLQPPIIASAFAIVIGIVPFLKNFVLTDEAPLFFFTDSCLILGEAMIPCILLAVGGNLVDGPGEGSKRLGVRTTVAIIFARLVLVPLAGVGIFMVVDKLGFIPKDDKMFKFVVLLQHSMPTSVLSGAVANLRGCGKESAAILFWVHIFAVFSMAGWIIVYLSLLF

SEQ ID NO: 26 - DNA, 5341 bp, Brachypodium distachyon
  source 1..5341: mol_type = genomic DNA
  misc_feature 1..5341: note = PIN-LIKES 6 gene (BRADI_2g53360v3) NC_016132 REGION: 52289841..52295181
agaaaccaaatcacacccaccctggccacgcctcgacgcgactcatctcgtcgtttccccttcggaggcctgcctgccagattcatccctccctcccctcccggtctcccccacccgacttgcctccaaaagcaaagccccccgccattcactactccggcttcaccttaatcacgctcttaatcctcgaaccctcacggagcgggggtttggtcggcaggactgcctcccgcgggctcggctacgctcactccgcccctccccgacccccgagcaggcgcaagtcccctcctcgacttcgcggtgggttctctcgctgatccccgtctcgtttgtctgacttgctctttgtttttcggcattgggaggcgcagagctcgggggatggaggtcgtcgtcgtggaccccgctctctcgcattggattatccttatcttaagtgtcgatttgtggtttttagttgcgtctgagctctgaagttccctcggggtgggtttccgtgcggtcgaaactgttgagttggtcgtggcgtgacgaatcgaacggagcaggcggatgcttacgtagctctaggggaatcgccctacgcgattattcgaatttgggggtagtgaacaccgacgctcttcagaatctggtaccgtcatttgtaactgtcgagctgtttggaatctagttgggtttcttctccccctagcttttttccccaattcgatctgaaactcttctcctcatgcatcagtggacgcgaggttttgcgcgaacgggaagttagttagtgcaagtttcgtcgtggctactttggttaattcgtcacgctgggttgtgggtattccagttgggctatctttagaacaaccaacaccaaaagctctcctccgattttcctgctgttgctcctatattttgccttctaagtcgtttttaatcgcagaattgagggaaggagagagaagacttaagtctagtccgggtcagccatgatggagaggaggtcgctgctggaggcgctggtcacagcggcgcagggaggttcctcacagacgtcggtgctgtccatgctcaagtacgccgtgatgcccatcgccaaggtattcaccgtctgcttcatgggtttcctcatggccaccaagtacgtcaacatcctccagcccaacggccgcaagcttctcaacggggtgagctttctttattctctttgtgataaatgccttgctaatgagacctatagccgcactgtgagcttgaaatatggtgtttgtccagagtaggaaggtataagctacttgacagtgtgcttacattcttactgtttggtgcagcttgtgttctcacttctgcttccttgccttatattttcccaactgggtagcgcaatcacgatcgagaagttgctgcagtggtgaggattgcttttggatgatttattactctcttttctaatgatttagtaattcagtcttctcctatgccttgttgaatgccctgtctatattatgtctttaattttgtcgtgtgagctactgactgagttgttgttaatattaattagatttttattactcaaagtatcattgtgtaattttactattgctgaatttcctgacaaaattaagcctggaacttccaaatataacaaagtaactcccatgacctaagtttcaagaaaggcaaacagggtaccatccaagagtgaaggaaatatgtagtagggattaattggttccagttggttttttagggttggaatggaattgaatcaagagtttctggactatttttatagcagcacttctttctaataatatactgaaatatttctataactagagacattatttgtcatctactaatagtacatggcccacccaacaccaagttagggagagtatccccgtgatctcttagttaagaagtttatcatttatgttaggaagatatcttttagcttgagccttaagaaaattagggcatcaacagtagtacagtcagtattgactctccacgtcatttagagttgacgagataataatatatacaatgggttgtgtgacttgtgtttttagacttaccatagatgatctgttagtattaaatgcgatgacgtacaatggtgaatatgttgactcatagctgactcttagtcgattcttgccttaagagtctgactcttcacgaagagtcaattctcttctctctcctatttctctttctttcacaccatcaaatttcaataagagtcaatcaagagtcagcctttgtggatggccttggagataaaatctaccatttgttacttagattggactgtaagtccatcataaatcaatataaagagaaccatctatgaggaattaatcaagaaagtattagaagaacaactcttctatcttggcctagggcaagaacgtccctcgaccccctcccatcccctcctcaaccctagccgattccctaccgccccaaacacgggccatagcactattccagtgcattaatttacctagaggtctttaccatatttgtttgttaccggcaatattcatctcgttagttgaacttcatctttcatgtcatccaatcttgtcgtagctgtttgtgtacgatgacataatgccttttgtgattgtgttcttcctatttataacatttctcccttttttgtactttgtaggtggtatattccagtaaatatcgttgtaggtgctgtgtccggttctctcattggctttgtggtggcgtctatcattcggcctccttatccgtacttcaagttcactgttatacacattggaataggtgcttgtttttcttcctgaagaaaggttgcatttcatttatgatcttctagctgatactgatattaccccttatttttttgacacaggaaatattggaaatatacctctggttctcattgcagcattatgtcgcgatccatccaatccttttggtgattctgataaatgcagccaagacggaaatgcatatatctcatttggtcaatgggtatgtgttttttcttttccatgggtgcatgtgttcagtgttttacttacaagccactaagtcagaatgactagcatgcaatataaagagaatagctcagcattttagaagaatatgaataacaactgagatcatttgctgaatgaattcttcacgtgaggcaggttggtgcgattattgtttacacgtatgtgttcaagatgctttctccaccaccaggagagacctttgatggtgaagaggagaaacttccagttatggcatctggagagaatacgttgcctgaactaggtaaatatccaacaagcactcggaatagtactgtacctgagaatgagcctttgttatctgttgagggggacaaaaaaggtgctacttctctaggatcaaaggtaagaaatgttagcagtgatttctattttaatttgttgcacttacatccccatgtgaaattaaataattgtgttctattgtcacagataattggctatgttagatgcgtggttaaattcctaaaagacaagcagcttcttcagccaccaattattgcctctgtatgtactcatgtgaatgtattctactggcaattattacagaaaaatatctcttcctgatgtgccatgaaaattattttacactatggaacaggtttttgcaattggcatcggtgttgttccattcttgaagggtttgatattcacggacgatgcacctctattcttcttcacagacagctgtctcattcttgggtatttgcatcttcctgtcagattcaatttccaaaacatagacctcatgccagttaaattatagaagtaaacatagggacaatgatttcgtttttctaaattctgtcacacattttcagggaagctatgatcccatgcattttgcttgctgtgggaggcaaccttgttgatggtaagtatctatttccatcgaaactgctgccagatcgaaccctgtaatggtcttgagatgcatttatccagatctcaaacactagccgttgtttattcaccaggtcctggtgaaggaagtaagagacttggtatgcgaaccaccattgcaattatttttgcacggttggtcttggttcctattgctggggttggcattgtcttgctagttgataaactcggtttcattcccaaagatgacaaaatgttcaagtttgtcctactactgcagcattctatgcccacatcagttttgtcaggtatgagataataagggccgctgcttatcacttaccatcatgtgctcatgtttcaagattcctctcataatgttttgtttgttgagatctgtaaaacagtgggtgtgggggcgccatgtgctgcatgttagcaaaccgcattatgagatctgataataagattgttctgtttacaggtgctgttgcaaacctgagagggtgtggaaaagaatcggccgccatcttgttctgggtgcacatttttgcagtgttttccatggcagcatggattattttctatttgaccttgctcttctaagtacgtggcttccagtatgcagttccttttctgttttgggcttacttctatgcatattaactgcggtttcttgattctcaaattattctggtttgatttacagagactaccgaacggcaatgcctgcaaaaattagttactgtcaaaaaaaaatatcgaagaagatagtgtcatgacttgtacaatagctactaaatccttgtggcagctctggaggtacgagttgtatcggtgcatatatggttcatcatgcccgcctgtgcaaatgccactgttatgttaaatttgcctcctggctgctgcaagtatggagggatgagaatcgcagcagctagtgccctgcccatatataccatcaggctaccttaacctcttagactgggtttgctgcccttattattgtcacctgtatttaaaatatttcgatccatttgcctacttggttcaggtgtaatttgggtggcgtatctgtgaattttgatggcccagtagtcagttcttgaattcagatagcaactatataagcatatcatcgggcgcattggacagcaaaccatgatgcatgattctgtgcccactgtctaccgatacatggatctgattatcacaagcgatgatgtgcaacagtgcaagcctcatgccagcagggtgtcaaagcatgcgtgcatgtccctggtgtagccaaaccgacagcaaaacattatggagaagttctgagatgaactgccgcgcctgatctctccatgtgcttgtatgtagatctgggactcggcagagcttgaatcattaattttgacaaccatgatacttttgaa

SEQ ID NO: 27 - DNA, 1299 bp, Brachypodium distachyon
  source 1..1299: mol_type = unassigned DNA
  misc_feature 1..1299: note = PIN-LIKES 6 coding sequence
atgatggagaggaggtcgctgctggaggcgctggtcacagcggcgcagggaggttcctcacagacgtcggtgctgtccatgctcaagtacgccgtgatgcccatcgccaaggtattcaccgtctgcttcatgggtttcctcatggccaccaagtacgtcaacatcctccagcccaacggccgcaagcttctcaacgggcttgtgttctcacttctgcttccttgccttatattttcccaactgggtagcgcaatcacgatcgagaagttgctgcagtggtggtatattccagtaaatatcgttgtaggtgctgtgtccggttctctcattggctttgtggtggcgtctatcattcggcctccttatccgtacttcaagttcactgttatacacattggaataggaaatattggaaatatacctctggttctcattgcagcattatgtcgcgatccatccaatccttttggtgattctgataaatgcagccaagacggaaatgcatatatctcatttggtcaatgggttggtgcgattattgtttacacgtatgtgttcaagatgctttctccaccaccaggagagacctttgatggtgaagaggagaaacttccagttatggcatctggagagaatacgttgcctgaactaggtaaatatccaacaagcactcggaatagtactgtacctgagaatgagcctttgttatctgttgagggggacaaaaaaggtgctacttctctaggatcaaagataattggctatgttagatgcgtggttaaattcctaaaagacaagcagcttcttcagccaccaattattgcctctgtttttgcaattggcatcggtgttgttccattcttgaagggtttgatattcacggacgatgcacctctattcttcttcacagacagctgtctcattcttggggaagctatgatcccatgcattttgcttgctgtgggaggcaaccttgttgatggtcctggtgaaggaagtaagagacttggtatgcgaaccaccattgcaattatttttgcacggttggtcttggttcctattgctggggttggcattgtcttgctagttgataaactcggtttcattcccaaagatgacaaaatgttcaagtttgtcctactactgcagcattctatgcccacatcagttttgtcaggtgctgttgcaaacctgagagggtgtggaaaagaatcggccgccatcttgttctgggtgcacatttttgcagtgttttccatggcagcatggattattttctatttgaccttgctcttctaa

SEQ ID NO: 28 - protein, 432 aa, Brachypodium distachyon
  REGION 1..432: note = PIN-LIKES 6 amino acid sequence
MMERRSLLEALVTAAQGGSSQTSVLSMLKYAVMPIAKVFTVCFMGFLMATKYVNILQPNGRKLLNGLVFSLLLPCLIFSQLGSAITIEKLLQWWYIPVNIVVGAVSGSLIGFVVASIIRPPYPYFKFTVIHIGIGNIGNIPLVLIAALCRDPSNPFGDSDKCSQDGNAYISFGQWVGAIIVYTYVFKMLSPPPGETFDGEEEKLPVMASGENTLPELGKYPTSTRNSTVPENEPLLSVEGDKKGATSLGSKIIGYVRCVVKFLKDKQLLQPPIIASVFAIGIGVVPFLKGLIFTDDAPLFFFTDSCLILGEAMIPCILLAVGGNLVDGPGEGSKRLGMRTTIAIIFARLVLVPIAGVGIVLLVDKLGFIPKDDKMFKFVLLLQHSMPTSVLSGAVANLRGCGKESAAILFWVHIFAVFSMAAWIIFYLTLLF

SEQ ID NO: 29 - DNA, 23 bp, synthetic construct
  source 1..23: mol_type = other DNA
  misc_feature 1..23: note = Maize PILS6 gRNA
cggcgcaaggagggaccgagggg

SEQ ID NO: 30 - DNA, 23 bp, synthetic construct
  source 1..23: mol_type = other DNA
  misc_feature 1..23: note = Maize PILS6 gRNA
gtcaacattctccagcccaacgg

SEQ ID NO: 31 - DNA, 23 bp, synthetic construct
  source 1..23: mol_type = other DNA
  misc_feature 1..23: note = Maize PILS6 gRNA
ctatcgagaagatgatacaatgg

SEQ ID NO: 32 - DNA, 23 bp, synthetic construct
  source 1..23: mol_type = other DNA
  misc_feature 1..23: note = Sorghum PILS6 gRNA
ccgttgctataatttttgcacgg

SEQ ID NO: 33 - DNA, 23 bp, synthetic construct
  source 1..23: mol_type = other DNA
  misc_feature 1..23: note = Sorghum PILS6 gRNA
gtatatctcatttggccaatggg

SEQ ID NO: 34 - DNA, 23 bp, synthetic construct
  source 1..23: mol_type = other DNA
  misc_feature 1..23: note = Sorghum PILS6 gRNA
gtcaacattctccagcccaacgg

SEQ ID NO: 35 - DNA, 23 bp, synthetic construct
  source 1..23: mol_type = other DNA
  misc_feature 1..23: note = Sorghum PILS6 gRNA
ctgcgcaaggagggaccgagggg

SEQ ID NO: 36 - DNA, 23 bp, synthetic construct
  source 1..23: mol_type = other DNA
  misc_feature 1..23: note = Rice PILS6-1 gRNA
gtcaatatcctccagccgaacgg

SEQ ID NO: 37 - DNA, 23 bp, synthetic construct
  source 1..23: mol_type = other DNA
  misc_feature 1..23: note = Rice PILS6-1 gRNA
agttgggaaaatataaggcaagg

SEQ ID NO: 38 - DNA, 23 bp, synthetic construct
  source 1..23: mol_type = other DNA
  misc_feature 1..23: note = Rice PILS6-1 gRNA
gacggtaaacaccttggcaatgg

SEQ ID NO: 39 - DNA, 23 bp, synthetic construct
  source 1..23: mol_type = other DNA
  misc_feature 1..23: note = Rice PILS6-1 gRNA
gatggagaggtcgctgatggagg

SEQ ID NO: 40 - DNA, 609 bp, synthetic construct
  source 1..609: mol_type = other DNA
  misc_feature 1..609: note = RNAi polynucleotide targeting PILS6 in Zea mays
aaggagggaccgaggggacgtcagtgctgagcatgctcaagtacgccgtgctgcccatcgccaaggtgttcactgtctgcttcatggggttccttatggcctccaagtacgtcaacattctccagcccaacggccgcaagcttctcaatgggcttgtgttttcccttctgcttccatgccttatattttcccaattgggtagagcaatcactatcgagaagatgatacaatggtggtatattccagtaaatattgttgttggcgcagtatcgggctctttgattggatttgttgtggcatttcaagagaatgccacaacaaatccaatcaaagagcccgatactgcgccaacaacaatatttactggaatataccaccattgtatcatcttctcgatagtgattgctctacccaattgggaaaatataaggcatggaagcagaagggaaaacacaagcccattgagaagcttgcggccgttgggctggagaatgttgacgtacttggaggccataaggaaccccatgaagcagacagtgaacaccttggcgatgggcagcacggcgtacttgagcatgctcagcactgacgtcccctcggtccctcctt

SEQ ID NO: 41 - DNA, 609 bp, synthetic construct
  source 1..609: mol_type = other DNA
  misc_feature 1..609: note = RNAi polynucleotide targeting PILS6 in Zea mays
gcgttatgtcgtgatccttctaacccatttggcgactctgataaatgcaatcaagatgggaatgcatatatctcatttggccaatgggttggtgcaattattgtttacacatatgtattcaaaatgcttgctccaccaccaggacagacctttgatggttctgaagaggatggaatcccaatcaaggcatctggagagaatacagtgccccaagtaggaaaatatcctatgaacactaacagtagtactgtaccagagaatgaacctttgttatctgctggggaagttcaaaaggagcgtttcaagagaacgctccttttgaacttccccagcagataacaaaggttcattctctggtacagtactactgttagtgttcataggatattttcctacttggggcactgtattctctccagatgccttgattgggattccatcctcttcagaaccatcaaaggtctgtcctggtggtggagcaagcattttgaatacatatgtgtaaacaataattgcaccaacccattggccaaatgagatatatgcattcccatcttgattgcatttatcagagtcgccaaatgggttagaaggatcacgacataacgc

SEQ ID NO: 42 - DNA, 609 bp, synthetic construct
  source 1..609: mol_type = other DNA
  misc_feature 1..609: note = RNAi polynucleotide targeting PILS6 in Sorghum bicolor
acggctgcgcaaggagggaccgaggggacgtcggtgctgagcatgctcaagtatgccgtgcttcccatcgccaaggtgttcactgtctgcttcatggggttcctcatggcctccaagtacgtcaacattctccagcccaacggccgcaagcttctcaatgggcttgtgttttcccttctgcttccatgccttatattttcccaattgggtagagcaatcactatcgagaagatgatacaatggtggtatattccagtaaatattgttgtaggtgcagtatccggctctttgattggatttttcaagagaaaatccaatcaaagagccggatactgcacctacaacaatatttactggaatataccaccattgtatcatcttctcgatagtgattgctctacccaattgggaaaatataaggcatggaagcagaagggaaaacacaagcccattgagaagcttgcggccgttgggctggagaatgttgacgtacttggaggccatgaggaaccccatgaagcagacagtgaacaccttggcgatgggaagcacggcatacttgagcatgctcagcaccgacgtcccctcggtccctccttgcgcagccgt

SEQ ID NO: 43 - DNA, 609 bp, synthetic construct
  source 1..609: mol_type = other DNA
  misc_feature 1..609: note = RNAi polynucleotide targeting PILS6 in Sorghum bicolor
ttgatggttctgaagaggatgaactcccaatcaaggcatctggagagaatacagtgccccaaataggaaattatcctatgaacactcacactagtactgtaccagagaatgaaccattgttatctgctggggatgttcaaaaggaacgtgccacttctgtaggaacaaagataatgggctttgttaaatgtgtggttaagttcctgaaagacaagcagcttctccagccaccgattattgcatctgcgtttgcaattgtaattggtgttatcccattcttgaagaattttgtccttacagttcaagagactgtaaggacaaaattcttcaagaatgggataacaccaattacaattgcaaacgcagatgcaataatcggtggctggagaagctgcttgtctttcaggaacttaaccacacatttaacaaagcccattatctttgttcctacagaagtggcacgttccttttgaacatccccagcagataacaatggttcattctctggtacagtactagtgtgagtgttcataggataatttcctatttggggcactgtattctctccagatgccttgattgggagttcatcctcttcagaaccatcaa

SEQ ID NO: 44 - DNA, 609 bp, synthetic construct
  source 1..609: mol_type = other DNA
  misc_feature 1..609: note = RNAi polynucleotide targeting PILS6-1 in Oryza sativa
gggaggcaccgtggggacgtcggttttcgacatgctcaagtacgccgtgctgcccattgccaaggtgtttaccgtctgtttcatggggttcctcatggcctctaagtatgtcaatatcctccagccgaacggccgcaagcttctcaatgggcttgtgttctcgcttctacttccttgccttatattttcccaactgggtagagcaatcacaatcgaaaagatgctgcaatggtggtatattccagtaaatattgttgtaggtgcagtgtcaggctctttgattggctttgtggtggcttcttcaagagagaagccaccacaaagccaatcaaagagcctgacactgcacctacaacaatatttactggaatataccaccattgcagcatcttttcgattgtgattgctctacccagttgggaaaatataaggcaaggaagtagaagcgagaacacaagcccattgagaagcttgcggccgttcggctggaggatattgacatacttagaggccatgaggaaccccatgaaacagacggtaaacaccttggcaatgggcagcacggcgtacttgagcatgtcgaaaaccgacgtccccacggtgcctccc

SEQ ID NO: 45 - DNA, 609 bp, synthetic construct
  source 1..609: mol_type = other DNA
  misc_feature 1..609: note = RNAi polynucleotide targeting PILS6-1 in Oryza sativa
ttgctccaccacctggtgaatcctttgatagcgctgaagaagatattcttccaattaaggcatctggagataatgtggtgcctgaaaaagggaaatatccaacaagcactcgcactagtactgtacctgaaaatgagcctttgttatcttctgaaggtgataaaaatgtttctacttctctaggatcgaagataatgggcattgttagaagcatggttaagttcctaaaagacaagcagcttcttcagccaccaattattgcctctgtttttgcaattgccattggtgttgttccagtctttcaagagaagactggaacaacaccaatggcaattgcaaaaacagaggcaataattggtggctgaagaagctgcttgtcttttaggaacttaaccatgcttctaacaatgcccattatcttcgatcctagagaagtagaaacatttttatcaccttcagaagataacaaaggctcattttcaggtacagtactagtgcgagtgcttgttggatatttccctttttcaggcaccacattatctccagatgccttaattggaagaatatcttcttcagcgctatcaaaggattcaccaggtggtggagcaa

SEQ ID NO: 46 - DNA, 20 bp, synthetic construct
  source 1..20: mol_type = other DNA
  misc_feature 1..20: note = CMS9
ctggaagaaagctcgtcgtt

SEQ ID NO: 47 - DNA, 20 bp, synthetic construct
  source 1..20: mol_type = other DNA
  misc_feature 1..20: note = CMS10
ttgatatggatgtgccgtgt

SEQ ID NO: 48 - DNA, 20 bp, synthetic construct
  source 1..20: mol_type = other DNA
  misc_feature 1..20: note = CMS11
taaggaaccccatgaagcag

SEQ ID NO: 49 - DNA, 20 bp, synthetic construct
  source 1..20: mol_type = other DNA
  misc_feature 1..20: note = CMS12
aacgacgagctttcttccag

SEQ ID NO: 50 - DNA, 20 bp, synthetic construct
  source 1..20: mol_type = other DNA
  misc_feature 1..20: note = DK1699
tgcctgccgtattgtgttac

SEQ ID NO: 51 - DNA, 22 bp, synthetic construct
  source 1..22: mol_type = other DNA
  misc_feature 1..22: note = DK1700
ttgtccattacacaaccttcca

SEQ ID NO: 52 - DNA, 32 bp, synthetic construct
  source 1..32: mol_type = other DNA
  misc_feature 1..32: note = TIR6
  misc_feature 17: note = W at position 17 is A or T
  misc_feature 24: note = Y at position 24 is C or T
agagaagccaacgccawcgcctcyatttcgtc

SEQ ID NO: 53 - DNA, 24 bp, synthetic construct
  source 1..24: mol_type = other DNA
  misc_feature 1..24: note = DK1308
caccatgatggagagatcgctgct

SEQ ID NO: 54 - DNA, 29 bp, synthetic construct
  source 1..29: mol_type = other DNA
  misc_feature 1..29: note = DK1309
ttagaagagcaaactcagatagaatataa

SEQ ID NO: 55 - DNA, 29 bp, synthetic construct
  source 1..29: mol_type = other DNA
  misc_feature 1..29: note = DK1310
gaagagcaaactcagatagaatataatcc

SEQ ID NO: 56 - DNA, 20 bp, synthetic construct
  source 1..20: mol_type = other DNA
  misc_feature 1..20: note = DK1691
acctctggtcctcattgcag

SEQ ID NO: 57 - DNA, 20 bp, synthetic construct
  source 1..20: mol_type = other DNA
  misc_feature 1..20: note = DK1692
cttttgaacttccccagcag

SEQ ID NO: 58 - DNA, 20 bp, synthetic construct
  source 1..20: mol_type = other DNA
  misc_feature 1..20: note = DK1695
atctcgttggggatgtcttg

SEQ ID NO: 59 - DNA, 20 bp, synthetic construct
  source 1..20: mol_type = other DNA
  misc_feature 1..20: note = DK1695
cccacgcgatcttattttgt

SEQ ID NO: 60 - DNA, 20 bp, synthetic construct
  source 1..20: mol_type = other DNA
  misc_feature 1..20: note = DK1570
ctgctggggaagttcaaaag

SEQ ID NO: 61 - DNA, 20 bp, synthetic construct
  source 1..20: mol_type = other DNA
  misc_feature 1..20: note = DK1571
taattggtggctggagaagc

SEQ ID NO: 62 - DNA, 20 bp, synthetic construct
  source 1..20: mol_type = other DNA
  misc_feature 1..20: note = N582
atctcgttggggatgtcttg

SEQ ID NO: 63 - DNA, 20 bp, synthetic construct
  source 1..20: mol_type = other DNA
  misc_feature 1..20: note = N583
agcaccgttcaaatgtctcc

SEQ ID NO: 64 - DNA, 5004 bp, Zea mays
  source 1..5004: mol_type = unassigned DNA
  misc_feature 1..5004: note = pils6-1 (Mu1047700)
ggatataaaatgcgcgtgtattatctagtccagtccaaccgacccggcatcgtctcgcgagccgcgactcgtctcgtcatttccatcgaacgcccagatcccacattcccacctagctctgcgcgatccatcccggccgccacgccaaggaggccctcgcctgacctcggatcaaagcctcctccatccgccttcatcccggccggtggttcgggccgtcacgggaggggtccatcactggttactcggtggagagctcggggctcggctcccatctcacgattctcaccccacacgcgcaccgggtcacctcgattcggcagcctcgcggtgagttgagttcttcgcttgtcgggccagtccgcctctcgcttggcgcggttatggattcgcgggtggcgtggcgtcgtccgcgatcgctctagctcgcattgagcgattcgcgagttgcgactccttgttttgtttgctctctgtttttttttatgtcgatggtgatatgtggcggcgggattctgagctcggttgcttctaaccttccgcggagtggtttagctaatcggttttgaagctgttgatatggatgtgccgtgtgcgtgacgaatcgaatgggccgcagcaggattcctacgcagctcggcagcagggggaattggatcctgcgaccatgcgaatttggaaccctggagcagtggagtgctaggaggtctcggtgtcgtcatttttttctaggggcgtaggagtcgacttggttggaatctagttagtcataatttcctttctgattctttctgatttcattggtctgtccatgcgtctgttttcgtcaaacaagaagtttgttattgccaagcatcccgctatggaggaagctgcctgccgtattgtgttacgcgaatccggtgagctgggctctgcgcgttcctcttgacgccctttgcgcgaacgacgagctttcttccagctcggttgcccctgaacaaccaacgccaaaagcagctccagaattttcctgatgtcgtctctatatttttgccttctaagtcatgcttaatggcagagtccttgttgcgtgggtcactgcctcactgggttctgaggcaaggaaaagaaggcttcagtagtcaggtctgggtcaagccatgatggagagatcgctgctggaggtgctggccacggcggcgcaaggagggaccgaggggacgtcagtgctgagcatgctcaagtacgccgtgctgcccatcgccaaggtgttcactgtctgcttcatggggttccttatggcctccaagtacgtcaacattctccagcccaacggccgcaagcttctcaatggggtgagctttgtctcttgtaataactaataagtgctagctaatggaaggttgtgtaatggacaattgtttttctattgaggaattattttattaagtcaagatgattacccatcttggattttgtttaaagtaggaatatgtgaagcagtagaggagttgattgtctcatttgttttctcgctgtatcaaattagatataacattttcactgtttgttgcagcttgtgttttcccttctgcttccatgccttatattttcccaattgggtagagcaatcactatcgagaagatgatacaatggtaaagataattttgattgttatcgtacttcagttttgttattataaacctagtgttatgcttcatccaagcgtgctatatttgctcttgattttttgatgtgtgagttattgaccgagatgttgttttcagctaatcatctatttgtatatgctgctgaccctctataagtaatacatcctacaaatattatattactgaccacacttgattggtgtgctattgtttatctttctatttggatcaatctttgagtaattaggttccatatggaacaaattagttttagaagctctagctttctacaaaaggacaaagaaaagactacctgaaggtgaagaaaatcagcgctcaagcattaatttggttccagttgaatttatggatcaatatgggattgacttcagattccaggattaatttagtagcactactttatgtttaactttttgaatactgcagtaaagattggagacactattccaagtagtttgtttccatatgatatagtaacctatatttttttcttactggcaatttatatcttgttgaaaatgatagaaatttatgtaatggactattcactattctgtcctatattttgacatacccctggattctttcccccttctggtgtgtatatgtataacaagtgtccacttttgtgcataagcttctttaggagtgtgtgtcctggaaattttcctttgtcaaataactgttctgcacttctgttctgcttttacttttttatatcatctatacttgcatgtttgcaggtggtatattccagtaaatattgttgttggcgcagtatcgggctctttgattggatttgttgtggcatctatcatcagacctccatatccatacttcaagttcactgttatccatataggaataggtaccacttttctgttttgaaaataagttttatttttatctatcatttgtttatccaataccaatccaggttctttttttgcaggaaatattggaaatatacctctggtcctcattgcagcgttatgtcgtgatccttctaacccatttggcgactctgataaatgcaatcaagatgggaatgcatatatctcatttggccaatgggtacgccatacttttctcttttctcggcttgtacatgctcatttttactcatgtcagcatagtgaccttgttgtttaaagattgccacttatcacctagactatgcatacaattagctgttgaggttattcactcggtgagtcctctatatgttgcaggttggtgcaattattgtttacacatatgtattcaaaatgcttgctccaccaccaggacagacctttgatggttctgaagaggatggaatcccaatcaaggcatctggagagaatacagtgccccaagtaggaaaatatcctatgaacactaacagtagtactgtaccagagaatgaacctttgttatctgctggggaagttcaaaaggagcgtgccacttctgtaggaacaaaggtaagaaattctaatagtggagctgatttcgaatatccatttggtgcgtttacttcccactgcaaaactaaatgattttgttgttttatgcagataatgggctatgttaaatgtgtggttaagtttctgaaagacaagcagcttctccagccaccaattattgcatctgtatgtattgctgctgtagtgttctactagcattttttaaaccaatttctttgagtaattatttatatactaactctacgtctttttttcttttggccattgtttaacacaggcttttgcaattgcaatcggtgttatcccattcttgaagaattttgtacttacggatgatgctcctctgttctttttcacagacagctgcctcattcttgggtatctacatgtttcatttctattttgtttttgcgaaaacatctctcttgccaataaagttatcaaactgagatgaatgtatctatcacctaattctgtggcatacttttcagagaagctatgatcccatgcattttacttgctgttgggggcaatcttgtcgatggtaagtagctattttagttaaaactttggagaattgcatggggtgcgataatccttagaatgaccatctttattggtttagagatatttctaatgtttttgcctttacccatgtttttttatggaatcaggccctggtgaaggaagtaagaggcttggcgtgcgtaccactgttgctataatttttgcacggttggtcttggtccctcttgctggggttggcattaccatgttagttgataaacttggtttcattcccgaaggtgatagaatgttcaagtttgtcctgctactgcagcattctatgcccacatcagtgttgtcaggtatgtgaagccaacaagggatgtatcttatcagctatcacttgtcttcatgtgctcatgcgtacagattcttgttaacaattttgtgtgttgagatctgtaaggaaaccacattatggactctgacgataggattgtttgttgcaggtgctgttgcaaatctgagaggttgtggaaaagaatcagctgcaattttgttctgggttcacatttttgctgtgttctccatggcgggatggattatattctatctgagtttgctcttctaagtatgtcacgccctgctaccacaacaatctgcaatttgtgcccgcattccagcctgtcagatgtaactgcattttcttgctagataatgttacttgatctgatttcagtccgctggttcagatgccttactgcgcaggaatatctttgagatcaaatatggtccaaaatgttgaggaagaaaccagtatcatgaactgtacagatatatcgttgcagaggtacaagtgtatctgtacagatatatggatacggtccctcctataatcccatattaccgtggtctcctgctgctacaagccgtgagcgatgggaacactttccctttccctccacgtatcaacaggttcacacccaaccctagattgggatgctggccttcttagttctccataatattatttcatttgtacttcctgtatattgtaaaccttgagatggctgtgttcaaatagtttcttattgggcgagggctgaagtagtagtatagatgtattatactacctatttgagtgttcggttatgccgcaaatgatgctttattatgcagaacttgtgcccgctgctggcccgtcggtcgtactaactaaaattgccaatcgcggagacttgcatgttgcaagctcaggccggcgcccagcggtttgcagcaaatttttttttttgactcggctatctccaatagaatactcaactcaatatttatatttaaactctactctatc

SEQ ID NO: 65 - DNA, 5004 bp, Zea mays
  source 1..5004: mol_type = unassigned DNA
  misc_feature 1..5004: note = pils6-2 (Mu1090629)
ggatataaaatgcgcgtgtattatctagtccagtccaaccgacccggcatcgtctcgcgagccgcgactcgtctcgtcatttccatcgaacgcccagatcccacattcccacctagctctgcgcgatccatcccggccgccacgccaaggaggccctcgcctgacctcggatcaaagcctcctccatccgccttcatcccggccggtggttcgggccgtcacgggaggggtccatcactggttactcggtggagagctcggggctcggctcccatctcacgattctcaccccacacgcgcaccgggtcacctcgattcggcagcctcgcggtgagttgagttcttcgcttgtcgggccagtccgcctctcgcttggcgcggttatggattcgcgggtggcgtggcgtcgtccgcgatcgctctagctcgcattgagcgattcgcgagttgcgactccttgttttgtttgctctctgtttttttttatgtcgatggtgatatgtggcggcgggattctgagctcggttgcttctaaccttccgcggagtggtttagctaatcggttttgaagctgttgatatggatgtgccgtgtgcgtgacgaatcgaatgggccgcagcaggattcctacgcagctcggcagcagggggaattggatcctgcgaccatgcgaatttggaaccctggagcagtggagtgctaggaggtctcggtgtcgtcatttttttctaggggcgtaggagtcgacttggttggaatctagttagtcataatttctctttctgatttcattggtctgtccatgcgtctgttttcgtcaaacaagaagtttgttattgccaagcatcccgctatggaggaagctgcctgccgtattgtgttacgcgaatccggtgagctgggctctgcgcgttcctcttgacgccctttgcgcgaacgacgagctttcttccagctcggttgcccctgaacaaccaacgccaaaagcagctccagaattttcctgatgtcgtctctatatttttgccttctaagtcatgcttaatggcagagtccttgttgcgtgggtcactgcctcactgggttctgaggcaaggaaaagggcttcagtaaggcttcagtagtcaggtctgggtcaagccatgatggagagatcgctgctggaggtgctggccacggcggcgcaaggagggaccgaggggacgtcagtgctgagcatgctcaagtacgccgtgctgcccatcgccaaggtgttcactgtctgcttcatggggttccttatggcctccaagtacgtcaacattctccagcccaacggccgcaagcttctcaatggggtgagctttgtctcttgtaataactaataagtgctagctaatggaaggttgtgtaatggacaattgtttttctattgaggaattattttattaagtcaagatgattacccatcttggattttgtttaaagtaggaatatgtgaagcagtagaggagttgattgtctcatttgttttctcgctgtatcaaattagatataacattttcactgtttgttgcagcttgtgttttcccttctgcttccatgccttatattttcccaattgggtagagcaatcactatcgagaagatgatacaatggtaaagataattttgattgttatcgtacttcagttttgttattataaacctagtgttatgcttcatccaagcgtgctatatttgctcttgattttttgatgtgtgagttattgaccgagatgttgttttcagctaatcatctatttgtatatgctgctgaccctctataagtaatacatcctacaaatattatattactgaccacacttgattggtgtgctattgtttatctttctatttggatcaatctttgagtaattaggttccatatggaacaaattagttttagaagctctagctttctacaaaaggacaaagaaaagactacctgaaggtgaagaaaatcagcgctcaagcattaatttggttccagttgaatttatggatcaatatgggattgacttcagattccaggattaatttagtagcactactttatgtttaactttttgaatactgcagtaaagattggagacactattccaagtagtttgtttccatatgatatagtaacctatatttttttcttactggcaatttatatcttgttgaaaatgatagaaatttatgtaatggactattcactattctgtcctatattttgacatacccctggattctttcccccttctggtgtgtatatgtataacaagtgtccacttttgtgcataagcttctttaggagtgtgtgtcctggaaattttcctttgtcaaataactgttctgcacttctgttctgcttttacttttttatatcatctatacttgcatgtttgcaggtggtatattccagtaaatattgttgttggcgcagtatcgggctctttgattggatttgttgtggcatctatcatcagacctccatatccatacttcaagttcactgttatccatataggaataggtaccacttttctgttttgaaaataagttttatttttatctatcatttgtttatccaataccaatccaggttctttttttgcaggaaatattggaaatatacctctggtcctcattgcagcgttatgtcgtgatccttctaacccatttggcgactctgataaatgcaatcaagatgggaatgcatatatctcatttggccaatgggtacgccatacttttctcttttctcggcttgtacatgctcatttttactcatgtcagcatagtgaccttgttgtttaaagattgccacttatcacctagactatgcatacaattagctgttgaggttattcactcggtgagtcctctatatgttgcaggttggtgcaattattgtttacacatatgtattcaaaatgcttgctccaccaccaggacagacctttgatggttctgaagaggatggaatcccaatcaaggcatctggagagaatacagtgccccaagtaggaaaatatcctatgaacactaacagtagtactgtaccagagaatgaacctttgttatctgctggggaagttcaaaaggagcgtgccacttctgtaggaacaaaggtaagaaattctaatagtggagctgatttcgaatatccatttggtgcgtttacttcccactgcaaaactaaatgattttgttgttttatgcagataatgggctatgttaaatgtgtggttaagtttctgaaagacaagcagcttctccagccaccaattattgcatctgtatgtattgctgctgtagtgttctactagcattttttaaaccaatttctttgagtaattatttatatactaactctacgtctttttttcttttggccattgtttaacacaggcttttgcaattgcaatcggtgttatcccattcttgaagaattttgtacttacggatgatgctcctctgttctttttcacagacagctgcctcattcttgggtatctacatgtttcatttctattttgtttttgcgaaaacatctctcttgccaataaagttatcaaactgagatgaatgtatctatcacctaattctgtggcatacttttcagagaagctatgatcccatgcattttacttgctgttgggggcaatcttgtcgatggtaagtagctattttagttaaaactttggagaattgcatggggtgcgataatccttagaatgaccatctttattggtttagagatatttctaatgtttttgcctttacccatgtttttttatggaatcaggccctggtgaaggaagtaagaggcttggcgtgcgtaccactgttgctataatttttgcacggttggtcttggtccctcttgctggggttggcattaccatgttagttgataaacttggtttcattcccgaaggtgatagaatgttcaagtttgtcctgctactgcagcattctatgcccacatcagtgttgtcaggtatgtgaagccaacaagggatgtatcttatcagctatcacttgtcttcatgtgctcatgcgtacagattcttgttaacaattttgtgtgttgagatctgtaaggaaaccacattatggactctgacgataggattgtttgttgcaggtgctgttgcaaatctgagaggttgtggaaaagaatcagctgcaattttgttctgggttcacatttttgctgtgttctccatggcgggatggattatattctatctgagtttgctcttctaagtatgtcacgccctgctaccacaacaatctgcaatttgtgcccgcattccagcctgtcagatgtaactgcattttcttgctagataatgttacttgatctgatttcagtccgctggttcagatgccttactgcgcaggaatatctttgagatcaaatatggtccaaaatgttgaggaagaaaccagtatcatgaactgtacagatatatcgttgcagaggtacaagtgtatctgtacagatatatggatacggtccctcctataatcccatattaccgtggtctcctgctgctacaagccgtgagcgatgggaacactttccctttccctccacgtatcaacaggttcacacccaaccctagattgggatgctggccttcttagttctccataatattatttcatttgtacttcctgtatattgtaaaccttgagatggctgtgttcaaatagtttcttattgggcgagggctgaagtagtagtatagatgtattatactacctatttgagtgttcggttatgccgcaaatgatgctttattatgcagaacttgtgcccgctgctggcccgtcggtcgtactaactaaaattgccaatcgcggagacttgcatgttgcaagctcaggccggcgcccagcggtttgcagcaaatttttttttttgactcggctatctccaatagaatactcaactcaatatttatatttaaactctactctatc

SEQ ID NO: 66 - DNA, 5005 bp, Zea mays
  source 1..5005: mol_type = unassigned DNA
  misc_feature 1..5005: note = pils6-3 (Mu_ill_221882.6)
ggatataaaatgcgcgtgtattatctagtccagtccaaccgacccggcatcgtctcgcgagccgcgactcgtctcgtcatttccatcgaacgcccagatcccacattcccacctagctctgcgcgatccatcccggccgccacgccaaggaggccctcgcctgacctcggatcaaagcctcctccatccgccttcatcccggccggtggttcgggccgtcacgggaggggtccatcactggttactcggtggagagctcggggctcggctcccatctcacgattctcaccccacacgcgcaccgggtcacctcgattcggcagcctcgcggtgagttgagttcttcgcttgtcgggccagtccgcctctcgcttggcgcggttatggattcgcgggtggcgtggcgtcgtccgcgatcgctctagctcgcattgagcgattcgcgagttgcgactccttgttttgtttgctctctgtttttttttatgtcgatggtgatatgtggcggcgggattctgagctcggttgcttctaaccttccgcggagtggtttagctaatcggttttgaagctgttgatatggatgtgccgtgtgcgtgacgaatcgaatgggccgcagcaggattcctacgcagctcggcagcagggggaattggatcctgcgaccatgcgaatttggaaccctggagcagtggagtgctaggaggtctcggtgtcgtcatttttttctaggggcgtaggagtcgacttggttggaatctagttagtcataatttctctttctgatttcattggtctgtccatgcgtctgttttcgtcaaacaagaagtttgttattgccaagcatcccgctatggaggaagctgcctgccgtattgtgttacgcgaatccggtgagctgggctctgcgcgttcctcttgacgccctttgcgcgaacgacgagctttcttccagctcggttgcccctgaacaaccaacgccaaaagcagctccagaattttcctgatgtcgtctctatatttttgccttctaagtcatgcttaatggcagagtccttgttgcgtgggtcactgcctcactgggttctgaggcaaggaaaagaaggcttcagtagtcaggtctgggtcaagccatgatggagagatcgctgctggaggtgctggccacggcggcgcaaggagggaccgaggggacgtcagtgctgagcatgctcaagtacgccgtgccttcatggggtgcccatcgccaaggtgttcactgtctgcttcatggggttccttatggcctccaagtacgtcaacattctccagcccaacggccgcaagcttctcaatggggtgagctttgtctcttgtaataactaataagtgctagctaatggaaggttgtgtaatggacaattgtttttctattgaggaattattttattaagtcaagatgattacccatcttggattttgtttaaagtaggaatatgtgaagcagtagaggagttgattgtctcatttgttttctcgctgtatcaaattagatataacattttcactgtttgttgcagcttgtgttttcccttctgcttccatgccttatattttcccaattgggtagagcaatcactatcgagaagatgatacaatggtaaagataattttgattgttatcgtacttcagttttgttattataaacctagtgttatgcttcatccaagcgtgctatatttgctcttgattttttgatgtgtgagttattgaccgagatgttgttttcagctaatcatctatttgtatatgctgctgaccctctataagtaatacatcctacaaatattatattactgaccacacttgattggtgtgctattgtttatctttctatttggatcaatctttgagtaattaggttccatatggaacaaattagttttagaagctctagctttctacaaaaggacaaagaaaagactacctgaaggtgaagaaaatcagcgctcaagcattaatttggttccagttgaatttatggatcaatatgggattgacttcagattccaggattaatttagtagcactactttatgtttaactttttgaatactgcagtaaagattggagacactattccaagtagtttgtttccatatgatatagtaacctatatttttttcttactggcaatttatatcttgttgaaaatgatagaaatttatgtaatggactattcactattctgtcctatattttgacatacccctggattctttcccccttctggtgtgtatatgtataacaagtgtccacttttgtgcataagcttctttaggagtgtgtgtcctggaaattttcctttgtcaaataactgttctgcacttctgttctgcttttacttttttatatcatctatacttgcatgtttgcaggtggtatattccagtaaatattgttgttggcgcagtatcgggctctttgattggatttgttgtggcatctatcatcagacctccatatccatacttcaagttcactgttatccatataggaataggtaccacttttctgttttgaaaataagttttatttttatctatcatttgtttatccaataccaatccaggttctttttttgcaggaaatattggaaatatacctctggtcctcattgcagcgttatgtcgtgatccttctaacccatttggcgactctgataaatgcaatcaagatgggaatgcatatatctcatttggccaatgggtacgccatacttttctcttttctcggcttgtacatgctcatttttactcatgtcagcatagtgaccttgttgtttaaagattgccacttatcacctagactatgcatacaattagctgttgaggttattcactcggtgagtcctctatatgttgcaggttggtgcaattattgtttacacatatgtattcaaaatgcttgctccaccaccaggacagacctttgatggttctgaagaggatggaatcccaatcaaggcatctggagagaatacagtgccccaagtaggaaaatatcctatgaacactaacagtagtactgtaccagagaatgaacctttgttatctgctggggaagttcaaaaggagcgtgccacttctgtaggaacaaaggtaagaaattctaatagtggagctgatttcgaatatccatttggtgcgtttacttcccactgcaaaactaaatgattttgttgttttatgcagataatgggctatgttaaatgtgtggttaagtttctgaaagacaagcagcttctccagccaccaattattgcatctgtatgtattgctgctgtagtgttctactagcattttttaaaccaatttctttgagtaattatttatatactaactctacgtctttttttcttttggccattgtttaacacaggcttttgcaattgcaatcggtgttatcccattcttgaagaattttgtacttacggatgatgctcctctgttctttttcacagacagctgcctcattcttgggtatctacatgtttcatttctattttgtttttgcgaaaacatctctcttgccaataaagttatcaaactgagatgaatgtatctatcacctaattctgtggcatacttttcagagaagctatgatcccatgcattttacttgctgttgggggcaatcttgtcgatggtaagtagctattttagttaaaactttggagaattgcatggggtgcgataatccttagaatgaccatctttattggtttagagatatttctaatgtttttgcctttacccatgtttttttatggaatcaggccctggtgaaggaagtaagaggcttggcgtgcgtaccactgttgctataatttttgcacggttggtcttggtccctcttgctggggttggcattaccatgttagttgataaacttggtttcattcccgaaggtgatagaatgttcaagtttgtcctgctactgcagcattctatgcccacatcagtgttgtcaggtatgtgaagccaacaagggatgtatcttatcagctatcacttgtcttcatgtgctcatgcgtacagattcttgttaacaattttgtgtgttgagatctgtaaggaaaccacattatggactctgacgataggattgtttgttgcaggtgctgttgcaaatctgagaggttgtggaaaagaatcagctgcaattttgttctgggttcacatttttgctgtgttctccatggcgggatggattatattctatctgagtttgctcttctaagtatgtcacgccctgctaccacaacaatctgcaatttgtgcccgcattccagcctgtcagatgtaactgcattttcttgctagataatgttacttgatctgatttcagtccgctggttcagatgccttactgcgcaggaatatctttgagatcaaatatggtccaaaatgttgaggaagaaaccagtatcatgaactgtacagatatatcgttgcagaggtacaagtgtatctgtacagatatatggatacggtccctcctataatcccatattaccgtggtctcctgctgctacaagccgtgagcgatgggaacactttccctttccctccacgtatcaacaggttcacacccaaccctagattgggatgctggccttcttagttctccataatattatttcatttgtacttcctgtatattgtaaaccttgagatggctgtgttcaaatagtttcttattgggcgagggctgaagtagtagtatagatgtattatactacctatttgagtgttcggttatgccgcaaatgatgctttattatgcagaacttgtgcccgctgctggcccgtcggtcgtactaactaaaattgccaatcgcggagacttgcatgttgcaagctcaggccggcgcccagcggtttgcagcaaatttttttttttgactcggctatctccaatagaatactcaactcaatatttatatttaaactctactctatc

SEQ ID NO: 67 - DNA, 10 bp, synthetic construct
  source 1..10: mol_type = other DNA
  misc_feature 1..10: note = Mu_ill_221882.6
cttcatgggg